SEQUENCE LISTING (1344 sequences)

SEQ ID NO: 1 - RNA, 27 nt, synthetic construct
  source 1..27: mol_type = other RNA
ggctttctggctccttaccggaaagcc

SEQ ID NO: 2 - RNA, 27 nt, synthetic construct
  source 1..27: mol_type = other RNA
ggctttctgggaggttaccggaaagcc

SEQ ID NO: 3 - RNA, 28 nt, synthetic construct
  source 1..28: mol_type = other RNA
ggctttctggcctccttaccggaaagcc

SEQ ID NO: 4 - RNA, 28 nt, synthetic construct
  source 1..28: mol_type = other RNA
ggctttctggggaggttaccggaaagcc

SEQ ID NO: 5 - RNA, 27 nt, synthetic construct
  source 1..27: mol_type = other RNA
ggctttctggctggctaccggaaagcc

SEQ ID NO: 6 - RNA, 25 nt, synthetic construct
  source 1..25: mol_type = other RNA
ggctttctggcttccccggaaagcc

SEQ ID NO: 7 - RNA, 27 nt, synthetic construct
  source 1..27: mol_type = other RNA
ggctttctggcttcttcccggaaagcc

SEQ ID NO: 8 - RNA, 27 nt, synthetic construct
  source 1..27: mol_type = other RNA
ggctttctggcaacttaccggaaagcc

SEQ ID NO: 9 - RNA, 27 nt, synthetic construct
  source 1..27: mol_type = other RNA
ggctttctggttcggtaccggaaagcc

SEQ ID NO: 10 - RNA, 28 nt, synthetic construct
  source 1..28: mol_type = other RNA
ggctttctggaagccttaccggaaagcc

SEQ ID NO: 11 - RNA, 27 nt, synthetic construct
  source 1..27: mol_type = other RNA
ggctttctggcttcttaccggaaagcc

SEQ ID NO: 12-23 - skipped (12 entries)

SEQ ID NO: 24 - DNA, 30 bp, synthetic construct
  source 1..30: mol_type = other DNA
caaactacagagccaagtgctatccacaga

SEQ ID NO: 25 - DNA, 30 bp, synthetic construct
  source 1..30: mol_type = other DNA
gagctttctgggttgccatctcaagcagac

SEQ ID NO: 26 - DNA, 30 bp, synthetic construct
  source 1..30: mol_type = other DNA
tacaaggccatcagctcatactcacaattg

SEQ ID NO: 27 - DNA, 100 bp, synthetic construct
  source 1..100: mol_type = other DNA
caaactacagagccaagtgctatccacagagagctttctgggttgccatctcaagcagactacaaggccatcagctcatactcacaattgactttgagag

SEQ ID NO: 28 - DNA, 100 bp, synthetic construct
  source 1..100: mol_type = other DNA
ttgaccacatacgtgctctttcaaagttctgtgtttgaagttatgttagtaacaactgatgcccatcctgcaatgacaaatccaattctcagtgcagctc

SEQ ID NO: 29 - DNA, 500 bp, synthetic construct
  source 1..500: mol_type = other DNA
caaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgt

SEQ ID NO: 30 - DNA, 500 bp, synthetic construct
  source 1..500: mol_type = other DNA
ttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaac

SEQ ID NO: 31-38 - skipped (8 entries)

SEQ ID NO: 39 - DNA, 288 bp, synthetic construct
  source 1..288: mol_type = other DNA
tactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 40 - DNA, 392 bp, Homo sapiens
  source 1..392: mol_type = genomic DNA
aaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 41 - DNA, 247 bp, Homo sapiens
  source 1..247: mol_type = genomic DNA
ccacgccctctgtgaaagggcggggcatgcaaattcgaaatgaaagcccgggaacgccggaagaagcacgggtgtaagatttcccttttcaaaggcagagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgc

SEQ ID NO: 42 - DNA, 250 bp, Homo sapiens
  source 1..250: mol_type = genomic DNA
ttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaatt

SEQ ID NO: 43 - DNA, 300 bp, Homo sapiens
  source 1..300: mol_type = genomic DNA
gcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggt

SEQ ID NO: 44 - DNA, 296 bp, Homo sapiens
  source 1..296: mol_type = genomic DNA
gtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 45 - DNA, 243 bp, Homo sapiens
  source 1..243: mol_type = genomic DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 46 - DNA, 106 bp, Homo sapiens
  source 1..106: mol_type = genomic DNA
caggactagtcttttaggtcaaaaagaagaagctttgtaaccgttggtttccgtagtgtagtggttatcacgttcgcctaacacgcgaaaggtccccggttcgaag

SEQ ID NO: 47 - DNA, 439 bp, Mus musculus
  source 1..439: mol_type = genomic DNA
ttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 48 - DNA, 258 bp, Mus musculus
  source 1..258: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaa

SEQ ID NO: 49 - DNA, 94 bp, Homo sapiens
  source 1..94: mol_type = genomic DNA
cctcttatgatgtttgttgccaatgatagattgttttcactgtgcaaaaattatgggtagttttggtggtcttgatgcagttgtaagcttggag

SEQ ID NO: 50 - DNA, 50 bp, Homo sapiens
  source 1..50: mol_type = genomic DNA
actttctggagtttcaaaaacagactgtacgccaagggtcatatcttttt

SEQ ID NO: 51 - DNA, 50 bp, Homo sapiens
  source 1..50: mol_type = genomic DNA
ccggggatacaacgtgtttcctaaaagtagagggaggtaagagacggtag

SEQ ID NO: 52 - DNA, 50 bp, Homo sapiens
  source 1..50: mol_type = genomic DNA
ctgaattttcttgcagttgaacaacagaggctttttttgtgtgtgtgggg

SEQ ID NO: 53 - DNA, 50 bp, Homo sapiens
  source 1..50: mol_type = genomic DNA
atatgtggtaatccaacaatagaaattatttttaagtttgtgtgttcctt

SEQ ID NO: 54-56 - skipped (3 entries)

SEQ ID NO: 57 - DNA, 206 bp, Mus musculus
  source 1..206: mol_type = genomic DNA
gtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagca

SEQ ID NO: 58 - DNA, 110 bp, Mus musculus
  source 1..110: mol_type = genomic DNA
cccaatttcactggtctacaatgaaagcaaaacagttctcttccccgctccccggtgtgtgagaggggctttgatccttctctggtttcctaggaaacgcgtatgtgtac

SEQ ID NO: 59 - RNA, 24 nt, synthetic construct
  source 1..24: mol_type = other RNA
cttttgaacatcttctctttcatc

SEQ ID NO: 60 - RNA, 21 nt, synthetic construct
  source 1..21: mol_type = other RNA
cttttgaacatcttctctttc

SEQ ID NO: 61 - RNA, 18 nt, synthetic construct
  source 1..18: mol_type = other RNA
taaaagttaggaagcaac

SEQ ID NO: 62 - RNA, 24 nt, synthetic construct
  source 1..24: mol_type = other RNA
catcttctctttcatctaaaatgc

SEQ ID NO: 63 - RNA, 24 nt, synthetic construct
  source 1..24: mol_type = other RNA
ccttagaaaattgtgcatttaccc

SEQ ID NO: 64 - RNA, 26 nt, synthetic construct
  source 1..26: mol_type = other RNA
gttttcttttgaacatcttctctttc

SEQ ID NO: 65 - RNA, 26 nt, synthetic construct
  source 1..26: mol_type = other RNA
ttcttttgaacatcttctctttcatc

SEQ ID NO: 66 - RNA, 22 nt, synthetic construct
  source 1..22: mol_type = other RNA
gttttcttttgaacatcttctc

SEQ ID NO: 67 - RNA, 26 nt, synthetic construct
  source 1..26: mol_type = other RNA
taccttagaaaattgtgcatttaccc

SEQ ID NO: 68 - RNA, 26 nt, synthetic construct
  source 1..26: mol_type = other RNA
cttagaaaattgtgcatttacccatt

SEQ ID NO: 69 - RNA, 30 nt, synthetic construct
  source 1..30: mol_type = other RNA
tgttttcttttgaacatcttctctttcatc

SEQ ID NO: 70 - RNA, 19 nt, synthetic construct
  source 1..19: mol_type = other RNA
cgccgccatttctcaacag

SEQ ID NO: 71 - RNA, 20 nt, synthetic construct
  source 1..20: mol_type = other RNA
tttgtatttagcatgttccc

SEQ ID NO: 72 - RNA, 24 nt, synthetic construct
  source 1..24: mol_type = other RNA
tgttcagcttctgttagccactga

SEQ ID NO: 73 - RNA, 31 nt, synthetic construct
  source 1..31: mol_type = other RNA
catttctcaacagatctgtcaaatcgcctgc

SEQ ID NO: 74 - RNA, 30 nt, synthetic construct
  source 1..30: mol_type = other RNA
gtgtctttctgagaaactgttcagcttctg

SEQ ID NO: 75 - RNA, 36 nt, synthetic construct
  source 1..36: mol_type = other RNA
gacttaccttaagataccatttgtatttagcatgtt

SEQ ID NO: 76 - RNA, 43 nt, synthetic construct
  source 1..43: mol_type = other RNA
atctgtcaaatcgcctgcaggtaaaagcatatggatcaagaaa

SEQ ID NO: 77 - RNA, 35 nt, synthetic construct
  source 1..35: mol_type = other RNA
ttctgagaaactgttcagcttctgttagccactga

SEQ ID NO: 78 - RNA, 43 nt, synthetic construct
  source 1..43: mol_type = other RNA
ttgtgtctttctgagaaactgttcagcttctgttagccactga

SEQ ID NO: 79 - RNA, 21 nt, synthetic construct
  source 1..21: mol_type = other RNA
cttaccttaagataccatttg

SEQ ID NO: 80 - RNA, 36 nt, synthetic construct
  source 1..36: mol_type = other RNA
gtgtctttctgagaaactgttcagcttctgttagcc

SEQ ID NO: 81 - RNA, 61 nt, synthetic construct
  source 1..61: mol_type = other RNA
catttctcaacagatctgtcaaatcgcctgcgtgtctttctgagaaactgttcagcttctg

SEQ ID NO: 82 - RNA, 61 nt, synthetic construct
  source 1..61: mol_type = other RNA
gtgtctttctgagaaactgttcagcttctgcatttctcaacagatctgtcaaatcgcctgc

SEQ ID NO: 83 - RNA, 66 nt, synthetic construct
  source 1..66: mol_type = other RNA
gtgtctttctgagaaactgttcagcttctggacttaccttaagataccatttgtatttagcatgtt

SEQ ID NO: 84 - RNA, 65 nt, synthetic construct
  source 1..65: mol_type = other RNA
acttaccttaagataccatttgtatttagcatgttgtgtctttctgagaaactgttcagcttctg

SEQ ID NO: 85 - RNA, 67 nt, synthetic construct
  source 1..67: mol_type = other RNA
catttctcaacagatctgtcaaatcgcctgcgacttaccttaagataccatttgtatttagcatgtt

SEQ ID NO: 86 - RNA, 67 nt, synthetic construct
  source 1..67: mol_type = other RNA
gacttaccttaagataccatttgtatttagcatgttcatttctcaacagatctgtcaaatcgcctgc

SEQ ID NO: 87 - RNA, 74 nt, synthetic construct
  source 1..74: mol_type = other RNA
catttctcaacagatctgtcaaatcgcctgcttgtgtctttctgagaaactgttcagcttctgttagccactga

SEQ ID NO: 88 - RNA, 74 nt, synthetic construct
  source 1..74: mol_type = other RNA
ttgtgtctttctgagaaactgttcagcttctgttagccactgacatttctcaacagatctgtcaaatcgcctgc

SEQ ID NO: 89 - RNA, 67 nt, synthetic construct
  source 1..67: mol_type = other RNA
catttctcaacagatctgtcaaatcgcctgcgtgtctttctgagaaactgttcagcttctgttagcc

SEQ ID NO: 90 - RNA, 67 nt, synthetic construct
  source 1..67: mol_type = other RNA
gtgtctttctgagaaactgttcagcttctgttagcccatttctcaacagatctgtcaaatcgcctgc

SEQ ID NO: 91 - RNA, 26 nt, synthetic construct
  source 1..26: mol_type = other RNA
tgccgctgcccaatgccatcctggag

SEQ ID NO: 92 - RNA, 25 nt, synthetic construct
  source 1..25: mol_type = other RNA
gcccaatgccatcctggagttcctg

SEQ ID NO: 93 - RNA, 52 nt, synthetic construct
  source 1..52: mol_type = other RNA
ccctacctcttttttctgtctgacagcgcccaatgccatcctggagttcctg

SEQ ID NO: 94 - RNA, 51 nt, synthetic construct
  source 1..51: mol_type = other RNA
cttccccagttgcattcaatgttctggcccaatgccatcctggagttcctg

SEQ ID NO: 95 - RNA, 51 nt, synthetic construct
  source 1..51: mol_type = other RNA
ctggcatctgtttttgaggattgctggcccaatgccatcctggagttcctg

SEQ ID NO: 96 - RNA, 57 nt, synthetic construct
  source 1..57: mol_type = other RNA
ccctacctcttttttctgtctgacagcagtttgccgctgcccaatgccatcctggag

SEQ ID NO: 97 - RNA, 56 nt, synthetic construct
  source 1..56: mol_type = other RNA
cttccccagttgcattcaatgttctgagtttgccgctgcccaatgccatcctggag

SEQ ID NO: 98 - RNA, 56 nt, synthetic construct
  source 1..56: mol_type = other RNA
ctggcatctgtttttgaggattgctgagtttgccgctgcccaatgccatcctggag

SEQ ID NO: 99 - RNA, 60 nt, synthetic construct
  source 1..60: mol_type = other RNA
tttgcagacctcctgccaccgcagattcagagtttgccgctgcccaatgccatcctggag

SEQ ID NO: 100 - RNA, 30 nt, synthetic construct
  source 1..30: mol_type = other RNA
agtttgccgctgcccaatgccatcctggag

SEQ ID NO: 101 - RNA, 30 nt, synthetic construct
  source 1..30: mol_type = other RNA
tttctgtctgacagctgtttgcagacctcc

SEQ ID NO: 102 - RNA, 30 nt, synthetic construct
  source 1..30: mol_type = other RNA
tgttctgacaacagtttgccgctgcccaat

SEQ ID NO: 103 - RNA, 30 nt, synthetic construct
  source 1..30: mol_type = other RNA
ttcaatgttctgacaacagtttgccgctgc

SEQ ID NO: 104 - RNA, 30 nt, synthetic construct
  source 1..30: mol_type = other RNA
ccaccgcagattcaggcttcccaatttttc

SEQ ID NO: 105 - RNA, 57 nt, synthetic construct
  source 1..57: mol_type = other RNA
agtttgccgctgcccaatgccatcctggagccctacctcttttttctgtctgacagc

SEQ ID NO: 106 - RNA, 56 nt, synthetic construct
  source 1..56: mol_type = other RNA
agtttgccgctgcccaatgccatcctggagcttccccagttgcattcaatgttctg

SEQ ID NO: 107 - RNA, 56 nt, synthetic construct
  source 1..56: mol_type = other RNA
agtttgccgctgcccaatgccatcctggagctggcatctgtttttgaggattgctg

SEQ ID NO: 108 - RNA, 60 nt, synthetic construct
  source 1..60: mol_type = other RNA
agtttgccgctgcccaatgccatcctggagtttgcagacctcctgccaccgcagattcag

SEQ ID NO: 109 - RNA, 30 nt, synthetic construct
  source 1..30: mol_type = other RNA
tttgcagacctcctgccaccgcagattcag

SEQ ID NO: 110 - RNA, 31 nt, synthetic construct
  source 1..31: mol_type = other RNA
atttcttccccagttgcattcaatgttctga

SEQ ID NO: 111 - RNA, 30 nt, synthetic construct
  source 1..30: mol_type = other RNA
tactggcatctgtttttgaggattgctgaa

SEQ ID NO: 112 - RNA, 35 nt, synthetic construct
  source 1..35: mol_type = other RNA
caatttttcctgtagaatactggcatctgtttttg

SEQ ID NO: 113 - RNA, 30 nt, synthetic construct
  source 1..30: mol_type = other RNA
tttcctgtagaatactggcatctgtttttg

SEQ ID NO: 114 - RNA, 30 nt, synthetic construct
  source 1..30: mol_type = other RNA
gcatctgtttttgaggattgctgaattatt

SEQ ID NO: 115 - RNA, 30 nt, synthetic construct
  source 1..30: mol_type = other RNA
tgtttttgaggattgctgaattatttcttc

SEQ ID NO: 116 - RNA, 31 nt, synthetic construct
  source 1..31: mol_type = other RNA
gaattatttcttccccagttgcattcaatgt

SEQ ID NO: 117 - RNA, 31 nt, synthetic construct
  source 1..31: mol_type = other RNA
tccccagttgcattcaatgttctgacaacag

SEQ ID NO: 118 - RNA, 35 nt, synthetic construct
  source 1..35: mol_type = other RNA
ttatttcttccccagttgcattcaatgttctgaca

SEQ ID NO: 119 - DNA, 110 bp, synthetic construct
  source 1..110: mol_type = other DNA
ctgcgcgctcgctcgctcactgaggccgcccgggcaaagcccgggcgtcgggcgacctttggtcgcccggcctcagtgagcgagcgagcgcgcagagagggagtggggtt

SEQ ID NO: 120 - DNA, 256 bp, Mus musculus
  source 1..256: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 121 - RNA, 62 nt, synthetic construct
  source 1..62: mol_type = other RNA
cattcaactgttgcctccggttctgaaggtgtcatcccactgattctgaattctttcaacta

SEQ ID NO: 122 - RNA, 43 nt, synthetic construct
  source 1..43: mol_type = other RNA
ttgctggtcttgtttttccaacttgggacgcctctgttccaaa

SEQ ID NO: 123 - DNA, 80 bp, Homo sapiens
  source 1..80: mol_type = genomic DNA
actttctggagtttcaaaaacagactgtacgccaagggtcatatctttttttgtattggtttgtgtcttggttggtgtct

SEQ ID NO: 124 - RNA, 32 nt, synthetic construct
  source 1..32: mol_type = other RNA
ggctgctttgccctcctcagagctcagatctt

SEQ ID NO: 125 - DNA, 80 bp, Homo sapiens
  source 1..80: mol_type = genomic DNA
atatgtggtaatccaacaatagaaattatttttaagtttgtgtgttcctttttctgttcaatggtgcttttgatattgtt

SEQ ID NO: 126 - RNA, 27 nt, synthetic construct
  source 1..27: mol_type = other RNA
ccctacctcttttttctgtctgacagc

SEQ ID NO: 127 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 128-129 - skipped (2 entries)

SEQ ID NO: 130 - DNA, 2556 bp, synthetic construct
  source 1..2556: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtcattcaactgttgcctccggttctgaaggtgtcatcccactgattctgaattctttcaactaaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgtaaccacaggttgtgtcaccagagtaacatacctccaacatcaaggaagatggcaaatttttggagtaggctttctggctccttaccggaaagccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagtttgctggtcttgtttttccaacttgggacgcctctgttccaaaaatttttggagtaggctttctggctccttaccggaaagccactttctggagtttcaaaaacagactgtacgccaagggtcatatctttttttgtattggtttgtgtcttggttggtgtctgcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggtggagtggctgctttgccctcctcagagctcagatcttaatttttggagcaggttttctgacctccgtcggaaaacccctatatgtggtaatccaacaatagaaattatttttaagtttgtgtgttcctttttctgttcaatggtgcttttgatattgttgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 131 - DNA, 1544 bp, synthetic construct
  source 1..1544: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtcatttctcaacagatctgtcaaatcgcctgcgacttaccttaagataccatttgtatttagcatgttaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtccctacctcttttttctgtctgacagcagtttgccgctgcccaatgccatcctggagaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 132 - DNA, 1541 bp, synthetic construct
  source 1..1541: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtgtgtctttctgagaaactgttcagcttctgcatttctcaacagatctgtcaaatcgcctgcaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttttgcagacctcctgccaccgcagattcagagtttgccgctgcccaatgccatcctggagaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 133 - DNA, 1544 bp, synthetic construct
  source 1..1544: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtcattcaactgttgcctccggttctgaaggtgtcatcccactgattctgaattctttcaactaaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtcattcaactgttgcctccggttctgaaggtgtcatcccactgattctgaattctttcaactaaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 134 - DNA, 1534 bp, synthetic construct
  source 1..1534: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacattcaactgttgcctccggttctgaaggtgtcatcccactgattctgaattctttcaactaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactccattcaactgttgcctccggttctgaaggtgtcatcccactgattctgaattctttcaactaaatttttggagcaggttttctgacttcggtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 135 - DNA, 1568 bp, synthetic construct
  source 1..1568: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtttgtgtctttctgagaaactgttcagcttctgttagccactgacatttctcaacagatctgtcaaatcgcctgcaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtttgtgtctttctgagaaactgttcagcttctgttagccactgacatttctcaacagatctgtcaaatcgcctgcaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 136 - DNA, 1554 bp, synthetic construct
  source 1..1554: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtgtgtctttctgagaaactgttcagcttctgttagcccatttctcaacagatctgtcaaatcgcctgcaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgtgtctttctgagaaactgttcagcttctgttagcccatttctcaacagatctgtcaaatcgcctgcaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 137 - DNA, 1540 bp, synthetic construct
  source 1..1540: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttttgcagacctcctgccaccgcagattcagagtttgccgctgcccaatgccatcctggagaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttttgcagacctcctgccaccgcagattcagagtttgccgctgcccaatgccatcctggagaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 138 - DNA, 1532 bp, synthetic construct
  source 1..1532: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtctggcatctgtttttgaggattgctgagtttgccgctgcccaatgccatcctggagaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtctggcatctgtttttgaggattgctgagtttgccgctgcccaatgccatcctggagaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 139 - RNA, 7086 nt, Homo sapiens
  source 1..7086: mol_type = genomic RNA
acactagcaatggcaaagctttgtgcggaggcattgctggctgctctgaactaaaagcatccgtggggaccgaaagaggtttttgcacaccttattaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttacaaaaaa

SEQ ID NO: 140 - RNA, 32357 nt, Homo sapiens
  source 1..32357: mol_type = genomic RNA
atgcgaaaggggagctgttggaattcaaatagactttctggttcccagcagtcggcagtaatagaatgctttcaggaagatgacagaatcaggagaaagatgctgttttgcactatcttgatttgttacagcagccaacttattggcatgatggagtgacaggaaaaacagctggcatggaaggtaggattattaaagctattacatcattacaaatacaattagaagctggccatgacaaagcatatgtttgaacaagcagctgttggtagctggggtttgttgccgagctcttcaaactctgcaaacagtgttgcttttacagaatggattttaaaattgccttgtgctgcgttagattttggggagggggtgtgcttgcctccaacctcaggaaaacacttctgggatagaaatcagtgttagaagattttagaataaagtattgttcaggagtctaggtttcttcaggtagatgatgacagggggaaaaaaatcttcggggaaatctaccccagattagttatatgcaatttagttgcaagaactgattgatgtattggcatattcagacagtgagagaaatgagaatgtacagaatgtttggataggttgaaaattgttatacttggtttctattttataaggagtttggctgtcagtcatttctcctcatcaatttctagcttttgacatttattcagtcattctgcattatatgaagaaaatatatcaatatctcctgcagggtttttcaacaactctagcatctgtttcagctattgaactgtattcacgttttgccaagttgatgtcgacccagctggtctatgatttctcgcttgattactgctcttaatttctctgtatcagcttgtttcagatagggctttgaaggctgttaatgaaaaatgacagaagtgataaattattgaaaatgtgagcatttatttatttatttatttatttatttatttattttgccctttaatgagttttgtcttacattctttcttgcctgcgtacgatttttagaaccctttctctgttttgcaatatttctttcccctacaatcagtggattataattataagtacttttaagcaaatggagcatgagcatggacagtatttggctccaaaaataaagtttttggagaaaggggagcaatttatgctttttgtataaactatgcatcgtttctataacctatcttcacatgctgtcatggggatctgtcagtgactgtaatcagactgaacatattgaaataatctttatcaccactagggtaacaagcaaataaaacattcaaacatttaacaagtgattgtttaaaacagatgttaaataatatagaaatagattttcctaagtgtgaaaagcctgattattaactgtctatgtaatacagaatctttcggctctctacccagtctcctccatagcctgaggagctaaatcatccaagttaatagactatcatatatgtacatctaatatgacctttcagtgtacacagtgctactcgtatgtgctgtgccttttctgtagtaattgtaatatcccagtcagtctggttaaggaacactattggctgttatgtaactttttttctgaatgttagtccatggttttcaattacgtattcctcttagaaagggtgagttaaaaacaacatgtatttaaatatcagtaaattaattagcaagtggtgggctaattacactaggatttctccaaaataattgctaacttctgttttgaaaaatagtcaaggatttggatgactactttttaaccttgaagcatacattaccgtggttgaatatcattcataattcatatatacagcaggaaagaaaatagttttctagatgtatcattttagcagcaaagaatttttttttataatgtggttcaggcactctgattatttccgtttaatacttttaagtactgtagatgcttttgtatagtgtgttacagaacttggttaatctttgggaagctcaaagactgacagaattttgcatccaaatgctacagggttattttgtggttggttgagaaaattcagctctgtctatactgttgtttaaaatacagccctctttggccaatgttacttttcaactttttataaccgttttaagtgaccattcaagactgtaaaaactcttttacactgcatctttagtaggactctccttttgtgaattgtctccttcgaaaaaatgtgagcttccttctagcctcactctcttgttaccatagttacttttaaaagaaggtttttctttttcaatgaagatagcgtactttaaatcagccactggaatccccaaactgtgtttttgtattttttagtttatagtttaggcttgcttcatttgcggttagaaagtgctctgtatttggtcccactgatgaatggattggacagacgctaggtggcaccagctgttgattattaatgaaatcgtaaagtctatatgccacgcacctgaagagcaggcaaacccttattgataaatgcattgtaagagctggaaatgtctgccaatatttcttgctctaaaacagggtcagcctgcagagtgatgagaccacgtgaagtcttcaaatcccaattgcttaatcctgtgtcttctgaagtgctttaaggaagaaaaaacgatgaaggagaaatgcataaaaatagaaggtgtgaaagagaagtgggacacaatgaaatagactgcaaacattttaatatatttttaaatccagaattagtgacttttataaatctatgtgagttttccccaattctctcaaactcagattgttttctctaaggcatttatattctaaaacaattatttatctcttttaggtaaaaattttattgagcatcattattttttaagtaaaaaaatctcccttatttctctttttgtgataagactgtcaagtgaaatttcccacatgagctagagtagctgcctctcaggtgttgctcttcagaaagatgagcaggggaagacactatcggttaagaaatatgctttgaaatgttttgcctgagaagcttgctccatcctattatcctcccattctcatataatccatcttttaagaaggaaaaagcaaataaaaacaaaccacttaagcatgtgcatacacacacacacacacacacacacgctttggaaaaaaaaaaaagtcagaactgtttaaaaagagaaagccacgacaatattttagttggtcctggagttaataaactaaagtgaaaaataggaaggaaagtccataattagattgtggagagtaaataattacttaaaactggaaatatttttccagaaaaatatgcaatattttttcttcactgggagtaactgacaattcctgaagaaggtgagatggtttagaaagagtgcaaagataatatatgctgtgattctccaatgttatctgaaaagtatgtaatatggctagttatcttgattatagcatggactttggtaaagatttttatgatgcccatatggtgaaaatggagaaatatttgctgtttgacataagagtctggtcaataaatcagctggctaaatgaccctgcctgtgatgtatcaattaatgaatcacagtaaaccaggaagagggcctttcacaggactatagcatagttctataaaattggttccatctgacttgaacctaatcttctgtaatgtgccttatgtctctgttcttgaacctacttggcctgtgaaatattaatgtccaaaaactgaaggacgatccagatagcctgtctatctgggaagcagaacaaatacattgaactattagggtaccaaaggattatgaccacctaaaaactgtggtgaggtcaaagagaataattgtaataggaataagtataacatcctaagctaaggttcataaaatcaatctggcaagtacaggacatgagagagttaaagcttagtaataattaattgaataaatcttactgattttgttggtcatcattattatcagtcataattcatatgacatcaaaatacaatgaaacaaagtaatattgttagacatgtatcctgaaaaaggaaatgaaaaaaatgagaacgaattttagaaggatatatatgatataatggtgtgtgtgtgtatatatatgataggtatggtatatgtatatattatatgtggtaatatatacatattttagaaggacaccatgatctaagaggaaccttaacatttgggggtatgttcagtgaaaagtgtctgaatgctaaaaggtcctacaaactataacatttgaggggcatggaaagtactagaaaagtgaaaagttgaagaatacttgaaggaaaaaagtagagaaaattaagctgatttcaaatattcgatgtgctataatgcagaagagggtttagactcacttgcacctctttggtagcaatggaaaagtattcttccagttaatgttgtgagcagagaattgattactttgtgagacactgcactccttattcctaaagtgttcagtgctaagaatggagtttctgcactaaatggaaattgaagaagatgtctgagataatttttaactaagaatctgtgagtccaatcatttcttttaaaaacatgttcttttttttagacatttatttctctgagggtctctactactccactcgcatcatccaggctaggggtcagtaaatgatagtccacattgtcacatctattactgtttgaccagtcatttaaaatggttttcacgtttctgaattgtttcatttcaagaatatttctgacacatgagattatataaaatttcaatttaagagtctatagtttatttggaagacagccacacccatttatttgtttatatgctatataggactcctttcatgctaaaacacagttgagtacttatgacagagaccatgtgggccacaaagcaggaatatttactatctggccctcgataggaaaagtttgctcacctcatctctagactataaagaaattttgttttttgcttattgactcaagtatctaaacaagcaatgtaacagttaaataacagttctataccataaacagaaattgtaaccacccatactacttaagtactagagacttggaaaagtagttcaaatatgcagcctaacagataaagatggtttcatttggctgttagtctattgagataatttacttgttgaggaatgtttatttaatgtttccataaaaaattgaagtctctgcaatcattgacttatatataatatctgcctagtttgactccattaatcttgctgaatttacctatttggtaggattctgttatttttattgctgcttttccgttagtgtatgatttcagtagacttacagaaagggtgaaagtcaatggaaaacaaggtaaagtgagtggaattttcttcttgcagctaccctcttctctcttcgtgggtttatagactctctcagcagctttggaattgcagttttagagacttacttttataacattatgagtgtgttcttgatcaaatatgttctagaagggatcaagataaaacaaatgcactgcactataaaagaacagttaagggttttagtaaatgattgataagacatgctaaatttaagaattagcaatgagttatttatattttcctgaacatagggatatgagaagggattttttttcatgtcataggagaaactataatcaaattctaagcataaagctgagggattctgaatttaccaccaaatattacaatctccatagttccataatgtgcaactaaaatccccacattttttttctagggaaagatataatttctgctcactggtagcgctggaaattcctgtaaaacccataggattcttattgtatttccatgaggatgttcctgagcagctactttgtgatgcctgtctctatggtaacagacacaaggcttaagggtctgaaagaaattcttcctgttggcttaggaatatggagcaatgagggagacccactgaagactataaaactgaggaaaggcaaaagaggcaattgagaaaaagttgtcgtgggggagctttttttgtgtatttgaaaataatttcacaatttcatttgctcttaatatatctctgaatgaaataagaaccaacatacaagtaaagttttacagaaagaaaaaattctgtgaaagtattgttttttaaccatctagtcaattttatgtaattgaagtcacacagattttttctttagttggcttgcctttcgctactcaagaaaagattgcttcttgcctgaaattggtaagaggacggctctacccctggcaaatttagttcttgttactgctatcaaagatcttgatcttttaaaggactttctttttcctgattccaagggtaagaatattgaactctggttcacattactcataatcactgagcttctcagggaattatgaaagtacactagccttccaagatgttcttttttaggtctatttctcttaaagaccagtaataaatagagcaaatagtactgcttatttgaaaattgggtatttcgtaagaggatgctgggtgagagtttgtgtttttacatgttttactttgtttctcttattaaaataacaaaaaagcaaacaaaaagctcctagaagtttaaaaatatttgattttccatttatttgacttcatcttactttcagcttagcaaatagggacacatcacatttgaattgttttagaaaggagataaatatgactctcctttcccttagattcaaagtatatgttcaaggtgtcatcaaatagccaaaagtctttaaaatatttatgctatgtaaatgtccttcaggaaaatataagtcagagggaggagatggccgtcttagtataaagctctcatgtgtatttgcagtgttaacatgagtgttgatgttttatttatcttcttaacatgttatatctgcacatgtcttcatttgttatatgttagggtcaacagaaaaaataataaagttattgcaagtaagcaaaaacttataacctgatcaataaaaattaatattttaggttggaagataaacacatctcaaatgtcattttataagatcaagttaacaaatgttagcatgcttaaagctttagaggaattaaatctttttatggtttattttctgaccttgagaaatttatagactctgtgcagatatatgttttcacaaatttaatccactctatatagatattagtaaaatgtaagacagtgtcttcagtacctgttcacactatctttaaattaaatgaaaaaactcaagttagctgtaatgtttcagtgtctcctttgatagtgttggaaagcaaattttttaaagaaaaccttttttataactccatacattttgacctatagatactaattgcaatataaaatgtgacatctgaataaaacaagaatactttaaatattcaatatatatactacttttgaaaagatgacttaaattataaacatataaattatatgcctaacatgactggcttccaaaaaacttattttcttgagctttggggttacagcctaaagtgcactgcagtaaataagcaaatgggtaaaaatattgtctagacaaaagtaaaactgagatgttcttttgagcaaacgtttatagaggaggtacaggatgacaactggaaacgtaggtggtacgcagagattaattgctgtcaatgtacacctgaaggtaaatggatggtgcttatcaaatttccagaagaaatattgacattatgttgcaatttgaagacagggttatgaatacaaagagctaagggaaaataaacttatcagcatcattcagctctggtcatgacattgttacaactgattataacactcccaggataaaatgtgataaggaccatcatagtctgtttttactagtacagatttcttttatacatgctataagtagaattgttcggtccagcaacaataagctggattattgaaagtgacactaatgctaacctgaaattgtatgggttttaggggagatgtgggactcatttatgttctatgacaagtgtaaaaaggagattaagtaaaagcctgcatgtgatggacacattagtgagtaggaatctcaggttcctctccctttggctgctgacccattcatgggctggttttggtggccacagtggcaggaaacagagtctatttctccattaggtcatccagacaggattagagcccaccaactgaaatgaaaggttgtttctgctgcttttcattcatccaacaaatatttattgataaattctaactgccaggctctgtgctagagcaagatgactaagatacagtccctgaggaatttagagacgaacgtggtttatgtggatgagaagagaaaaatatttacagtgaatagctaagcagagagcaacagcaactgtgaacccagacagtaaagctgacttaggactgagctagaggtttccggagttcctagtgtgagtgatgtggaaaaactggcaagtccatcaaagagcaaaatgataactgaacagaaagaaagcaaatccagtgaaaaatagtaattcaaagtaaagggtaaagcgacatatttgaagtgtcacttgaaataaaaaggagcatgccttccacctgttgctcatcttcactggaatcaaacctagaaggaaaatgtctaggctggtctaaaagccagttgcatacaatgatattaaatatgattaaagctagcatgagcatgaccatgcaaactttcaggaggatttgaaaacagttacctcccattggtagtttatgtacgtctatccctggaaactttttcctatgctatctttagagaccttgtcatattttatctcaatgtctaacactctttaattagaagctacctgaaaatgtttcactcatgtataaagttaaattaatcacagaatcagacctcagaggttacaaaggagggctgaaaggataacatctcagatagaatagatctattgccaaataaacctgtaacaataagaaattgtgttagataatccgtaaggttttatccaatttcaacatcttccaatttaattgatctccagcctcacacccaactcctcctaggaaattaaaaagagacaggtaaacaagatattttctgaagaatcgaatcatccagaaagtcattctctctcaagcagtacattttctaaagaatttaattatcgaaaaaagtctctctctctcctctttctctctcccttcttctgacatatatagatatgcatatatacacatatatgtatatatttatctatatatctatttatatctctgactcgctgaagtaacttagatatataggctaaaggcaaagaaaaattcctcccaacattcattagtaatttccctttatagattaaggaaagtatttctgtcctccttgatttttaaaacattttactgaactataacacgaatatataaaagttcacaaatcaataggtgtccagcttcatgaattttcacagattgggcacaccccagtaattactcaaggcaagaaaagaatattaccagcatccaaaaactttgtcttaccacttcctagtcaatactctcccagaggtaagtggaatactgacatctaacacaccattgattaggacagacagttcctgaaatgtatgtacttgaagccatacagctgtactcttttgtttctggctcctaaatctcaacattctgtttgtgagatctatctaatttgtttcttgtagttgtggtcccataattttcagtgttgtgtagtatgccatcatataaatgtcctataatttacttattccatctactctggatgaacatttaaattgattccatttgtgaactcttaaaaataattccactttgaatattcttgcaaatttactgcacatatgcatgtagttctgttatgtgccaagaagtagaattattaggtcatagaatagtgaaatactcatctttagttgaaaatgccacgtagtttttcaaggtaggtaaacaaagaaccatcattactttatagaagttctggacatgtcaatgcttagcattattagtctttaatttcagcctttctggctgttatactatagtatctcattgtgtttctcatttccatttttttctagtgactaattaatttgagaaccattttttgtgtttattgctcatgtggatgttatctatagtaaggataacatttcctcccaactttcattagtaatttccctttgtagattaaagaaagtaattctatcaacttgatttttaaaatgttttactgaactataacacaaatatataaaagtgcacttttctaatctcatgctcatttttctattgatttacaattttggtttttataggtatgagatctttacatttttatatggtccctacatttattgtatgtgttgcaaacatctagacagatatcttttataggaacttagaatttcaaaacccggagaccattaaagaatatttagtcccatatcttagtttgaaagatgaggaaagtgtgacttatagagaaattgtcatttcttttaagtttcttccagttagtagtaaggccagatacgtaataaaagtctcctgacactgaagtaaagggtttttttctagtaaaccatgtagggtctttagtcttgtctaatactgtgactcaaactccttgtctcttttggaaggccacaaaatgcgtgacatagtaattttaatagcaagtatagagtgatgcgtgattaatctcattgattttgtgaacaaaaatatcttcagctaacaaactgttgtatagcatggccatgcaatatttaaacctgtatgggccgggcgtggtggctcatgcctgtaatcccagcattttgggaggtcgaggtgggtgtatcatgaggtcaggagttcgagaccagcctggccaatatggtgaaaccccatctctactaaaaacacaaaaattaggtgggctcacgtgcctgtagtcccagctactcgggaggctgaggcagaagaatcgcttgaacccaggaggctgaggttgcaatgagtcaagatcgtgccagtgcactccaacctgggtgacagagtgagattctgtctcaaaaaacaaaggaaaacaaaacaaaacaaaaaaaacctatatgttcttttcggcatcagattttatttttcctctttcataactttattattatattcagtttcttttcacatttattcttattacttggatgttcccattccaaatttggttctaattcaaaaagttttcattttctatagtggatgaagaacaacataaattttacaatactttcttttagtgagataaaatttagtttacaaaacataattactagtacattaatatttactatttatttttaacatgctaggtgcagttcatgcgttattttagctaatctttggaacaacccccgccaaaatgttttattatgcctagtttatagatctagaagctgagcctaagtgagattaagctacttgtgaaacagctctaatcagaaccagatctttataagattctatgcatttcactccttccatgatctacttctgtacctgcttggaaggaaattgagttagtgatacagatttgggtcactacctgcctttagttcttctgccatacataggtcttagggataacctctttttccaatatcattaaataattcattttctgtatcttttttttccaaaacggtaattagagaagtcacttcgttggccttgattttattcattgtattgaataaatacaattttctaaatacggaactcttattcatgtgcaacctaaatacggattaggtgagttaatatccagagaccagattattactaaacttttcttgcatttagagactgaacttattataagaatcttttaattctttttagcaacctattccaaggttgattacttctcatgcatattttagttccaaatatttctttctctttcttgttcattggagaagcattccacagatgcctgagaaacaacttgctctcagtctctctatcttctagtccttggctagagtccccttgccacacttccctagcttcatgcagagtagatactcaatagtacttccacttagaaatttaaatccagatgagttacctagcatgctactttcctcttaaggagtaagtgaagcaaatgtttatagttgtttcactgtatgtgaacaaataggaatgtcaataaaacaagtacatttaattcataagtacttgataatcttaattccttaaatgtataaagctttaaaattaacaaaatactttcaagtttaatttattcacataggaaccatatgaggtaagcaaggtaggaagtcttatttcctctttatagagagccatgtaactaatttctcctatttgtatcctaaatttcttttagctctgccatcccccctccatatctcctgacagtcaccaccttagtttagatccccatatctgacttcgaatacactgatagcttcctaatgtaccttccttcttgcttctctccagtttacccttcacaaggctgctaggcttatctctctcaaagacaaaacttaataagttattccactgcttaaagtccacaggtggctttatattatttatagattataattcaaataacttgcatgaagagaaggtactttacaacaatttccctgccatgtgctcagtctaatcttatatccaccatgtgaatgcctcaccctagccatacaaacagttctctgactgtaacacacttccttttcatgtgcctttggaaatgttgactccatcccacagtcataaacaattcttattcacatatatttacttggtacatgctttataatcctccaaaatagtgtagctagtgcctcttctttacttttggctgaatcatgcacccctgtgcatagaatcttcctaatacttgtttcaccacttattttgctgtgatgtacttttcaggttattgatttattcataaactattagtgcctgagggccatgaattctctattctttttttttttttaccctttgcttagctcttctggagggagctactcaagtttaacttaaagtagtaatgagttacacagccagtaagacatttgtttgagtcctaaggtgacatcatttccattgcatccttcttccctctaatattactaaccatagtacacgagcagtactattgtcaagattcctcctcattcagtttagggaaatgcgttgacccaagttaccatttttgtctatttcacaccgtaaaagtcgggatatttagttatgatggtatgatagttaccctacatggtattttaaaggctctatttcctttgtgacttacacatgacctccccagaggtcttaatttaatggaatcatcacctaaaataatagaagagtaaattgacagtttcgtttataaggtacatggattcaggcatattggtaaagtaacacaaagcaaaaatgtggttcttcatttgtcttgtttcatcactaatagaaaatgatggagttcaccaatcacccaattgtactgtgcagcatggttattgtaaagtatacctgaaattaaaagcaaatatctacttccattttttccactgaattttcatcagaaaatgagaactacatactgaaggatttggggttggcgaatagcagatgtataatgccagcaatatgaaagcagagcaaatgcgtgattggtttacagcctcaacttcagtagtactctgcaaagtctgccagcacacaggtgccttgaggctgcaaagagcgtaggaattcagtggatgtatcaaatgatatgagatgttaatacctacttccttctaatgcatagctttgtgatattagaaaattaataaagaagtgaacaaattaattactttataatctcctaaatcttctctgtggaaggaaaatctcaaagatgcattaagtatccagcaaattggttttgaaattatccattattaaaaagcacaattctgtgtaacaggaagccacatgccaagagctatgtatatttaatcatttatgtcgatgctttttgtagcaattctgtttattctctattgacattatatacagtcctttttatacatagtggaatttaatgtgctgtgtttaaaagacacatcaagcctgacagaatccaattgtatacattatacttttaatttattagaaaatgaaatgatcatttgtgggcttatattagccaaagcagaggataaattttattatattaagaaaatcaaatactgtgactacatttacaaacattgtaaacatgacacttgttgcataattaataaatgaacatattgacctttaatataaaacggcatgaaaggaaagggaaggtggaagatataaccacaatcagaagtggaaatgaactcaaaatctgtaactgtatattaaagaaacatgtgattcatgattgcttttttgccttttccattttgaggcaatagtgccatgttgatattcatttgtgctgtcaaattaagggaaaacattgacaatctttaatataacaaagatagagaataaatgcaggtcaaaagtgtgtgggagaataagagaattgatcacattttaagaacaattacccatgtcactgcttaacccaacgtaatgatgtcaaccagtaggatggaagccagcatcaggaagaatcattttccaagcagatggatctgctgatatttttaattgattgaaggagcgttccattatttttaatcaaaaactaatttcaaataaaatggaaacttccatgaagtttgcaaaaatctgtatgcattaaggctcagactaatagaactacaatgttgtgatagttaataatccattatttaattataatgattattgttcttaacacattacacatgggagtttgtgggaactaccctacctttcttgcttctcaggtgagacaaatcattctcctagtggtctcaagtggtactgaacctcattttatcactgatgacatgctcatgaaactcacactgtactggtgttcttttctccgtctcactcacttctttgctgtgcttcctgggatcacctccgaaataagttgctggcactcaaataatttgctcagggtctacttctggtaaaaggcaacccaagacaacatgatattttattattaatagttttcctcatttaattttatatataatcatttttttctgacagggaagtatatttgaggaaataatgagggcaattatcaaaatatgcattggtttgggagttagagaaactgtcagaagtgacacaccaattttgctacatcgtgacttggtaacagtcattaccattctgggccttttattctcttctgaaaaaatggaagcattcgattacaaatcattaaagaataaagaagatagaaagtgatagaaaagataacaaatgttgatcacaccatgtgcaattgcttgatagtagtttccaggagtactataacagactgagactgtatctacttggaactcgtgggataaaataccattatttgatgccgtatgtgctcggcatgaatgcagtgggaaggatagaaaatatttcatatatttgccaactgtagaatatactgtaatagatcaagtttcattttaaaaatacacaggacacatttttttaaaaatctcatatgaaagcccagcaaagaaaagaagaattgaaggtgtagacatcttgaatgaaaacaagaggacgatttgattcaactgccatttattccaactttcaccctataaccccatagacacagctgtagaaaactgagatcttgaagaaagccactgaattagataatatctacgactacttccaactacaaatgtagaaataattgtctttacctactcatcctcagctagaaagcagctaatgggctactttaaatgtgtgatgctttcacaaattaagcaagactgagtacaacaagcacaacaagtccaacagcacaagttttcacaaacagtattggatgtaacaatacatccaaccagtattagatgtaacaaaaattggttaatttcctaatcaattaattaggaaattttattttctaacctgttggttgccagttatgggcttaagatggaagccagcatcaataggtttaaataatacctttgttttaaaaagaagaagtctaattcatagtcaaacatgtgctttggctaagtggctttgttctgggttttgcatgacagctggtgataaattgggaaaatgtgtaagtagcactgtggtaaggataatgttgatgaagatagaatgtttgaggccactttcagggagaataaaaacgacataaagtaatacttaagactgaattttcttagagatgataatctcatcaccaagcctcattttgccattgaaaaatttcttatgaattcaactatcatatttacatcacaatagacagtattataactctttagcatagggattttgctttaacttactttcaaccaatttatatgttaaagctatttatatctctgctgtttcatgaataaagcccagaggtatcttctttcttgactcactgtcatatcttcacatttcatcacatttttgcagttcaaatatcattcacttagtaacactgaaagcatagaaagattaatcagaaatgatttctgccctcaacaagctgacactggttagagtacaaaagccacactgaaagaatatatggagatctatgatctatgatcaatgatctttgaagttactattgtcatttttgggggcaacatgagtcatgcccatatgaaataacaaacttaatcaatgttgtgtgtgttctgagtgctctaccaaccggccacccccacccccccacatctgtctccctctccttggacctttctattctgagacaaaccaatattgaaatgaagccagttaacaaccccacagtggtctctaagtgttcaagtgaaaggaagagtctcacatgtctcatcttaaatgaaaagctagagatgattatgtttagtgaggagggcatgtcaaaagctgagttaggctgaaatctaggcgtcttgcaccagacagttagtcaaggtgtgaatgcaaaggaaaaattattgaaggaaattaaaagtgttacccccatgcacacatgaatgaaaagaaagcaaagcagtcttactgctgatatggataaaatttaaatggtctagacagatcaaaccagccacaatattcccttaaccaagagctaatccagaggaagatcccaactctcttcaattctacgaaggctgagaggtaaggaagctgcagaagaaaagttggaagctagcagaagttggtttatgaagtttaaggaaagaagccacctccaaacataaaagtgcaaggtgaggcagcaagtgcagttggagaagctgcagcaagttatccagaagttctagctaagatcattgataaaagtggttatactaaatgacagattttttatgtagatgaaatagccttctatttgaagaagatgccatctaggattttcatagctagaaaggagaagtcaatgcctggcttcaaagcttcaaaggacagggtgattcttttgctagagtctaatgcagctggtgactttaagttgaagcaatgctcatttaccattctgaaaatcctagggcccttaagaattatgctaaatagacttagcctttgctctataaaagtaacaataaagcctggatgagagcacatctgtttacagcatgatttactgaatattttaagcccggggttgagaccttgtgctcagaaaaatatattttttttttcaaaaatattactgctcattaataatgtagctggtcatccaagagctcatttggagatgtacaaggagactcatgttgttttcatacctgctaatataacatccattctgtagcccatgggtcaaagagtaagttcaacatttatgtctcattatttaagaaatacatttagtaaggttatagctgctgtaaataataaatcttctgatggatatgagcaaaggaaattgaaagccttctgtaaggaattcaccattctagatgccattaataacattcatgattcatcggaggaggtcaaaatatcacatttaacaggagtttagaagaagctgatttcaaccctcatggatgactttaaggagctcaagttgtcactggaagaagtcactggagatatggtgacaatagcaagagaattaaaatgagaagtggagcctgaagatgttgctgaattcctaaaatctcataataaagcgtgaatggatgaggaattgcatctgatggatgagcaaagaaagtgctttcttgagataaaatttacttctggtgaagatgctgagaacattgttgagatgacaacaaaggatttagaatattacacatacttagctgataaagtagtggcagaatttgagaagactgactccaattttgaaagaagttctactgtgagtaaagtgctatcaaacagcagcacatgctacaaagaaatgttttgtgaaagaaagagtcaattgatgcagcaaaattcattgttgtcttattttacaaagttgtcacagtcactcccaccttcagcaaccccaccctgatcaatcagcagccatccacattgaggcaagagcctccactagcaaaaagattacaactcactaaaggctcagatgattgctagcttttttagcaataaattatgtttttgttaaggtatgtacattttttaacataatgctttgcacacttaggctacaaaataatgtaaatataacttttatatacactgggaaaccgaataattcatgtgactcactttattttggtgatctggaactgaacctgcagtatctccaaggtatacctgtacatattcattcaacttttaccctctaacccgcttacttctgtccctgtggctattgctccaacatcatgataaaaacaaaacatcacatagaagtatggcaattgtactaaacgtattgtaccattggcatttggtgtctgtcgtaccatcacaattgagcctcatccttctttgttaacgcttaattcagagaaaattttcattgagattttataaatgagtaatatggctctgattattgccctgtctgactgcttttatctctatacaatattgtgagagttgctaaagagacttctgatgctgccatatatgtaattggatatttttaaattccatacagtcatctaactgacctctcacccactcaccaaaaattacaactcactccatttgcataaagtaaactgtggaaatatatatattttacctttttcaatctaaattattgctcatggagagtctatttcaccacaaacaatgctatatccttaccttaatggagtattacaatgtaattagccatggaaatggaaaaaatgaaagaaactgttatgaacacatcttcccttttaagccaacttgtcagattttgatagaagaaaaactttcaaagtcttcttacgtgagtcaggtcccattttcttatatttgcattctgctagatttacatctgtctatgacgatgatagtaaaggtgataatgatgataaaggttatgcttatgataattcctcataatgggtaactgtaatatcagataatacacgataagatctttaaaagaaagatgacaagtgaatctcataggagttcagagcatggtgaggatggccctgatttttcatttctgtctctctagtgcattttcatacacactcatcatgggggaggagggcactcaactcaattcagtttatgggatttcatcccagatttctaaatacattatacttatgactctgttcattatcccacccctcccttctctgattatggatgtagtgatgctgattatttttatattcttcactatgaaataactatcattcttcatccatttgggcttctataaaaaaaaataaataataaactgggtggcgtaaactaaaaaaataataaactgggtggcgtagaaacaacagagattcattgctcaaagttctggaggctgggaagtccaagatcaagatgaaggcacactctgtgcctgatgacagaccgtttcctagctactagatggtaccttgttgctgtgtccttatatgatgaagaggcgagctggctctctagggtctcttttataagggtactaatcccaaacaagaaggctctgtactcataacctaatcacaacctaaagacctcaccccctaataccaacctcttggggattagctttcaacatttaaattttggaggaacacaaatattcatactatagcaacattgcaagcaaaattagaataatgtattagtactgttgtgtgggatataacatgagcatgttaagaagagaatcactggattttggaatgtggtaaggccaattatctaattctcaggggaaaatactcagtaagactgaggaagtaggccaggcaaggtgactcatgcctgtaaccccagcactttgggaggctgaggcaggtggatcatttgaggtcaggagttcgagaccagcttggccaacatggtgaaaccctgtctctactaagaaatacaaacaaacaaaaaaaaaattttgctgggcatggtggcacactttagggtggctgaggctggagaatcactcgaacctgggaggtgggggttgcagtgagcggaaatggtgtcactgcacttcaccctgggtgacagagcaagactctgtctcgaaaaaaaaaaaaaatacactgagtaattaaatgtgtaaacatcattttaaaaaaacttttaaaactagtttttaaacttttaatactttaaaattttaaatcatctaatcgaacatgtatataaagatcaaaacaaattaattgtttcagtagaacagtggtgcctagagaactttatttctctggacccaattattcttaactcatcaaatcatatatatatctgtttattcatacgccactccatatatacctaagatagatacatagattgaatcatatgaatttgctgatattggtccattttgaccaaaaaatgataagtttttaggattcaaactaatagctctattactgtattgattaagtggagtgataaggtaccagtgcaatatgttatcactccacttaatcagcacagtaagcccaagaaacataaaacataaagaatattttagaaaatgatgatgatgatttcattttatcaaagctatatgctcctttactgaaatcagacaagggaaaatgttcccttctgtagaaactttattaatcccttatgatcttaatactgttaataatggactagactgtaaaggattaggtaactgaggtgttcaatatggatacttgttttatgtttaatcaaatttatctgactctaaataaatttggctgaaactagacagattccttttctattgagacagtgggctctattaagcactatgtctgcataggaaacattacaattaattttgcagtgacaatatgctttttaactaacagactaatggaaggaggactgatgtaaagcataattctcaaaaagaggggggtaaaaccttggtttctactacaattattcattttatagtattgtttgataacacatactttattaaattgtagatgcattgtaagtttttatagtatatgggaatctaaataaaaggagttatttggttgttatgccatatttagcataaatattaccatccatgtatgttgttgacttaaaacactgttattttctaaaatgtaagcatagaaaaagaataaaattcttagttgatattgcagaaatatattgtagtgtttgcagcatgaaaaggttttatatataataatatacacttaataattaatttccaaaggctgcctgtggtcagccttctttgaaagcatggattctggcaaatgagcaatataatctctttaagaccatttaagctcttaatctcttcaaaccagtaccaaagtctgttcattttgttgtaatagttattgtgtattgtttctttttaattgtgtaagtgagattcaacatcacttgtcagataagaaaaaaaaccttcaaaataaacgttaatttttcccattattgctagaaggaacttggcgtttgaaattttagatattcttaaaatttctttttaaaaatttagaattaaaaacacaaaaagaaaaaagaaaaaaatagcatttactattattccccaaaaatgaaatagtaagatattaacaatatatatatgagatatatatcctgaaaattacaaaatgctgataaaagaaatcaagacctaaataatttcagggatatattgcattcatggattggaagactcagcatagtaaagatgtcaattcttcccaaatcgatctatagatttcatgactgaaaacgattaatgtgggaggaattgctctactcaacatgaagaccttagtatatagctacagtaatgaagacaatgtgatattggtggtgagacagaaacatggagcaatgtaataaaatacagaacccagaaatagaaggaaattattatgccaaactgattctttgcaaaggtgtaaaagcaattcaatgaaggaaactatcattttcaacaaatgatgctagggtatttggatacacataggcagagaaataaacttgacctgaacacctcatactttattcaaacattaactcaaaatagatcatggatactaatataaaacctaacgcttatagatttttcatatatgtatgtgtgtgtgtatgtatacctggattatttccatctatgggtatatatgatttattcctgcctttcctttaaacccaatataattgagaattagtgcaggggtttttcatctttcctagttcaattttacattaataatctttatccctttaaagatattgatattgaattgttggagtttggttttcagaacagatgttttacagtctctggtagctaattgagttttcccattcccttgattacttcttgtactcttagatgaaagttgagtatgtttttcagtgtttattgctaggaaatagttgattacagagtcatttactagataacaaacatggaaagtaatatgacaggtttttgcttttaattgtgcttttaatttttattctacatataggaaagggtagttttcaagaggtatattgcaaaacatttgataaaaatctcttaagaaaactgaatcttgactcaataaaggaacatatgtttccaagcttttttgacaatattaaaaaaaaaactagtcctctggtcaaataaattgaagaagtttctacacaagagtcatgtattttatgtactgttgtaggcatagtaatgatcccccaaagacatacatgttctaatcttaggaagctatgaatatgttgccttatatggcgaaatttaggagtcctcaagtctactctcaggttcaattattccttaggaggactcatataattcttaaaagcagttacactcatgattacagtttattacaacaaaaagataaagactgaagtgaacaaagaaaaaagttgcatacaaccgggttcaggaaagaccaggtgccaccttccagttttcctctcctcatgaagtcatctggacagcatttaattatcccaacagtgatgtgtgacaacacacacatcgtattgccaacgaaaagttgcctaagtcttggtgtctagaattgtattggagtttgtagtaggcatggaacactcacatggctgactttagtcatctccagcctctccagagttcaaactgatacagtgtaacctaaaagctgcaccatacattattaaaataaactatctggtattgcccagaagccagtcaagagccagatatttctttggaatgtacagagtttgaacaacccagtcccgttaagttaatattttaccataaaaaggcaaagtttactttgcagatttgattaaattagggactttgagatggtaagattatcctggattatgtggatggaaccaattaaaaataagcacaaacacacaaagatccttcaaggtggctaggtgcggtggctcatgcctgtaatcccagcactttgggaggctgaggcgggtggatcacctgaggtcaggagttcaagaccagcttggccaacatggtgaaaccccttctctactacaaaatacaaaattagccaggcatggtggtgcatgcctgtaatcccagctacttgggaggctgaggcaggagaattgcttgaacccaggaggcggaagttgtagtgagccaagatcacgccattgcactccagcctgggcaacaagagtgaaactccgtatcaaaaaaaaggaagaaaaaaatccttcaaggagtcaaaggagatgttggaatgaaaagcagtgtgtcagagtaatgtgatgtagactggaagatgctatactgcttgctttgaaaatggaggaaaggggccatgaatcaaggaatgtaggcagcctctagaagctaggaaatggattctcccctagagtctccagaatgaacagcgccccattgacaccttggttttagtccagaactctaagaaagataaaaaaaaaaaaaaaaacaaaaaaaaaacaggctgggcacagtggatcacgcatgtaatcccaacactttgcgaggccaaggtggacggatcacatgaggtcaggagtttgagacctgcctggccaaaatggtaaaagcccatctctactaaaaatacaaaaatttgcttggggtggtgatgcacgcctgtagtcccagcgacttgggaagctgaggcatcagaattgcttgggcctgggagatagaggttgcagtgagccgagatcgtgccaccacactccagcctgggcgacagagtgactgagactctgtctcaaaaaaataaaagataaaaaatatatgtacatttaaatcactaagttcatggtaatttattatatgagcaataggaaactagtagagtttaatataattattatgtctatgtttgtgtagatatatacataaatgggggttgtgtgtgtgtgtgtgtgcgtgtgtaatgtcaaagcaaactttccaaatgcttacttcttgtacatcttaacctcaaggcacacaaacacacactcagttgcataaatgcacattctcacattacacacacacacacacacatatatatgacatactacattctgttctagcttcctttaatgacagctcacaaatatgtgagtagaatacactgggtttgacttgggattatgaaattcatggtaaacatcatcaaggcagatggatatctaggtgtttgattatattgatgaacaggttttggtatacaaagtgacacttgttattaaaattttgatttcatacctatttcaaactaaattgcacctacagtccctttgcaagctccactttttagaaattcagctttgtattcactgcatttatgacattcctttgcattttttttttttttacatccacttcgcttaacaagaagacactgaagttctaagtagcactgtataggactgaattattaatacagttcttcagttggtaacaagaatgctcttggaaatatcttgtacatttgtcttctcctggttacttagtagattgagcatgtaaaaaaatcttttgcttaaaattgcagtacatcttaactgacaatacttcttaaccatagtgcctgacaaaacctggatattataatctcgacgtacaatttggtccagcagttgatctctgctcagatcattcaaaagggcaattagagaaagtcaacaatacacatgtctgtaatttagttttctgtacaaacaccataaccaaatgggatgaaaggatttaaccatttgcaactaaaaacagtatagtgctttataaaaagggcctaatcaagcagtatattgattgaaactttgtacagatggttctcattattcacacattctgtgtttgggagttctcaaaacttgctaaatttaatttgtatccctaaaatcaatgctcatagcattttcacagccatttgtgtgtatgcatagagtggtgaaaaatttaagtcacccaccacacatgttcccagctgagatcaaaaaaggaacactctgcattcttgcttcaggtctcatacggtaaacaagtgtcctttcagcggtgtatttaatgctatgtttttcatatttttatgctttttcttggtgattttgctgctttaaatggccccaagcatgatggtgaatttctgtctagtgttcctaagcttaagaaggctgtgaagtaccttagaaagaaaataggttccttaggtaagtttccttcaggcttgagttacagtgctgttggctgtgtattgaatgttaatggatcaataatatatattcaataagatgtttttaaacagaaaaacaggcaaaatgggattatacattgctggttcacaaaaatgttatgagcagaggctcccagaaatctatcattgtatttctcctgggaggaatgcttcagtattcactaattcagtgttcacagtgactttatagaaacacagtgaataacaagacttgactgcacattgtcatccttcatccaacccacttgcatagtgtagggtctacactttccaacagtactaaaaacccagaacgacattcaagttatgcaggtgtattatttagctgtatttgggttctaagacatgagtaaagttatatatttgtacagctcccaactatatacagttgtccctcagcatgcacgtggtattggttccagtactctcatgtataccaaaatctgaacatactcacatttcacagccaactctgcagaacccgcacacaggcaaaattggccctctatatacgtaggtttcacattctgcgaatactgtatttttgatctgagtctggttgaaaaaaatggcatgtaagtggacccatgcagttcaaacccgtgttttctcaggactcaactatgttatttatccttttacttttttattccttttttattcctgtgtataaaaaattgtaccaccagtacaaggaatgtcatgaaattgaaatgacaccattctgtgtttcttaaataaatgtcatacaggttctatgaagaataaattctcttgctaaaaaaaaaaacttgctatttttcatcatttgattttctcatattaacatttttcatcttaaatagtctttctctttactatttcccttcacatttcttgaaaattctttttcttgcccatctgttttgtatctccttattattttctttctgtttttattatgctagccatggtaatgataaaatctccgacttccaactccctgcaccatttggcacttggcaaatggcttttttatgtatttatatttttgttatttatatgtctatatcctaaacagaaccctacgcataagagcaggtggccagtaactaatggcattgataaaatagagccctttacctctattttcttgaaatatgctttcaagaaattccttgtactagatcatcgtgtatcataacattatgaacacagcagcaaaaaacaagaaaaaaattcagactttagtattcgtaagtaatgtcaatacaatatttggtttaacagacatacgatcattttgtttgctatttcaccatattattttttatgtaaataaggtagattatttccaaagtctttctgaatttactaaaatctcattttacatgaccctactttagtaccttaactaccaccctcctaggaatatatctgtggtaagaacagacaggtgtatatatattatagataagtgtctgtctgtgtgtatgcatacatatgtgtgtatctatatattaaagtatgtatatgcaatatgaaggtatgtatatatgcatatatatgcatgaattatattaagcatatgtatatatatattatagttaagtgtgtgtatgtgtgtatgcatacatatgtgtatctatatattaaagtaggtatatacagtatgaagtatgtatgtatatatgcatatatatgcatgaattatattaagtatatgtatatgtgttagatatttatgtatatatgtatatttataaaattatgaagttctacattgaactgggatataaagtccatgatatctttacataacttagaacagcaaaactctcgattcattcttgttttcaaaatccctttagcttcattttctctggtgcatgaaacaacataaaaatccctcttcttgccattaatctcttcaaatcccatgccagatattgtcaacattacagctataaaaattttagaatgttttagggacatcttgagatctttaaattaaaatatccactaaattaacatcataagcctagactcgtaatctttatttttgatctgactttctgtggcttgcaaatttagaatcaggaaatgaaacaatactcaatttttgcagaattagggcttacttatgatgcaaggctaatatggataatttcttaattgactgtatctgctaagacaaaatcaggaaatgacttaattaaaatgaataatttaggtgatattctctatattgtaatccaaattttaaagaagcgaacttctcttagaagaaaaatatacatttaataattggttcaatgttattatagaccttttgtgttctttcagaacataataatggttgctaattttctgggcagaatctcttgataagagagttaaattgactcagattatctgcaacccactggactattgtggtaatggcagatattttttaaatttaattcattgatataaaatatttagtcttttataataagcaattaaacctttgtacttaagccacttcaacaaaaatattacaaaaccattaggcagctacaataggctccatgcatttttaagtacggtttatacagaataaattatttctgtattggaatatcttcttaataatattgatgtttcaagaaaacatccaagctattttataagttatttgaatggagactgaattatatagtagttttttgttgttgttattttaattcatacaccttttcagtagaaaaaggtgccttggttttaactttgttaccccacaagcacagatattaatttttattgcattcttcactaaagtacatcttgcatttgattttcagacatggataagaccatgatagctaatgaatgactaaaataaatcaatactccaaatacttctatgcttcatttttgatggaagtatgagtaagggatacaagctgaaggcctcctatatatagtacttctacattactttttttgttataattttgtaataataatataatcacaccacatacttgtaataaaaatataatattcatctaggctaattggtcaggtcctgaagtaaatatatctcttcacaaaaggccaatgagtgacccacctactggtagattttatgcaaatggttgcatcttgtacacctttattattttttaagtatatttgtttcctacttgtaacctattctaaaataaaaagtttactatctctttgcagtaaaaataaatatcacatatctatatatataaagatagatatggtatacacctcagtatagatataaagctctttatttatttatttgttcatttatttatttatatttatgtttaaatacagacagatttccagattttaaaatgcatttcttctcactgaatattaagggacttaaaccatatactcaatttgtcaaagctgactggggaatataaagaaagggctagatatgtaatatgtgttgctaatattaactagaatcattattttgaagtacagatcttagttcaataacatagcgcgctagagcagaccaaagttattaacttcaattgataaagaattatgaatataattataatcctgttaattatttaacaccgtgaattatgcaaatgcagcaggtattattaatcatctgcaggaaccagaacaatttccctaaaagaacatataaatattaaaaagtctgacttggctctgttatgtactgttagagctttagtgttcccatatgaacactgtttggattcaattatcctttataacatgaaaaatgccccgaatgcatcctctccaaaggccaaatgaatgtcattagagttctccttttgtctataaaatgtttactgactgtgtggtctcgcatgtttgtttatatcaaaatatctttcatcctattaggctaatactggtctgtggcggctggcatcctacttgctaatagcacgttgtgtggatatccttgctgtgagactaagctacatgtgatagtcttgcagtatctaaaaaaattcccaaaattaacatatgttgaaccttccctcaatactaaggaacaacagagcattcaatggagtaattttaactaatttcctaagtatgctcaccctttacagaaaggaaaaaaagaaaaagaaaaggaattccaaattggacctttggaaaacactcaattggggagaggaattggtaaggggcttgaagatctagtttggttaacagattgttgtaaattttcctgaaggaaatattagccctttcctttaaagagttatttgtggaaacctttaaaaaggattttcaaaataaagactgtttgcctcaatatattggagtttaattcctatccatttttactttatttaaagttagacgattaattgattctcagaaaaattatttgttttaaaaggaagaattatttatttcgaaatctcaaaatatttccaaataccactgtgattcttggaatgcaatctttcttgggaaaatgccaaagttgggcaaagactggggtaggagttaggagagcaggcaaggacatgtaggaaggaaggcaaactagggaactattgtgtttttgaatcttttaaactcttagcctttagaacaatgtctggcatagagacttccaaaaagaatggttcaattcaatgtcataagtagcttgtcatttatggcacattcacaataaatgacagtataaatcaactgcgcattggtagacataccagagtcatgactatttcaacattcattttatccatgtgtttccaagtatgataatagctgaacattacgtggtactcaatgaaatttagatttatgtttatgcctcacatcttagcaattatatttgaaaaactggtgtctgtacagaatgttacagtttaaaaaggattttggatgagtaacttttgaaggaggtaggatttggggactctaaaatatttttcttcttatttttattgaatttctgcctgtgactgctttctaccctaacaatagaggcgctgcttactgtgctgcctgctgtatctcactggaaccattagtacttacaatgggcctttctgatctttatttctatatatttatattttttctgagatggagtctcgctctgtcacccaggccggagtgcagtggcgcatggctcactgtaacttctgcctcccgggttcaagtgattctcctgcctcagcctcccaagtagctgggactacaggcgtatgccacgacacccagctaattttttgtatttttagtagagacggggtttcactgtgttagccaggatggtctcgaactcctgacctcaggtgatccgcccgccttggcctcccaaagttctgggattacaggcgtgagccaccgtgcccagcctctgatcttttactctttctaaggatgactcgtgagtgaaatgtcaaactcttttatttatttatttatttttacaaaattaaaaaagatataatctgggaaaaatataacatctgaatttttattatatgtaagtctattaggtggaaggtagaaatagggccagccagtagaaattgaattctttgcatgaaggagagcaaatttaggtgtaatataagaaagaactttctaatagctaaatctgtctcgagttgcaatgatcatgcattcttctctcctcttttatttttggccactgctggatttcagggatcatcatctg

SEQ ID NO: 141 - protein, 1115 aa, Homo sapiens
MPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 142 - RNA, 29 nt, synthetic construct
  source 1..29: mol_type = other RNA
ggctttccggcctccgccggaaagcccct

SEQ ID NO: 143 - RNA, 26 nt, synthetic construct
  source 1..26: mol_type = other RNA
ggctttccggcctccgccggaaagcc

SEQ ID NO: 144 - RNA, 26 nt, synthetic construct
  source 1..26: mol_type = other RNA
ggttttctgacttcggtcggaaaacc

SEQ ID NO: 145 - RNA, 29 nt, synthetic construct
  source 1..29: mol_type = other RNA
ggttttctgacttcggtcggaaaacccct

SEQ ID NO: 146 - RNA, 29 nt, synthetic construct
  source 1..29: mol_type = other RNA
ggttttctgacctccgtcggaaaacccct

SEQ ID NO: 147 - RNA, 33 nt, synthetic construct
  source 1..33: mol_type = other RNA
ggttttctgacctccttcggtcggaaaacccct

SEQ ID NO: 148 - RNA, 26 nt, synthetic construct
  source 1..26: mol_type = other RNA
ggttttctgacctccgtcggaaaacc

SEQ ID NO: 149-150 - skipped (2 entries)

SEQ ID NO: 151 - DNA, 256 bp, Mus musculus; identical to SEQ ID NO: 120
  source 1..256: mol_type = genomic DNA

SEQ ID NO: 152 - DNA, 440 bp, Mus musculus
  source 1..440: mol_type = genomic DNA
tgccaacagctttgaaatcctctgtgctcttgtgccaatcccaccctcagaacagggagtctgtcggttatgttttcgtcctttacagttcttcgccgtctaatccattggaaacctcctttatttaaaggaaaccgtgtccaaattcagtgtacagaggtgcaaacctccgccccgctccttgtaggaagcccacgggaactgggccaatcggacggcgcttactcggctcatttacatacccataacacaccgcggctaatgcaaatattttcgtgaaaagatcttccattactcgaagttttaaaactaaaaacaaaatattatttcaaaagatcatgaagaaatttctgtcggtaacagcagtttcaattgataaatcaccatcacttatctaggaggttctgttactctagaagtgaattaagcaggacagctgt

SEQ ID NO: 153 - DNA, 231 bp, Mus musculus
  source 1..231: mol_type = genomic DNA
gggcggggcatgcaaataactgctctgtggaactctgggagcaaaaacaaaaaactgcaaccaaaacttctcggcctccttgaatcttacaggcttttcgtggcgtaaaggtggtgtactcaatgaagaggagagtctgtgttggctgcatgtttgagtcggttggttggtgactgtgaattaaaggtgtggtcggtgttgagtgtatggggcgtgtgggcgtagttcggt

SEQ ID NO: 154 - DNA, 311 bp, Mus musculus
  source 1..311: mol_type = genomic DNA
cgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttt

SEQ ID NO: 155 - DNA, 97 bp, Mus musculus
  source 1..97: mol_type = genomic DNA
catgcaaattacgcgctgtgctttgtgggaaatcaccctaaacgtaaaatttattcctctttcgagccttatagtggcggccggtctacaccctaaa

SEQ ID NO: 156 - DNA, 107 bp, Mus musculus
  source 1..107: mol_type = genomic DNA
cccaatttcactggtctacaatgaaagcaaaacagttctcttccccgctccccggtgtgtgagaggggctttgatccttctctggtttcctaggaaacgcgtatgtg

SEQ ID NO: 157 - DNA, 200 bp, Mus musculus
  source 1..200: mol_type = genomic DNA
ccacattttgtgtttaaaaatagaaatatttaagtgagatcagtttaaatctgctttatctagggtgtctaactgcttgcatctttttaacttttccttatttgtgagaaggtctgtaaaacttattatatgttagtacactgtagctgtcttcagacactccagaagagggattcagatcaccttacagatggttttga

SEQ ID NO: 158 - DNA, 78 bp, Mus musculus
  source 1..78: mol_type = genomic DNA
ccctctggggagtaaagttggttttaaagtcagagcatggtgattgtagggcagtccaacttttttaaatatgctgtg

SEQ ID NO: 159-160 - skipped (2 entries)

SEQ ID NO: 161 - RNA, 13 nt, synthetic construct
  source 1..13: mol_type = other RNA
aatttttggagca

SEQ ID NO: 162 - skipped

SEQ ID NO: 163 - RNA, 13 nt, synthetic construct
  source 1..13: mol_type = other RNA
aatttttggagta

SEQ ID NO: 164 - RNA, 30 nt, synthetic construct
  source 1..30: mol_type = other RNA
ggctttctggctccttaccggaaagcccct

SEQ ID NO: 165 - DNA, 420 bp, synthetic construct
  source 1..420: mol_type = other DNA
ttacttcatactaaaggctgtgcatccgactcctaagttgatgaaggaaaatgcctagtgttcttggaggctacaaaacaaaagacaaagctaactaccatctgcttatgggttcattggtattttccagctggcagggaggcgggtttccgagtacaggaaatgagtctctatggaggcggtgctatgtagatgagaattcaggagcaaactgggaaaagcagctgcttccaaatatttgtgatttttggagtgtagttttggggaaactcgcagcctaccaattctcctaagtgctttagaatatggagagacactgtacataaagatatagagctttttaatggaggcttaaatttataccgtatctacaaaatgctacattcacaatgcagttcaggctctgtggcattgcaactc

SEQ ID NO: 166 - DNA, 306 bp, synthetic construct
  source 1..306: mol_type = other DNA
gcgcaggagccgccgagctcttgctgcgaagccttgtctgcgttcttaaaaactaaagggggcgtgactggcttccttatcagccaatcggcatcgggtcatttgcatagggccccatccaccgttcgtaaactctagccgcgaaggatttaagagagttccttagaacacggtcttcccctccgaaagtcgcagtctgcctcgttatctgtagagcaataggagctaggaaccggccgtcggggctcaccctcactgacagcggggttggtggctatgccgtgcgatctccgggtgctacgtctg

SEQ ID NO: 167 - DNA, 420 bp, synthetic construct
  source 1..420: mol_type = other DNA
cgccgtgccctgctctgccctccgcacgctgctcagactccacacccgtaacgaagctccaccgaatttttcgtctgtctgtgaagacgaagaattgaaaaatctcagctacacttggtctttggggtcggcgccaggcagagctagaacgaactagacgcttgtccgaaagcagtgacgtcaaatgacaagggacaagaggtggggatatgtagatgaggggagcggtggctgctggctggatttcgtctttcgccgggtagaatttcccctgaacgtccaaaggatgtatcttggtcactgtgaaataaataatccttcaggttcccatcgaaggccgaaatagcttggaatataattgaccataactctgttgtgtgctgagttcgagggtgacgtcccagactaagcttacaactc

SEQ ID NO: 168 - DNA, 420 bp, synthetic construct
  source 1..420: mol_type = other DNA
tgccgcgcagaatgcagactcatcctgacaagtccacgccagaacctttgcgtctgcccggaaaggcaaggaagtttaaagcctttccagatacccctatggatgattgtcttttagatcgtcctgagggctagctagagcgaattcaccgcttctccgagaagcagaatgacgtcgagtgacgacagtgatgacaagaggcggggatatgtagatgaaagacacggtgactgccggttggagtgagttcttcatggggaacactttccactgaatatccaaaaggatgtaccctgatctctgtcatataaataacccttcaatttcgaacttaaggcctaaagaactcggaatataattgaccatgactttatagtgtgttgaagacgatggtgaggtgccaggcgaggggagcaactc

SEQ ID NO: 169 - DNA, 200 bp, synthetic construct
  source 1..200: mol_type = other DNA
atttgtttggtactaaagatagttatcagccgaaccagaaggctaaaatggcttctgatacttacttggccaatgccttttccctttatactgctattgctttgtattctgaaaagcatcttagtggtttttaactttctctacgttccctctgcacgttgagtcttgagttatgttaaatggtacggtacctaggattg

SEQ ID NO: 170 - DNA, 200 bp, synthetic construct
  source 1..200: mol_type = other DNA
cccacacaagtgtttatgaacaatagaataatatgtgggtgggaagcttggttttttttgatacgggttttttttttttttttggtctacgtgtttagtccgttcttgacggagaagacccgggaggttgggattcttgtctggaaatttccggctctgtctgatgtagtttcccgttcgcctcccttctttgctgcgag

SEQ ID NO: 171 - DNA, 200 bp, synthetic construct
  source 1..200: mol_type = other DNA
gcgtggtttgtgcaaggaaagacatagatgctaagtttggagctctgctaatttcctgtttttgtagtcgttttcgggtttatgaggcagaggcagtaagcagttttcctgcaacatggataagctagtttggacttttggtaaaggatttttttgtaatgtgggagacttgcttttgatccctggtttgggaaattctc

SEQ ID NO: 172 - DNA, 200 bp, synthetic construct
  source 1..200: mol_type = other DNA
acaaacttggtcttaaaagaagactacttgtttatgttcttctttctctaacttttatttggggtttagtggctttctaacatggtcattatgaggtaagaccttggggttggcctattaattaacaccttggggctggccgtatggctgagtggttaagtttgtgtgctctgcttcgggtttggcctgttctaattctg

SEQ ID NO: 173 - DNA, 181 bp, synthetic construct
  source 1..181: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 174 - DNA, 234 bp, synthetic construct
  source 1..234: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 175-185 - skipped (11 entries)

SEQ ID NO: 186 - RNA, 29 nt, synthetic construct
  source 1..29: mol_type = other RNA
ggctttctggcctccgccggaaagcccct

SEQ ID NO: 187-192 - skipped (6 entries)

SEQ ID NO: 193 - RNA, 29 nt, synthetic construct
  source 1..29: mol_type = other RNA
ggttttccggtctccaccggaaagccccc

SEQ ID NO: 194 - RNA, 29 nt, synthetic construct
  source 1..29: mol_type = other RNA
ggctttccggtctccaccggaaagcccct

SEQ ID NO: 195 - RNA, 29 nt, synthetic construct
  source 1..29: mol_type = other RNA
ggctttccggcctccgccggaaagcccct

SEQ ID NO: 196 - RNA, 29 nt, synthetic construct
  source 1..29: mol_type = other RNA
ggttttccggtctccaccggaaaaccctt

SEQ ID NO: 197 - RNA, 29 nt, synthetic construct
  source 1..29: mol_type = other RNA
ggttttccgtgctcccacggaaaaccctt

SEQ ID NO: 198 - RNA, 29 nt, synthetic construct
  source 1..29: mol_type = other RNA
ggttttccggcctccgccggaaaaccctt

SEQ ID NO: 199 - RNA, 30 nt, synthetic construct
  source 1..30: mol_type = other RNA
ggttttccgtgactcccacggaaaaccctt

SEQ ID NO: 200 - RNA, 30 nt, synthetic construct
  source 1..30: mol_type = other RNA
ggttttccggcactccgccggaaaaccctt

SEQ ID NO: 201 - RNA, 29 nt, synthetic construct
  source 1..29: mol_type = other RNA
ggtcttccggtctcctccggaaggccccc

SEQ ID NO: 202 - RNA, 27 nt, synthetic construct
  source 1..27: mol_type = other RNA
ggtcttccggctccccggaaggccccc

SEQ ID NO: 203 - RNA, 29 nt, synthetic construct
  source 1..29: mol_type = other RNA
ggctttccgtgctcccacggaaagcccct

SEQ ID NO: 204 - RNA, 30 nt, synthetic construct
  source 1..30: mol_type = other RNA
ggctttccgtgactcccacggaaagcccct

SEQ ID NO: 205 - RNA, 30 nt, synthetic construct
  source 1..30: mol_type = other RNA
ggctttccggcactccgccggaaagcccct

SEQ ID NO: 206 - RNA, 26 nt, synthetic construct
  source 1..26: mol_type = other RNA
tacctccaacatcaaggaagatggca

SEQ ID NO: 207 - RNA, 30 nt, synthetic construct
  source 1..30: mol_type = other RNA
gtaaccacaggttgtgtcaccagagtaaca

SEQ ID NO: 208 - RNA, 58 nt, synthetic construct
  source 1..58: mol_type = other RNA
cataccttctgcttgatgatcatctcgcaccagagtaacagtctgagtaggagctaaa

SEQ ID NO: 209 - RNA, 53 nt, synthetic construct
  source 1..53: mol_type = other RNA
cataccttctgcttgatgatcatctcgtgccgctgcccaatgccatcctggag

SEQ ID NO: 210 - RNA, 52 nt, synthetic construct
  source 1..52: mol_type = other RNA
cataccttctgcttgatgatcatctcggcccaatgccatcctggagttcctg

SEQ ID NO: 211 - RNA, 56 nt, synthetic construct
  source 1..56: mol_type = other RNA
tacctccaacatcaaggaagatggcagtaaccacaggttgtgtcaccagagtaaca

SEQ ID NO: 212 - RNA, 56 nt, synthetic construct
  source 1..56: mol_type = other RNA
aacatcaaggaagatggcatttctaggtaaccacaggttgtgtcaccagagtaaca

SEQ ID NO: 213 - RNA, 52 nt, synthetic construct
  source 1..52: mol_type = other RNA
aacatcaaggaagatggcatttctagtgccgctgcccaatgccatcctggag

SEQ ID NO: 214 - RNA, 52 nt, synthetic construct
  source 1..52: mol_type = other RNA
tttttctcataccttctgcttgatgtctgagtaggagctaaaatattttggg

SEQ ID NO: 215 - RNA, 56 nt, synthetic construct
  source 1..56: mol_type = other RNA
gtaaccacaggttgtgtcaccagagtaacatacctccaacatcaaggaagatggca

SEQ ID NO: 216 - RNA, 58 nt, synthetic construct
  source 1..58: mol_type = other RNA
cgagatgatcatcaagcagaaggtatgcaccagagtaacagtctgagtaggagctaaa

SEQ ID NO: 217 - RNA, 57 nt, synthetic construct
  source 1..57: mol_type = other RNA
caccagagtaacagtctgagtaggagctaaatacctccaacatcaaggaagatggca

SEQ ID NO: 218 - RNA, 57 nt, synthetic construct
  source 1..57: mol_type = other RNA
tacctccaacatcaaggaagatggcacaccagagtaacagtctgagtaggagctaaa

SEQ ID NO: 219 - RNA, 54 nt, synthetic construct
  source 1..54: mol_type = other RNA
gtctgagtaggagctaaaatattttgggtacctccaacatcaaggaagatggca

SEQ ID NO: 220 - RNA, 54 nt, synthetic construct
  source 1..54: mol_type = other RNA
tacctccaacatcaaggaagatggcagtctgagtaggagctaaaatattttggg

SEQ ID NO: 221 - RNA, 52 nt, synthetic construct
  source 1..52: mol_type = other RNA
gtctgagtaggagctaaaatattttgggtttttctcataccttctgcttgat

SEQ ID NO: 222 - RNA, 45 nt, synthetic construct
  source 1..45: mol_type = other RNA
ccttctgcttgatgatcatctcggagtaacagtctgagtaggagc

SEQ ID NO: 223 - RNA, 48 nt, synthetic construct
  source 1..48: mol_type = other RNA
ccttctgcttgatgatcatctcgccagagtaacagtctgagtaggagc

SEQ ID NO: 224 - RNA, 43 nt, synthetic construct
  source 1..43: mol_type = other RNA
ccttctgcttgatgatcatctcgcagtctgagtaggagctaaa

SEQ ID NO: 225 - RNA, 42 nt, synthetic construct
  source 1..42: mol_type = other RNA
ctcataccttctgcttgatggagtaacagtctgagtaggagc

SEQ ID NO: 226 - RNA, 45 nt, synthetic construct
  source 1..45: mol_type = other RNA
ctcataccttctgcttgatgccagagtaacagtctgagtaggagc

SEQ ID NO: 227 - RNA, 40 nt, synthetic construct
  source 1..40: mol_type = other RNA
ctcataccttctgcttgatgcagtctgagtaggagctaaa

SEQ ID NO: 228 - RNA, 30 nt, synthetic construct
  source 1..30: mol_type = other RNA
ggttttctgacactccgtcggaaaacccct

SEQ ID NO: 229 - RNA, 29 nt, synthetic construct
  source 1..29: mol_type = other RNA
ggttttctgatctccatcggaaaacccct

SEQ ID NO: 230 - RNA, 29 nt, synthetic construct
  source 1..29: mol_type = other RNA
ggttttccgacctccgtcggaaaacccct

SEQ ID NO: 231-233 - skipped (3 entries)

SEQ ID NO: 234 - RNA, 91 nt, Homo sapiens
  source 1..91: mol_type = genomic RNA
cttatgatgtttgttgccaatgatagattgttttcactgtgcaaaaattatgggtagttttggtggtcttgatgcagttgtaagcttggag

SEQ ID NO: 235 - RNA, 27 nt, Homo sapiens
  source 1..27: mol_type = genomic RNA
ggctttctggctttttaccggaaagcc

SEQ ID NO: 236 - RNA, 30 nt, Homo sapiens
  source 1..30: mol_type = genomic RNA
ggctttctggctttttaccggaaagcccct

SEQ ID NO: 237 - RNA, 28 nt, synthetic construct
  source 1..28: mol_type = other RNA
gttgcctccggttctgaaggtgttcttg

SEQ ID NO: 238 - RNA, 25 nt, synthetic construct
  source 1..25: mol_type = other RNA
ctccggttctgaaggtgttcttgta

SEQ ID NO: 239 - RNA, 28 nt, synthetic construct
  source 1..28: mol_type = other RNA
atttcattcaactgttgcctccggttct

SEQ ID NO: 240 - RNA, 25 nt, synthetic construct
  source 1..25: mol_type = other RNA
tgaaggtgttcttgtacttcatccc

SEQ ID NO: 241 - RNA, 25 nt, synthetic construct
  source 1..25: mol_type = other RNA
gttgcctccggttctgaaggtgttc

SEQ ID NO: 242 - RNA, 25 nt, synthetic construct
  source 1..25: mol_type = other RNA
ctgaaggtgttcttgtacttcatcc

SEQ ID NO: 243 - RNA, 30 nt, synthetic construct
  source 1..30: mol_type = other RNA
ctgttgcctccggttctgaaggtgttcttg

SEQ ID NO: 244 - RNA, 33 nt, synthetic construct
  source 1..33: mol_type = other RNA
caactgttgcctccggttctgaaggtgttcttg

SEQ ID NO: 245 - RNA, 27 nt, synthetic construct
  source 1..27: mol_type = other RNA
ctgattctgaattctttcaactagaat

SEQ ID NO: 246 - RNA, 27 nt, synthetic construct
  source 1..27: mol_type = other RNA
ctgaattctttcaactagaataaaagg

SEQ ID NO: 247 - RNA, 29 nt, synthetic construct
  source 1..29: mol_type = other RNA
ggtatctttgatactaaccttggtttctg

SEQ ID NO: 248 - RNA, 28 nt, synthetic construct
  source 1..28: mol_type = other RNA
ctttgatactaaccttggtttctgtgat

SEQ ID NO: 249 - RNA, 62 nt, synthetic construct; identical to SEQ ID NO: 121
  source 1..62: mol_type = other RNA

SEQ ID NO: 250 - RNA, 62 nt, synthetic construct
  source 1..62: mol_type = other RNA
tcatcccactgattctgaattctttcaactacattcaactgttgcctccggttctgaaggtg

SEQ ID NO: 251 - RNA, 31 nt, synthetic construct
  source 1..31: mol_type = other RNA
cattcaactgttgcctccggttctgaaggtg

SEQ ID NO: 252 - RNA, 57 nt, synthetic construct
  source 1..57: mol_type = other RNA
tcatcccactgattctgaattctttccattcaactgttgcctccggttctgaaggtg

SEQ ID NO: 253 - RNA, 58 nt, synthetic construct
  source 1..58: mol_type = other RNA
cattcaactgttgcctccggttctgaaggtgggtttctgtgattttcttttggattgc

SEQ ID NO: 254 - RNA, 61 nt, synthetic construct
  source 1..61: mol_type = other RNA
cattcaactgttgcctccggttctgaaggtggctcagcttcttccttagcttccagccatt

SEQ ID NO: 255 - RNA, 61 nt, synthetic construct
  source 1..61: mol_type = other RNA
cattcaactgttgcctccggttctgaaggtgcttccttagcttccagccattgtgttgaat

SEQ ID NO: 256 - RNA, 61 nt, synthetic construct
  source 1..61: mol_type = other RNA
cattcaactgttgcctccggttctgaaggtgctctggcctgtcctaagacctgctcagctt

SEQ ID NO: 257 - RNA, 57 nt, synthetic construct
  source 1..57: mol_type = other RNA
cattcaactgttgcctccggttctgaaggtgacctgctcagcttcttccttagcttc

SEQ ID NO: 258 - RNA, 57 nt, synthetic construct
  source 1..57: mol_type = other RNA
cattcaactgttgcctccggttctgaaggtgagcttcttccttagcttccagccatt

SEQ ID NO: 259 - RNA, 30 nt, synthetic construct
  source 1..30: mol_type = other RNA
tttcattcaactgttgcctccggttctgaa

SEQ ID NO: 260 - RNA, 30 nt, synthetic construct
  source 1..30: mol_type = other RNA
gaaggtgttcttgtacttcatcccactgat

SEQ ID NO: 261 - RNA, 30 nt, synthetic construct
  source 1..30: mol_type = other RNA
ttcattcaactgttgcctccggttctgaag

SEQ ID NO: 262 - RNA, 57 nt, synthetic construct
  source 1..57: mol_type = other RNA
cattcaactgttgcctccggttctgaaggtgattgcatctactgtatagggaccctc

SEQ ID NO: 263 - RNA, 31 nt, synthetic construct; identical to SEQ ID NO: 251
  source 1..31: mol_type = other RNA

SEQ ID NO: 264 - RNA, 57 nt, synthetic construct
  source 1..57: mol_type = other RNA
attgcatctactgtatagggaccctccattcaactgttgcctccggttctgaaggtg

SEQ ID NO: 265 - RNA, 61 nt, synthetic construct
  source 1..61: mol_type = other RNA
gctcagcttcttccttagcttccagccattcattcaactgttgcctccggttctgaaggtg

SEQ ID NO: 266 - RNA, 62 nt, synthetic construct; identical to SEQ ID NO: 121
  source 1..62: mol_type = other RNA

SEQ ID NO: 267 - RNA, 57 nt, synthetic construct
  source 1..57: mol_type = other RNA
attgcatctactgtatagggaccctctcatcccactgattctgaattctttcaacta

SEQ ID NO: 268 - RNA, 61 nt, synthetic construct
  source 1..61: mol_type = other RNA
tttcattcaactgttgcctccggttctgaatcatcccactgattctgaattctttcaacta

SEQ ID NO: 269 - RNA, 62 nt, synthetic construct
  source 1..62: mol_type = other RNA
cattcaactgttgcctccggttctgaaggtgattgcatctactgtatagggaccctccttcc

SEQ ID NO: 270 - RNA, 61 nt, synthetic construct
  source 1..61: mol_type = other RNA
cattcaactgttgcctccggttctgaaggtgggattgcatctactgtatagggaccctcct

SEQ ID NO: 271 - RNA, 61 nt, synthetic construct
  source 1..61: mol_type = other RNA
cattcaactgttgcctccggttctgaaggtgttggattgcatctactgtatagggaccctc

SEQ ID NO: 272 - RNA, 61 nt, synthetic construct
  source 1..61: mol_type = other RNA
cattcaactgttgcctccggttctgaaggtgtctactgtatagggaccctccttccatgac

SEQ ID NO: 273 - RNA, 62 nt, synthetic construct
  source 1..62: mol_type = other RNA
attgcatctactgtatagggaccctccttcccattcaactgttgcctccggttctgaaggtg

SEQ ID NO: 274 - RNA, 61 nt, synthetic construct
  source 1..61: mol_type = other RNA
ggattgcatctactgtatagggaccctcctcattcaactgttgcctccggttctgaaggtg

SEQ ID NO: 275 - RNA, 61 nt, synthetic construct
  source 1..61: mol_type = other RNA
ttggattgcatctactgtatagggaccctccattcaactgttgcctccggttctgaaggtg

SEQ ID NO: 276 - RNA, 31 nt, synthetic construct
  source 1..31: mol_type = other RNA
tcatcccactgattctgaattctttcaacta

SEQ ID NO: 277 - RNA, 23 nt, synthetic construct
  source 1..23: mol_type = other RNA
atactaaccttggtttctgtgat

SEQ ID NO: 278 - RNA, 35 nt, synthetic construct
  source 1..35: mol_type = other RNA
accttggtttctgtgattttcttttggattgcatc

SEQ ID NO: 279 - RNA, 37 nt, synthetic construct
  source 1..37: mol_type = other RNA
cattcaactgttgcctccggttctgaaggtgttcttg

SEQ ID NO: 280 - RNA, 37 nt, synthetic construct
  source 1..37: mol_type = other RNA
cccactgattctgaattctttcaactagaataaaagg

SEQ ID NO: 281 - RNA, 31 nt, synthetic construct
  source 1..31: mol_type = other RNA
catcccactgattctgaattctttcaactag

SEQ ID NO: 282 - RNA, 34 nt, synthetic construct
  source 1..34: mol_type = other RNA
tcatcccactgattctgaattctttcaactagaa

SEQ ID NO: 283 - RNA, 37 nt, synthetic construct
  source 1..37: mol_type = other RNA
gtacttcatcccactgattctgaattctttcaactag

SEQ ID NO: 284 - RNA, 35 nt, synthetic construct
  source 1..35: mol_type = other RNA
tacttcatcccactgattctgaattctttcaacta

SEQ ID NO: 285 - RNA, 37 nt, synthetic construct
  source 1..37: mol_type = other RNA
tttcattcaactgttgcctccggttctgaaggtgttc

SEQ ID NO: 286 - RNA, 39 nt, synthetic construct
  source 1..39: mol_type = other RNA
catttcattcaactgttgcctccggttctgaaggtgttc

SEQ ID NO: 287 - RNA, 35 nt, synthetic construct
  source 1..35: mol_type = other RNA
atttcattcaactgttgcctccggttctgaaggtg

SEQ ID NO: 288 - RNA, 35 nt, synthetic construct
  source 1..35: mol_type = other RNA
cttggctctggcctgtcctaagacctgctcagctt

SEQ ID NO: 289 - RNA, 35 nt, synthetic construct
  source 1..35: mol_type = other RNA
ctctggcctgtcctaagacctgctcagcttcttcc

SEQ ID NO: 290 - RNA, 40 nt, synthetic construct
  source 1..40: mol_type = other RNA
cttggctctggcctgtcctaagacctgctcagcttcttcc

SEQ ID NO: 291 - RNA, 30 nt, synthetic construct
  source 1..30: mol_type = other RNA
tgactcaagcttggctctggcctgtcctaa

SEQ ID NO: 292 - RNA, 36 nt, synthetic construct
  source 1..36: mol_type = other RNA
gactcaagcttggctctggcctgtcctaagacctgc

SEQ ID NO: 293 - RNA, 35 nt, synthetic construct
  source 1..35: mol_type = other RNA
caagcttggctctggcctgtcctaagacctgctca

SEQ ID NO: 294 - RNA, 32 nt, synthetic construct
  source 1..32: mol_type = other RNA
cttccttagcttccagccattgtgttgaatcc

SEQ ID NO: 295 - RNA, 36 nt, synthetic construct
  source 1..36: mol_type = other RNA
gcttcttccttagcttccagccattgtgttgaatcc

SEQ ID NO: 296 - RNA, 38 nt, synthetic construct
  source 1..38: mol_type = other RNA
cttccttagcttccagccattgtgttgaatcctttaac

SEQ ID NO: 297 - RNA, 35 nt, synthetic construct
  source 1..35: mol_type = other RNA
agcttggctctggcctgtcctaagacctgctcagc

SEQ ID NO: 298 - RNA, 33 nt, synthetic construct
  source 1..33: mol_type = other RNA
ggattgcatctactgtatagggaccctccttcc

SEQ ID NO: 299 - RNA, 39 nt, synthetic construct
  source 1..39: mol_type = other RNA
attgcatctactgtatagggaccctccttccatgactca

SEQ ID NO: 300 - RNA, 39 nt, synthetic construct
  source 1..39: mol_type = other RNA
catctactgtatagggaccctccttccatgactcaagct

SEQ ID NO: 301 - RNA, 40 nt, synthetic construct
  source 1..40: mol_type = other RNA
actgtatagggaccctccttccatgactcaagcttggctc

SEQ ID NO: 302 - RNA, 37 nt, synthetic construct
  source 1..37: mol_type = other RNA
cttttggattgcatctactgtatagggaccctccttc

SEQ ID NO: 303 - RNA, 40 nt, synthetic construct
  source 1..40: mol_type = other RNA
cagcttcttccttagcttccagccattgtgttgaatcctt

SEQ ID NO: 304 - RNA, 40 nt, synthetic construct
  source 1..40: mol_type = other RNA
ccctccttccatgactcaagcttggctctggcctgtccta

SEQ ID NO: 305 - RNA, 35 nt, synthetic construct
  source 1..35: mol_type = other RNA
ggattgcatctactgtatagggaccctccttccat

SEQ ID NO: 306 - RNA, 26 nt, synthetic construct
  source 1..26: mol_type = other RNA
attgcatctactgtatagggaccctc

SEQ ID NO: 307 - RNA, 68 nt, synthetic construct
  source 1..68: mol_type = other RNA
tttcattcaactgttgcctccggttctgaaggtgttctcatcccactgattctgaattctttcaacta

SEQ ID NO: 308 - RNA, 72 nt, synthetic construct
  source 1..72: mol_type = other RNA
tttcattcaactgttgcctccggttctgaaggtgttctacttcatcccactgattctgaattctttcaacta

SEQ ID NO: 309 - RNA, 71 nt, synthetic construct
  source 1..71: mol_type = other RNA
tttcattcaactgttgcctccggttctgaaggtgttctacttcatcccactgattctgaattctttcaact

SEQ ID NO: 310 - RNA, 75 nt, synthetic construct
  source 1..75: mol_type = other RNA
tttcattcaactgttgcctccggttctgaaggtgttcttgtacttcatcccactgattctgaattctttcaacta

SEQ ID NO: 311 - RNA, 70 nt, synthetic construct
  source 1..70: mol_type = other RNA
ggattgcatctactgtatagggaccctccttcctttcattcaactgttgcctccggttctgaaggtgttc

SEQ ID NO: 312 - RNA, 77 nt, synthetic construct
  source 1..77: mol_type = other RNA
cagcttcttccttagcttccagccattgtgttgaatcctttttcattcaactgttgcctccggttctgaaggtgttc

SEQ ID NO: 313 - RNA, 77 nt, synthetic construct
  source 1..77: mol_type = other RNA
tttcattcaactgttgcctccggttctgaaggtgttccagcttcttccttagcttccagccattgtgttgaatcctt

SEQ ID NO: 314 - RNA, 75 nt, synthetic construct
  source 1..75: mol_type = other RNA
cttccttagcttccagccattgtgttgaatcctttaactttcattcaactgttgcctccggttctgaaggtgttc

SEQ ID NO: 315 - RNA, 75 nt, synthetic construct
  source 1..75: mol_type = other RNA
cttccttagcttccagccattgtgttgaatcctttaaccattcaactgttgcctccggttctgaaggtgttcttg

SEQ ID NO: 316 - RNA, 73 nt, synthetic construct
  source 1..73: mol_type = other RNA
gcttcttccttagcttccagccattgtgttgaatcctttcattcaactgttgcctccggttctgaaggtgttc

SEQ ID NO: 317 - RNA, 74 nt, synthetic construct
  source 1..74: mol_type = other RNA
cttttggattgcatctactgtatagggaccctccttctttcattcaactgttgcctccggttctgaaggtgttc

SEQ ID NO: 318 - RNA, 76 nt, synthetic construct
  source 1..76: mol_type = other RNA
attgcatctactgtatagggaccctccttccatgactcatttcattcaactgttgcctccggttctgaaggtgttc

SEQ ID NO: 319 - RNA, 77 nt, synthetic construct
  source 1..77: mol_type = other RNA
cttttggattgcatctactgtatagggaccctccttccagcttcttccttagcttccagccattgtgttgaatcctt

SEQ ID NO: 320 - RNA, 79 nt, synthetic construct
  source 1..79: mol_type = other RNA
attgcatctactgtatagggaccctccttccatgactcacagcttcttccttagcttccagccattgtgttgaatcctt

SEQ ID NO: 321 - RNA, 68 nt, synthetic construct; identical to SEQ ID NO: 307
  source 1..68: mol_type = other RNA

SEQ ID NO: 322 - RNA, 72 nt, synthetic construct; identical to SEQ ID NO: 308
  source 1..72: mol_type = other RNA

SEQ ID NO: 323 - RNA, 71 nt, synthetic construct; identical to SEQ ID NO: 309
  source 1..71: mol_type = other RNA

SEQ ID NO: 324 - RNA, 70 nt, synthetic construct; identical to SEQ ID NO: 311
  source 1..70: mol_type = other RNA

SEQ ID NO: 325 - RNA, 77 nt, synthetic construct; identical to SEQ ID NO: 312
  source 1..77: mol_type = other RNA

SEQ ID NO: 326 - RNA, 77 nt, synthetic construct; identical to SEQ ID NO: 313
  source 1..77: mol_type = other RNA

SEQ ID NO: 327 - RNA, 75 nt, synthetic construct; identical to SEQ ID NO: 314
  source 1..75: mol_type = other RNA

SEQ ID NO: 328 - RNA, 75 nt, synthetic construct; identical to SEQ ID NO: 315
  source 1..75: mol_type = other RNA

SEQ ID NO: 329 - RNA, 73 nt, synthetic construct; identical to SEQ ID NO: 316
  source 1..73: mol_type = other RNA

SEQ ID NO: 330 - RNA, 74 nt, synthetic construct; identical to SEQ ID NO: 317
  source 1..74: mol_type = other RNA

SEQ ID NO: 331 - RNA, 76 nt, synthetic construct; identical to SEQ ID NO: 318
  source 1..76: mol_type = other RNA

SEQ ID NO: 332 - RNA, 77 nt, synthetic construct; identical to SEQ ID NO: 319
  source 1..77: mol_type = other RNA

SEQ ID NO: 333 - RNA, 79 nt, synthetic construct; identical to SEQ ID NO: 320
  source 1..79: mol_type = other RNA

SEQ ID NO: 334 - RNA, 22 nt, synthetic construct
  source 1..22: mol_type = other RNA
gagtgaacataatatttccaga

SEQ ID NO: 335 - RNA, 22 nt, synthetic construct
  source 1..22: mol_type = other RNA
agtgaacataatatttccagat

SEQ ID NO: 336 - RNA, 22 nt, synthetic construct
  source 1..22: mol_type = other RNA
gtgaacataatatttccagatt

SEQ ID NO: 337 - RNA, 22 nt, synthetic construct
  source 1..22: mol_type = other RNA
tgaacataatatttccagattt

SEQ ID NO: 338 - RNA, 22 nt, synthetic construct
  source 1..22: mol_type = other RNA
gaacataatatttccagatttg

SEQ ID NO: 339 - RNA, 22 nt, synthetic construct
  source 1..22: mol_type = other RNA
aacataatatttccagatttgc

SEQ ID NO: 340 - RNA, 22 nt, synthetic construct
  source 1..22: mol_type = other RNA
acataatatttccagatttgca

SEQ ID NO: 341 - RNA, 22 nt, synthetic construct
  source 1..22: mol_type = other RNA
cataatatttccagatttgcac

SEQ ID NO: 342 - RNA, 22 nt, synthetic construct
  source 1..22: mol_type = other RNA
ataatatttccagatttgcaca

SEQ ID NO: 343 - RNA, 22 nt, synthetic construct
  source 1..22: mol_type = other RNA
taatatttccagatttgcacag

SEQ ID NO: 344 - RNA, 22 nt, synthetic construct
  source 1..22: mol_type = other RNA
aatatttccagatttgcacagc

SEQ ID NO: 345 - RNA, 22 nt, synthetic construct
  source 1..22: mol_type = other RNA
atatttccagatttgcacagct

SEQ ID NO: 346 - RNA, 22 nt, synthetic construct
  source 1..22: mol_type = other RNA
tatttccagatttgcacagcta

SEQ ID NO: 347 - RNA, 22 nt, synthetic construct
  source 1..22: mol_type = other RNA
atttccagatttgcacagctaa

SEQ ID NO: 348 - RNA, 22 nt, synthetic construct
  source 1..22: mol_type = other RNA
tttccagatttgcacagctaaa

SEQ ID NO: 349 - RNA, 22 nt, synthetic construct
  source 1..22: mol_type = other RNA
ttccagatttgcacagctaaaa

SEQ ID NO: 350 - RNA, 22 nt, synthetic construct
  source 1..22: mol_type = other RNA
tccagatttgcacagctaaaat

SEQ ID NO: 351 - RNA, 22 nt, synthetic construct
  source 1..22: mol_type = other RNA
ccagatttgcacagctaaaata

SEQ ID NO: 352 - RNA, 22 nt, synthetic construct
  source 1..22: mol_type = other RNA
cagatttgcacagctaaaataa

SEQ ID NO: 353 - RNA, 22 nt, synthetic construct
  source 1..22: mol_type = other RNA
agatttgcacagctaaaataaa

SEQ ID NO: 354 - RNA, 22 nt, synthetic construct
  source 1..22: mol_type = other RNA
gatttgcacagctaaaataaaa

SEQ ID NO: 355 - RNA, 22 nt, synthetic construct
  source 1..22: mol_type = other RNA
atttgcacagctaaaataaaat

SEQ ID NO: 356 - RNA, 22 nt, synthetic construct
  source 1..22: mol_type = other RNA
tttgcacagctaaaataaaatg

SEQ ID NO: 357 - RNA, 22 nt, synthetic construct
  source 1..22: mol_type = other RNA
ttgcacagctaaaataaaatga

SEQ ID NO: 358 - RNA, 22 nt, synthetic construct
  source 1..22: mol_type = other RNA
tgcacagctaaaataaaatgac

SEQ ID NO: 359 - RNA, 22 nt, synthetic construct
  source 1..22: mol_type = other RNA
gcacagctaaaataaaatgaca

SEQ ID NO: 360 - RNA, 22 nt, synthetic construct
  source 1..22: mol_type = other RNA
cacagctaaaataaaatgacac

SEQ ID NO: 361 - RNA, 22 nt, synthetic construct
  source 1..22: mol_type = other RNA
acagctaaaataaaatgacact

SEQ ID NO: 362 - RNA, 22 nt, synthetic construct
  source 1..22: mol_type = other RNA
cagctaaaataaaatgacacta

SEQ ID NO: 363 - RNA, 22 nt, synthetic construct
  source 1..22: mol_type = other RNA
agctaaaataaaatgacactat

SEQ ID NO: 364 - RNA, 22 nt, synthetic construct
  source 1..22: mol_type = other RNA
gctaaaataaaatgacactatg

SEQ ID NO: 365 - RNA, 22 nt, synthetic construct
  source 1..22: mol_type = other RNA
ctaaaataaaatgacactatga

SEQ ID NO: 366 - RNA, 22 nt, synthetic construct
  source 1..22: mol_type = other RNA
taaaataaaatgacactatgag

SEQ ID NO: 367 - RNA, 22 nt, synthetic construct
  source 1..22: mol_type = other RNA
aaaataaaatgacactatgaga

SEQ ID NO: 368 - RNA, 22 nt, synthetic construct
  source 1..22: mol_type = other RNA
aaataaaatgacactatgagag

SEQ ID NO: 369 - RNA, 22 nt, synthetic construct
  source 1..22: mol_type = other RNA
aataaaatgacactatgagaga

SEQ ID NO: 370 - RNA, 22 nt, synthetic construct
  source 1..22: mol_type = other RNA
ataaaatgacactatgagagaa

SEQ ID NO: 371 - RNA, 22 nt, synthetic construct
  source 1..22: mol_type = other RNA
taaaatgacactatgagagaaa

SEQ ID NO: 372 - RNA, 22 nt, synthetic construct
  source 1..22: mol_type = other RNA
aaaatgacactatgagagaaat

SEQ ID NO: 373 - RNA, 22 nt, synthetic construct
  source 1..22: mol_type = other RNA
aaatgacactatgagagaaata

SEQ ID NO: 374 - RNA, 22 nt, synthetic construct
  source 1..22: mol_type = other RNA
aatgacactatgagagaaataa

SEQ ID NO: 375 - RNA, 22 nt, synthetic construct
  source 1..22: mol_type = other RNA
atgacactatgagagaaataaa

SEQ ID NO: 376 - RNA, 22 nt, synthetic construct
  source 1..22: mol_type = other RNA
tgacactatgagagaaataaaa

SEQ ID NO: 377 - RNA, 22 nt, synthetic construct
  source 1..22: mol_type = other RNA
gacactatgagagaaataaaac

SEQ ID NO: 378 - RNA, 22 nt, synthetic construct
  source 1..22: mol_type = other RNA
acactatgagagaaataaaacg

SEQ ID NO: 379 - RNA, 22 nt, synthetic construct
  source 1..22: mol_type = other RNA
cactatgagagaaataaaacgg

SEQ ID NO: 380 - RNA, 22 nt, synthetic construct
  source 1..22: mol_type = other RNA
actatgagagaaataaaacgga

SEQ ID NO: 381 - RNA, 22 nt, synthetic construct
  source 1..22: mol_type = other RNA
ctatgagagaaataaaacggat

SEQ ID NO: 382 - RNA, 22 nt, synthetic construct
  source 1..22: mol_type = other RNA
tatgagagaaataaaacggatt

SEQ ID NO: 383 - RNA, 22 nt, synthetic construct
  source 1..22: mol_type = other RNA
atgagagaaataaaacggattt

SEQ ID NO: 384 - RNA, 22 nt, synthetic construct
  source 1..22: mol_type = other RNA
tgagagaaataaaacggatttt

SEQ ID NO: 385 - RNA, 22 nt, synthetic construct
  source 1..22: mol_type = other RNA
gagagaaataaaacggattttt

SEQ ID NO: 386 - RNA, 22 nt, synthetic construct
  source 1..22: mol_type = other RNA
agagaaataaaacggattttta

SEQ ID NO: 387 - RNA, 22 nt, synthetic construct
  source 1..22: mol_type = other RNA
gagaaataaaacggatttttaa

SEQ ID NO: 388 - RNA, 22 nt, synthetic construct
  source 1..22: mol_type = other RNA
agaaataaaacggatttttaag

SEQ ID NO: 389 - RNA, 22 nt, synthetic construct
  source 1..22: mol_type = other RNA
gaaataaaacggatttttaaga

SEQ ID NO: 390 - RNA, 22 nt, synthetic construct
  source 1..22: mol_type = other RNA
aaataaaacggatttttaagat

SEQ ID NO: 391 - RNA, 22 nt, synthetic construct
  source 1..22: mol_type = other RNA
aataaaacggatttttaagata

SEQ ID NO: 392 - RNA, 22 nt, synthetic construct
  source 1..22: mol_type = other RNA
ataaaacggatttttaagatac

SEQ ID NO: 393 - RNA, 22 nt, synthetic construct
  source 1..22: mol_type = other RNA
taaaacggatttttaagataca

SEQ ID NO: 394 - RNA, 22 nt, synthetic construct
  source 1..22: mol_type = other RNA
aaaacggatttttaagatacac

SEQ ID NO: 395 - RNA, 22 nt, synthetic construct
  source 1..22: mol_type = other RNA
aaacggatttttaagatacaca

SEQ ID NO: 396 - RNA, 22 nt, synthetic construct
  source 1..22: mol_type = other RNA
aacggatttttaagatacacag

SEQ ID NO: 397 - RNA, 22 nt, synthetic construct
  source 1..22: mol_type = other RNA
acggatttttaagatacacagg

SEQ ID NO: 398 - RNA, 22 nt, synthetic construct
  source 1..22: mol_type = other RNA
cggatttttaagatacacaggt

SEQ ID NO: 399 - RNA, 22 nt, synthetic construct
  source 1..22: mol_type = other RNA
ggatttttaagatacacaggta

SEQ ID NO: 400 - RNA, 22 nt, synthetic construct
  source 1..22: mol_type = other RNA
gatttttaagatacacaggtac

SEQ ID NO: 401 - RNA, 22 nt, synthetic construct
  source 1..22: mol_type = other RNA
atttttaagatacacaggtaca

SEQ ID NO: 402 - RNA, 22 nt, synthetic construct
  source 1..22: mol_type = other RNA
tttttaagatacacaggtacat

SEQ ID NO: 403 - RNA, 22 nt, synthetic construct
  source 1..22: mol_type = other RNA
ttttaagatacacaggtacata

SEQ ID NO: 404 - RNA, 22 nt, synthetic construct
  source 1..22: mol_type = other RNA
tttaagatacacaggtacatag

SEQ ID NO: 405 - RNA, 22 nt, synthetic construct
  source 1..22: mol_type = other RNA
ttaagatacacaggtacatagt

SEQ ID NO: 406 - RNA, 22 nt, synthetic construct
  source 1..22: mol_type = other RNA
taagatacacaggtacatagtc

SEQ ID NO: 407 - RNA, 22 nt, synthetic construct
  source 1..22: mol_type = other RNA
aagatacacaggtacatagtcc

SEQ ID NO: 408 - RNA, 22 nt, synthetic construct
  source 1..22: mol_type = other RNA
agatacacaggtacatagtcca

SEQ ID NO: 409 - RNA, 22 nt, synthetic construct
  source 1..22: mol_type = other RNA
gatacacaggtacatagtccat

SEQ ID NO: 410 - RNA, 22 nt, synthetic construct
  source 1..22: mol_type = other RNA
atacacaggtacatagtccatt

SEQ ID NO: 411 - RNA, 22 nt, synthetic construct
  source 1..22: mol_type = other RNA
tacacaggtacatagtccattt

SEQ ID NO: 412 - RNA, 22 nt, synthetic construct
  source 1..22: mol_type = other RNA
acacaggtacatagtccatttt

SEQ ID NO: 413 - RNA, 22 nt, synthetic construct
  source 1..22: mol_type = other RNA
cacaggtacatagtccattttg

SEQ ID NO: 414 - RNA, 22 nt, synthetic construct
  source 1..22: mol_type = other RNA
acaggtacatagtccattttga

SEQ ID NO: 415 - RNA, 22 nt, synthetic construct
  source 1..22: mol_type = other RNA
caggtacatagtccattttgaa

SEQ ID NO: 416 - RNA, 22 nt, synthetic construct
  source 1..22: mol_type = other RNA
aggtacatagtccattttgaaa

SEQ ID NO: 417 - RNA, 22 nt, synthetic construct
  source 1..22: mol_type = other RNA
ggtacatagtccattttgaaaa

SEQ ID NO: 418 - RNA, 22 nt, synthetic construct
  source 1..22: mol_type = other RNA
gtacatagtccattttgaaaat

SEQ ID NO: 419 - RNA, 22 nt, synthetic construct
  source 1..22: mol_type = other RNA
tacatagtccattttgaaaatt

SEQ ID NO: 420 - RNA, 22 nt, synthetic construct
  source 1..22: mol_type = other RNA
acatagtccattttgaaaattt

SEQ ID NO: 421 - RNA, 22 nt, synthetic construct
  source 1..22: mol_type = other RNA
catagtccattttgaaaatttc

SEQ ID NO: 422 - RNA, 22 nt, synthetic construct
  source 1..22: mol_type = other RNA
atagtccattttgaaaatttca

SEQ ID NO: 423 - RNA, 22 nt, synthetic construct
  source 1..22: mol_type = other RNA
tagtccattttgaaaatttcac

SEQ ID NO: 424 - RNA, 22 nt, synthetic construct
  source 1..22: mol_type = other RNA
agtccattttgaaaatttcaca

SEQ ID NO: 425 - RNA, 22 nt, synthetic construct
  source 1..22: mol_type = other RNA
gtccattttgaaaatttcacaa

SEQ ID NO: 426 - RNA, 22 nt, synthetic construct
  source 1..22: mol_type = other RNA
tccattttgaaaatttcacaac

SEQ ID NO: 427 - RNA, 22 nt, synthetic construct
  source 1..22: mol_type = other RNA
ccattttgaaaatttcacaact

SEQ ID NO: 428 - RNA, 22 nt, synthetic construct
  source 1..22: mol_type = other RNA
cattttgaaaatttcacaactt

SEQ ID NO: 429 - RNA, 22 nt, synthetic construct
  source 1..22: mol_type = other RNA
attttgaaaatttcacaactta

SEQ ID NO: 430 - RNA, 22 nt, synthetic construct
  source 1..22: mol_type = other RNA
ttttgaaaatttcacaacttag

SEQ ID NO: 431 - RNA, 22 nt, synthetic construct
  source 1..22: mol_type = other RNA
tttgaaaatttcacaacttaga

SEQ ID NO: 432 - RNA, 22 nt, synthetic construct
  source 1..22: mol_type = other RNA
ttgaaaatttcacaacttagat

SEQ ID NO: 433 - RNA, 22 nt, synthetic construct
  source 1..22: mol_type = other RNA
tgaaaatttcacaacttagatc

SEQ ID NO: 434 - RNA, 22 nt, synthetic construct
  source 1..22: mol_type = other RNA
gaaaatttcacaacttagatct

SEQ ID NO: 435 - RNA, 22 nt, synthetic construct
  source 1..22: mol_type = other RNA
aaaatttcacaacttagatctt

SEQ ID NO: 436 - RNA, 22 nt, synthetic construct
  source 1..22: mol_type = other RNA
aaatttcacaacttagatctta

SEQ ID NO: 437 - RNA, 22 nt, synthetic construct
  source 1..22: mol_type = other RNA
aatttcacaacttagatcttaa

SEQ ID NO: 438 - RNA, 22 nt, synthetic construct
  source 1..22: mol_type = other RNA
atttcacaacttagatcttaaa

SEQ ID NO: 439 - RNA, 22 nt, synthetic construct
  source 1..22: mol_type = other RNA
tttcacaacttagatcttaaaa

SEQ ID NO: 440 - RNA, 22 nt, synthetic construct
  source 1..22: mol_type = other RNA
ttcacaacttagatcttaaaag

SEQ ID NO: 441 - RNA, 22 nt, synthetic construct
  source 1..22: mol_type = other RNA
tcacaacttagatcttaaaagt

SEQ ID NO: 442 - RNA, 22 nt, synthetic construct
  source 1..22: mol_type = other RNA
cacaacttagatcttaaaagta

SEQ ID NO: 443 - RNA, 22 nt, synthetic construct
  source 1..22: mol_type = other RNA
acaacttagatcttaaaagtaa

SEQ ID NO: 444 - RNA, 22 nt, synthetic construct
  source 1..22: mol_type = other RNA
caacttagatcttaaaagtaaa

SEQ ID NO: 445 - RNA, 22 nt, synthetic construct
  source 1..22: mol_type = other RNA
aacttagatcttaaaagtaaag

SEQ ID NO: 446 - RNA, 22 nt, synthetic construct
  source 1..22: mol_type = other RNA
acttagatcttaaaagtaaagt

SEQ ID NO: 447 - RNA, 22 nt, synthetic construct
  source 1..22: mol_type = other RNA
cttagatcttaaaagtaaagta

SEQ ID NO: 448 - RNA, 22 nt, synthetic construct
  source 1..22: mol_type = other RNA
ttagatcttaaaagtaaagtaa

SEQ ID NO: 449 - RNA, 22 nt, synthetic construct
  source 1..22: mol_type = other RNA
tagatcttaaaagtaaagtaac

SEQ ID NO: 450 - RNA, 22 nt, synthetic construct
  source 1..22: mol_type = other RNA
agatcttaaaagtaaagtaaca

SEQ ID NO: 451 - RNA, 22 nt, synthetic construct
  source 1..22: mol_type = other RNA
gatcttaaaagtaaagtaacaa

SEQ ID NO: 452 - RNA, 22 nt, synthetic construct
  source 1..22: mol_type = other RNA
atcttaaaagtaaagtaacaaa

SEQ ID NO: 453 - RNA, 22 nt, synthetic construct
  source 1..22: mol_type = other RNA
tcttaaaagtaaagtaacaaac

SEQ ID NO: 454 - RNA, 22 nt, synthetic construct
  source 1..22: mol_type = other RNA
cttaaaagtaaagtaacaaacc

SEQ ID NO: 455 - RNA, 22 nt, synthetic construct
  source 1..22: mol_type = other RNA
ttaaaagtaaagtaacaaacca

SEQ ID NO: 456 - RNA, 22 nt, synthetic construct
  source 1..22: mol_type = other RNA
taaaagtaaagtaacaaaccat

SEQ ID NO: 457 - RNA, 22 nt, synthetic construct
  source 1..22: mol_type = other RNA
aaaagtaaagtaacaaaccatt

SEQ ID NO: 458 - RNA, 22 nt, synthetic construct
  source 1..22: mol_type = other RNA
aaagtaaagtaacaaaccattc

SEQ ID NO: 459 - RNA, 22 nt, synthetic construct
  source 1..22: mol_type = other RNA
aagtaaagtaacaaaccattct

SEQ ID NO: 460 - RNA, 22 nt, synthetic construct
  source 1..22: mol_type = other RNA
agtaaagtaacaaaccattctt

SEQ ID NO: 461 - RNA, 22 nt, synthetic construct
  source 1..22: mol_type = other RNA
gtaaagtaacaaaccattctta

SEQ ID NO: 462 - RNA, 22 nt, synthetic construct
  source 1..22: mol_type = other RNA
taaagtaacaaaccattcttac

SEQ ID NO: 463 - RNA, 22 nt, synthetic construct
  source 1..22: mol_type = other RNA
aaagtaacaaaccattcttacc

SEQ ID NO: 464 - RNA, 22 nt, synthetic construct
  source 1..22: mol_type = other RNA
aagtaacaaaccattcttacct

SEQ ID NO: 465 - RNA, 22 nt, synthetic construct
  source 1..22: mol_type = other RNA
agtaacaaaccattcttacctt

SEQ ID NO: 466 - RNA, 22 nt, synthetic construct
  source 1..22: mol_type = other RNA
gtaacaaaccattcttacctta

SEQ ID NO: 467 - RNA, 22 nt, synthetic construct
  source 1..22: mol_type = other RNA
taacaaaccattcttaccttag

SEQ ID NO: 468 - RNA, 22 nt, synthetic construct
  source 1..22: mol_type = other RNA
aacaaaccattcttaccttaga

SEQ ID NO: 469 - RNA, 22 nt, synthetic construct
  source 1..22: mol_type = other RNA
acaaaccattcttaccttagaa

SEQ ID NO: 470 - RNA, 22 nt, synthetic construct
  source 1..22: mol_type = other RNA
caaaccattcttaccttagaaa

SEQ ID NO: 471 - RNA, 22 nt, synthetic construct
  source 1..22: mol_type = other RNA
aaaccattcttaccttagaaaa

SEQ ID NO: 472 - RNA, 22 nt, synthetic construct
  source 1..22: mol_type = other RNA
aaccattcttaccttagaaaat

SEQ ID NO: 473 - RNA, 22 nt, synthetic construct
  source 1..22: mol_type = other RNA
accattcttaccttagaaaatt

SEQ ID NO: 474 - RNA, 22 nt, synthetic construct
  source 1..22: mol_type = other RNA
ccattcttaccttagaaaattg

SEQ ID NO: 475 - RNA, 22 nt, synthetic construct
  source 1..22: mol_type = other RNA
cattcttaccttagaaaattgt

SEQ ID NO: 476 - RNA, 22 nt, synthetic construct
  source 1..22: mol_type = other RNA
attcttaccttagaaaattgtg

SEQ ID NO: 477 - RNA, 22 nt, synthetic construct
  source 1..22: mol_type = other RNA
ttcttaccttagaaaattgtgc

SEQ ID NO: 478 - RNA, 22 nt, synthetic construct
  source 1..22: mol_type = other RNA
tcttaccttagaaaattgtgca

SEQ ID NO: 479 - RNA, 22 nt, synthetic construct
  source 1..22: mol_type = other RNA
cttaccttagaaaattgtgcat

SEQ ID NO: 480 - RNA, 22 nt, synthetic construct
  source 1..22: mol_type = other RNA
ttaccttagaaaattgtgcatt

SEQ ID NO: 481 - RNA, 22 nt, synthetic construct
  source 1..22: mol_type = other RNA
taccttagaaaattgtgcattt

SEQ ID NO: 482 - RNA, 22 nt, synthetic construct
  source 1..22: mol_type = other RNA
accttagaaaattgtgcattta

SEQ ID NO: 483 - RNA, 22 nt, synthetic construct
  source 1..22: mol_type = other RNA
ccttagaaaattgtgcatttac

SEQ ID NO: 484 - RNA, 22 nt, synthetic construct
  source 1..22: mol_type = other RNA
cttagaaaattgtgcatttacc

SEQ ID NO: 485 - RNA, 22 nt, synthetic construct
  source 1..22: mol_type = other RNA
ttagaaaattgtgcatttaccc

SEQ ID NO: 486 - RNA, 22 nt, synthetic construct
  source 1..22: mol_type = other RNA
tagaaaattgtgcatttaccca

SEQ ID NO: 487 - RNA, 22 nt, synthetic construct
  source 1..22: mol_type = other RNA
agaaaattgtgcatttacccat

SEQ ID NO: 488 - RNA, 22 nt, synthetic construct
  source 1..22: mol_type = other RNA
gaaaattgtgcatttacccatt

SEQ ID NO: 489 - RNA, 22 nt, synthetic construct
  source 1..22: mol_type = other RNA
aaaattgtgcatttacccattt

SEQ ID NO: 490 - RNA, 22 nt, synthetic construct
  source 1..22: mol_type = other RNA
aaattgtgcatttacccatttt

SEQ ID NO: 491 - RNA, 22 nt, synthetic construct
  source 1..22: mol_type = other RNA
aattgtgcatttacccattttg

SEQ ID NO: 492 - RNA, 22 nt, synthetic construct
  source 1..22: mol_type = other RNA
attgtgcatttacccattttgt

SEQ ID NO: 493 - RNA, 22 nt, synthetic construct
  source 1..22: mol_type = other RNA
ttgtgcatttacccattttgtg

SEQ ID NO: 494 - RNA, 22 nt, synthetic construct
  source 1..22: mol_type = other RNA
tgtgcatttacccattttgtga

SEQ ID NO: 495 - RNA, 22 nt, synthetic construct
  source 1..22: mol_type = other RNA
gtgcatttacccattttgtgaa

SEQ ID NO: 496 - RNA, 22 nt, synthetic construct
  source 1..22: mol_type = other RNA
tgcatttacccattttgtgaat

SEQ ID NO: 497 - RNA, 22 nt, synthetic construct
  source 1..22: mol_type = other RNA
gcatttacccattttgtgaatg

SEQ ID NO: 498 - RNA, 22 nt, synthetic construct
  source 1..22: mol_type = other RNA
catttacccattttgtgaatgt

SEQ ID NO: 499 - RNA, 22 nt, synthetic construct
  source 1..22: mol_type = other RNA
atttacccattttgtgaatgtt

SEQ ID NO: 500 - RNA, 22 nt, synthetic construct
  source 1..22: mol_type = other RNA
tttacccattttgtgaatgttt

SEQ ID NO: 501 - RNA, 22 nt, synthetic construct
  source 1..22: mol_type = other RNA
ttacccattttgtgaatgtttt

SEQ ID NO: 502 - RNA, 22 nt, synthetic construct
  source 1..22: mol_type = other RNA
tacccattttgtgaatgttttc

SEQ ID NO: 503 - RNA, 22 nt, synthetic construct
  source 1..22: mol_type = other RNA
acccattttgtgaatgttttct

SEQ ID NO: 504 - RNA, 22 nt, synthetic construct
  source 1..22: mol_type = other RNA
cccattttgtgaatgttttctt

SEQ ID NO: 505 - RNA, 22 nt, synthetic construct
  source 1..22: mol_type = other RNA
ccattttgtgaatgttttcttt

SEQ ID NO: 506 - RNA, 22 nt, synthetic construct
  source 1..22: mol_type = other RNA
cattttgtgaatgttttctttt

SEQ ID NO: 507 - RNA, 22 nt, synthetic construct
  source 1..22: mol_type = other RNA
attttgtgaatgttttcttttg

SEQ ID NO: 508 - RNA, 22 nt, synthetic construct
  source 1..22: mol_type = other RNA
ttttgtgaatgttttcttttga

SEQ ID NO: 509 - RNA, 22 nt, synthetic construct
  source 1..22: mol_type = other RNA
tttgtgaatgttttcttttgaa

SEQ ID NO: 510 - RNA, 22 nt, synthetic construct
  source 1..22: mol_type = other RNA
ttgtgaatgttttcttttgaac

SEQ ID NO: 511 - RNA, 22 nt, synthetic construct
  source 1..22: mol_type = other RNA
tgtgaatgttttcttttgaaca

SEQ ID NO: 512 - RNA, 22 nt, synthetic construct
  source 1..22: mol_type = other RNA
gtgaatgttttcttttgaacat

SEQ ID NO: 513 - RNA, 22 nt, synthetic construct
  source 1..22: mol_type = other RNA
tgaatgttttcttttgaacatc

SEQ ID NO: 514 - RNA, 22 nt, synthetic construct
  source 1..22: mol_type = other RNA
gaatgttttcttttgaacatct

SEQ ID NO: 515 - RNA, 22 nt, synthetic construct
  source 1..22: mol_type = other RNA
aatgttttcttttgaacatctt

SEQ ID NO: 516 - RNA, 22 nt, synthetic construct
  source 1..22: mol_type = other RNA
atgttttcttttgaacatcttc

SEQ ID NO: 517 - RNA, 22 nt, synthetic construct
  source 1..22: mol_type = other RNA
tgttttcttttgaacatcttct

SEQ ID NO: 518 - RNA, 22 nt, synthetic construct
  source 1..22: mol_type = other RNA
gttttcttttgaacatcttctc

SEQ ID NO: 519 - RNA, 22 nt, synthetic construct
  source 1..22: mol_type = other RNA
ttttcttttgaacatcttctct

SEQ ID NO: 520 - RNA, 22 nt, synthetic construct
  source 1..22: mol_type = other RNA
tttcttttgaacatcttctctt

SEQ ID NO: 521 - RNA, 22 nt, synthetic construct
  source 1..22: mol_type = other RNA
ttcttttgaacatcttctcttt

SEQ ID NO: 522 - RNA, 22 nt, synthetic construct
  source 1..22: mol_type = other RNA
tcttttgaacatcttctctttc

SEQ ID NO: 523 - RNA, 22 nt, synthetic construct
  source 1..22: mol_type = other RNA
cttttgaacatcttctctttca

SEQ ID NO: 524 - RNA, 22 nt, synthetic construct
  source 1..22: mol_type = other RNA
ttttgaacatcttctctttcat

SEQ ID NO: 525 - RNA, 22 nt, synthetic construct
  source 1..22: mol_type = other RNA
tttgaacatcttctctttcatc

SEQ ID NO: 526 - RNA, 22 nt, synthetic construct
  source 1..22: mol_type = other RNA
ttgaacatcttctctttcatct

SEQ ID NO: 527 - RNA, 22 nt, synthetic construct
  source 1..22: mol_type = other RNA
tgaacatcttctctttcatcta

SEQ ID NO: 528 - RNA, 22 nt, synthetic construct
  source 1..22: mol_type = other RNA
gaacatcttctctttcatctaa

SEQ ID NO: 529 - RNA, 22 nt, synthetic construct
  source 1..22: mol_type = other RNA
aacatcttctctttcatctaaa

SEQ ID NO: 530 - RNA, 22 nt, synthetic construct
  source 1..22: mol_type = other RNA
acatcttctctttcatctaaaa

SEQ ID NO: 531 - RNA, 22 nt, synthetic construct
  source 1..22: mol_type = other RNA
catcttctctttcatctaaaat

SEQ ID NO: 532 - RNA, 22 nt, synthetic construct
  source 1..22: mol_type = other RNA
atcttctctttcatctaaaatg

SEQ ID NO: 533 - RNA, 22 nt, synthetic construct
  source 1..22: mol_type = other RNA
tcttctctttcatctaaaatgc

SEQ ID NO: 534 - RNA, 22 nt, synthetic construct
  source 1..22: mol_type = other RNA
cttctctttcatctaaaatgca

SEQ ID NO: 535 - RNA, 22 nt, synthetic construct
  source 1..22: mol_type = other RNA
ttctctttcatctaaaatgcaa

SEQ ID NO: 536 - RNA, 22 nt, synthetic construct
  source 1..22: mol_type = other RNA
tctctttcatctaaaatgcaaa

SEQ ID NO: 537 - RNA, 22 nt, synthetic construct
  source 1..22: mol_type = other RNA
ctctttcatctaaaatgcaaaa

SEQ ID NO: 538 - RNA, 22 nt, synthetic construct
  source 1..22: mol_type = other RNA
tctttcatctaaaatgcaaaat

SEQ ID NO: 539 - RNA, 22 nt, synthetic construct
  source 1..22: mol_type = other RNA
ctttcatctaaaatgcaaaata

SEQ ID NO: 540 - RNA, 22 nt, synthetic construct
  source 1..22: mol_type = other RNA
tttcatctaaaatgcaaaataa

SEQ ID NO: 541 - RNA, 22 nt, synthetic construct
  source 1..22: mol_type = other RNA
ttcatctaaaatgcaaaataaa

SEQ ID NO: 542 - RNA, 22 nt, synthetic construct
  source 1..22: mol_type = other RNA
tcatctaaaatgcaaaataaaa

SEQ ID NO: 543 - RNA, 22 nt, synthetic construct
  source 1..22: mol_type = other RNA
catctaaaatgcaaaataaaaa

SEQ ID NO: 544 - RNA, 22 nt, synthetic construct
  source 1..22: mol_type = other RNA
atctaaaatgcaaaataaaaaa

SEQ ID NO: 545 - RNA, 22 nt, synthetic construct
  source 1..22: mol_type = other RNA
tctaaaatgcaaaataaaaaaa

SEQ ID NO: 546 - RNA, 22 nt, synthetic construct
  source 1..22: mol_type = other RNA
ctaaaatgcaaaataaaaaaat

SEQ ID NO: 547 - RNA, 22 nt, synthetic construct
  source 1..22: mol_type = other RNA
taaaatgcaaaataaaaaaata

SEQ ID NO: 548 - RNA, 22 nt, synthetic construct
  source 1..22: mol_type = other RNA
aaaatgcaaaataaaaaaataa

SEQ ID NO: 549 - RNA, 22 nt, synthetic construct
  source 1..22: mol_type = other RNA
aaatgcaaaataaaaaaataaa

SEQ ID NO: 550 - RNA, 22 nt, synthetic construct
  source 1..22: mol_type = other RNA
aatgcaaaataaaaaaataaaa

SEQ ID NO: 551 - RNA, 22 nt, synthetic construct
  source 1..22: mol_type = other RNA
atgcaaaataaaaaaataaaag

SEQ ID NO: 552 - RNA, 22 nt, synthetic construct
  source 1..22: mol_type = other RNA
tgcaaaataaaaaaataaaagt

SEQ ID NO: 553 - RNA, 22 nt, synthetic construct
  source 1..22: mol_type = other RNA
gcaaaataaaaaaataaaagtt

SEQ ID NO: 554 - RNA, 22 nt, synthetic construct
  source 1..22: mol_type = other RNA
caaaataaaaaaataaaagtta

SEQ ID NO: 555 - RNA, 22 nt, synthetic construct
  source 1..22: mol_type = other RNA
aaaataaaaaaataaaagttag

SEQ ID NO: 556 - RNA, 22 nt, synthetic construct
  source 1..22: mol_type = other RNA
aaataaaaaaataaaagttagg

SEQ ID NO: 557 - RNA, 22 nt, synthetic construct
  source 1..22: mol_type = other RNA
aataaaaaaataaaagttagga

SEQ ID NO: 558 - RNA, 22 nt, synthetic construct
  source 1..22: mol_type = other RNA
ataaaaaaataaaagttaggaa

SEQ ID NO: 559 - RNA, 22 nt, synthetic construct
  source 1..22: mol_type = other RNA
taaaaaaataaaagttaggaag

SEQ ID NO: 560 - RNA, 22 nt, synthetic construct
  source 1..22: mol_type = other RNA
aaaaaaataaaagttaggaagc

SEQ ID NO: 561 - RNA, 22 nt, synthetic construct
  source 1..22: mol_type = other RNA
aaaaaataaaagttaggaagca

SEQ ID NO: 562 - RNA, 22 nt, synthetic construct
  source 1..22: mol_type = other RNA
aaaaataaaagttaggaagcaa

SEQ ID NO: 563 - RNA, 22 nt, synthetic construct
  source 1..22: mol_type = other RNA
aaaataaaagttaggaagcaac

SEQ ID NO: 564 - RNA, 22 nt, synthetic construct
  source 1..22: mol_type = other RNA
aaataaaagttaggaagcaact

SEQ ID NO: 565 - RNA, 22 nt, synthetic construct
  source 1..22: mol_type = other RNA
aataaaagttaggaagcaactt

SEQ ID NO: 566 - RNA, 22 nt, synthetic construct
  source 1..22: mol_type = other RNA
ataaaagttaggaagcaacttt

SEQ ID NO: 567 - RNA, 22 nt, synthetic construct
  source 1..22: mol_type = other RNA
taaaagttaggaagcaacttta

SEQ ID NO: 568 - RNA, 22 nt, synthetic construct
  source 1..22: mol_type = other RNA
aaaagttaggaagcaactttaa

SEQ ID NO: 569 - RNA, 22 nt, synthetic construct
  source 1..22: mol_type = other RNA
aaagttaggaagcaactttaaa

SEQ ID NO: 570 - RNA, 22 nt, synthetic construct
  source 1..22: mol_type = other RNA
aagttaggaagcaactttaaat

SEQ ID NO: 571 - RNA, 22 nt, synthetic construct
  source 1..22: mol_type = other RNA
agttaggaagcaactttaaata

SEQ ID NO: 572 - RNA, 22 nt, synthetic construct
  source 1..22: mol_type = other RNA
gttaggaagcaactttaaatat

SEQ ID NO: 573 - RNA, 22 nt, synthetic construct
  source 1..22: mol_type = other RNA
ttaggaagcaactttaaatata

SEQ ID NO: 574 - RNA, 22 nt, synthetic construct
  source 1..22: mol_type = other RNA
taggaagcaactttaaatataa

SEQ ID NO: 575 - RNA, 22 nt, synthetic construct
  source 1..22: mol_type = other RNA
aggaagcaactttaaatataat

SEQ ID NO: 576 - RNA, 22 nt, synthetic construct
  source 1..22: mol_type = other RNA
ggaagcaactttaaatataatt

SEQ ID NO: 577 - RNA, 22 nt, synthetic construct
  source 1..22: mol_type = other RNA
gaagcaactttaaatataattc

SEQ ID NO: 578 - RNA, 22 nt, synthetic construct
  source 1..22: mol_type = other RNA
aagcaactttaaatataattca

SEQ ID NO: 579 - RNA, 22 nt, synthetic construct
  source 1..22: mol_type = other RNA
agcaactttaaatataattcat

SEQ ID NO: 580 - RNA, 22 nt, synthetic construct
  source 1..22: mol_type = other RNA
gcaactttaaatataattcata

SEQ ID NO: 581 - RNA, 22 nt, synthetic construct
  source 1..22: mol_type = other RNA
caactttaaatataattcatat

SEQ ID NO: 582 - RNA, 22 nt, synthetic construct
  source 1..22: mol_type = other RNA
aactttaaatataattcatatt

SEQ ID NO: 583 - RNA, 22 nt, synthetic construct
  source 1..22: mol_type = other RNA
actttaaatataattcatattt

SEQ ID NO: 584 - RNA, 22 nt, synthetic construct
  source 1..22: mol_type = other RNA
ctttaaatataattcatatttt

SEQ ID NO: 585 - RNA, 22 nt, synthetic construct
  source 1..22: mol_type = other RNA
tttaaatataattcatatttta

SEQ ID NO: 586 - RNA, 22 nt, synthetic construct
  source 1..22: mol_type = other RNA
ttaaatataattcatattttac

SEQ ID NO: 587 - RNA, 22 nt, synthetic construct
  source 1..22: mol_type = other RNA
taaatataattcatattttaca

SEQ ID NO: 588 - RNA, 22 nt, synthetic construct
  source 1..22: mol_type = other RNA
aaatataattcatattttacaa

SEQ ID NO: 589 - RNA, 22 nt, synthetic construct
  source 1..22: mol_type = other RNA
aatataattcatattttacaac

SEQ ID NO: 590 - RNA, 22 nt, synthetic construct
  source 1..22: mol_type = other RNA
atataattcatattttacaacc

SEQ ID NO: 591 - RNA, 22 nt, synthetic construct
  source 1..22: mol_type = other RNA
tataattcatattttacaacca

SEQ ID NO: 592 - RNA, 22 nt, synthetic construct
  source 1..22: mol_type = other RNA
ataattcatattttacaaccaa

SEQ ID NO: 593 - RNA, 22 nt, synthetic construct
  source 1..22: mol_type = other RNA
taattcatattttacaaccaaa

SEQ ID NO: 594 - RNA, 22 nt, synthetic construct
  source 1..22: mol_type = other RNA
aattcatattttacaaccaaag

SEQ ID NO: 595 - RNA, 22 nt, synthetic construct
  source 1..22: mol_type = other RNA
attcatattttacaaccaaagt

SEQ ID NO: 596 - RNA, 22 nt, synthetic construct
  source 1..22: mol_type = other RNA
ttcatattttacaaccaaagta

SEQ ID NO: 597 - RNA, 22 nt, synthetic construct
  source 1..22: mol_type = other RNA
tcatattttacaaccaaagtaa

SEQ ID NO: 598 - RNA, 22 nt, synthetic construct
  source 1..22: mol_type = other RNA
catattttacaaccaaagtaac

SEQ ID NO: 599 - RNA, 22 nt, synthetic construct
  source 1..22: mol_type = other RNA
atattttacaaccaaagtaact

SEQ ID NO: 600 - RNA, 22 nt, synthetic construct
  source 1..22: mol_type = other RNA
tattttacaaccaaagtaactt

SEQ ID NO: 601 - RNA, 22 nt, synthetic construct
  source 1..22: mol_type = other RNA
attttacaaccaaagtaacttt

SEQ ID NO: 602 - RNA, 22 nt, synthetic construct
  source 1..22: mol_type = other RNA
ttttacaaccaaagtaactttc

SEQ ID NO: 603 - RNA, 22 nt, synthetic construct
  source 1..22: mol_type = other RNA
tttacaaccaaagtaactttcc

SEQ ID NO: 604 - RNA, 22 nt, synthetic construct
  source 1..22: mol_type = other RNA
ttacaaccaaagtaactttcca

SEQ ID NO: 605 - RNA, 22 nt, synthetic construct
  source 1..22: mol_type = other RNA
tacaaccaaagtaactttccat

SEQ ID NO: 606 - RNA, 22 nt, synthetic construct
  source 1..22: mol_type = other RNA
acaaccaaagtaactttccatt

SEQ ID NO: 607 - RNA, 22 nt, synthetic construct
  source 1..22: mol_type = other RNA
caaccaaagtaactttccatta

SEQ ID NO: 608 - RNA, 22 nt, synthetic construct
  source 1..22: mol_type = other RNA
aaccaaagtaactttccattat

SEQ ID NO: 609 - RNA, 22 nt, synthetic construct
  source 1..22: mol_type = other RNA
accaaagtaactttccattatg

SEQ ID NO: 610 - RNA, 22 nt, synthetic construct
  source 1..22: mol_type = other RNA
ccaaagtaactttccattatga

SEQ ID NO: 611 - RNA, 22 nt, synthetic construct
  source 1..22: mol_type = other RNA
caaagtaactttccattatgat

SEQ ID NO: 612 - RNA, 22 nt, synthetic construct
  source 1..22: mol_type = other RNA
aaagtaactttccattatgatg

SEQ ID NO: 613 - RNA, 22 nt, synthetic construct
  source 1..22: mol_type = other RNA
aagtaactttccattatgatgt

SEQ ID NO: 614 - RNA, 22 nt, synthetic construct
  source 1..22: mol_type = other RNA
agtaactttccattatgatgtg

SEQ ID NO: 615 - RNA, 22 nt, synthetic construct
  source 1..22: mol_type = other RNA
gtaactttccattatgatgtgt

SEQ ID NO: 616 - RNA, 22 nt, synthetic construct
  source 1..22: mol_type = other RNA
taactttccattatgatgtgtt

SEQ ID NO: 617 - RNA, 22 nt, synthetic construct
  source 1..22: mol_type = other RNA
aactttccattatgatgtgtta

SEQ ID NO: 618 - RNA, 22 nt, synthetic construct
  source 1..22: mol_type = other RNA
actttccattatgatgtgttag

SEQ ID NO: 619 - RNA, 22 nt, synthetic construct
  source 1..22: mol_type = other RNA
ctttccattatgatgtgttagt

SEQ ID NO: 620 - RNA, 22 nt, synthetic construct
  source 1..22: mol_type = other RNA
tttccattatgatgtgttagtg

SEQ ID NO: 621 - RNA, 22 nt, synthetic construct
  source 1..22: mol_type = other RNA
ttccattatgatgtgttagtgt

SEQ ID NO: 622 - RNA, 22 nt, synthetic construct
  source 1..22: mol_type = other RNA
tccattatgatgtgttagtgtt

SEQ ID NO: 623 - RNA, 22 nt, synthetic construct
  source 1..22: mol_type = other RNA
ccattatgatgtgttagtgttt

SEQ ID NO: 624 - RNA, 22 nt, synthetic construct
  source 1..22: mol_type = other RNA
cattatgatgtgttagtgtttt

SEQ ID NO: 625 - RNA, 22 nt, synthetic construct
  source 1..22: mol_type = other RNA
attatgatgtgttagtgttttt

SEQ ID NO: 626 - RNA, 22 nt, synthetic construct
  source 1..22: mol_type = other RNA
ttatgatgtgttagtgtttttt

SEQ ID NO: 627 - RNA, 22 nt, synthetic construct
  source 1..22: mol_type = other RNA
tatgatgtgttagtgttttttt

SEQ ID NO: 628 - RNA, 22 nt, synthetic construct
  source 1..22: mol_type = other RNA
atgatgtgttagtgttttttta

SEQ ID NO: 629 - RNA, 22 nt, synthetic construct
  source 1..22: mol_type = other RNA
tgatgtgttagtgtttttttac

SEQ ID NO: 630 - RNA, 22 nt, synthetic construct
  source 1..22: mol_type = other RNA
gatgtgttagtgtttttttaca

SEQ ID NO: 631 - RNA, 22 nt, synthetic construct
  source 1..22: mol_type = other RNA
atgtgttagtgtttttttacat

SEQ ID NO: 632 - RNA, 22 nt, synthetic construct
  source 1..22: mol_type = other RNA
tgtgttagtgtttttttacatt

SEQ ID NO: 633 - RNA, 22 nt, synthetic construct
  source 1..22: mol_type = other RNA
gtgttagtgtttttttacattt

SEQ ID NO: 634 - RNA, 22 nt, synthetic construct
  source 1..22: mol_type = other RNA
tgttagtgtttttttacattta

SEQ ID NO: 635 - RNA, 22 nt, synthetic construct
  source 1..22: mol_type = other RNA
gttagtgtttttttacatttag

SEQ ID NO: 636 - RNA, 22 nt, synthetic construct
  source 1..22: mol_type = other RNA
ttagtgtttttttacatttagt

SEQ ID NO: 637 - RNA, 22 nt, synthetic construct
  source 1..22: mol_type = other RNA
tagtgtttttttacatttagta

SEQ ID NO: 638 - RNA, 22 nt, synthetic construct
  source 1..22: mol_type = other RNA
agtgtttttttacatttagtat

SEQ ID NO: 639 - RNA, 22 nt, synthetic construct
  source 1..22: mol_type = other RNA
gtgtttttttacatttagtatt

SEQ ID NO: 640 - RNA, 22 nt, synthetic construct
  source 1..22: mol_type = other RNA
tgtttttttacatttagtatta

SEQ ID NO: 641 - RNA, 22 nt, synthetic construct
  source 1..22: mol_type = other RNA
gtttttttacatttagtattac

SEQ ID NO: 642 - RNA, 22 nt, synthetic construct
  source 1..22: mol_type = other RNA
tttttttacatttagtattaca

SEQ ID NO: 643 - RNA, 22 nt, synthetic construct
  source 1..22: mol_type = other RNA
ttttttacatttagtattacat

SEQ ID NO: 644 - RNA, 22 nt, synthetic construct
  source 1..22: mol_type = other RNA
tttttacatttagtattacatt

SEQ ID NO: 645 - RNA, 22 nt, synthetic construct
  source 1..22: mol_type = other RNA
ttttacatttagtattacattc

SEQ ID NO: 646 - RNA, 22 nt, synthetic construct
  source 1..22: mol_type = other RNA
tttacatttagtattacattca

SEQ ID NO: 647 - RNA, 22 nt, synthetic construct
  source 1..22: mol_type = other RNA
ttacatttagtattacattcat

SEQ ID NO: 648 - RNA, 22 nt, synthetic construct
  source 1..22: mol_type = other RNA
tacatttagtattacattcatt

SEQ ID NO: 649 - RNA, 22 nt, synthetic construct
  source 1..22: mol_type = other RNA
acatttagtattacattcattg

SEQ ID NO: 650 - RNA, 22 nt, synthetic construct
  source 1..22: mol_type = other RNA
catttagtattacattcattga

SEQ ID NO: 651 - RNA, 22 nt, synthetic construct
  source 1..22: mol_type = other RNA
atttagtattacattcattgat

SEQ ID NO: 652 - RNA, 22 nt, synthetic construct
  source 1..22: mol_type = other RNA
tttagtattacattcattgata

SEQ ID NO: 653 - RNA, 22 nt, synthetic construct
  source 1..22: mol_type = other RNA
ttagtattacattcattgataa

SEQ ID NO: 654 - RNA, 22 nt, synthetic construct
  source 1..22: mol_type = other RNA
tagtattacattcattgataaa

SEQ ID NO: 655 - RNA, 22 nt, synthetic construct
  source 1..22: mol_type = other RNA
agtattacattcattgataaat

SEQ ID NO: 656 - RNA, 22 nt, synthetic construct
  source 1..22: mol_type = other RNA
gtattacattcattgataaatg

SEQ ID NO: 657 - RNA, 22 nt, synthetic construct
  source 1..22: mol_type = other RNA
tattacattcattgataaatgg

SEQ ID NO: 658 - RNA, 22 nt, synthetic construct
  source 1..22: mol_type = other RNA
attacattcattgataaatgga

SEQ ID NO: 659 - RNA, 22 nt, synthetic construct
  source 1..22: mol_type = other RNA
ttacattcattgataaatggaa

SEQ ID NO: 660 - RNA, 22 nt, synthetic construct
  source 1..22: mol_type = other RNA
tacattcattgataaatggaat

SEQ ID NO: 661 - RNA, 22 nt, synthetic construct
  source 1..22: mol_type = other RNA
acattcattgataaatggaatt

SEQ ID NO: 662 - RNA, 22 nt, synthetic construct
  source 1..22: mol_type = other RNA
cattcattgataaatggaatta

SEQ ID NO: 663 - RNA, 22 nt, synthetic construct
  source 1..22: mol_type = other RNA
attcattgataaatggaattaa

SEQ ID NO: 664 - RNA, 22 nt, synthetic construct
  source 1..22: mol_type = other RNA
ttcattgataaatggaattaaa

SEQ ID NO: 665 - RNA, 22 nt, synthetic construct
  source 1..22: mol_type = other RNA
tcattgataaatggaattaaac

SEQ ID NO: 666 - RNA, 22 nt, synthetic construct
  source 1..22: mol_type = other RNA
cattgataaatggaattaaaca

SEQ ID NO: 667 - RNA, 22 nt, synthetic construct
  source 1..22: mol_type = other RNA
attgataaatggaattaaacat

SEQ ID NO: 668 - RNA, 22 nt, synthetic construct
  source 1..22: mol_type = other RNA
ttgataaatggaattaaacatg

SEQ ID NO: 669 - RNA, 22 nt, synthetic construct
  source 1..22: mol_type = other RNA
tgataaatggaattaaacatga

SEQ ID NO: 670 - RNA, 22 nt, synthetic construct
  source 1..22: mol_type = other RNA
gataaatggaattaaacatgag

SEQ ID NO: 671 - RNA, 22 nt, synthetic construct
  source 1..22: mol_type = other RNA
ataaatggaattaaacatgagt

SEQ ID NO: 672 - RNA, 22 nt, synthetic construct
  source 1..22: mol_type = other RNA
taaatggaattaaacatgagtg

SEQ ID NO: 673 - RNA, 22 nt, synthetic construct
  source 1..22: mol_type = other RNA
aaatggaattaaacatgagtga

SEQ ID NO: 674 - RNA, 22 nt, synthetic construct
  source 1..22: mol_type = other RNA
aatggaattaaacatgagtgat

SEQ ID NO: 675 - RNA, 26 nt, synthetic construct
  source 1..26: mol_type = other RNA
gagtgaacataatatttccagatttg

SEQ ID NO: 676 - RNA, 26 nt, synthetic construct
  source 1..26: mol_type = other RNA
agtgaacataatatttccagatttgc

SEQ ID NO: 677 - RNA, 26 nt, synthetic construct
  source 1..26: mol_type = other RNA
gtgaacataatatttccagatttgca

SEQ ID NO: 678 - RNA, 26 nt, synthetic construct
  source 1..26: mol_type = other RNA
tgaacataatatttccagatttgcac

SEQ ID NO: 679 - RNA, 26 nt, synthetic construct
  source 1..26: mol_type = other RNA
gaacataatatttccagatttgcaca

SEQ ID NO: 680 - RNA, 26 nt, synthetic construct
  source 1..26: mol_type = other RNA
aacataatatttccagatttgcacag

SEQ ID NO: 681 - RNA, 26 nt, synthetic construct
  source 1..26: mol_type = other RNA
acataatatttccagatttgcacagc

SEQ ID NO: 682 - RNA, 26 nt, synthetic construct
  source 1..26: mol_type = other RNA
cataatatttccagatttgcacagct

SEQ ID NO: 683 - RNA, 26 nt, synthetic construct
  source 1..26: mol_type = other RNA
ataatatttccagatttgcacagcta

SEQ ID NO: 684 - RNA, 26 nt, synthetic construct
  source 1..26: mol_type = other RNA
taatatttccagatttgcacagctaa

SEQ ID NO: 685 - RNA, 26 nt, synthetic construct
  source 1..26: mol_type = other RNA
aatatttccagatttgcacagctaaa

SEQ ID NO: 686 - RNA, 26 nt, synthetic construct
  source 1..26: mol_type = other RNA
atatttccagatttgcacagctaaaa

SEQ ID NO: 687 - RNA, 26 nt, synthetic construct
  source 1..26: mol_type = other RNA
tatttccagatttgcacagctaaaat

SEQ ID NO: 688 - RNA, 26 nt, synthetic construct
  source 1..26: mol_type = other RNA
atttccagatttgcacagctaaaata

SEQ ID NO: 689 - RNA, 26 nt, synthetic construct
  source 1..26: mol_type = other RNA
tttccagatttgcacagctaaaataa

SEQ ID NO: 690 - RNA, 26 nt, synthetic construct
  source 1..26: mol_type = other RNA
ttccagatttgcacagctaaaataaa

SEQ ID NO: 691 - RNA, 26 nt, synthetic construct
  source 1..26: mol_type = other RNA
tccagatttgcacagctaaaataaaa

SEQ ID NO: 692 - RNA, 26 nt, synthetic construct
  source 1..26: mol_type = other RNA
ccagatttgcacagctaaaataaaat

SEQ ID NO: 693 - RNA, 26 nt, synthetic construct
  source 1..26: mol_type = other RNA
cagatttgcacagctaaaataaaatg

SEQ ID NO: 694 - RNA, 26 nt, synthetic construct
  source 1..26: mol_type = other RNA
agatttgcacagctaaaataaaatga

SEQ ID NO: 695 - RNA, 26 nt, synthetic construct
  source 1..26: mol_type = other RNA
gatttgcacagctaaaataaaatgac

SEQ ID NO: 696 - RNA, 26 nt, synthetic construct
  source 1..26: mol_type = other RNA
atttgcacagctaaaataaaatgaca

SEQ ID NO: 697 - RNA, 26 nt, synthetic construct
  source 1..26: mol_type = other RNA
tttgcacagctaaaataaaatgacac

SEQ ID NO: 698 - RNA, 26 nt, synthetic construct
  source 1..26: mol_type = other RNA
ttgcacagctaaaataaaatgacact

SEQ ID NO: 699 - RNA, 26 nt, synthetic construct
  source 1..26: mol_type = other RNA
tgcacagctaaaataaaatgacacta

SEQ ID NO: 700 - RNA, 26 nt, synthetic construct
  source 1..26: mol_type = other RNA
gcacagctaaaataaaatgacactat

SEQ ID NO: 701 - RNA, 26 nt, synthetic construct
  source 1..26: mol_type = other RNA
cacagctaaaataaaatgacactatg

SEQ ID NO: 702 - RNA, 26 nt, synthetic construct
  source 1..26: mol_type = other RNA
acagctaaaataaaatgacactatga

SEQ ID NO: 703 - RNA, 26 nt, synthetic construct
  source 1..26: mol_type = other RNA
cagctaaaataaaatgacactatgag

SEQ ID NO: 704 - RNA, 26 nt, synthetic construct
  source 1..26: mol_type = other RNA
agctaaaataaaatgacactatgaga

SEQ ID NO: 705 - RNA, 26 nt, synthetic construct
  source 1..26: mol_type = other RNA
gctaaaataaaatgacactatgagag

SEQ ID NO: 706 - RNA, 26 nt, synthetic construct
  source 1..26: mol_type = other RNA
ctaaaataaaatgacactatgagaga

SEQ ID NO: 707 - RNA, 26 nt, synthetic construct
  source 1..26: mol_type = other RNA
taaaataaaatgacactatgagagaa

SEQ ID NO: 708 - RNA, 26 nt, synthetic construct
  source 1..26: mol_type = other RNA
aaaataaaatgacactatgagagaaa

SEQ ID NO: 709 - RNA, 26 nt, synthetic construct
  source 1..26: mol_type = other RNA
aaataaaatgacactatgagagaaat

SEQ ID NO: 710 - RNA, 26 nt, synthetic construct
  source 1..26: mol_type = other RNA
aataaaatgacactatgagagaaata

SEQ ID NO: 711 - RNA, 26 nt, synthetic construct
  source 1..26: mol_type = other RNA
ataaaatgacactatgagagaaataa

SEQ ID NO: 712 - RNA, 26 nt, synthetic construct
  source 1..26: mol_type = other RNA
taaaatgacactatgagagaaataaa

SEQ ID NO: 713 - RNA, 26 nt, synthetic construct
  source 1..26: mol_type = other RNA
aaaatgacactatgagagaaataaaa

SEQ ID NO: 714 - RNA, 26 nt, synthetic construct
  source 1..26: mol_type = other RNA
aaatgacactatgagagaaataaaac

SEQ ID NO: 715 - RNA, 26 nt, synthetic construct
  source 1..26: mol_type = other RNA
aatgacactatgagagaaataaaacg

SEQ ID NO: 716 - RNA, 26 nt, synthetic construct
  source 1..26: mol_type = other RNA
atgacactatgagagaaataaaacgg

SEQ ID NO: 717 - RNA, 26 nt, synthetic construct
  source 1..26: mol_type = other RNA
tgacactatgagagaaataaaacgga

SEQ ID NO: 718 - RNA, 26 nt, synthetic construct
  source 1..26: mol_type = other RNA
gacactatgagagaaataaaacggat

SEQ ID NO: 719 - RNA, 26 nt, synthetic construct
  source 1..26: mol_type = other RNA
acactatgagagaaataaaacggatt

SEQ ID NO: 720 - RNA, 26 nt, synthetic construct
  source 1..26: mol_type = other RNA
cactatgagagaaataaaacggattt

SEQ ID NO: 721 - RNA, 26 nt, synthetic construct
  source 1..26: mol_type = other RNA
actatgagagaaataaaacggatttt

SEQ ID NO: 722 - RNA, 26 nt, synthetic construct
  source 1..26: mol_type = other RNA
ctatgagagaaataaaacggattttt

SEQ ID NO: 723 - RNA, 26 nt, synthetic construct
  source 1..26: mol_type = other RNA
tatgagagaaataaaacggattttta

SEQ ID NO: 724 - RNA, 26 nt, synthetic construct
  source 1..26: mol_type = other RNA
atgagagaaataaaacggatttttaa

SEQ ID NO: 725 - RNA, 26 nt, synthetic construct
  source 1..26: mol_type = other RNA
tgagagaaataaaacggatttttaag

SEQ ID NO: 726 - RNA, 26 nt, synthetic construct
  source 1..26: mol_type = other RNA
gagagaaataaaacggatttttaaga

SEQ ID NO: 727 - RNA, 26 nt, synthetic construct
  source 1..26: mol_type = other RNA
agagaaataaaacggatttttaagat

SEQ ID NO: 728 - RNA, 26 nt, synthetic construct
  source 1..26: mol_type = other RNA
gagaaataaaacggatttttaagata

SEQ ID NO: 729 - RNA, 26 nt, synthetic construct
  source 1..26: mol_type = other RNA
agaaataaaacggatttttaagatac

SEQ ID NO: 730 - RNA, 26 nt, synthetic construct
  source 1..26: mol_type = other RNA
gaaataaaacggatttttaagataca

SEQ ID NO: 731 - RNA, 26 nt, synthetic construct
  source 1..26: mol_type = other RNA
aaataaaacggatttttaagatacac

SEQ ID NO: 732 - RNA, 26 nt, synthetic construct
  source 1..26: mol_type = other RNA
aataaaacggatttttaagatacaca

SEQ ID NO: 733 - RNA, 26 nt, synthetic construct
  source 1..26: mol_type = other RNA
ataaaacggatttttaagatacacag

SEQ ID NO: 734 - RNA, 26 nt, synthetic construct
  source 1..26: mol_type = other RNA
taaaacggatttttaagatacacagg

SEQ ID NO: 735 - RNA, 26 nt, synthetic construct
  source 1..26: mol_type = other RNA
aaaacggatttttaagatacacaggt

SEQ ID NO: 736 - RNA, 26 nt, synthetic construct
  source 1..26: mol_type = other RNA
aaacggatttttaagatacacaggta

SEQ ID NO: 737 - RNA, 26 nt, synthetic construct
  source 1..26: mol_type = other RNA
aacggatttttaagatacacaggtac

SEQ ID NO: 738 - RNA, 26 nt, synthetic construct
  source 1..26: mol_type = other RNA
acggatttttaagatacacaggtaca

SEQ ID NO: 739 - RNA, 26 nt, synthetic construct
  source 1..26: mol_type = other RNA
cggatttttaagatacacaggtacat

SEQ ID NO: 740 - RNA, 26 nt, synthetic construct
  source 1..26: mol_type = other RNA
ggatttttaagatacacaggtacata

SEQ ID NO: 741 - RNA, 26 nt, synthetic construct
  source 1..26: mol_type = other RNA
gatttttaagatacacaggtacatag

SEQ ID NO: 742 - RNA, 26 nt, synthetic construct
  source 1..26: mol_type = other RNA
atttttaagatacacaggtacatagt

SEQ ID NO: 743 - RNA, 26 nt, synthetic construct
  source 1..26: mol_type = other RNA
tttttaagatacacaggtacatagtc

SEQ ID NO: 744 - RNA, 26 nt, synthetic construct
  source 1..26: mol_type = other RNA
ttttaagatacacaggtacatagtcc

SEQ ID NO: 745 - RNA, 26 nt, synthetic construct
  source 1..26: mol_type = other RNA
tttaagatacacaggtacatagtcca

SEQ ID NO: 746 - RNA, 26 nt, synthetic construct
  source 1..26: mol_type = other RNA
ttaagatacacaggtacatagtccat

SEQ ID NO: 747 - RNA, 26 nt, synthetic construct
  source 1..26: mol_type = other RNA
taagatacacaggtacatagtccatt

SEQ ID NO: 748 - RNA, 26 nt, synthetic construct
  source 1..26: mol_type = other RNA
aagatacacaggtacatagtccattt

SEQ ID NO: 749 - RNA, 26 nt, synthetic construct
  source 1..26: mol_type = other RNA
agatacacaggtacatagtccatttt

SEQ ID NO: 750 - RNA, 26 nt, synthetic construct
  source 1..26: mol_type = other RNA
gatacacaggtacatagtccattttg

SEQ ID NO: 751 - RNA, 26 nt, synthetic construct
  source 1..26: mol_type = other RNA
atacacaggtacatagtccattttga

SEQ ID NO: 752 - RNA, 26 nt, synthetic construct
  source 1..26: mol_type = other RNA
tacacaggtacatagtccattttgaa

SEQ ID NO: 753 - RNA, 26 nt, synthetic construct
  source 1..26: mol_type = other RNA
acacaggtacatagtccattttgaaa

SEQ ID NO: 754 - RNA, 26 nt, synthetic construct
  source 1..26: mol_type = other RNA
cacaggtacatagtccattttgaaaa

SEQ ID NO: 755 - RNA, 26 nt, synthetic construct
  source 1..26: mol_type = other RNA
acaggtacatagtccattttgaaaat

SEQ ID NO: 756 - RNA, 26 nt, synthetic construct
  source 1..26: mol_type = other RNA
caggtacatagtccattttgaaaatt

SEQ ID NO: 757 - RNA, 26 nt, synthetic construct
  source 1..26: mol_type = other RNA
aggtacatagtccattttgaaaattt

SEQ ID NO: 758 - RNA, 26 nt, synthetic construct
  source 1..26: mol_type = other RNA
ggtacatagtccattttgaaaatttc

SEQ ID NO: 759 - RNA, 26 nt, synthetic construct
  source 1..26: mol_type = other RNA
gtacatagtccattttgaaaatttca

SEQ ID NO: 760 - RNA, 26 nt, synthetic construct
  source 1..26: mol_type = other RNA
tacatagtccattttgaaaatttcac

SEQ ID NO: 761 - RNA, 26 nt, synthetic construct
  source 1..26: mol_type = other RNA
acatagtccattttgaaaatttcaca

SEQ ID NO: 762 - RNA, 26 nt, synthetic construct
  source 1..26: mol_type = other RNA
catagtccattttgaaaatttcacaa

SEQ ID NO: 763 - RNA, 26 nt, synthetic construct
  source 1..26: mol_type = other RNA
atagtccattttgaaaatttcacaac

SEQ ID NO: 764 - RNA, 26 nt, synthetic construct
  source 1..26: mol_type = other RNA
tagtccattttgaaaatttcacaact

SEQ ID NO: 765 - RNA, 26 nt, synthetic construct
  source 1..26: mol_type = other RNA
agtccattttgaaaatttcacaactt

SEQ ID NO: 766 - RNA, 26 nt, synthetic construct
  source 1..26: mol_type = other RNA
gtccattttgaaaatttcacaactta

SEQ ID NO: 767 - RNA, 26 nt, synthetic construct
  source 1..26: mol_type = other RNA
tccattttgaaaatttcacaacttag

SEQ ID NO: 768 - RNA, 26 nt, synthetic construct
  source 1..26: mol_type = other RNA
ccattttgaaaatttcacaacttaga

SEQ ID NO: 769 - RNA, 26 nt, synthetic construct
  source 1..26: mol_type = other RNA
cattttgaaaatttcacaacttagat

SEQ ID NO: 770 - RNA, 26 nt, synthetic construct
  source 1..26: mol_type = other RNA
attttgaaaatttcacaacttagatc

SEQ ID NO: 771 - RNA, 26 nt, synthetic construct
  source 1..26: mol_type = other RNA
ttttgaaaatttcacaacttagatct

SEQ ID NO: 772 - RNA, 26 nt, synthetic construct
  source 1..26: mol_type = other RNA
tttgaaaatttcacaacttagatctt

SEQ ID NO: 773 - RNA, 26 nt, synthetic construct
  source 1..26: mol_type = other RNA
ttgaaaatttcacaacttagatctta

SEQ ID NO: 774 - RNA, 26 nt, synthetic construct
  source 1..26: mol_type = other RNA
tgaaaatttcacaacttagatcttaa

SEQ ID NO: 775 - RNA, 26 nt, synthetic construct
  source 1..26: mol_type = other RNA
gaaaatttcacaacttagatcttaaa

SEQ ID NO: 776 - RNA, 26 nt, synthetic construct
  source 1..26: mol_type = other RNA
aaaatttcacaacttagatcttaaaa

SEQ ID NO: 777 - RNA, 26 nt, synthetic construct
  source 1..26: mol_type = other RNA
aaatttcacaacttagatcttaaaag

SEQ ID NO: 778 - RNA, 26 nt, synthetic construct
  source 1..26: mol_type = other RNA
aatttcacaacttagatcttaaaagt

SEQ ID NO: 779 - RNA, 26 nt, synthetic construct
  source 1..26: mol_type = other RNA
atttcacaacttagatcttaaaagta

SEQ ID NO: 780 - RNA, 26 nt, synthetic construct
  source 1..26: mol_type = other RNA
tttcacaacttagatcttaaaagtaa

SEQ ID NO: 781 - RNA, 26 nt, synthetic construct
  source 1..26: mol_type = other RNA
ttcacaacttagatcttaaaagtaaa

SEQ ID NO: 782 - RNA, 26 nt, synthetic construct
  source 1..26: mol_type = other RNA
tcacaacttagatcttaaaagtaaag

SEQ ID NO: 783 - RNA, 26 nt, synthetic construct
  source 1..26: mol_type = other RNA
cacaacttagatcttaaaagtaaagt

SEQ ID NO: 784 - RNA, 26 nt, synthetic construct
  source 1..26: mol_type = other RNA
acaacttagatcttaaaagtaaagta

SEQ ID NO: 785 - RNA, 26 nt, synthetic construct
  source 1..26: mol_type = other RNA
caacttagatcttaaaagtaaagtaa

SEQ ID NO: 786 - RNA, 26 nt, synthetic construct
  source 1..26: mol_type = other RNA
aacttagatcttaaaagtaaagtaac

SEQ ID NO: 787 - RNA, 26 nt, synthetic construct
  source 1..26: mol_type = other RNA
acttagatcttaaaagtaaagtaaca

SEQ ID NO: 788 - RNA, 26 nt, synthetic construct
  source 1..26: mol_type = other RNA
cttagatcttaaaagtaaagtaacaa

SEQ ID NO: 789 - RNA, 26 nt, synthetic construct
  source 1..26: mol_type = other RNA
ttagatcttaaaagtaaagtaacaaa

SEQ ID NO: 790 - RNA, 26 nt, synthetic construct
  source 1..26: mol_type = other RNA
tagatcttaaaagtaaagtaacaaac

SEQ ID NO: 791 - RNA, 26 nt, synthetic construct
  source 1..26: mol_type = other RNA
agatcttaaaagtaaagtaacaaacc

SEQ ID NO: 792 - RNA, 26 nt, synthetic construct
  source 1..26: mol_type = other RNA
gatcttaaaagtaaagtaacaaacca

SEQ ID NO: 793 - RNA, 26 nt, synthetic construct
  source 1..26: mol_type = other RNA
atcttaaaagtaaagtaacaaaccat

SEQ ID NO: 794 - RNA, 26 nt, synthetic construct
  source 1..26: mol_type = other RNA
tcttaaaagtaaagtaacaaaccatt

SEQ ID NO: 795 - RNA, 26 nt, synthetic construct
  source 1..26: mol_type = other RNA
cttaaaagtaaagtaacaaaccattc

SEQ ID NO: 796 - RNA, 26 nt, synthetic construct
  source 1..26: mol_type = other RNA
ttaaaagtaaagtaacaaaccattct

SEQ ID NO: 797 - RNA, 26 nt, synthetic construct
  source 1..26: mol_type = other RNA
taaaagtaaagtaacaaaccattctt

SEQ ID NO: 798 - RNA, 26 nt, synthetic construct
  source 1..26: mol_type = other RNA
aaaagtaaagtaacaaaccattctta

SEQ ID NO: 799 - RNA, 26 nt, synthetic construct
  source 1..26: mol_type = other RNA
aaagtaaagtaacaaaccattcttac

SEQ ID NO: 800 - RNA, 26 nt, synthetic construct
  source 1..26: mol_type = other RNA
aagtaaagtaacaaaccattcttacc

SEQ ID NO: 801 - RNA, 26 nt, synthetic construct
  source 1..26: mol_type = other RNA
agtaaagtaacaaaccattcttacct

SEQ ID NO: 802 - RNA, 26 nt, synthetic construct
  source 1..26: mol_type = other RNA
gtaaagtaacaaaccattcttacctt

SEQ ID NO: 803 - RNA, 26 nt, synthetic construct
  source 1..26: mol_type = other RNA
taaagtaacaaaccattcttacctta

SEQ ID NO: 804 - RNA, 26 nt, synthetic construct
  source 1..26: mol_type = other RNA
aaagtaacaaaccattcttaccttag

SEQ ID NO: 805 - RNA, 26 nt, synthetic construct
  source 1..26: mol_type = other RNA
aagtaacaaaccattcttaccttaga

SEQ ID NO: 806 - RNA, 26 nt, synthetic construct
  source 1..26: mol_type = other RNA
agtaacaaaccattcttaccttagaa

SEQ ID NO: 807 - RNA, 26 nt, synthetic construct
  source 1..26: mol_type = other RNA
gtaacaaaccattcttaccttagaaa

SEQ ID NO: 808 - RNA, 26 nt, synthetic construct
  source 1..26: mol_type = other RNA
taacaaaccattcttaccttagaaaa

SEQ ID NO: 809 - RNA, 26 nt, synthetic construct
  source 1..26: mol_type = other RNA
aacaaaccattcttaccttagaaaat

SEQ ID NO: 810 - RNA, 26 nt, synthetic construct
  source 1..26: mol_type = other RNA
acaaaccattcttaccttagaaaatt

SEQ ID NO: 811 - RNA, 26 nt, synthetic construct
  source 1..26: mol_type = other RNA
caaaccattcttaccttagaaaattg

SEQ ID NO: 812 - RNA, 26 nt, synthetic construct
  source 1..26: mol_type = other RNA
aaaccattcttaccttagaaaattgt

SEQ ID NO: 813 - RNA, 26 nt, synthetic construct
  source 1..26: mol_type = other RNA
aaccattcttaccttagaaaattgtg

SEQ ID NO: 814 - RNA, 26 nt, synthetic construct
  source 1..26: mol_type = other RNA
accattcttaccttagaaaattgtgc

SEQ ID NO: 815 - RNA, 26 nt, synthetic construct
  source 1..26: mol_type = other RNA
ccattcttaccttagaaaattgtgca

SEQ ID NO: 816 - RNA, 26 nt, synthetic construct
  source 1..26: mol_type = other RNA
cattcttaccttagaaaattgtgcat

SEQ ID NO: 817 - RNA, 26 nt, synthetic construct
  source 1..26: mol_type = other RNA
attcttaccttagaaaattgtgcatt

SEQ ID NO: 818 - RNA, 26 nt, synthetic construct
  source 1..26: mol_type = other RNA
ttcttaccttagaaaattgtgcattt

SEQ ID NO: 819 - RNA, 26 nt, synthetic construct
  source 1..26: mol_type = other RNA
tcttaccttagaaaattgtgcattta

SEQ ID NO: 820 - RNA, 26 nt, synthetic construct
  source 1..26: mol_type = other RNA
cttaccttagaaaattgtgcatttac

SEQ ID NO: 821 - RNA, 26 nt, synthetic construct
  source 1..26: mol_type = other RNA
ttaccttagaaaattgtgcatttacc

SEQ ID NO: 822 - RNA, 26 nt, synthetic construct
  source 1..26: mol_type = other RNA
taccttagaaaattgtgcatttaccc

SEQ ID NO: 823 - RNA, 26 nt, synthetic construct
  source 1..26: mol_type = other RNA
accttagaaaattgtgcatttaccca

SEQ ID NO: 824 - RNA, 26 nt, synthetic construct
  source 1..26: mol_type = other RNA
ccttagaaaattgtgcatttacccat

SEQ ID NO: 825 - RNA, 26 nt, synthetic construct
  source 1..26: mol_type = other RNA
cttagaaaattgtgcatttacccatt

SEQ ID NO: 826 - RNA, 26 nt, synthetic construct
  source 1..26: mol_type = other RNA
ttagaaaattgtgcatttacccattt

SEQ ID NO: 827 - RNA, 26 nt, synthetic construct
  source 1..26: mol_type = other RNA
tagaaaattgtgcatttacccatttt

SEQ ID NO: 828 - RNA, 26 nt, synthetic construct
  source 1..26: mol_type = other RNA
agaaaattgtgcatttacccattttg

SEQ ID NO: 829 - RNA, 26 nt, synthetic construct
  source 1..26: mol_type = other RNA
gaaaattgtgcatttacccattttgt

SEQ ID NO: 830 - RNA, 26 nt, synthetic construct
  source 1..26: mol_type = other RNA
aaaattgtgcatttacccattttgtg

SEQ ID NO: 831 - RNA, 26 nt, synthetic construct
  source 1..26: mol_type = other RNA
aaattgtgcatttacccattttgtga

SEQ ID NO: 832 - RNA, 26 nt, synthetic construct
  source 1..26: mol_type = other RNA
aattgtgcatttacccattttgtgaa

SEQ ID NO: 833 - RNA, 26 nt, synthetic construct
  source 1..26: mol_type = other RNA
attgtgcatttacccattttgtgaat

SEQ ID NO: 834 - RNA, 26 nt, synthetic construct
  source 1..26: mol_type = other RNA
ttgtgcatttacccattttgtgaatg

SEQ ID NO: 835 - RNA, 26 nt, synthetic construct
  source 1..26: mol_type = other RNA
tgtgcatttacccattttgtgaatgt

SEQ ID NO: 836 - RNA, 26 nt, synthetic construct
  source 1..26: mol_type = other RNA
gtgcatttacccattttgtgaatgtt

SEQ ID NO: 837 - RNA, 26 nt, synthetic construct
  source 1..26: mol_type = other RNA
tgcatttacccattttgtgaatgttt

SEQ ID NO: 838 - RNA, 26 nt, synthetic construct
  source 1..26: mol_type = other RNA
gcatttacccattttgtgaatgtttt

SEQ ID NO: 839 - RNA, 26 nt, synthetic construct
  source 1..26: mol_type = other RNA
catttacccattttgtgaatgttttc

SEQ ID NO: 840 - RNA, 26 nt, synthetic construct
  source 1..26: mol_type = other RNA
atttacccattttgtgaatgttttct

SEQ ID NO: 841 - RNA, 26 nt, synthetic construct
  source 1..26: mol_type = other RNA
tttacccattttgtgaatgttttctt

SEQ ID NO: 842 - RNA, 26 nt, synthetic construct
  source 1..26: mol_type = other RNA
ttacccattttgtgaatgttttcttt

SEQ ID NO: 843 - RNA, 26 nt, synthetic construct
  source 1..26: mol_type = other RNA
tacccattttgtgaatgttttctttt

SEQ ID NO: 844 - RNA, 26 nt, synthetic construct
  source 1..26: mol_type = other RNA
acccattttgtgaatgttttcttttg

SEQ ID NO: 845 - RNA, 26 nt, synthetic construct
  source 1..26: mol_type = other RNA
cccattttgtgaatgttttcttttga

SEQ ID NO: 846 - RNA, 26 nt, synthetic construct
  source 1..26: mol_type = other RNA
ccattttgtgaatgttttcttttgaa

SEQ ID NO: 847 - RNA, 26 nt, synthetic construct
  source 1..26: mol_type = other RNA
cattttgtgaatgttttcttttgaac

SEQ ID NO: 848 - RNA, 26 nt, synthetic construct
  source 1..26: mol_type = other RNA
attttgtgaatgttttcttttgaaca

SEQ ID NO: 849 - RNA, 26 nt, synthetic construct
  source 1..26: mol_type = other RNA
ttttgtgaatgttttcttttgaacat

SEQ ID NO: 850 - RNA, 26 nt, synthetic construct
  source 1..26: mol_type = other RNA
tttgtgaatgttttcttttgaacatc

SEQ ID NO: 851 - RNA, 26 nt, synthetic construct
  source 1..26: mol_type = other RNA
ttgtgaatgttttcttttgaacatct

SEQ ID NO: 852 - RNA, 26 nt, synthetic construct
  source 1..26: mol_type = other RNA
tgtgaatgttttcttttgaacatctt

SEQ ID NO: 853 - RNA, 26 nt, synthetic construct
  source 1..26: mol_type = other RNA
gtgaatgttttcttttgaacatcttc

SEQ ID NO: 854 - RNA, 26 nt, synthetic construct
  source 1..26: mol_type = other RNA
tgaatgttttcttttgaacatcttct

SEQ ID NO: 855 - RNA, 26 nt, synthetic construct
  source 1..26: mol_type = other RNA
gaatgttttcttttgaacatcttctc

SEQ ID NO: 856 - RNA, 26 nt, synthetic construct
  source 1..26: mol_type = other RNA
aatgttttcttttgaacatcttctct

SEQ ID NO: 857 - RNA, 26 nt, synthetic construct
  source 1..26: mol_type = other RNA
atgttttcttttgaacatcttctctt

SEQ ID NO: 858 - RNA, 26 nt, synthetic construct
  source 1..26: mol_type = other RNA
tgttttcttttgaacatcttctcttt

SEQ ID NO: 859 - RNA, 26 nt, synthetic construct
  source 1..26: mol_type = other RNA
gttttcttttgaacatcttctctttc

SEQ ID NO: 860 - RNA, 26 nt, synthetic construct
  source 1..26: mol_type = other RNA
ttttcttttgaacatcttctctttca

SEQ ID NO: 861 - RNA, 26 nt, synthetic construct
  source 1..26: mol_type = other RNA
tttcttttgaacatcttctctttcat

SEQ ID NO: 862 - RNA, 26 nt, synthetic construct
  source 1..26: mol_type = other RNA
ttcttttgaacatcttctctttcatc

SEQ ID NO: 863 - RNA, 26 nt, synthetic construct
  source 1..26: mol_type = other RNA
tcttttgaacatcttctctttcatct

SEQ ID NO: 864 - RNA, 26 nt, synthetic construct
  source 1..26: mol_type = other RNA
cttttgaacatcttctctttcatcta

SEQ ID NO: 865 - RNA, 26 nt, synthetic construct
  source 1..26: mol_type = other RNA
ttttgaacatcttctctttcatctaa

SEQ ID NO: 866 - RNA, 26 nt, synthetic construct
  source 1..26: mol_type = other RNA
tttgaacatcttctctttcatctaaa

SEQ ID NO: 867 - RNA, 26 nt, synthetic construct
  source 1..26: mol_type = other RNA
ttgaacatcttctctttcatctaaaa

SEQ ID NO: 868 - RNA, 26 nt, synthetic construct
  source 1..26: mol_type = other RNA
tgaacatcttctctttcatctaaaat

SEQ ID NO: 869 - RNA, 26 nt, synthetic construct
  source 1..26: mol_type = other RNA
gaacatcttctctttcatctaaaatg

SEQ ID NO: 870 - RNA, 26 nt, synthetic construct
  source 1..26: mol_type = other RNA
aacatcttctctttcatctaaaatgc

SEQ ID NO: 871 - RNA, 26 nt, synthetic construct
  source 1..26: mol_type = other RNA
acatcttctctttcatctaaaatgca

SEQ ID NO: 872 - RNA, 26 nt, synthetic construct
  source 1..26: mol_type = other RNA
catcttctctttcatctaaaatgcaa

SEQ ID NO: 873 - RNA, 26 nt, synthetic construct
  source 1..26: mol_type = other RNA
atcttctctttcatctaaaatgcaaa

SEQ ID NO: 874 - RNA, 26 nt, synthetic construct
  source 1..26: mol_type = other RNA
tcttctctttcatctaaaatgcaaaa

SEQ ID NO: 875 - RNA, 26 nt, synthetic construct
  source 1..26: mol_type = other RNA
cttctctttcatctaaaatgcaaaat

SEQ ID NO: 876 - RNA, 26 nt, synthetic construct
  source 1..26: mol_type = other RNA
ttctctttcatctaaaatgcaaaata

SEQ ID NO: 877 - RNA, 26 nt, synthetic construct
  source 1..26: mol_type = other RNA
tctctttcatctaaaatgcaaaataa

SEQ ID NO: 878 - RNA, 26 nt, synthetic construct
  source 1..26: mol_type = other RNA
ctctttcatctaaaatgcaaaataaa

SEQ ID NO: 879 - RNA, 26 nt, synthetic construct
  source 1..26: mol_type = other RNA
tctttcatctaaaatgcaaaataaaa

SEQ ID NO: 880 - RNA, 26 nt, synthetic construct
  source 1..26: mol_type = other RNA
ctttcatctaaaatgcaaaataaaaa

SEQ ID NO: 881 - RNA, 26 nt, synthetic construct
  source 1..26: mol_type = other RNA
tttcatctaaaatgcaaaataaaaaa

SEQ ID NO: 882 - RNA, 26 nt, synthetic construct
  source 1..26: mol_type = other RNA
ttcatctaaaatgcaaaataaaaaaa

SEQ ID NO: 883 - RNA, 26 nt, synthetic construct
  source 1..26: mol_type = other RNA
tcatctaaaatgcaaaataaaaaaat

SEQ ID NO: 884 - RNA, 26 nt, synthetic construct
  source 1..26: mol_type = other RNA
catctaaaatgcaaaataaaaaaata

SEQ ID NO: 885 - RNA, 26 nt, synthetic construct
  source 1..26: mol_type = other RNA
atctaaaatgcaaaataaaaaaataa

SEQ ID NO: 886 - RNA, 26 nt, synthetic construct
  source 1..26: mol_type = other RNA
tctaaaatgcaaaataaaaaaataaa

SEQ ID NO: 887 - RNA, 26 nt, synthetic construct
  source 1..26: mol_type = other RNA
ctaaaatgcaaaataaaaaaataaaa

SEQ ID NO: 888 - RNA, 26 nt, synthetic construct
  source 1..26: mol_type = other RNA
taaaatgcaaaataaaaaaataaaag

SEQ ID NO: 889 - RNA, 26 nt, synthetic construct
  source 1..26: mol_type = other RNA
aaaatgcaaaataaaaaaataaaagt

SEQ ID NO: 890 - RNA, 26 nt, synthetic construct
  source 1..26: mol_type = other RNA
aaatgcaaaataaaaaaataaaagtt

SEQ ID NO: 891 - RNA, 26 nt, synthetic construct
  source 1..26: mol_type = other RNA
aatgcaaaataaaaaaataaaagtta

SEQ ID NO: 892 - RNA, 26 nt, synthetic construct
  source 1..26: mol_type = other RNA
atgcaaaataaaaaaataaaagttag

SEQ ID NO: 893 - RNA, 26 nt, synthetic construct
  source 1..26: mol_type = other RNA
tgcaaaataaaaaaataaaagttagg

SEQ ID NO: 894 - RNA, 26 nt, synthetic construct
  source 1..26: mol_type = other RNA
gcaaaataaaaaaataaaagttagga

SEQ ID NO: 895 - RNA, 26 nt, synthetic construct
  source 1..26: mol_type = other RNA
caaaataaaaaaataaaagttaggaa

SEQ ID NO: 896 - RNA, 26 nt, synthetic construct
  source 1..26: mol_type = other RNA
aaaataaaaaaataaaagttaggaag

SEQ ID NO: 897 - RNA, 26 nt, synthetic construct
  source 1..26: mol_type = other RNA
aaataaaaaaataaaagttaggaagc

SEQ ID NO: 898 - RNA, 26 nt, synthetic construct
  source 1..26: mol_type = other RNA
aataaaaaaataaaagttaggaagca

SEQ ID NO: 899 - RNA, 26 nt, synthetic construct
  source 1..26: mol_type = other RNA
ataaaaaaataaaagttaggaagcaa

SEQ ID NO: 900 - RNA, 26 nt, synthetic construct
  source 1..26: mol_type = other RNA
taaaaaaataaaagttaggaagcaac

SEQ ID NO: 901 - RNA, 26 nt, synthetic construct
  source 1..26: mol_type = other RNA
aaaaaaataaaagttaggaagcaact

SEQ ID NO: 902 - RNA, 26 nt, synthetic construct
  source 1..26: mol_type = other RNA
aaaaaataaaagttaggaagcaactt

SEQ ID NO: 903 - RNA, 26 nt, synthetic construct
  source 1..26: mol_type = other RNA
aaaaataaaagttaggaagcaacttt

SEQ ID NO: 904 - RNA, 26 nt, synthetic construct
  source 1..26: mol_type = other RNA
aaaataaaagttaggaagcaacttta

SEQ ID NO: 905 - RNA, 26 nt, synthetic construct
  source 1..26: mol_type = other RNA
aaataaaagttaggaagcaactttaa

SEQ ID NO: 906 - RNA, 26 nt, synthetic construct
  source 1..26: mol_type = other RNA
aataaaagttaggaagcaactttaaa

SEQ ID NO: 907 - RNA, 26 nt, synthetic construct
  source 1..26: mol_type = other RNA
ataaaagttaggaagcaactttaaat

SEQ ID NO: 908 - RNA, 26 nt, synthetic construct
  source 1..26: mol_type = other RNA
taaaagttaggaagcaactttaaata

SEQ ID NO: 909 - RNA, 26 nt, synthetic construct
  source 1..26: mol_type = other RNA
aaaagttaggaagcaactttaaatat

SEQ ID NO: 910 - RNA, 26 nt, synthetic construct
  source 1..26: mol_type = other RNA
aaagttaggaagcaactttaaatata

SEQ ID NO: 911 - RNA, 26 nt, synthetic construct
  source 1..26: mol_type = other RNA
aagttaggaagcaactttaaatataa

SEQ ID NO: 912 - RNA, 26 nt, synthetic construct
  source 1..26: mol_type = other RNA
agttaggaagcaactttaaatataat

SEQ ID NO: 913 - RNA, 26 nt, synthetic construct
  source 1..26: mol_type = other RNA
gttaggaagcaactttaaatataatt

SEQ ID NO: 914 - RNA, 26 nt, synthetic construct
  source 1..26: mol_type = other RNA
ttaggaagcaactttaaatataattc

SEQ ID NO: 915 - RNA, 26 nt, synthetic construct
  source 1..26: mol_type = other RNA
taggaagcaactttaaatataattca

SEQ ID NO: 916 - RNA, 26 nt, synthetic construct
  source 1..26: mol_type = other RNA
aggaagcaactttaaatataattcat

SEQ ID NO: 917 - RNA, 26 nt, synthetic construct
  source 1..26: mol_type = other RNA
ggaagcaactttaaatataattcata

SEQ ID NO: 918 - RNA, 26 nt, synthetic construct
  source 1..26: mol_type = other RNA
gaagcaactttaaatataattcatat

SEQ ID NO: 919 - RNA, 26 nt, synthetic construct
  source 1..26: mol_type = other RNA
aagcaactttaaatataattcatatt

SEQ ID NO: 920 - RNA, 26 nt, synthetic construct
  source 1..26: mol_type = other RNA
agcaactttaaatataattcatattt

SEQ ID NO: 921 - RNA, 26 nt, synthetic construct
  source 1..26: mol_type = other RNA
gcaactttaaatataattcatatttt

SEQ ID NO: 922 - RNA, 26 nt, synthetic construct
  source 1..26: mol_type = other RNA
caactttaaatataattcatatttta

SEQ ID NO: 923 - RNA, 26 nt, synthetic construct
  source 1..26: mol_type = other RNA
aactttaaatataattcatattttac

SEQ ID NO: 924 - RNA, 26 nt, synthetic construct
  source 1..26: mol_type = other RNA
actttaaatataattcatattttaca

SEQ ID NO: 925 - RNA, 26 nt, synthetic construct
  source 1..26: mol_type = other RNA
ctttaaatataattcatattttacaa

SEQ ID NO: 926 - RNA, 26 nt, synthetic construct
  source 1..26: mol_type = other RNA
tttaaatataattcatattttacaac

SEQ ID NO: 927 - RNA, 26 nt, synthetic construct
  source 1..26: mol_type = other RNA
ttaaatataattcatattttacaacc

SEQ ID NO: 928 - RNA, 26 nt, synthetic construct
  source 1..26: mol_type = other RNA
taaatataattcatattttacaacca

SEQ ID NO: 929 - RNA, 26 nt, synthetic construct
  source 1..26: mol_type = other RNA
aaatataattcatattttacaaccaa

SEQ ID NO: 930 - RNA, 26 nt, synthetic construct
  source 1..26: mol_type = other RNA
aatataattcatattttacaaccaaa

SEQ ID NO: 931 - RNA, 26 nt, synthetic construct
  source 1..26: mol_type = other RNA
atataattcatattttacaaccaaag

SEQ ID NO: 932 - RNA, 26 nt, synthetic construct
  source 1..26: mol_type = other RNA
tataattcatattttacaaccaaagt

SEQ ID NO: 933 - RNA, 26 nt, synthetic construct
  source 1..26: mol_type = other RNA
ataattcatattttacaaccaaagta

SEQ ID NO: 934 - RNA, 26 nt, synthetic construct
  source 1..26: mol_type = other RNA
taattcatattttacaaccaaagtaa

SEQ ID NO: 935 - RNA, 26 nt, synthetic construct
  source 1..26: mol_type = other RNA
aattcatattttacaaccaaagtaac

SEQ ID NO: 936 - RNA, 26 nt, synthetic construct
  source 1..26: mol_type = other RNA
attcatattttacaaccaaagtaact

SEQ ID NO: 937 - RNA, 26 nt, synthetic construct
  source 1..26: mol_type = other RNA
ttcatattttacaaccaaagtaactt

SEQ ID NO: 938 - RNA, 26 nt, synthetic construct
  source 1..26: mol_type = other RNA
tcatattttacaaccaaagtaacttt

SEQ ID NO: 939 - RNA, 26 nt, synthetic construct
  source 1..26: mol_type = other RNA
catattttacaaccaaagtaactttc

SEQ ID NO: 940 - RNA, 26 nt, synthetic construct
  source 1..26: mol_type = other RNA
atattttacaaccaaagtaactttcc

SEQ ID NO: 941 - RNA, 26 nt, synthetic construct
  source 1..26: mol_type = other RNA
tattttacaaccaaagtaactttcca

SEQ ID NO: 942 - RNA, 26 nt, synthetic construct
  source 1..26: mol_type = other RNA
attttacaaccaaagtaactttccat

SEQ ID NO: 943 - RNA, 26 nt, synthetic construct
  source 1..26: mol_type = other RNA
ttttacaaccaaagtaactttccatt

SEQ ID NO: 944 - RNA, 26 nt, synthetic construct
  source 1..26: mol_type = other RNA
tttacaaccaaagtaactttccatta

SEQ ID NO: 945 - RNA, 26 nt, synthetic construct
  source 1..26: mol_type = other RNA
ttacaaccaaagtaactttccattat

SEQ ID NO: 946 - RNA, 26 nt, synthetic construct
  source 1..26: mol_type = other RNA
tacaaccaaagtaactttccattatg

SEQ ID NO: 947 - RNA, 26 nt, synthetic construct
  source 1..26: mol_type = other RNA
acaaccaaagtaactttccattatga

SEQ ID NO: 948 - RNA, 26 nt, synthetic construct
  source 1..26: mol_type = other RNA
caaccaaagtaactttccattatgat

SEQ ID NO: 949 - RNA, 26 nt, synthetic construct
  source 1..26: mol_type = other RNA
aaccaaagtaactttccattatgatg

SEQ ID NO: 950 - RNA, 26 nt, synthetic construct
  source 1..26: mol_type = other RNA
accaaagtaactttccattatgatgt

SEQ ID NO: 951 - RNA, 26 nt, synthetic construct
  source 1..26: mol_type = other RNA
ccaaagtaactttccattatgatgtg

SEQ ID NO: 952 - RNA, 26 nt, synthetic construct
  source 1..26: mol_type = other RNA
caaagtaactttccattatgatgtgt

SEQ ID NO: 953 - RNA, 26 nt, synthetic construct
  source 1..26: mol_type = other RNA
aaagtaactttccattatgatgtgtt

SEQ ID NO: 954 - RNA, 26 nt, synthetic construct
  source 1..26: mol_type = other RNA
aagtaactttccattatgatgtgtta

SEQ ID NO: 955 - RNA, 26 nt, synthetic construct
  source 1..26: mol_type = other RNA
agtaactttccattatgatgtgttag

SEQ ID NO: 956 - RNA, 26 nt, synthetic construct
  source 1..26: mol_type = other RNA
gtaactttccattatgatgtgttagt

SEQ ID NO: 957 - RNA, 26 nt, synthetic construct
  source 1..26: mol_type = other RNA
taactttccattatgatgtgttagtg

SEQ ID NO: 958 - RNA, 26 nt, synthetic construct
  source 1..26: mol_type = other RNA
aactttccattatgatgtgttagtgt

SEQ ID NO: 959 - RNA, 26 nt, synthetic construct
  source 1..26: mol_type = other RNA
actttccattatgatgtgttagtgtt

SEQ ID NO: 960 - RNA, 26 nt, synthetic construct
  source 1..26: mol_type = other RNA
ctttccattatgatgtgttagtgttt

SEQ ID NO: 961 - RNA, 26 nt, synthetic construct
  source 1..26: mol_type = other RNA
tttccattatgatgtgttagtgtttt

SEQ ID NO: 962 - RNA, 26 nt, synthetic construct
  source 1..26: mol_type = other RNA
ttccattatgatgtgttagtgttttt

SEQ ID NO: 963 - RNA, 26 nt, synthetic construct
  source 1..26: mol_type = other RNA
tccattatgatgtgttagtgtttttt

SEQ ID NO: 964 - RNA, 26 nt, synthetic construct
  source 1..26: mol_type = other RNA
ccattatgatgtgttagtgttttttt

SEQ ID NO: 965 - RNA, 26 nt, synthetic construct
  source 1..26: mol_type = other RNA
cattatgatgtgttagtgttttttta

SEQ ID NO: 966 - RNA, 26 nt, synthetic construct
  source 1..26: mol_type = other RNA
attatgatgtgttagtgtttttttac

SEQ ID NO: 967 - RNA, 26 nt, synthetic construct
  source 1..26: mol_type = other RNA
ttatgatgtgttagtgtttttttaca

SEQ ID NO: 968 - RNA, 26 nt, synthetic construct
  source 1..26: mol_type = other RNA
tatgatgtgttagtgtttttttacat

SEQ ID NO: 969 - RNA, 26 nt, synthetic construct
  source 1..26: mol_type = other RNA
atgatgtgttagtgtttttttacatt

SEQ ID NO: 970 - RNA, 26 nt, synthetic construct
  source 1..26: mol_type = other RNA
tgatgtgttagtgtttttttacattt

SEQ ID NO: 971 - RNA, 26 nt, synthetic construct
  source 1..26: mol_type = other RNA
gatgtgttagtgtttttttacattta

SEQ ID NO: 972 - RNA, 26 nt, synthetic construct
  source 1..26: mol_type = other RNA
atgtgttagtgtttttttacatttag

SEQ ID NO: 973 - RNA, 26 nt, synthetic construct
  source 1..26: mol_type = other RNA
tgtgttagtgtttttttacatttagt

SEQ ID NO: 974 - RNA, 26 nt, synthetic construct
  source 1..26: mol_type = other RNA
gtgttagtgtttttttacatttagta

SEQ ID NO: 975 - RNA, 26 nt, synthetic construct
  source 1..26: mol_type = other RNA
tgttagtgtttttttacatttagtat

SEQ ID NO: 976 - RNA, 26 nt, synthetic construct
  source 1..26: mol_type = other RNA
gttagtgtttttttacatttagtatt

SEQ ID NO: 977 - RNA, 26 nt, synthetic construct
  source 1..26: mol_type = other RNA
ttagtgtttttttacatttagtatta

SEQ ID NO: 978 - RNA, 26 nt, synthetic construct
  source 1..26: mol_type = other RNA
tagtgtttttttacatttagtattac

SEQ ID NO: 979 - RNA, 26 nt, synthetic construct
  source 1..26: mol_type = other RNA
agtgtttttttacatttagtattaca

SEQ ID NO: 980 - RNA, 26 nt, synthetic construct
  source 1..26: mol_type = other RNA
gtgtttttttacatttagtattacat

SEQ ID NO: 981 - RNA, 26 nt, synthetic construct
  source 1..26: mol_type = other RNA
tgtttttttacatttagtattacatt

SEQ ID NO: 982 - RNA, 26 nt, synthetic construct
  source 1..26: mol_type = other RNA
gtttttttacatttagtattacattc

SEQ ID NO: 983 - RNA, 26 nt, synthetic construct
  source 1..26: mol_type = other RNA
tttttttacatttagtattacattca

SEQ ID NO: 984 - RNA, 26 nt, synthetic construct
  source 1..26: mol_type = other RNA
ttttttacatttagtattacattcat

SEQ ID NO: 985 - RNA, 26 nt, synthetic construct
  source 1..26: mol_type = other RNA
tttttacatttagtattacattcatt

SEQ ID NO: 986 - RNA, 26 nt, synthetic construct
  source 1..26: mol_type = other RNA
ttttacatttagtattacattcattg

SEQ ID NO: 987 - RNA, 26 nt, synthetic construct
  source 1..26: mol_type = other RNA
tttacatttagtattacattcattga

SEQ ID NO: 988 - RNA, 26 nt, synthetic construct
  source 1..26: mol_type = other RNA
ttacatttagtattacattcattgat

SEQ ID NO: 989 - RNA, 26 nt, synthetic construct
  source 1..26: mol_type = other RNA
tacatttagtattacattcattgata

SEQ ID NO: 990 - RNA, 26 nt, synthetic construct
  source 1..26: mol_type = other RNA
acatttagtattacattcattgataa

SEQ ID NO: 991 - RNA, 26 nt, synthetic construct
  source 1..26: mol_type = other RNA
catttagtattacattcattgataaa

SEQ ID NO: 992 - RNA, 26 nt, synthetic construct
  source 1..26: mol_type = other RNA
atttagtattacattcattgataaat

SEQ ID NO: 993 - RNA, 26 nt, synthetic construct
  source 1..26: mol_type = other RNA
tttagtattacattcattgataaatg

SEQ ID NO: 994 - RNA, 26 nt, synthetic construct
  source 1..26: mol_type = other RNA
ttagtattacattcattgataaatgg

SEQ ID NO: 995 - RNA, 26 nt, synthetic construct
  source 1..26: mol_type = other RNA
tagtattacattcattgataaatgga

SEQ ID NO: 996 - RNA, 26 nt, synthetic construct
  source 1..26: mol_type = other RNA
agtattacattcattgataaatggaa

SEQ ID NO: 997 - RNA, 26 nt, synthetic construct
  source 1..26: mol_type = other RNA
gtattacattcattgataaatggaat

SEQ ID NO: 998 - RNA, 26 nt, synthetic construct
  source 1..26: mol_type = other RNA
tattacattcattgataaatggaatt

SEQ ID NO: 999 - RNA, 26 nt, synthetic construct
  source 1..26: mol_type = other RNA
attacattcattgataaatggaatta

SEQ ID NO: 1000 - RNA, 26 nt, synthetic construct
  source 1..26: mol_type = other RNA
ttacattcattgataaatggaattaa

SEQ ID NO: 1001 - RNA, 26 nt, synthetic construct
  source 1..26: mol_type = other RNA
tacattcattgataaatggaattaaa

SEQ ID NO: 1002 - RNA, 26 nt, synthetic construct
  source 1..26: mol_type = other RNA
acattcattgataaatggaattaaac

SEQ ID NO: 1003 - RNA, 26 nt, synthetic construct
  source 1..26: mol_type = other RNA
cattcattgataaatggaattaaaca

SEQ ID NO: 1004 - RNA, 26 nt, synthetic construct
  source 1..26: mol_type = other RNA
attcattgataaatggaattaaacat

SEQ ID NO: 1005 - RNA, 26 nt, synthetic construct
  source 1..26: mol_type = other RNA
ttcattgataaatggaattaaacatg

SEQ ID NO: 1006 - RNA, 26 nt, synthetic construct
  source 1..26: mol_type = other RNA
tcattgataaatggaattaaacatga

SEQ ID NO: 1007 - RNA, 26 nt, synthetic construct
  source 1..26: mol_type = other RNA
cattgataaatggaattaaacatgag

SEQ ID NO: 1008 - RNA, 26 nt, synthetic construct
  source 1..26: mol_type = other RNA
attgataaatggaattaaacatgagt

SEQ ID NO: 1009 - RNA, 26 nt, synthetic construct
  source 1..26: mol_type = other RNA
ttgataaatggaattaaacatgagtg

SEQ ID NO: 1010 - RNA, 26 nt, synthetic construct
  source 1..26: mol_type = other RNA
tgataaatggaattaaacatgagtga

SEQ ID NO: 1011 - RNA, 26 nt, synthetic construct
  source 1..26: mol_type = other RNA
gataaatggaattaaacatgagtgat

SEQ ID NO: 1012 - RNA, 30 nt, synthetic construct
  source 1..30: mol_type = other RNA
gagtgaacataatatttccagatttgcaca

SEQ ID NO: 1013 - RNA, 30 nt, synthetic construct
  source 1..30: mol_type = other RNA
agtgaacataatatttccagatttgcacag

SEQ ID NO: 1014 - RNA, 30 nt, synthetic construct
  source 1..30: mol_type = other RNA
gtgaacataatatttccagatttgcacagc

SEQ ID NO: 1015 - RNA, 30 nt, synthetic construct
  source 1..30: mol_type = other RNA
tgaacataatatttccagatttgcacagct

SEQ ID NO: 1016 - RNA, 30 nt, synthetic construct
  source 1..30: mol_type = other RNA
gaacataatatttccagatttgcacagcta

SEQ ID NO: 1017 - RNA, 30 nt, synthetic construct
  source 1..30: mol_type = other RNA
aacataatatttccagatttgcacagctaa

SEQ ID NO: 1018 - RNA, 30 nt, synthetic construct
  source 1..30: mol_type = other RNA
acataatatttccagatttgcacagctaaa

SEQ ID NO: 1019 - RNA, 30 nt, synthetic construct
  source 1..30: mol_type = other RNA
cataatatttccagatttgcacagctaaaa

SEQ ID NO: 1020 - RNA, 30 nt, synthetic construct
  source 1..30: mol_type = other RNA
ataatatttccagatttgcacagctaaaat

SEQ ID NO: 1021 - RNA, 30 nt, synthetic construct
  source 1..30: mol_type = other RNA
taatatttccagatttgcacagctaaaata

SEQ ID NO: 1022 - RNA, 30 nt, synthetic construct
  source 1..30: mol_type = other RNA
aatatttccagatttgcacagctaaaataa

SEQ ID NO: 1023 - RNA, 30 nt, synthetic construct
  source 1..30: mol_type = other RNA
atatttccagatttgcacagctaaaataaa

SEQ ID NO: 1024 - RNA, 30 nt, synthetic construct
  source 1..30: mol_type = other RNA
tatttccagatttgcacagctaaaataaaa

SEQ ID NO: 1025 - RNA, 30 nt, synthetic construct
  source 1..30: mol_type = other RNA
atttccagatttgcacagctaaaataaaat

SEQ ID NO: 1026 - RNA, 30 nt, synthetic construct
  source 1..30: mol_type = other RNA
tttccagatttgcacagctaaaataaaatg

SEQ ID NO: 1027 - RNA, 30 nt, synthetic construct
  source 1..30: mol_type = other RNA
ttccagatttgcacagctaaaataaaatga

SEQ ID NO: 1028 - RNA, 30 nt, synthetic construct
  source 1..30: mol_type = other RNA
tccagatttgcacagctaaaataaaatgac

SEQ ID NO: 1029 - RNA, 30 nt, synthetic construct
  source 1..30: mol_type = other RNA
ccagatttgcacagctaaaataaaatgaca

SEQ ID NO: 1030 - RNA, 30 nt, synthetic construct
  source 1..30: mol_type = other RNA
cagatttgcacagctaaaataaaatgacac

SEQ ID NO: 1031 - RNA, 30 nt, synthetic construct
  source 1..30: mol_type = other RNA
agatttgcacagctaaaataaaatgacact

SEQ ID NO: 1032 - RNA, 30 nt, synthetic construct
  source 1..30: mol_type = other RNA
gatttgcacagctaaaataaaatgacacta

SEQ ID NO: 1033 - RNA, 30 nt, synthetic construct
  source 1..30: mol_type = other RNA
atttgcacagctaaaataaaatgacactat

SEQ ID NO: 1034 - RNA, 30 nt, synthetic construct
  source 1..30: mol_type = other RNA
tttgcacagctaaaataaaatgacactatg

SEQ ID NO: 1035 - RNA, 30 nt, synthetic construct
  source 1..30: mol_type = other RNA
ttgcacagctaaaataaaatgacactatga

SEQ ID NO: 1036 - RNA, 30 nt, synthetic construct
  source 1..30: mol_type = other RNA
tgcacagctaaaataaaatgacactatgag

SEQ ID NO: 1037 - RNA, 30 nt, synthetic construct
  source 1..30: mol_type = other RNA
gcacagctaaaataaaatgacactatgaga

SEQ ID NO: 1038 - RNA, 30 nt, synthetic construct
  source 1..30: mol_type = other RNA
cacagctaaaataaaatgacactatgagag

SEQ ID NO: 1039 - RNA, 30 nt, synthetic construct
  source 1..30: mol_type = other RNA
acagctaaaataaaatgacactatgagaga

SEQ ID NO: 1040 - RNA, 30 nt, synthetic construct
  source 1..30: mol_type = other RNA
cagctaaaataaaatgacactatgagagaa

SEQ ID NO: 1041 - RNA, 30 nt, synthetic construct
  source 1..30: mol_type = other RNA
agctaaaataaaatgacactatgagagaaa

SEQ ID NO: 1042 - RNA, 30 nt, synthetic construct
  source 1..30: mol_type = other RNA
gctaaaataaaatgacactatgagagaaat

SEQ ID NO: 1043 - RNA, 30 nt, synthetic construct
  source 1..30: mol_type = other RNA
ctaaaataaaatgacactatgagagaaata

SEQ ID NO: 1044 - RNA, 30 nt, synthetic construct
  source 1..30: mol_type = other RNA
taaaataaaatgacactatgagagaaataa

SEQ ID NO: 1045 - RNA, 30 nt, synthetic construct
  source 1..30: mol_type = other RNA
aaaataaaatgacactatgagagaaataaa

SEQ ID NO: 1046 - RNA, 30 nt, synthetic construct
  source 1..30: mol_type = other RNA
aaataaaatgacactatgagagaaataaaa

SEQ ID NO: 1047 - RNA, 30 nt, synthetic construct
  source 1..30: mol_type = other RNA
aataaaatgacactatgagagaaataaaac

SEQ ID NO: 1048 - RNA, 30 nt, synthetic construct
  source 1..30: mol_type = other RNA
ataaaatgacactatgagagaaataaaacg

SEQ ID NO: 1049 - RNA, 30 nt, synthetic construct
  source 1..30: mol_type = other RNA
taaaatgacactatgagagaaataaaacgg

SEQ ID NO: 1050 - RNA, 30 nt, synthetic construct
  source 1..30: mol_type = other RNA
aaaatgacactatgagagaaataaaacgga

SEQ ID NO: 1051 - RNA, 30 nt, synthetic construct
  source 1..30: mol_type = other RNA
aaatgacactatgagagaaataaaacggat

SEQ ID NO: 1052 - RNA, 30 nt, synthetic construct
  source 1..30: mol_type = other RNA
aatgacactatgagagaaataaaacggatt

SEQ ID NO: 1053 - RNA, 30 nt, synthetic construct
  source 1..30: mol_type = other RNA
atgacactatgagagaaataaaacggattt

SEQ ID NO: 1054 - RNA, 30 nt, synthetic construct
  source 1..30: mol_type = other RNA
tgacactatgagagaaataaaacggatttt

SEQ ID NO: 1055 - RNA, 30 nt, synthetic construct
  source 1..30: mol_type = other RNA
gacactatgagagaaataaaacggattttt

SEQ ID NO: 1056 - RNA, 30 nt, synthetic construct
  source 1..30: mol_type = other RNA
acactatgagagaaataaaacggattttta

SEQ ID NO: 1057 - RNA, 30 nt, synthetic construct
  source 1..30: mol_type = other RNA
cactatgagagaaataaaacggatttttaa

SEQ ID NO: 1058 - RNA, 30 nt, synthetic construct
  source 1..30: mol_type = other RNA
actatgagagaaataaaacggatttttaag

SEQ ID NO: 1059 - RNA, 30 nt, synthetic construct
  source 1..30: mol_type = other RNA
ctatgagagaaataaaacggatttttaaga

SEQ ID NO: 1060 - RNA, 30 nt, synthetic construct
  source 1..30: mol_type = other RNA
tatgagagaaataaaacggatttttaagat

SEQ ID NO: 1061 - RNA, 30 nt, synthetic construct
  source 1..30: mol_type = other RNA
atgagagaaataaaacggatttttaagata

SEQ ID NO: 1062 - RNA, 30 nt, synthetic construct
  source 1..30: mol_type = other RNA
tgagagaaataaaacggatttttaagatac

SEQ ID NO: 1063 - RNA, 30 nt, synthetic construct
  source 1..30: mol_type = other RNA
gagagaaataaaacggatttttaagataca

SEQ ID NO: 1064 - RNA, 30 nt, synthetic construct
  source 1..30: mol_type = other RNA
agagaaataaaacggatttttaagatacac

SEQ ID NO: 1065 - RNA, 30 nt, synthetic construct
  source 1..30: mol_type = other RNA
gagaaataaaacggatttttaagatacaca

SEQ ID NO: 1066 - RNA, 30 nt, synthetic construct
  source 1..30: mol_type = other RNA
agaaataaaacggatttttaagatacacag

SEQ ID NO: 1067 - RNA, 30 nt, synthetic construct
  source 1..30: mol_type = other RNA
gaaataaaacggatttttaagatacacagg

SEQ ID NO: 1068 - RNA, 30 nt, synthetic construct
  source 1..30: mol_type = other RNA
aaataaaacggatttttaagatacacaggt

SEQ ID NO: 1069 - RNA, 30 nt, synthetic construct
  source 1..30: mol_type = other RNA
aataaaacggatttttaagatacacaggta

SEQ ID NO: 1070 - RNA, 30 nt, synthetic construct
  source 1..30: mol_type = other RNA
ataaaacggatttttaagatacacaggtac

SEQ ID NO: 1071 - RNA, 30 nt, synthetic construct
  source 1..30: mol_type = other RNA
taaaacggatttttaagatacacaggtaca

SEQ ID NO: 1072 - RNA, 30 nt, synthetic construct
  source 1..30: mol_type = other RNA
aaaacggatttttaagatacacaggtacat

SEQ ID NO: 1073 - RNA, 30 nt, synthetic construct
  source 1..30: mol_type = other RNA
aaacggatttttaagatacacaggtacata

SEQ ID NO: 1074 - RNA, 30 nt, synthetic construct
  source 1..30: mol_type = other RNA
aacggatttttaagatacacaggtacatag

SEQ ID NO: 1075 - RNA, 30 nt, synthetic construct
  source 1..30: mol_type = other RNA
acggatttttaagatacacaggtacatagt

SEQ ID NO: 1076 - RNA, 30 nt, synthetic construct
  source 1..30: mol_type = other RNA
cggatttttaagatacacaggtacatagtc

SEQ ID NO: 1077 - RNA, 30 nt, synthetic construct
  source 1..30: mol_type = other RNA
ggatttttaagatacacaggtacatagtcc

SEQ ID NO: 1078 - RNA, 30 nt, synthetic construct
  source 1..30: mol_type = other RNA
gatttttaagatacacaggtacatagtcca

SEQ ID NO: 1079 - RNA, 30 nt, synthetic construct
  source 1..30: mol_type = other RNA
atttttaagatacacaggtacatagtccat

SEQ ID NO: 1080 - RNA, 30 nt, synthetic construct
  source 1..30: mol_type = other RNA
tttttaagatacacaggtacatagtccatt

SEQ ID NO: 1081 - RNA, 30 nt, synthetic construct
  source 1..30: mol_type = other RNA
ttttaagatacacaggtacatagtccattt

SEQ ID NO: 1082 - RNA, 30 nt, synthetic construct
  source 1..30: mol_type = other RNA
tttaagatacacaggtacatagtccatttt

SEQ ID NO: 1083 - RNA, 30 nt, synthetic construct
  source 1..30: mol_type = other RNA
ttaagatacacaggtacatagtccattttg

SEQ ID NO: 1084 - RNA, 30 nt, synthetic construct
  source 1..30: mol_type = other RNA
taagatacacaggtacatagtccattttga

SEQ ID NO: 1085 - RNA, 30 nt, synthetic construct
  source 1..30: mol_type = other RNA
aagatacacaggtacatagtccattttgaa

SEQ ID NO: 1086 - RNA, 30 nt, synthetic construct
  source 1..30: mol_type = other RNA
agatacacaggtacatagtccattttgaaa

SEQ ID NO: 1087 - RNA, 30 nt, synthetic construct
  source 1..30: mol_type = other RNA
gatacacaggtacatagtccattttgaaaa

SEQ ID NO: 1088 - RNA, 30 nt, synthetic construct
  source 1..30: mol_type = other RNA
atacacaggtacatagtccattttgaaaat

SEQ ID NO: 1089 - RNA, 30 nt, synthetic construct
  source 1..30: mol_type = other RNA
tacacaggtacatagtccattttgaaaatt

SEQ ID NO: 1090 - RNA, 30 nt, synthetic construct
  source 1..30: mol_type = other RNA
acacaggtacatagtccattttgaaaattt

SEQ ID NO: 1091 - RNA, 30 nt, synthetic construct
  source 1..30: mol_type = other RNA
cacaggtacatagtccattttgaaaatttc

SEQ ID NO: 1092 - RNA, 30 nt, synthetic construct
  source 1..30: mol_type = other RNA
acaggtacatagtccattttgaaaatttca

SEQ ID NO: 1093 - RNA, 30 nt, synthetic construct
  source 1..30: mol_type = other RNA
caggtacatagtccattttgaaaatttcac

SEQ ID NO: 1094 - RNA, 30 nt, synthetic construct
  source 1..30: mol_type = other RNA
aggtacatagtccattttgaaaatttcaca

SEQ ID NO: 1095 - RNA, 30 nt, synthetic construct
  source 1..30: mol_type = other RNA
ggtacatagtccattttgaaaatttcacaa

SEQ ID NO: 1096 - RNA, 30 nt, synthetic construct
  source 1..30: mol_type = other RNA
gtacatagtccattttgaaaatttcacaac

SEQ ID NO: 1097 - RNA, 30 nt, synthetic construct
  source 1..30: mol_type = other RNA
tacatagtccattttgaaaatttcacaact

SEQ ID NO: 1098 - RNA, 30 nt, synthetic construct
  source 1..30: mol_type = other RNA
acatagtccattttgaaaatttcacaactt

SEQ ID NO: 1099 - RNA, 30 nt, synthetic construct
  source 1..30: mol_type = other RNA
catagtccattttgaaaatttcacaactta

SEQ ID NO: 1100 - RNA, 30 nt, synthetic construct
  source 1..30: mol_type = other RNA
atagtccattttgaaaatttcacaacttag

SEQ ID NO: 1101 - RNA, 30 nt, synthetic construct
  source 1..30: mol_type = other RNA
tagtccattttgaaaatttcacaacttaga

SEQ ID NO: 1102 - RNA, 30 nt, synthetic construct
  source 1..30: mol_type = other RNA
agtccattttgaaaatttcacaacttagat

SEQ ID NO: 1103 - RNA, 30 nt, synthetic construct
  source 1..30: mol_type = other RNA
gtccattttgaaaatttcacaacttagatc

SEQ ID NO: 1104 - RNA, 30 nt, synthetic construct
  source 1..30: mol_type = other RNA
tccattttgaaaatttcacaacttagatct

SEQ ID NO: 1105 - RNA, 30 nt, synthetic construct
  source 1..30: mol_type = other RNA
ccattttgaaaatttcacaacttagatctt

SEQ ID NO: 1106 - RNA, 30 nt, synthetic construct
  source 1..30: mol_type = other RNA
cattttgaaaatttcacaacttagatctta

SEQ ID NO: 1107 - RNA, 30 nt, synthetic construct
  source 1..30: mol_type = other RNA
attttgaaaatttcacaacttagatcttaa

SEQ ID NO: 1108 - RNA, 30 nt, synthetic construct
  source 1..30: mol_type = other RNA
ttttgaaaatttcacaacttagatcttaaa

SEQ ID NO: 1109 - RNA, 30 nt, synthetic construct
  source 1..30: mol_type = other RNA
tttgaaaatttcacaacttagatcttaaaa

SEQ ID NO: 1110 - RNA, 30 nt, synthetic construct
  source 1..30: mol_type = other RNA
ttgaaaatttcacaacttagatcttaaaag

SEQ ID NO: 1111 - RNA, 30 nt, synthetic construct
  source 1..30: mol_type = other RNA
tgaaaatttcacaacttagatcttaaaagt

SEQ ID NO: 1112 - RNA, 30 nt, synthetic construct
  source 1..30: mol_type = other RNA
gaaaatttcacaacttagatcttaaaagta

SEQ ID NO: 1113 - RNA, 30 nt, synthetic construct
  source 1..30: mol_type = other RNA
aaaatttcacaacttagatcttaaaagtaa

SEQ ID NO: 1114 - RNA, 30 nt, synthetic construct
  source 1..30: mol_type = other RNA
aaatttcacaacttagatcttaaaagtaaa

SEQ ID NO: 1115 - RNA, 30 nt, synthetic construct
  source 1..30: mol_type = other RNA
aatttcacaacttagatcttaaaagtaaag

SEQ ID NO: 1116 - RNA, 30 nt, synthetic construct
  source 1..30: mol_type = other RNA
atttcacaacttagatcttaaaagtaaagt

SEQ ID NO: 1117 - RNA, 30 nt, synthetic construct
  source 1..30: mol_type = other RNA
tttcacaacttagatcttaaaagtaaagta

SEQ ID NO: 1118 - RNA, 30 nt, synthetic construct
  source 1..30: mol_type = other RNA
ttcacaacttagatcttaaaagtaaagtaa

SEQ ID NO: 1119 - RNA, 30 nt, synthetic construct
  source 1..30: mol_type = other RNA
tcacaacttagatcttaaaagtaaagtaac

SEQ ID NO: 1120 - RNA, 30 nt, synthetic construct
  source 1..30: mol_type = other RNA
cacaacttagatcttaaaagtaaagtaaca

SEQ ID NO: 1121 - RNA, 30 nt, synthetic construct
  source 1..30: mol_type = other RNA
acaacttagatcttaaaagtaaagtaacaa

SEQ ID NO: 1122 - RNA, 30 nt, synthetic construct
  source 1..30: mol_type = other RNA
caacttagatcttaaaagtaaagtaacaaa

SEQ ID NO: 1123 - RNA, 30 nt, synthetic construct
  source 1..30: mol_type = other RNA
aacttagatcttaaaagtaaagtaacaaac

SEQ ID NO: 1124 - RNA, 30 nt, synthetic construct
  source 1..30: mol_type = other RNA
acttagatcttaaaagtaaagtaacaaacc

SEQ ID NO: 1125 - RNA, 30 nt, synthetic construct
  source 1..30: mol_type = other RNA
cttagatcttaaaagtaaagtaacaaacca

SEQ ID NO: 1126 - RNA, 30 nt, synthetic construct
  source 1..30: mol_type = other RNA
ttagatcttaaaagtaaagtaacaaaccat

SEQ ID NO: 1127 - RNA, 30 nt, synthetic construct
  source 1..30: mol_type = other RNA
tagatcttaaaagtaaagtaacaaaccatt

SEQ ID NO: 1128 - RNA, 30 nt, synthetic construct
  source 1..30: mol_type = other RNA
agatcttaaaagtaaagtaacaaaccattc

SEQ ID NO: 1129 - RNA, 30 nt, synthetic construct
  source 1..30: mol_type = other RNA
gatcttaaaagtaaagtaacaaaccattct

SEQ ID NO: 1130 - RNA, 30 nt, synthetic construct
  source 1..30: mol_type = other RNA
atcttaaaagtaaagtaacaaaccattctt

SEQ ID NO: 1131 - RNA, 30 nt, synthetic construct
  source 1..30: mol_type = other RNA
tcttaaaagtaaagtaacaaaccattctta

SEQ ID NO: 1132 - RNA, 30 nt, synthetic construct
  source 1..30: mol_type = other RNA
cttaaaagtaaagtaacaaaccattcttac

SEQ ID NO: 1133 - RNA, 30 nt, synthetic construct
  source 1..30: mol_type = other RNA
ttaaaagtaaagtaacaaaccattcttacc

SEQ ID NO: 1134 - RNA, 30 nt, synthetic construct
  source 1..30: mol_type = other RNA
taaaagtaaagtaacaaaccattcttacct

SEQ ID NO: 1135 - RNA, 30 nt, synthetic construct
  source 1..30: mol_type = other RNA
aaaagtaaagtaacaaaccattcttacctt

SEQ ID NO: 1136 - RNA, 30 nt, synthetic construct
  source 1..30: mol_type = other RNA
aaagtaaagtaacaaaccattcttacctta

SEQ ID NO: 1137 - RNA, 30 nt, synthetic construct
  source 1..30: mol_type = other RNA
aagtaaagtaacaaaccattcttaccttag

SEQ ID NO: 1138 - RNA, 30 nt, synthetic construct
  source 1..30: mol_type = other RNA
agtaaagtaacaaaccattcttaccttaga

SEQ ID NO: 1139 - RNA, 30 nt, synthetic construct
  source 1..30: mol_type = other RNA
gtaaagtaacaaaccattcttaccttagaa

SEQ ID NO: 1140 - RNA, 30 nt, synthetic construct
  source 1..30: mol_type = other RNA
taaagtaacaaaccattcttaccttagaaa

SEQ ID NO: 1141 - RNA, 30 nt, synthetic construct
  source 1..30: mol_type = other RNA
aaagtaacaaaccattcttaccttagaaaa

SEQ ID NO: 1142 - RNA, 30 nt, synthetic construct
  source 1..30: mol_type = other RNA
aagtaacaaaccattcttaccttagaaaat

SEQ ID NO: 1143 - RNA, 30 nt, synthetic construct
  source 1..30: mol_type = other RNA
agtaacaaaccattcttaccttagaaaatt

SEQ ID NO: 1144 - RNA, 30 nt, synthetic construct
  source 1..30: mol_type = other RNA
gtaacaaaccattcttaccttagaaaattg

SEQ ID NO: 1145 - RNA, 30 nt, synthetic construct
  source 1..30: mol_type = other RNA
taacaaaccattcttaccttagaaaattgt

SEQ ID NO: 1146 - RNA, 30 nt, synthetic construct
  source 1..30: mol_type = other RNA
aacaaaccattcttaccttagaaaattgtg

SEQ ID NO: 1147 - RNA, 30 nt, synthetic construct
  source 1..30: mol_type = other RNA
acaaaccattcttaccttagaaaattgtgc

SEQ ID NO: 1148 - RNA, 30 nt, synthetic construct
  source 1..30: mol_type = other RNA
caaaccattcttaccttagaaaattgtgca

SEQ ID NO: 1149 - RNA, 30 nt, synthetic construct
  source 1..30: mol_type = other RNA
aaaccattcttaccttagaaaattgtgcat

SEQ ID NO: 1150 - RNA, 30 nt, synthetic construct
  source 1..30: mol_type = other RNA
aaccattcttaccttagaaaattgtgcatt

SEQ ID NO: 1151 - RNA, 30 nt, synthetic construct
  source 1..30: mol_type = other RNA
accattcttaccttagaaaattgtgcattt

SEQ ID NO: 1152 - RNA, 30 nt, synthetic construct
  source 1..30: mol_type = other RNA
ccattcttaccttagaaaattgtgcattta

SEQ ID NO: 1153 - RNA, 30 nt, synthetic construct
  source 1..30: mol_type = other RNA
cattcttaccttagaaaattgtgcatttac

SEQ ID NO: 1154 - RNA, 30 nt, synthetic construct
  source 1..30: mol_type = other RNA
attcttaccttagaaaattgtgcatttacc

SEQ ID NO: 1155 - RNA, 30 nt, synthetic construct
  source 1..30: mol_type = other RNA
ttcttaccttagaaaattgtgcatttaccc

SEQ ID NO: 1156 - RNA, 30 nt, synthetic construct
  source 1..30: mol_type = other RNA
tcttaccttagaaaattgtgcatttaccca

SEQ ID NO: 1157 - RNA, 30 nt, synthetic construct
  source 1..30: mol_type = other RNA
cttaccttagaaaattgtgcatttacccat

SEQ ID NO: 1158 - RNA, 30 nt, synthetic construct
  source 1..30: mol_type = other RNA
ttaccttagaaaattgtgcatttacccatt

SEQ ID NO: 1159 - RNA, 30 nt, synthetic construct
  source 1..30: mol_type = other RNA
taccttagaaaattgtgcatttacccattt

SEQ ID NO: 1160 - RNA, 30 nt, synthetic construct
  source 1..30: mol_type = other RNA
accttagaaaattgtgcatttacccatttt

SEQ ID NO: 1161 - RNA, 30 nt, synthetic construct
  source 1..30: mol_type = other RNA
ccttagaaaattgtgcatttacccattttg

SEQ ID NO: 1162 - RNA, 30 nt, synthetic construct
  source 1..30: mol_type = other RNA
cttagaaaattgtgcatttacccattttgt

SEQ ID NO: 1163 - RNA, 30 nt, synthetic construct
  source 1..30: mol_type = other RNA
ttagaaaattgtgcatttacccattttgtg

SEQ ID NO: 1164 - RNA, 30 nt, synthetic construct
  source 1..30: mol_type = other RNA
tagaaaattgtgcatttacccattttgtga

SEQ ID NO: 1165 - RNA, 30 nt, synthetic construct
  source 1..30: mol_type = other RNA
agaaaattgtgcatttacccattttgtgaa

SEQ ID NO: 1166 - RNA, 30 nt, synthetic construct
  source 1..30: mol_type = other RNA
gaaaattgtgcatttacccattttgtgaat

SEQ ID NO: 1167 - RNA, 30 nt, synthetic construct
  source 1..30: mol_type = other RNA
aaaattgtgcatttacccattttgtgaatg

SEQ ID NO: 1168 - RNA, 30 nt, synthetic construct
  source 1..30: mol_type = other RNA
aaattgtgcatttacccattttgtgaatgt

SEQ ID NO: 1169 - RNA, 30 nt, synthetic construct
  source 1..30: mol_type = other RNA
aattgtgcatttacccattttgtgaatgtt

SEQ ID NO: 1170 - RNA, 30 nt, synthetic construct
  source 1..30: mol_type = other RNA
attgtgcatttacccattttgtgaatgttt

SEQ ID NO: 1171 - RNA, 30 nt, synthetic construct
  source 1..30: mol_type = other RNA
ttgtgcatttacccattttgtgaatgtttt

SEQ ID NO: 1172 - RNA, 30 nt, synthetic construct
  source 1..30: mol_type = other RNA
tgtgcatttacccattttgtgaatgttttc

SEQ ID NO: 1173 - RNA, 30 nt, synthetic construct
  source 1..30: mol_type = other RNA
gtgcatttacccattttgtgaatgttttct

SEQ ID NO: 1174 - RNA, 30 nt, synthetic construct
  source 1..30: mol_type = other RNA
tgcatttacccattttgtgaatgttttctt

SEQ ID NO: 1175 - RNA, 30 nt, synthetic construct
  source 1..30: mol_type = other RNA
gcatttacccattttgtgaatgttttcttt

SEQ ID NO: 1176 - RNA, 30 nt, synthetic construct
  source 1..30: mol_type = other RNA
catttacccattttgtgaatgttttctttt

SEQ ID NO: 1177 - RNA, 30 nt, synthetic construct
  source 1..30: mol_type = other RNA
atttacccattttgtgaatgttttcttttg

SEQ ID NO: 1178 - RNA, 30 nt, synthetic construct
  source 1..30: mol_type = other RNA
tttacccattttgtgaatgttttcttttga

SEQ ID NO: 1179 - RNA, 30 nt, synthetic construct
  source 1..30: mol_type = other RNA
ttacccattttgtgaatgttttcttttgaa

SEQ ID NO: 1180 - RNA, 30 nt, synthetic construct
  source 1..30: mol_type = other RNA
tacccattttgtgaatgttttcttttgaac

SEQ ID NO: 1181 - RNA, 30 nt, synthetic construct
  source 1..30: mol_type = other RNA
acccattttgtgaatgttttcttttgaaca

SEQ ID NO: 1182 - RNA, 30 nt, synthetic construct
  source 1..30: mol_type = other RNA
cccattttgtgaatgttttcttttgaacat

SEQ ID NO: 1183 - RNA, 30 nt, synthetic construct
  source 1..30: mol_type = other RNA
ccattttgtgaatgttttcttttgaacatc

SEQ ID NO: 1184 - RNA, 30 nt, synthetic construct
  source 1..30: mol_type = other RNA
cattttgtgaatgttttcttttgaacatct

SEQ ID NO: 1185 - RNA, 30 nt, synthetic construct
  source 1..30: mol_type = other RNA
attttgtgaatgttttcttttgaacatctt

SEQ ID NO: 1186 - RNA, 30 nt, synthetic construct
  source 1..30: mol_type = other RNA
ttttgtgaatgttttcttttgaacatcttc

SEQ ID NO: 1187 - RNA, 30 nt, synthetic construct
  source 1..30: mol_type = other RNA
tttgtgaatgttttcttttgaacatcttct

SEQ ID NO: 1188 - RNA, 30 nt, synthetic construct
  source 1..30: mol_type = other RNA
ttgtgaatgttttcttttgaacatcttctc

SEQ ID NO: 1189 - RNA, 30 nt, synthetic construct
  source 1..30: mol_type = other RNA
tgtgaatgttttcttttgaacatcttctct

SEQ ID NO: 1190 - RNA, 30 nt, synthetic construct
  source 1..30: mol_type = other RNA
gtgaatgttttcttttgaacatcttctctt

SEQ ID NO: 1191 - RNA, 30 nt, synthetic construct
  source 1..30: mol_type = other RNA
tgaatgttttcttttgaacatcttctcttt

SEQ ID NO: 1192 - RNA, 30 nt, synthetic construct
  source 1..30: mol_type = other RNA
gaatgttttcttttgaacatcttctctttc

SEQ ID NO: 1193 - RNA, 30 nt, synthetic construct
  source 1..30: mol_type = other RNA
aatgttttcttttgaacatcttctctttca

SEQ ID NO: 1194 - RNA, 30 nt, synthetic construct
  source 1..30: mol_type = other RNA
atgttttcttttgaacatcttctctttcat

SEQ ID NO: 1195 - RNA, 30 nt, synthetic construct; identical to SEQ ID NO: 69
  source 1..30: mol_type = other RNA

SEQ ID NO: 1196 - RNA, 30 nt, synthetic construct
  source 1..30: mol_type = other RNA
gttttcttttgaacatcttctctttcatct

SEQ ID NO: 1197 - RNA, 30 nt, synthetic construct
  source 1..30: mol_type = other RNA
ttttcttttgaacatcttctctttcatcta

SEQ ID NO: 1198 - RNA, 30 nt, synthetic construct
  source 1..30: mol_type = other RNA
tttcttttgaacatcttctctttcatctaa

SEQ ID NO: 1199 - RNA, 30 nt, synthetic construct
  source 1..30: mol_type = other RNA
ttcttttgaacatcttctctttcatctaaa

SEQ ID NO: 1200 - RNA, 30 nt, synthetic construct
  source 1..30: mol_type = other RNA
tcttttgaacatcttctctttcatctaaaa

SEQ ID NO: 1201 - RNA, 30 nt, synthetic construct
  source 1..30: mol_type = other RNA
cttttgaacatcttctctttcatctaaaat

SEQ ID NO: 1202 - RNA, 30 nt, synthetic construct
  source 1..30: mol_type = other RNA
ttttgaacatcttctctttcatctaaaatg

SEQ ID NO: 1203 - RNA, 30 nt, synthetic construct
  source 1..30: mol_type = other RNA
tttgaacatcttctctttcatctaaaatgc

SEQ ID NO: 1204 - RNA, 30 nt, synthetic construct
  source 1..30: mol_type = other RNA
ttgaacatcttctctttcatctaaaatgca

SEQ ID NO: 1205 - RNA, 30 nt, synthetic construct
  source 1..30: mol_type = other RNA
tgaacatcttctctttcatctaaaatgcaa

SEQ ID NO: 1206 - RNA, 30 nt, synthetic construct
  source 1..30: mol_type = other RNA
gaacatcttctctttcatctaaaatgcaaa

SEQ ID NO: 1207 - RNA, 30 nt, synthetic construct
  source 1..30: mol_type = other RNA
aacatcttctctttcatctaaaatgcaaaa

SEQ ID NO: 1208 - RNA, 30 nt, synthetic construct
  source 1..30: mol_type = other RNA
acatcttctctttcatctaaaatgcaaaat

SEQ ID NO: 1209 - RNA, 30 nt, synthetic construct
  source 1..30: mol_type = other RNA
catcttctctttcatctaaaatgcaaaata

SEQ ID NO: 1210 - RNA, 30 nt, synthetic construct
  source 1..30: mol_type = other RNA
atcttctctttcatctaaaatgcaaaataa

SEQ ID NO: 1211 - RNA, 30 nt, synthetic construct
  source 1..30: mol_type = other RNA
tcttctctttcatctaaaatgcaaaataaa

SEQ ID NO: 1212 - RNA, 30 nt, synthetic construct
  source 1..30: mol_type = other RNA
cttctctttcatctaaaatgcaaaataaaa

SEQ ID NO: 1213 - RNA, 30 nt, synthetic construct
  source 1..30: mol_type = other RNA
ttctctttcatctaaaatgcaaaataaaaa

SEQ ID NO: 1214 - RNA, 30 nt, synthetic construct
  source 1..30: mol_type = other RNA
tctctttcatctaaaatgcaaaataaaaaa

SEQ ID NO: 1215 - RNA, 30 nt, synthetic construct
  source 1..30: mol_type = other RNA
ctctttcatctaaaatgcaaaataaaaaaa

SEQ ID NO: 1216 - RNA, 30 nt, synthetic construct
  source 1..30: mol_type = other RNA
tctttcatctaaaatgcaaaataaaaaaat

SEQ ID NO: 1217 - RNA, 30 nt, synthetic construct
  source 1..30: mol_type = other RNA
ctttcatctaaaatgcaaaataaaaaaata

SEQ ID NO: 1218 - RNA, 30 nt, synthetic construct
  source 1..30: mol_type = other RNA
tttcatctaaaatgcaaaataaaaaaataa

SEQ ID NO: 1219 - RNA, 30 nt, synthetic construct
  source 1..30: mol_type = other RNA
ttcatctaaaatgcaaaataaaaaaataaa

SEQ ID NO: 1220 - RNA, 30 nt, synthetic construct
  source 1..30: mol_type = other RNA
tcatctaaaatgcaaaataaaaaaataaaa

SEQ ID NO: 1221 - RNA, 30 nt, synthetic construct
  source 1..30: mol_type = other RNA
catctaaaatgcaaaataaaaaaataaaag

SEQ ID NO: 1222 - RNA, 30 nt, synthetic construct
  source 1..30: mol_type = other RNA
atctaaaatgcaaaataaaaaaataaaagt

SEQ ID NO: 1223 - RNA, 30 nt, synthetic construct
  source 1..30: mol_type = other RNA
tctaaaatgcaaaataaaaaaataaaagtt

SEQ ID NO: 1224 - RNA, 30 nt, synthetic construct
  source 1..30: mol_type = other RNA
ctaaaatgcaaaataaaaaaataaaagtta

SEQ ID NO: 1225 - RNA, 30 nt, synthetic construct
  source 1..30: mol_type = other RNA
taaaatgcaaaataaaaaaataaaagttag

SEQ ID NO: 1226 - RNA, 30 nt, synthetic construct
  source 1..30: mol_type = other RNA
aaaatgcaaaataaaaaaataaaagttagg

SEQ ID NO: 1227 - RNA, 30 nt, synthetic construct
  source 1..30: mol_type = other RNA
aaatgcaaaataaaaaaataaaagttagga

SEQ ID NO: 1228 - RNA, 30 nt, synthetic construct
  source 1..30: mol_type = other RNA
aatgcaaaataaaaaaataaaagttaggaa

SEQ ID NO: 1229 - RNA, 30 nt, synthetic construct
  source 1..30: mol_type = other RNA
atgcaaaataaaaaaataaaagttaggaag

SEQ ID NO: 1230 - RNA, 30 nt, synthetic construct
  source 1..30: mol_type = other RNA
tgcaaaataaaaaaataaaagttaggaagc

SEQ ID NO: 1231 - RNA, 30 nt, synthetic construct
  source 1..30: mol_type = other RNA
gcaaaataaaaaaataaaagttaggaagca

SEQ ID NO: 1232 - RNA, 30 nt, synthetic construct
  source 1..30: mol_type = other RNA
caaaataaaaaaataaaagttaggaagcaa

SEQ ID NO: 1233 - RNA, 30 nt, synthetic construct
  source 1..30: mol_type = other RNA
aaaataaaaaaataaaagttaggaagcaac

SEQ ID NO: 1234 - RNA, 30 nt, synthetic construct
  source 1..30: mol_type = other RNA
aaataaaaaaataaaagttaggaagcaact

SEQ ID NO: 1235 - RNA, 30 nt, synthetic construct
  source 1..30: mol_type = other RNA
aataaaaaaataaaagttaggaagcaactt

SEQ ID NO: 1236 - RNA, 30 nt, synthetic construct
  source 1..30: mol_type = other RNA
ataaaaaaataaaagttaggaagcaacttt

SEQ ID NO: 1237 - RNA, 30 nt, synthetic construct
  source 1..30: mol_type = other RNA
taaaaaaataaaagttaggaagcaacttta

SEQ ID NO: 1238 - RNA, 30 nt, synthetic construct
  source 1..30: mol_type = other RNA
aaaaaaataaaagttaggaagcaactttaa

SEQ ID NO: 1239 - RNA, 30 nt, synthetic construct
  source 1..30: mol_type = other RNA
aaaaaataaaagttaggaagcaactttaaa

SEQ ID NO: 1240 - RNA, 30 nt, synthetic construct
  source 1..30: mol_type = other RNA
aaaaataaaagttaggaagcaactttaaat

SEQ ID NO: 1241 - RNA, 30 nt, synthetic construct
  source 1..30: mol_type = other RNA
aaaataaaagttaggaagcaactttaaata

SEQ ID NO: 1242 - RNA, 30 nt, synthetic construct
  source 1..30: mol_type = other RNA
aaataaaagttaggaagcaactttaaatat

SEQ ID NO: 1243 - RNA, 30 nt, synthetic construct
  source 1..30: mol_type = other RNA
aataaaagttaggaagcaactttaaatata

SEQ ID NO: 1244 - RNA, 30 nt, synthetic construct
  source 1..30: mol_type = other RNA
ataaaagttaggaagcaactttaaatataa

SEQ ID NO: 1245 - RNA, 30 nt, synthetic construct
  source 1..30: mol_type = other RNA
taaaagttaggaagcaactttaaatataat

SEQ ID NO: 1246 - RNA, 30 nt, synthetic construct
  source 1..30: mol_type = other RNA
aaaagttaggaagcaactttaaatataatt

SEQ ID NO: 1247 - RNA, 30 nt, synthetic construct
  source 1..30: mol_type = other RNA
aaagttaggaagcaactttaaatataattc

SEQ ID NO: 1248 - RNA, 30 nt, synthetic construct
  source 1..30: mol_type = other RNA
aagttaggaagcaactttaaatataattca

SEQ ID NO: 1249 - RNA, 30 nt, synthetic construct
  source 1..30: mol_type = other RNA
agttaggaagcaactttaaatataattcat

SEQ ID NO: 1250 - RNA, 30 nt, synthetic construct
  source 1..30: mol_type = other RNA
gttaggaagcaactttaaatataattcata

SEQ ID NO: 1251 - RNA, 30 nt, synthetic construct
  source 1..30: mol_type = other RNA
ttaggaagcaactttaaatataattcatat

SEQ ID NO: 1252 - RNA, 30 nt, synthetic construct
  source 1..30: mol_type = other RNA
taggaagcaactttaaatataattcatatt

SEQ ID NO: 1253 - RNA, 30 nt, synthetic construct
  source 1..30: mol_type = other RNA
aggaagcaactttaaatataattcatattt

SEQ ID NO: 1254 - RNA, 30 nt, synthetic construct
  source 1..30: mol_type = other RNA
ggaagcaactttaaatataattcatatttt

SEQ ID NO: 1255 - RNA, 30 nt, synthetic construct
  source 1..30: mol_type = other RNA
gaagcaactttaaatataattcatatttta

SEQ ID NO: 1256 - RNA, 30 nt, synthetic construct
  source 1..30: mol_type = other RNA
aagcaactttaaatataattcatattttac

SEQ ID NO: 1257 - RNA, 30 nt, synthetic construct
  source 1..30: mol_type = other RNA
agcaactttaaatataattcatattttaca

SEQ ID NO: 1258 - RNA, 30 nt, synthetic construct
  source 1..30: mol_type = other RNA
gcaactttaaatataattcatattttacaa

SEQ ID NO: 1259 - RNA, 30 nt, synthetic construct
  source 1..30: mol_type = other RNA
caactttaaatataattcatattttacaac

SEQ ID NO: 1260 - RNA, 30 nt, synthetic construct
  source 1..30: mol_type = other RNA
aactttaaatataattcatattttacaacc

SEQ ID NO: 1261 - RNA, 30 nt, synthetic construct
  source 1..30: mol_type = other RNA
actttaaatataattcatattttacaacca

SEQ ID NO: 1262 - RNA, 30 nt, synthetic construct
  source 1..30: mol_type = other RNA
ctttaaatataattcatattttacaaccaa

SEQ ID NO: 1263 - RNA, 30 nt, synthetic construct
  source 1..30: mol_type = other RNA
tttaaatataattcatattttacaaccaaa

SEQ ID NO: 1264 - RNA, 30 nt, synthetic construct
  source 1..30: mol_type = other RNA
ttaaatataattcatattttacaaccaaag

SEQ ID NO: 1265 - RNA, 30 nt, synthetic construct
  source 1..30: mol_type = other RNA
taaatataattcatattttacaaccaaagt

SEQ ID NO: 1266 - RNA, 30 nt, synthetic construct
  source 1..30: mol_type = other RNA
aaatataattcatattttacaaccaaagta

SEQ ID NO: 1267 - RNA, 30 nt, synthetic construct
  source 1..30: mol_type = other RNA
aatataattcatattttacaaccaaagtaa

SEQ ID NO: 1268 - RNA, 30 nt, synthetic construct
  source 1..30: mol_type = other RNA
atataattcatattttacaaccaaagtaac

SEQ ID NO: 1269 - RNA, 30 nt, synthetic construct
  source 1..30: mol_type = other RNA
tataattcatattttacaaccaaagtaact

SEQ ID NO: 1270 - RNA, 30 nt, synthetic construct
  source 1..30: mol_type = other RNA
ataattcatattttacaaccaaagtaactt

SEQ ID NO: 1271 - RNA, 30 nt, synthetic construct
  source 1..30: mol_type = other RNA
taattcatattttacaaccaaagtaacttt

SEQ ID NO: 1272 - RNA, 30 nt, synthetic construct
  source 1..30: mol_type = other RNA
aattcatattttacaaccaaagtaactttc

SEQ ID NO: 1273 - RNA, 30 nt, synthetic construct
  source 1..30: mol_type = other RNA
attcatattttacaaccaaagtaactttcc

SEQ ID NO: 1274 - RNA, 30 nt, synthetic construct
  source 1..30: mol_type = other RNA
ttcatattttacaaccaaagtaactttcca

SEQ ID NO: 1275 - RNA, 30 nt, synthetic construct
  source 1..30: mol_type = other RNA
tcatattttacaaccaaagtaactttccat

SEQ ID NO: 1276 - RNA, 30 nt, synthetic construct
  source 1..30: mol_type = other RNA
catattttacaaccaaagtaactttccatt

SEQ ID NO: 1277 - RNA, 30 nt, synthetic construct
  source 1..30: mol_type = other RNA
atattttacaaccaaagtaactttccatta

SEQ ID NO: 1278 - RNA, 30 nt, synthetic construct
  source 1..30: mol_type = other RNA
tattttacaaccaaagtaactttccattat

SEQ ID NO: 1279 - RNA, 30 nt, synthetic construct
  source 1..30: mol_type = other RNA
attttacaaccaaagtaactttccattatg

SEQ ID NO: 1280 - RNA, 30 nt, synthetic construct
  source 1..30: mol_type = other RNA
ttttacaaccaaagtaactttccattatga

SEQ ID NO: 1281 - RNA, 30 nt, synthetic construct
  source 1..30: mol_type = other RNA
tttacaaccaaagtaactttccattatgat

SEQ ID NO: 1282 - RNA, 30 nt, synthetic construct
  source 1..30: mol_type = other RNA
ttacaaccaaagtaactttccattatgatg

SEQ ID NO: 1283 - RNA, 30 nt, synthetic construct
  source 1..30: mol_type = other RNA
tacaaccaaagtaactttccattatgatgt

SEQ ID NO: 1284 - RNA, 30 nt, synthetic construct
  source 1..30: mol_type = other RNA
acaaccaaagtaactttccattatgatgtg

SEQ ID NO: 1285 - RNA, 30 nt, synthetic construct
  source 1..30: mol_type = other RNA
caaccaaagtaactttccattatgatgtgt

SEQ ID NO: 1286 - RNA, 30 nt, synthetic construct
  source 1..30: mol_type = other RNA
aaccaaagtaactttccattatgatgtgtt

SEQ ID NO: 1287 - RNA, 30 nt, synthetic construct
  source 1..30: mol_type = other RNA
accaaagtaactttccattatgatgtgtta

SEQ ID NO: 1288 - RNA, 30 nt, synthetic construct
  source 1..30: mol_type = other RNA
ccaaagtaactttccattatgatgtgttag

SEQ ID NO: 1289 - RNA, 30 nt, synthetic construct
  source 1..30: mol_type = other RNA
caaagtaactttccattatgatgtgttagt

SEQ ID NO: 1290 - RNA, 30 nt, synthetic construct
  source 1..30: mol_type = other RNA
aaagtaactttccattatgatgtgttagtg

SEQ ID NO: 1291 - RNA, 30 nt, synthetic construct
  source 1..30: mol_type = other RNA
aagtaactttccattatgatgtgttagtgt

SEQ ID NO: 1292 - RNA, 30 nt, synthetic construct
  source 1..30: mol_type = other RNA
agtaactttccattatgatgtgttagtgtt

SEQ ID NO: 1293 - RNA, 30 nt, synthetic construct
  source 1..30: mol_type = other RNA
gtaactttccattatgatgtgttagtgttt

SEQ ID NO: 1294 - RNA, 30 nt, synthetic construct
  source 1..30: mol_type = other RNA
taactttccattatgatgtgttagtgtttt

SEQ ID NO: 1295 - RNA, 30 nt, synthetic construct
  source 1..30: mol_type = other RNA
aactttccattatgatgtgttagtgttttt

SEQ ID NO: 1296 - RNA, 30 nt, synthetic construct
  source 1..30: mol_type = other RNA
actttccattatgatgtgttagtgtttttt

SEQ ID NO: 1297 - RNA, 30 nt, synthetic construct
  source 1..30: mol_type = other RNA
ctttccattatgatgtgttagtgttttttt

SEQ ID NO: 1298 - RNA, 30 nt, synthetic construct
  source 1..30: mol_type = other RNA
tttccattatgatgtgttagtgttttttta

SEQ ID NO: 1299 - RNA, 30 nt, synthetic construct
  source 1..30: mol_type = other RNA
ttccattatgatgtgttagtgtttttttac

SEQ ID NO: 1300 - RNA, 30 nt, synthetic construct
  source 1..30: mol_type = other RNA
tccattatgatgtgttagtgtttttttaca

SEQ ID NO: 1301 - RNA, 30 nt, synthetic construct
  source 1..30: mol_type = other RNA
ccattatgatgtgttagtgtttttttacat

SEQ ID NO: 1302 - RNA, 30 nt, synthetic construct
  source 1..30: mol_type = other RNA
cattatgatgtgttagtgtttttttacatt

SEQ ID NO: 1303 - RNA, 30 nt, synthetic construct
  source 1..30: mol_type = other RNA
attatgatgtgttagtgtttttttacattt

SEQ ID NO: 1304 - RNA, 30 nt, synthetic construct
  source 1..30: mol_type = other RNA
ttatgatgtgttagtgtttttttacattta

SEQ ID NO: 1305 - RNA, 30 nt, synthetic construct
  source 1..30: mol_type = other RNA
tatgatgtgttagtgtttttttacatttag

SEQ ID NO: 1306 - RNA, 30 nt, synthetic construct
  source 1..30: mol_type = other RNA
atgatgtgttagtgtttttttacatttagt

SEQ ID NO: 1307 - RNA, 30 nt, synthetic construct
  source 1..30: mol_type = other RNA
tgatgtgttagtgtttttttacatttagta

SEQ ID NO: 1308 - RNA, 30 nt, synthetic construct
  source 1..30: mol_type = other RNA
gatgtgttagtgtttttttacatttagtat

SEQ ID NO: 1309 - RNA, 30 nt, synthetic construct
  source 1..30: mol_type = other RNA
atgtgttagtgtttttttacatttagtatt

SEQ ID NO: 1310 - RNA, 30 nt, synthetic construct
  source 1..30: mol_type = other RNA
tgtgttagtgtttttttacatttagtatta

SEQ ID NO: 1311 - RNA, 30 nt, synthetic construct
  source 1..30: mol_type = other RNA
gtgttagtgtttttttacatttagtattac

SEQ ID NO: 1312 - RNA, 30 nt, synthetic construct
  source 1..30: mol_type = other RNA
tgttagtgtttttttacatttagtattaca

SEQ ID NO: 1313 - RNA, 30 nt, synthetic construct
  source 1..30: mol_type = other RNA
gttagtgtttttttacatttagtattacat

SEQ ID NO: 1314 - RNA, 30 nt, synthetic construct
  source 1..30: mol_type = other RNA
ttagtgtttttttacatttagtattacatt

SEQ ID NO: 1315 - RNA, 30 nt, synthetic construct
  source 1..30: mol_type = other RNA
tagtgtttttttacatttagtattacattc

SEQ ID NO: 1316 - RNA, 30 nt, synthetic construct
  source 1..30: mol_type = other RNA
agtgtttttttacatttagtattacattca

SEQ ID NO: 1317 - RNA, 30 nt, synthetic construct
  source 1..30: mol_type = other RNA
gtgtttttttacatttagtattacattcat

SEQ ID NO: 1318 - RNA, 30 nt, synthetic construct
  source 1..30: mol_type = other RNA
tgtttttttacatttagtattacattcatt

SEQ ID NO: 1319 - RNA, 30 nt, synthetic construct
  source 1..30: mol_type = other RNA
gtttttttacatttagtattacattcattg

SEQ ID NO: 1320 - RNA, 30 nt, synthetic construct
  source 1..30: mol_type = other RNA
tttttttacatttagtattacattcattga

SEQ ID NO: 1321 - RNA, 30 nt, synthetic construct
  source 1..30: mol_type = other RNA
ttttttacatttagtattacattcattgat

SEQ ID NO: 1322 - RNA, 30 nt, synthetic construct
  source 1..30: mol_type = other RNA
tttttacatttagtattacattcattgata

SEQ ID NO: 1323 - RNA, 30 nt, synthetic construct
  source 1..30: mol_type = other RNA
ttttacatttagtattacattcattgataa

SEQ ID NO: 1324 - RNA, 30 nt, synthetic construct
  source 1..30: mol_type = other RNA
tttacatttagtattacattcattgataaa

SEQ ID NO: 1325 - RNA, 30 nt, synthetic construct
  source 1..30: mol_type = other RNA
ttacatttagtattacattcattgataaat

SEQ ID NO: 1326 - RNA, 30 nt, synthetic construct
  source 1..30: mol_type = other RNA
tacatttagtattacattcattgataaatg

SEQ ID NO: 1327 - RNA, 30 nt, synthetic construct
  source 1..30: mol_type = other RNA
acatttagtattacattcattgataaatgg

SEQ ID NO: 1328 - RNA, 30 nt, synthetic construct
  source 1..30: mol_type = other RNA
catttagtattacattcattgataaatgga

SEQ ID NO: 1329 - RNA, 30 nt, synthetic construct
  source 1..30: mol_type = other RNA
atttagtattacattcattgataaatggaa

SEQ ID NO: 1330 - RNA, 30 nt, synthetic construct
  source 1..30: mol_type = other RNA
tttagtattacattcattgataaatggaat

SEQ ID NO: 1331 - RNA, 30 nt, synthetic construct
  source 1..30: mol_type = other RNA
ttagtattacattcattgataaatggaatt

SEQ ID NO: 1332 - RNA, 30 nt, synthetic construct
  source 1..30: mol_type = other RNA
tagtattacattcattgataaatggaatta

SEQ ID NO: 1333 - RNA, 30 nt, synthetic construct
  source 1..30: mol_type = other RNA
agtattacattcattgataaatggaattaa

SEQ ID NO: 1334 - RNA, 30 nt, synthetic construct
  source 1..30: mol_type = other RNA
gtattacattcattgataaatggaattaaa

SEQ ID NO: 1335 - RNA, 30 nt, synthetic construct
  source 1..30: mol_type = other RNA
tattacattcattgataaatggaattaaac

SEQ ID NO: 1336 - RNA, 30 nt, synthetic construct
  source 1..30: mol_type = other RNA
attacattcattgataaatggaattaaaca

SEQ ID NO: 1337 - RNA, 30 nt, synthetic construct
  source 1..30: mol_type = other RNA
ttacattcattgataaatggaattaaacat

SEQ ID NO: 1338 - RNA, 30 nt, synthetic construct
  source 1..30: mol_type = other RNA
tacattcattgataaatggaattaaacatg

SEQ ID NO: 1339 - RNA, 30 nt, synthetic construct
  source 1..30: mol_type = other RNA
acattcattgataaatggaattaaacatga

SEQ ID NO: 1340 - RNA, 30 nt, synthetic construct
  source 1..30: mol_type = other RNA
cattcattgataaatggaattaaacatgag

SEQ ID NO: 1341 - RNA, 30 nt, synthetic construct
  source 1..30: mol_type = other RNA
attcattgataaatggaattaaacatgagt

SEQ ID NO: 1342 - RNA, 30 nt, synthetic construct
  source 1..30: mol_type = other RNA
ttcattgataaatggaattaaacatgagtg

SEQ ID NO: 1343 - RNA, 30 nt, synthetic construct
  source 1..30: mol_type = other RNA
tcattgataaatggaattaaacatgagtga

SEQ ID NO: 1344 - RNA, 30 nt, synthetic construct
  source 1..30: mol_type = other RNA
cattgataaatggaattaaacatgagtgat

(19) United States
(12) Patent Application Publication
(10) Pub. No.: US 2026/0201382 A1
(43) Pub. Date: Jul. 16, 2026

(54) COMPOSITIONS AND METHODS COMPRISING SMALL NUCLEAR RNA (SNRNA) FOR TREATING DMD

(71) Applicant: Regeneron Pharmaceuticals, Inc., Tarrytown, NY (US)

(72) Inventors: Rea LARDELLI MARKMILLER, San Diego, CA (US); Daniela ROTH, San Diego, CA (US); Ranjan BATRA, San Diego, CA (US); William Henry BRADFORD, San Diego, CA (US); Daniel A. KNOWLAND, San Diego, CA (US); Gregory Thomas NACHTRAB, San Diego, CA (US); Alberto CARRENO, San Diego, CA (US)

(21) Appl. No.: 19/576,731

(22) Filed: Mar. 24, 2026

Related U.S. Application Data
(63) Continuation of application No. PCT/US2024/048670, filed on Sep. 26, 2024.
(60) Provisional application No. 63/585,293, filed on Sep. 26, 2023.

(51) Int. Cl. C12N 15/113; C12N 15/86

(52) U.S. Cl. CPC C12N 15/113; C12N 15/86; C12N 2320/33; C12N 2750/14143

(57) ABSTRACT

SnRNA systems targeting DMA RNA sequences are disclosed herein.

### RELATED APPLICATIONS

This application is a bypass continuation of International Application No. PCT/US2024/048670, filed Sep. 26, 2024, which claims the priority to, and benefit of, U.S. Provisional Application No. 63/585,293, filed on Sep. 26, 2023, the entire contents of each of which are incorporated by reference in their entireties.

### REFERENCE TO AN ELECTRONIC SEQUENCE LISTING

The contents of the electronic sequence listing (REGE_034_C01US_SeqList_ST26.xml; Size: 1,227,594 bytes; and Date of Creation: Mar. 23, 2026) is herein incorporated by reference in its entirety.

### FIELD OF THE DISCLOSURE

The disclosure is directed to molecular biology, gene therapy, and compositions and methods for modifying expression and activity of RNA molecules.

### BACKGROUND

There are long-felt but unmet needs in the art for providing effective therapies for correcting dysfunctional messenger RNA.

Small nuclear RNA (snRNA) is one of the smallest types of RNA with an average size of about 150 nucleotides. snRNAs are functional non-coding RNAs. Eucaryotic genomes code for a variety of non-coding RNA such as snRNA, a class of highly abundant RNA, localized in the nucleus with important functions in intron splicing and RNA processing. snRNA, in the pre-mRNA splicing process, are capable of forming ribonucleoprotein particles (snRNPs) along with other proteins. These snRNPs and additional proteins form a large particulate complex (spliceosome) bound to the unspliced pre-mRNA transcripts. In addition to splicing, snRNAs function in nuclear maturation of nascent transcripts, gene expression regulation, as a splice donor in non-canonical systems, and in 3′ end processing of replication-dependent histone mRNAs. U7 snRNA can be programmed to bind and modulate mRNA without exogenous protein expression, this will ultimately decrease the risk of immunogenicity, observed with other protein-based gene therapy approaches. Furthermore, the small size of these programmed snRNAs creates an opportunity to develop single vector, highly specific (allele-specific), single target and multi-targeting gene therapy approaches.

Duchenne Muscular Dystrophy is caused by mutations in the DMD gene. DMD-associated monogenic disease or disorder is Duchenne Muscular Dystrophy or Becker Muscular Dystrophy. DMD is the largest gene in the human genome having 79 exons separated by introns that are up to 250 kb. DMD encodes dystrophin protein, which stabilizes the plasma membrane in striated muscle. Exon skipping to restore frame (e.g., caused by exon 50 deletion frameshift) reduces the severity of Duchenne Muscular Dystrophy to that exhibited by Becker Muscular Dystrophy patients. Therefore, exon skipping to restore near full-length dystrophin protein presents a promising opportunity to treat Duchenne Muscular Dystrophy (DMD) patients, who have mRNA frame-shifting deletions in the DMD gene, however, exon skipping has generally been limited to antisense oligonucleotides, which have poor uptake in disease tissues. Recent efforts with AAV9-U7 snRNA gene therapy targeting exon 2 and use of modified U7 snRNA to convert an out-of-frame mutation into an in-frame mutation (Goyenvalle et al., 2009, 17(7): 1234-1240) have shown promise, but these early strategies struggle with low titers and inconsistent transgene expression. Accordingly, the disclosure provides compositions and methods comprising a new therapeutic RNA-targeting platform comprised of engineered snRNAs.

### SUMMARY

The disclosure provides an RNA-targeting nucleic acid molecule comprising an small nuclear RNA (snRNA), wherein the snRNA comprises a targeting sequence that binds a dystrophin (DMD) RNA sequence, wherein the DMD RNA sequence comprises at least one splicing regulatory sequence selected from: a splice acceptor sequence, a splice donor sequence, and an exon splice enhancer sequence.

In some aspects, the DMD RNA sequence is at least one of exon 2, exon 44, exon 45, exon 51, and/or exon 53.

In some aspects, the targeting sequence comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one of SEQ ID NO: 59-118, 126, 206-227 and 237-1344.

In some aspects, the snRNA comprises a stem loop (SL) comprising a nucleic acid sequence at least 65%, 70%, 75%, 80%, 85%, 90%, 95%, 96%, 97%, 98%, 99% or 100% (or any percentage in between) identical to one or more nucleic acid sequences set forth in any one of SEQ ID NO: 1-SEQ ID NO: 11, SEQ ID NO: 144-SEQ ID NO: 148, SEQ ID NO: 164, SEQ ID NO: 186, SEQ ID NO: 190-SEQ ID NO: 205, SEQ ID NO: 228-SEQ ID NO: 230, SEQ ID NO: 235, or SEQ ID NO: 236.

The RNA-targeting nucleic acid molecule of claim 1, wherein the stem loop (eSL) comprising a nucleic acid sequence at least 65%, 70%, 75%, 80%, 85%, 90%, 95%, 96%, 97%, 98%, 99% or 100% (or any percentage in between) identical to an engineered stem loop that comprises a nucleic acid sequence at least 65%, 70%, 75%, 80%, 85%, 90%, 95%, 96%, 97%, 98%, 99% or 100% (or any percentage in between) identical to one or more nucleic acid sequences set forth in any one of SEQ ID NO: 1-SEQ ID NO: 11

In some aspects, the snRNA comprises an engineered stem loop (eSL) comprising one or more nucleic acid sequences set forth in any one of SEQ ID NO: 1-SEQ ID NO: 11.

In some aspects, DMD RNA sequence is a pre-mRNA or mRNA sequence.

In some aspects, the snRNA comprises two targeting sequences that target two RNAs of interest. In some aspects, the two targeting sequences are a fusion sequence.

In some aspects, the snRNA comprises an Sm binding domain (SmBD) selected from the group consisting of a U1, U2, U4, and U5 SmBD. In some aspects, the SmBD comprises a nucleic acid sequence set forth in any one of SEQ ID NO: 31-SEQ ID NO: 38.

In some aspects, the snRNA comprises a 5′ interaction stabilizer domain (5′ISD) comprising a nucleotide sequence selected from any one of SEQ ID NO: 12-SEQ ID NO: 23.

In some aspects, the snRNA comprises a nucleic acid sequence set forth in the disclosure.

A vector comprising one or more snRNA of any embodiment of the disclosure. In some aspects, the vector is an AAV vector.

In some aspects, the snRNA is operably linked to a promoter. In some aspects, the snRNA is operably linked to a U7 promoter or a U1 promoter. In some aspects, the snRNA is operably linked to a downstream terminator (DT). In some aspects, the snRNA is operably linked to a U7 downstream terminator or a U1 downstream terminator.

In some aspects, the vector comprises at least one, at least two, at least three, at least four, or at least five snRNA. In some aspects, the least one, at least two, at least three, at least four, or at least five snRNA each target the same target RNA sequences. In some aspects, the least one, at least two, at least three, at least four, or at least five snRNA target two or more target RNA sequences

In some aspects, each snRNA is separated by a buffer sequence. In some aspects, the buffer sequence comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one SEQ ID NO: 24-SEQ ID NO: 30.

In some aspects, the vector comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one of SEQ ID NO: 130-SEQ ID NO: 138.

The disclosure provides a method of targeting one or more target RNAs of interest and blocking, knocking down, editing, exon-skipping or splicing the one or more target RNAs, comprising contacting an snRNA of the disclosure with a cell comprising the one or more target RNAs.

The disclosure provides a DMD RNA-targeting nucleic acid molecule comprising a targeting sequence set forth in any one of 59-118, 206-227 and 237-333.

The disclosure provides a method of treating a disease or disorder in a subject comprising administering an RNA-targeting nucleic acid molecule of the disclosure or an AAV vector of the disclosure.

In some aspects, the disease or disorder is Duchenne muscular dystrophy.

In some aspects, the administration is administration is intravenous, intramuscular, subpial, intrathecal, intraparenchymal, intrathecal, intrastriatal, subcutaneous, intradermal, intraperitoneal, intratumoral, intravenous, intraocular, and/or parenteral administration.

### BRIEF DESCRIPTION OF THE DRAWINGS

FIGS. 1A-1C show that fusion spacers promote higher levels of exon skipping than single spacers alone. U7 snRNAs modified to comprise spacers which bind splicing regulatory sequences (SA=splice acceptor, SD=splice donor, and/or ESE=exon enhancer sequences) to skip exon 53 leads to restoration of the DMD mRNA reading frame in the case of a DMD patient del52 mutation, and to restoration of dystrophin protein. FIG. 1A provides a schematic depicting expected PCR product sizes using primers in exon 51 and exon 54. FIG. 1B shows a TapeStation image of RT-PCR products after U7 snRNA treatments using single and fusion U7 synthetic RNAs, showing the DMD exon 53 PCR non-skipped product (top band, 365 bp) and skipped product (bottom band-153 bp). Untreated cells were used as negative control samples. FIG. 1C provides the quantification results from FIG. 1B shown as percentage of exon 53 skipping (skipped band/skipped band+ non-skipped band).

FIG. 2 shows AAV9 snRNA-mediated exon 53 skipping in human del45-52 DMD myotubes by restoration of dystrophin protein staining. Images depict immunofluorescence for dystrophin protein after transduction of DMD myotubes with A05014 (scAAV9-dual snRNA cassette, targeting exon 53), 1E5 vector genomes/cell after 1 week of treatment. Untreated cells shown on the right serve as negative control. DAPI stain represents nuclei.

FIGS. 3A-3B show AAV9 snRNA-mediated exon 53 skipping in vitro and in vivo. FIG. 3A provides a graph depicting percent exon 53 skipping in human DMD myotubes 7 days following treatment with A05211 with MOIs of 1E3, 1E4, and 1E5 vg/cell. FIG. 3B provides a graph depicting percent exon 53 skipping across multiple tissues ((gastrocnemius (gastroc), heart, liver, tibialis anterior (TA), brain, diaphragm, and triceps (tricep)) 4 weeks following retro-orbital administration with 2.4E12 vg/animal AAV9-A05211.

FIGS. 4A-4C show the DMD exon 44 targeting with synthetic fusion U7 snRNAs in exon 45 deleted patient myotubes. FIG. 4A illustrates the mechanism of action of snRNAs to promote DMD exon 44 skipping and restoration of frame on patient mutations such as deletion of exon 45. Accordingly, U7 snRNAs were modified to bind splicing regulatory sequences to promote skipping of exon 44. FIG. 4B shows a Tapestation image of the RT-PCR products after U7 snRNA treatments using single and fusion U7 synthetic RNAs in A45 Inserm myotubes, showing the DMD exon 44 PCR non-skipped (top band), and skipped products (bottom band). Non-targeting U7 (NT) and untreated cells (U) were used as negative control samples. FIG. 4C depicts the quantification results from 4B shown as percentage of exon 44 skipping (intensity of skipped band/intensity of skipped band+ intensity of non-skipped band).

FIGS. 5A-5B show increased levels of exon 44 skipping using 2^{nd} generation fusion spacers targeting exon 44 in 445 myotubes. FIG. 5A shows a Tapestation image of the RT-PCR products after U7 snRNA treatments (synthetic RNAs) using optimized 2^{nd} generation fusion spacers. The gel image shows the DMD exon 44 PCR non-skipped (top band) and skipped products (bottom band). Non-targeting U7 (NT) and untreated cells (U) were used as negative control samples. FIG. 5B depicts the quantification results from FIG. 5A shown as percentage of exon 44 skipping (intensity of skipped band/intensity of skipped band+ intensity of non-skipped band).

FIGS. 6A-6D show AAV9-mediated snRNA exon 44 skipping in patient myotubes. FIG. 6A provides a graph depicting percentage of exon 44 skipping (intensity of skipped band/intensity of skipped band+ intensity of non-skipped band) in 445 Inserm myotubes transduced with varying MOIs of A05374 and A05375 (scAAV9 vector with 2× U7 snRNA cassettes). Synthetic U7 sR220334 was used as a positive control, untreated (UNT) and AAV null (empty capsid) are negative controls. FIG. 6B shows U7 snRNA expression at varying MOIs shown as snRNA copies per nanogram of total RNA by ddPCR for A05374 and A05375. FIG. 6C shows a Jess blot of dystrophin protein expression following treatment with either A05374 or A05375 at various MOIs. AAV null and untreated (NT) are negative controls. Lysate from wild type (WT) myotubes were used to generate 0-50% standard curve for dystrophin (Dys) expression. Alpha-actinin (α-Act) is loading control. FIG. 6D provides a graph showing quantification of dystrophin expression relative to WT levels and normalized to alpha-actinin.

FIG. 7A-7C show a synthetic snRNA screen to target exon 45 for skipping. U7 snRNAs were engineered to bind splicing regulatory sequences (SA, splice acceptor; SD splice donor; and/or ESE, exon splice enhancer sequences) to promote skipping of exon 45 which will lead to restoration of DMD mRNA reading frame in the case of DMD patient 444 mutation, and restoration of dystrophin protein. FIG. 7A provides a schematic depicting expected RT-PCR product sizes using primers in exon 43 and exons 46/47 following transfection of human myotubes with 100 nM of synthetic snRNA. The predicted size for exon 45 included is 487 bp while exon 45 excluded is 311 bp. FIG. 7B shows a TapeStation image of RT-PCR products after transfecting synthetic RNAs. sR04, which is a synthetic snRNA that carries a non-targeting sequence and untreated cells (unt) were used as negative control samples. FIG. 7C provides a graph depicting the quantification of FIG. 7B shown as percentage of exon 45 skipping (skipped band/skipped band+ non-skipped band).

FIGS. 8A-8B show a screening of synthetic snRNAs to identify potent spacer sequences at a lower concentration of synthetic snRNA (25 nM). FIG. 8A shows a TapeStation image of RT-PCR products after transfecting U7 snRNA synthetic RNAs. sR04, which is a synthetic snRNA that carries a non-targeting sequence and untreated (unt) cells were used as negative control samples. Exon 45 included is predicted to be 487 bp while exon 45 excluded is predicted to be 311 bp. FIG. 8B provides a graph depicting the quantification of FIG. 8A shown as percentage of exon 45 skipping (skipped band/skipped band+ non-skipped band).

FIGS. 9A-9B show AAV9-mediated exon 45 skipping in human A44 (deletion of exon 44) myotubes. FIG. 9A provides a graph depicting the percent exon 45 skipping 7 days following transduction with varying MOIs of A05189 and A05190 (scAAV9-dual snRNA cassette). FIG. 9B provides a graph depicting the copies of snRNA per nanogram of total RNA as assessed by ddPCR for the snRNAs expressed from A05189 and A05190.

FIGS. 10A-10B show single and multi-exon skipping of DMD exons 50-53. FIG. 10A provides a TapeStation image of RT-PCR extracted RNA 7 days post transduction of human del52 myotubes with AAV-based 4× construct, A05178, to identify exon skipping. A05178 carries engineered snRNA that target exon 50, exon 51, exon 52, and exon 53. FIG. 10B provides a graph of percentage of skipping of the exons. Exon skipped bands were quantified by dividing the peak molarity of the amplicon (representing single or multi-exon skipping) by the signal for all DMD amplicons.

FIGS. 11A-11B show a multi-exon skipping strategy for skipping exons 44 and 45. FIG. 11A provides a TapeStation image of RT-PCR products to identify skipping post 24 hours transfection of human wild-type myotubes with U7 synthetic snRNAs targeting exon 44 and exon 45. FIG. 11B provides a graph of percentage of skipping of the exons. To determine % multi-skipping, the signal for exon 44 and exon 45 skipped band was divided by the signal for all the DMD amplicons.

### DETAILED DESCRIPTION

The disclosure provides gene therapy compositions comprising a therapeutic RNA-targeting platform comprised of short nuclear RNA (snRNA) targeting precursor mRNA (pre-mRNA) or mRNA sequences encoding dystrophin (DMD). The targeted pre-mRNA or mRNA sequences can include exonic regions of DMD and/or splicing regulatory sequences of DMD.

Disclosed herein are compositions comprising nucleic acid molecules, and vectors comprising the snRNA construct or constructs targeting DMD. snRNA molecules of the disclosure can be non-natural, modified and/or engineered snRNA (esnRNA). esnRNA targeting DMD of the disclosure comprise a mutated snRNA stem loop. In some aspects snRNA targeting DMD of the disclosure comprises a native stem loop.

Small nuclear ribonucleic acids (snRNAs) are essential components of small nuclear ribonucleoprotein complexes (snRNPs) which, when assembled with additional proteins, form the large ribonucleoprotein complex known as the spliceosome, the cell machinery appointed to mediate the entire mRNA maturation process. The spliceosome is responsible for precursor mRNA splicing; the process that removes introns from RNA transcripts before protein production. An individual snRNA is generally about 250 nucleotides or less in size. For example, U1 snRNA is 164 nucleotides in length and is encoded by genes that occur in several copies within the human genome. U1 snRNA represents the ribonucleic component of the nuclear particle U1 snRNP. The U1 snRNA has a stem and loop tridimensional structure and within the 5′ region there is a single-stranded sequence, generally about 9 nucleotides in length, capable of binding by complementary base pairing to the splicing donor site on the pre-mRNA molecule. (Horowitz et al., 1994, Trends Genet., 10(3): 100-6.) The various spliceosomal snRNAs have been designated as U1, U2, U4, U5, U6, U4ATAC, U6ATAC, U7, U11 and U12, due to the generous amount of uridylic acid they contain. (Mattaj et al., 1993, FASEB J, 15, 7:47-53.)

snRNA systems can be used for treating toxic mutations. For example, antisense oligonucleotides that interfere with splice sites and regulatory elements within an exon containing toxic mutations can induce skipping of specific exons at the pre-RNA level. Such antisense sequences can be packaged in an snRNA sequence delivered using viral vectors carrying a nucleic acid sequence from which the snRNA can be transcribed. U7 snRNA is endogenously involved in histone pre-mRNA 3′-end processing but can be converted into a versatile tool for splicing modulation by a small change in the binding site for Sm/Lsm proteins. One such therapeutic strategy for treating Duchenne muscular dystrophy has used modified U7 snRNA to convert an out-of-frame mutation into an in-frame mutation, which gives rise to internally deleted toxic RNA, but still functional dystrophin. (Goyenvalle et al., 2009, 17(7): 1234-1240.)

Most U-rich snRNPs are complexes that mediate the splicing of pre-mRNAs. U7 snRNP is an exception. U7 is not involved in splicing but rather is a key factor in the unique 3′-end processing of replication-dependent histone mRNAs. By modifying the U7 snRNA histone binding sequence and the Sm motif, U7 can no longer be involved in processing the histone pre-mRNA and instead targets pre-mRNAs or smRNA for blocking or splicing modulation. In this manner, U7 snRNA can be used as an effective gene therapy platform. A U7 snRNA platform also has the additional advantages of being a compact size, having the capability to accumulate in the nucleus without causing cellular toxicity, and possesses little to no immunoreactivity. (Gadgil et al., 2021, J Gene Med, 23(4): e3321.)

In some aspects disclosed herein are esnRNA comprising an engineered stem loop (eSL). Compensatory modifications made to the native stem loop sequence create an engineered stem loop (eSL) which more effectively communicates (folds and anneals) with the snRNA interaction stabilization domain (ISD) which in turn creates a snRNA platform with increased stability. U7 snRNAs have been previously shown to be programmable to modulate mRNAs. Disclosed herein are programmed engineered snRNA improvements which are capable of being used as a gene therapy tool.

snRNA systems disclosed herein are configured to bind target DMD RNA sequences to modulate RNA splicing which can lead to single or multiple exon skipping or exon inclusion of targeted sequences of the DMD RNA. DMD-targeting snRNA are configured to bind to DMD pre-mRNA molecules at sites that regulate RNA splicing. Splicing regulatory sites can include splice acceptor sequences, splice donor sequences, and exon splice enhancer sequences. snRNA sequences of the disclosure can induce exon skipping (of single or multiple exons) of targeted exonic sequences.

In one embodiment, these snRNAs are human snRNAs. In another embodiment, these snRNAs are mouse snRNAs. In another embodiment, the snRNAs are of any species. In another embodiment, the snRNAs are a combination of human and mouse snRNAs. In one embodiment, the U7 snRNA is a human U7 snRNA or a mouse U7 snRNA. In another embodiment disclosed herein, snRNA comprises varying types of snRNAs (U1-U12, etc.) by combining domains of endogenous snRNAs to fine tune stabilization of the platform and/or to reduce off-target effects. For example, in one embodiment, the snRNA system comprises a combination of human or mouse U7 snRNA and human or mouse U1 snRNA components.

Additional elements that can tune the processing and abundance of the RNA can be further engineered into the snRNAs or esnRNAs comprising eSLs. In one embodiment, additional elements that can tune the processing, stability, and abundance of the esnRNA can be further engineered into the esnRNAs at the 5′ or 3′ ends. In another embodiment, such elements may include but are not limited to stem loops, hairpins, G-C clamps, kissing loops, triplexes, quadruplexes, and protein binding sites.

The snRNA platform and portions thereof disclosed herein can be used in any therapeutic setting and context so long as a suitable spacer(s) or target sequence(s) TS(s) is included in the design of the therapeutic composition. In certain embodiments, a therapeutic snRNA composition is used to treat a disease associated with dysregulated, mutated, or non-functional dystrophin. In some aspects the disease or disorder is Duchenne Muscular Dystrophy.

### Targeting Sequences

The snRNA systems can be programmed to comprise a targeting sequence (TS) (also termed “spacer”) that targets an RNA of interest. The snRNA systems can be programmed with one or more targeting sequences targeting one or more RNAs of interest. In some aspects, the targeting sequence is a 5′ targeting sequence (5′TS) (also termed “spacer”) that targets one or more RNAs of interest. In this context, 5′ is in reference to the snRNA insert's 5′ end and not necessarily to the overall vector configuration comprising the snRNA insert or inserts. The TS can be located in or near the 5′ end of the snRNA. In an alternative embodiment, the targeting sequence(s) (TS) can be located in or near a 3′ position in the snRNA construct, thereby generating a 3′ targeting sequence (3′ TS), particularly if the snRNA construct is not a U7-based snRNA.

Targeting sequences of the disclosure, including 5′ TS, and 3′TS can be between about 1 and about 200 nucleotides in length. In some aspects, targeting sequences of the disclosure are between about 10 and about 150 nucleotides in length. In some aspects, targeting sequences of the disclosure are between about 10 and about 100 nucleotides in length. In some aspects, targeting sequences of the disclosure are between about 20 and about 60 nucleotides in length. In some aspects, targeting sequences of the disclosure are at least about 10, 20, 30, 40, 50, 60, or about 70 nucleotides in length.

snRNA compositions of the disclosure can comprise more than one targeting sequence, wherein each targeting sequence binds a distinct RNA sequence. In some aspects, esnRNA of the disclosure comprise a fusion targeting sequence. In some aspects, a fusion targeting sequence is a nucleic acid sequence comprising two targeting sequences directly connected or connected by one or more linker nucleic acid sequences, wherein each targeting sequence binds a different target RNA sequence.

In one example, U7 snRNA can be programmed by replacing the histone mRNA binding sequence with a sequence complementary to a target of interest. In some aspects snRNA systems of the disclosure bind a target mRNA or pre-mRNA sequence of interest. The exemplary snRNA systems shown herein lead to exon skipping (for treating DMD, e.g., DMD exon skipping).

In some embodiments, snRNAs of the disclosure target a pre-mRNA or mRNA sequence encoding the DMD gene. DMD is a gene encoding the protein dystrophin. Mutations in DMD are associated with Duchene muscular dystrophy. In some embodiments, the DMD RNA sequence targeted by snRNA compositions of the disclosure can be any exonic or intronic DMD RNA sequence. In some embodiments, the DMD RNA sequence targeted by snRNA compositions of the disclosure is an exon 2, exon 44, exon 45, exon 51, or exon 53 DMD RNA sequence. In some embodiments, the DMD RNA sequences targeted by snRNA compositions of the disclosure is a combination of sequences from the group consisting of exon 1, exon 2, exon 3, exon 4, exon 5, exon 44, exon 45, exon 50, exon 51, exon 52, exon 53, exon 54, and exon 55 DMD RNA sequence in a manner such that multiple exons are skipped. In some embodiments, the targeting sequences target RNA of interest to skip DMD exons 50, 51, 52, and 53. In some embodiments, the targeting sequences target RNA of interest to skip exons 44 and 45. In some embodiments, the DMD RNA sequences targeted by snRNA compositions of the disclosure is a combination of sequences from the group consisting of exon 1, exon 2, exon 3, exon 4, and exon 5. In some embodiments, the DMD RNA sequence targeted by the snRNA of the disclosure is a splicing regulatory sequence selected from: a splice acceptor sequence, a splice donor sequence, and/or an exon splice enhancer sequence.

In some embodiments, the nucleic acid sequence encoding wild-type human DMD mRNA comprises or consists of SEQ ID NO: 139. In some embodiments, the nucleic acid sequence encoding wild-type human DMD mRNA comprises a sequence that is at least 65%, 70%, 75%, 80%, 85%, 90%, 95%, 96%, 97%, 98%, or 99% identical to the sequence set forth in SEQ ID NO: 139. In some embodiments, the nucleic acid sequence encoding wild-type human DMD pre-mRNA comprises or consists of SEQ ID NO: 140. In some embodiments, the nucleic acid sequence encoding wild-type human DMD pre-mRNA comprises a sequence that is at least 65%, 70%, 75%, 80%, 85%, 90%, 95%, 96%, 97%, 98%, or 99% identical to the sequence set forth in SEQ ID NO: 140. In some embodiments, the nucleic acid sequence encoding human DMD comprises a deletion. In some embodiments, the deletion is a single nucleotide. In some embodiments, the deletion can be one or more nucleotides. In some embodiments, the deletion is an exonic or intronic sequence. In some embodiments, the deletion comprises an intronic and exonic sequence. In some embodiments, the deletion occurs across a region comprising one or more of exon 44, exon 45, exon 46, exon 47, exon 48, exon 49, exon 50, exon 51, exon 52, exon 53, exon 54, and exon 55 and any introns upstream downstream of therebetween of these exons. In some embodiments, the amino acid sequence encoding of dystrophin comprises or consists of SEQ ID NO: 141. In some embodiments, the amino acid sequence of human dystrophin comprises a sequence that is at least 65%, 70%, 75%, 80%, 85%, 90%, 95%, 96%, 97%, 98%, or 99% identical to the sequence set forth in SEQ ID NO: 141.

Target sequence that binds DMD exon 2 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 1 which follows:

TABLE 1
DMD Exon 2 RNA Targeting Sequences

| UID | target site | Sequence | SEQ ID NO: |
|---|---|---|---|
| | SA and ESE | CTTTTGAACATCTTCTCTTTCATC | 59 |
| | ESE | CTTTTGAACATCTTCTCTTTC | 60 |
| | BpA | taaaaGTTAGGAAGCAAC | 61 |
| | SA and ESE | CATCTTCTCTTTCATCTaaaatgc | 62 |
| | SD | CCTTAGAAAATTGTGCATTTACCC | 63 |
| | ESE | GTTTTCTTTTGAACATCTTCTCTTTC | 64 |
| | SA and ESE | TTCTTTTGAACATCTTCTCTTTCATC | 65 |
| | ESE | GTTTTCTTTTGAACATCTTCTC | 66 |
| | SD | TACCTTAGAAAATTGTGCATTTACCC | 67 |
| | SD | CTTAGAAAATTGTGCATTTACCCATT | 68 |
| | SA and ESE | TGTTTTCTTTTGAACATCTTCTCTTTCATC | 69 |

| | |
|---|---|
| DMD Exon 2 22 nucleotide targeting sequences | 334-674 |
| DMD Exon 2 26 nucleotide targeting sequences | 675-1011 |
| DMD Exon 2 30 nucleotide targeting sequences | 1012-1344 |

In some embodiments, the target sequence that binds human DMD exon 2 comprises the sequence set forth in SEQ ID NO: 59. In some embodiments, the target sequence that binds human DMD exon 2 comprises the sequence set forth in SEQ ID NO: 60. In some embodiments, the target sequence that binds human DMD exon 2 comprises the sequence set forth in SEQ ID NO: 61. In some embodiments, the target sequence that binds human DMD exon 2 comprises the sequence set forth in SEQ ID NO: 62. In some embodiments, the target sequence that binds human DMD exon 2 comprises the sequence set forth in SEQ ID NO: 63. In some embodiments, the target sequence that binds human DMD exon 2 comprises the sequence set forth in SEQ ID NO: 64. In some embodiments, the target sequence that binds human DMD exon 2 comprises the sequence set forth in SEQ ID NO: 65. In some embodiments, the target sequence that binds human DMD exon 2 comprises the sequence set forth in SEQ ID NO: 66. In some embodiments, the target sequence that binds human DMD exon 2 comprises the sequence set forth in SEQ ID NO: 67. In some embodiments, the target sequence that binds human DMD exon 2 comprises the sequence set forth in SEQ ID NO: 68. In some embodiments, the target sequence that binds human DMD exon 2 comprises the sequence set forth in SEQ ID NO: 69. In some embodiments, the target sequence that binds human DMD exon 2 comprises one or more sequence set forth in any of SEQ ID NO: 334-674. In some embodiments, the target sequence that binds human DMD exon 2 comprises one or more sequence set forth in any of SEQ ID NO: 675-1011. In some embodiments, the target sequence that binds human DMD exon 2 comprises one or more sequence set forth in any of SEQ ID NO: 1012-1344.

Target sequences that bind DMD exon 44 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 2 which follows:

TABLE 2
DMD Exon 44 RNA Targeting Sequences

| | | SEQ ID |
|---|---|---|
| UID | Sequence | NO: |
| SR220265; | CGCCGCCATTTCTCAACAG | 70 |
| z1 | | |
| SR220266; | TTTGTATTTAGCATGTTCCC | 71 |
| z2 | | |
| SR220267; | TGTTCAGCTTCTGTTAGCCACTGA | 72 |
| z3 | | |
| SR220268; | CATTTCTCAACAGATCTGTCAAATCGCctgc | 73 |
| z4 | | |
| SR220269; | GTGTCTTTCTGAGAAACTGTTCAGCTTCTG | 74 |
| z5 | | |
| SR220270; | gacttacCTTAAGATACCATTTGTATTTAGCATGTT | 75 |
| z6 | | |
| SR220281; | ATCTGTCAAATCGCctgcaggtaaaagcatatggatcaagaaa | 76 |
| z7 | | |
| SR220282; | TTCTGAGAAACTGTTCAGCTTCTGTTAGCCACTGA | 77 |
| z8 | | |
| SR220283; | TTGTGTCTTTCTGAGAAACTGTTCAGCTTCTGTTAGC | 78 |
| z9 | CACTGA | |
| SR220284; | cttacCTTAAGATACCATTTG | 79 |
| z10 | | |
| z11 | GTGTCTTTCTGAGAAACTGTTCAGCTTCTGTTAGCC | 80 |
| SR220316; | CATTTCTCAACAGATCTGTCAAATCGCctgcGTGTCTTT | 81 |
| z4z5 | CTGAGAAACTGTTCAGCTTCTG | |
| fusion | | |
| SR220317; | GTGTCTTTCTGAGAAACTGTTCAGCTTCTGCATTTCT | 82 |
| z5z4 fusion | CAACAGATCTGTCAAATCGCCTGC | |
| SR220318; | GTGTCTTTCTGAGAAACTGTTCAGCTTCTGGACTTAC | 83 |
| z5z6 fusion | CTTAAGATACCATTTGTATTTAGCATGTT | |
| SR220319; | GACTTACCTTAAGATACCATTTGTATTTAGCATGTTG | 84 |
| z6z5 fusion | TGTCTTTCTGAGAAACTGTTCAGCTTCTG | |
| SR220320; | CATTTCTCAACAGATCTGTCAAATCGCCTGCGACTTA | 85 |
| z4z6 fusion | CCTTAAGATACCATTTGTATTTAGCATGTT | |
| SR220321; | GACTTACCTTAAGATACCATTTGTATTTAGCATGTTC | 86 |
| z6z4 fusion | ATTTCTCAACAGATCTGTCAAATCGCCTGC | |
| SR220331; | CATTTCTCAACAGATCTGTCAAATCGCctgcTTGTGTCT | 87 |
| z4z9 fusion | TTCTGAGAAACTGTTCAGCTTCTGTTAGCCACTGA | |
| SR220332; | TTGTGTCTTTCTGAGAAACTGTTCAGCTTCTGTTAGC | 88 |
| z9z4 fusion | CACTGACATTTCTCAACAGATCTGTCAAATCGCctgc | |
| SR220333; | CATTTCTCAACAGATCTGTCAAATCGCctgcGTGTCTTT | 89 |
| z4z11 | CTGAGAAACTGTTCAGCTTCTGTTAGCC | |
| fusion | | |
| SR220334; | GTGTCTTTCTGAGAAACTGTTCAGCTTCTGTTAGCCC | 90 |
| z11z4 | ATTTCTCAACAGATCTGTCAAATCGCCTGC | |
| fusion | | |

In some embodiments, the target sequence that binds human DMD exon 44 comprises the sequence set forth in SEQ ID NO: 70. In some embodiments, the target sequence that binds human DMD exon 44 comprises the sequence set forth in SEQ ID NO: 71. In some embodiments, the target sequence that binds human DMD exon 44 comprises the sequence set forth in SEQ ID NO: 72. In some embodiments, the target sequence that binds human DMD exon 44 comprises the sequence set forth in SEQ ID NO: 73. In some embodiments, the target sequence that binds human DMD exon 44 comprises the sequence set forth in SEQ ID NO: 74. In some embodiments, the target sequence that binds human DMD exon 44 comprises the sequence set forth in SEQ ID NO: 75. In some embodiments, the target sequence that binds human DMD exon 44 comprises the sequence set forth in SEQ ID NO: 76. In some embodiments, the target sequence that binds human DMD exon 44 comprises the sequence set forth in SEQ ID NO: 77. In some embodiments, the target sequence that binds human DMD exon 44 comprises the sequence set forth in SEQ ID NO: 78. In some embodiments, the target sequence that binds human DMD exon 44 comprises the sequence set forth in SEQ ID NO: 79. In some embodiments, the target sequence that binds human DMD exon 44 comprises the sequence set forth in SEQ ID NO: 80. In some embodiments, the target sequence that binds human DMD exon 44 comprises the sequence set forth in SEQ ID NO: 81. In some embodiments, the target sequence that binds human DMD exon 44 comprises the sequence set forth in SEQ ID NO: 82. In some embodiments, the target sequence that binds human DMD exon 44 comprises the sequence set forth in SEQ ID NO: 83. In some embodiments, the target sequence that binds human DMD exon 44 comprises the sequence set forth in SEQ ID NO: 84 In some embodiments, the target sequence that binds human DMD exon 44 comprises the sequence set forth in SEQ ID NO: 85. In some embodiments, the target sequence that binds human DMD exon 44 comprises the sequence set forth in SEQ ID NO: 86. In some embodiments, the target sequence that binds human DMD exon 44 comprises the sequence set forth in SEQ ID NO: 87. In some embodiments, the target sequence that binds human DMD exon 44 comprises the sequence set forth in SEQ ID NO: 88. In some embodiments, the target sequence that binds human DMD exon 44 comprises the sequence set forth in SEQ ID NO: 89. In some embodiments, the target sequence that binds human DMD exon 44 comprises the sequence set forth in SEQ ID NO: 90.

Target sequences that bind DMD exon 45 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 3 which follows:

TABLE 3
DMD Exon 45 RNA Targeting Sequences

| | target | | SEQ ID |
|---|---|---|---|
| UID | site | Sequence | NO: |
| SR220068; | 5′ ese | TGCCGCTGCCCAATGCCATCCTGGAG | 91 |
| z3 | | | |
| SR220069; | 3 ′ss | GCCCAATGCCATCCTGGAGTTCctg | 92 |
| z1 | | | |
| SR220255; | | ccctacCTCTTTTTTCTGTCTGACAGCGCCCAATG | 93 |
| z1z2 fusion | | CCATCCTGGAGTTCctg | |
| SR220256; | | CTTCCCCAGTTGCATTCAATGTTCTGGCCCAA | 94 |
| z1z4 fusion | | TGCCATCCTGGAGTTCctg | |
| SR220257; | | CTGGCATCTGTTTTTGAGGATTGCTGGCCCAA | 95 |
| z1z9 fusion | | TGCCATCCTGGAGTTCctg | |
| SR220273; | | ccctacCTCTTTTTTCTGTCTGACAGCAGTTTGCC | 96 |
| z8z2 fusion | | GCTGCCCAATGCCATCCTGGAG | |
| SR220274; | | CTTCCCCAGTTGCATTCAATGTTCTGAGTTTGC | 97 |
| z8z4 fusion | | CGCTGCCCAATGCCATCCTGGAG | |
| SR220275; | | CTGGCATCTGTTTTTGAGGATTGCTGAGTTTG | 98 |
| z8z9 fusion | | CCGCTGCCCAATGCCATCCTGGAG | |
| SR220276; | | TTTGCAGACCTCCTGCCACCGCAGATTCAGAG | 99 |
| z8z10 | | TTTGCCGCTGCCCAATGCCATCCTGGAG | |
| fusion | | | |
| SR220303; | | AGTTTGCCGCTGCCCAATGCCATCCTGGAG | 100 |
| z8 | | | |
| SR220304; | | TTTCTGTCTGACAGCTGTTTGCAGACCTCC | 101 |
| z11 | | | |
| SR220305; | | TGTTCTGACAACAGTTTGCCGCTGCCCAAT | 102 |
| 12 | | | |
| SR220306; | | TTCAATGTTCTGACAACAGTTTGCCGCTGC | 103 |
| z13 | | | |
| SR220307; | | CCACCGCAGATTCAGGCTTCCCAATTTTTC | 104 |
| z14 | | | |
| SR220335; | | AGTTTGCCGCTGCCCAATGCCATCCTGGAGccct | 105 |
| z2z8 fusion | | acCTCTTTTTTCTGTCTGACAGC | |
| SR220336; | | AGTTTGCCGCTGCCCAATGCCATCCTGGAGCT | 106 |
| z4z8 fusion | | TCCCCAGTTGCATTCAATGTTCTG | |
| SR220337; | | AGTTTGCCGCTGCCCAATGCCATCCTGGAGCT | 107 |
| z9z8 fusion | | GGCATCTGTTTTTGAGGATTGCTG | |
| SR220338; | | AGTTTGCCGCTGCCCAATGCCATCCTGGAGTT | 108 |
| z10z8 | | TGCAGACCTCCTGCCACCGCAGATTCAG | |
| fusion | | | |
| SR220379; | | TTTGCAGACCTCCTGCCACCGCAGATTCAG | 109 |
| z10 | | | |
| SR220442; | | ATTTCTTCCCCAGTTGCATTCAATGTTCTGA | 110 |
| z45-15 | | | |
| SR220443; | | TACTGGCATCTGTTTTTGAGGATTGCTGAA | 111 |
| z45-16 | | | |
| SR220444; | | CAATTTTTCCTGTAGAATACTGGCATCTGTTTT | 112 |
| z45-17 | | TG | |
| SR220445; | | TTTCCTGTAGAATACTGGCATCTGTTTTTG | 113 |
| z45-18 | | | |
| SR220446; | | GCATCTGTTTTTGAGGATTGCTGAATTATT | 114 |
| z45-19 | | | |
| SR220447; | | TGTTTTTGAGGATTGCTGAATTATTTCTTC | 115 |
| z45-20 | | | |
| SR220448; | | GAATTATTTCTTCCCCAGTTGCATTCAATGT | 116 |
| z45-21 | | | |
| SR220449; | | TCCCCAGTTGCATTCAATGTTCTGACAACAG | 117 |
| z45-22 | | | |
| SR220450; | | TTATTTCTTCCCCAGTTGCATTCAATGTTCTGA | 118 |
| z45-23 | | CA | |
| z2 | | ccctacCTCTTTTTTCTGTCTGACAGC | 126 |

In some embodiments, the target sequence that binds human DMD exon 45 comprises the sequence set forth in SEQ ID NO: 91. In some embodiments, the target sequence that binds human DMD exon 45 comprises the sequence set forth in SEQ ID NO: 92. In some embodiments, the target sequence that binds human DMD exon 45 comprises the sequence set forth in SEQ ID NO: 93. In some embodiments, the target sequence that binds human DMD exon 45 comprises the sequence set forth in SEQ ID NO: 94. In some embodiments, the target sequence that binds human DMD exon 45 comprises the sequence set forth in SEQ ID NO: 95. In some embodiments, the target sequence that binds human DMD exon 45 comprises the sequence set forth in SEQ ID NO: 96. In some embodiments, the target sequence that binds human DMD exon 45 comprises the sequence set forth in SEQ ID NO: 97. In some embodiments, the target sequence that binds human DMD exon 45 comprises the sequence set forth in SEQ ID NO: 98. In some embodiments, the target sequence that binds human DMD exon 45 comprises the sequence set forth in SEQ ID NO: 99. In some embodiments, the target sequence that binds human DMD exon 45 comprises the sequence set forth in SEQ ID NO: 100. In some embodiments, the target sequence that binds human DMD exon 45 comprises the sequence set forth in SEQ ID NO: 101. In some embodiments, the target sequence that binds human DMD exon 45 comprises the sequence set forth in SEQ ID NO: 102. In some embodiments, the target sequence that binds human DMD exon 45 comprises the sequence set forth in SEQ ID NO: 103. In some embodiments, the target sequence that binds human DMD exon 45 comprises the sequence set forth in SEQ ID NO: 104. In some embodiments, the target sequence that binds human DMD exon 45 comprises the sequence set forth in SEQ ID NO: 105. In some embodiments, the target sequence that binds human DMD exon 45 comprises the sequence set forth in SEQ ID NO: 106. In some embodiments, the target sequence that binds human DMD exon 45 comprises the sequence set forth in SEQ ID NO: 107. In some embodiments, the target sequence that binds human DMD exon 45 comprises the sequence set forth in SEQ ID NO: 108. In some embodiments, the target sequence that binds human DMD exon 45 comprises the sequence set forth in SEQ ID NO: 109. In some embodiments, the target sequence that binds human DMD exon 45 comprises the sequence set forth in SEQ ID NO: 110. In some embodiments, the target sequence that binds human DMD exon 45 comprises the sequence set forth in SEQ ID NO: 111. In some embodiments, the target sequence that binds human DMD exon 45 comprises the sequence set forth in SEQ ID NO: 112. In some embodiments, the target sequence that binds human DMD exon 45 comprises the sequence set forth in SEQ ID NO: 113. In some embodiments, the target sequence that binds human DMD exon 45 comprises the sequence set forth in SEQ ID NO: 114. In some embodiments, the target sequence that binds human DMD exon 45 comprises the sequence set forth in SEQ ID NO: 115. In some embodiments, the target sequence that binds human DMD exon 45 comprises the sequence set forth in SEQ ID NO: 116. In some embodiments, the target sequence that binds human DMD exon 45 comprises the sequence set forth in SEQ ID NO: 117. In some embodiments, the target sequence that binds human DMD exon 45 comprises the sequence set forth in SEQ ID NO: 118. In some embodiments, the target sequence that binds human DMD exon 45 comprises the sequence set forth in SEQ ID NO: 126.

Target sequences that bind DMD exon 51 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 4 which follows:

TABLE 4
DMD exon 51 RNA Targeting Sequences

| | | SEQ ID |
|---|---|---|
| UID | Targeting Sequences | NO: |
| z38 | TACCTCCAACATCAAGGAAGATGGCA | 206 |
| z42 | GTAACCACAGGTTGTGTCACCAGAGTAACA | 207 |

Fusion Sequences

| | | |
|---|---|---|
| z70 | catacCTTCTGCTTGATGATCATCTCGCACCAGAGTAACA | 208 |
| | GTCTGAGTAGGAGctaaa | |
| z71 | catacCTTCTGCTTGATGATCATCTCGTGCCGCTGCCCAA | 209 |
| | TGCCATCCTGGAG | |
| z72 | catacCTTCTGCTTGATGATCATCTCGGCCCAATGCCATC | 210 |
| | CTGGAGTTCctg | |
| z73 | TACCTCCAACATCAAGGAAGATGGCAGTAACCACAGG | 211 |
| (z38/z42) | TTGTGTCACCAGAGTAACA | |
| z74 | AACATCAAGGAAGATGGCATTTCTAGGTAACCACAGG | 212 |
| | TTGTGTCACCAGAGTAACA | |
| z75 | AACATCAAGGAAGATGGCATTTCTAGTGCCGCTGCCC | 213 |
| | AATGCCATCCTGGAG | |
| z76 | TTTTTCTCATACCTTCTGCTTGATGTCTGAGTAGGAGC | 214 |
| | TAAAATATTTTGGG | |
| Z187 | GTAACCACAGGTTGTGTCACCAGAGTAACATACCTCC | 215 |
| (z42/z38) | AACATCAAGGAAGATGGCA | |
| z230 | CGAGATGATCATCAAGCAGAAGgtatgCACCAGAGTAAC | 216 |
| | AGTCTGAGTAGGAGctaaa | |
| z231 | CACCAGAGTAACAGTCTGAGTAGGAGctaaaTACCTCCA | 217 |
| | ACATCAAGGAAGATGGCA | |
| z232 | TACCTCCAACATCAAGGAAGATGGCACACCAGAGTAA | 218 |
| | CAGTCTGAGTAGGAGctaaa | |
| z233 | GTCTGAGTAGGAGctaaaatattttgggTACCTCCAACATCAA | 219 |
| | GGAAGATGGCA | |
| z234 | TACCTCCAACATCAAGGAAGATGGCAGTCTGAGTAGG | 220 |
| | AGctaaaatattttggg | |
| z235 | GTCTGAGTAGGAGctaaaatattttgggtttttctcatacCTTCTGCTTG | 221 |
| | AT | |
| z237 | cCTTCTGCTTGATGATCATCTCGGAGTAACAGTCTGAG | 222 |
| | TAGGAGc | |
| z238 | cCTTCTGCTTGATGATCATCTCGCCAGAGTAACAGTCT | 223 |
| | GAGTAGGAGc | |
| z239 | cCTTCTGCTTGATGATCATCTCGCAGTCTGAGTAGGAG | 224 |
| | ctaaa | |
| z240 | ctcatacCTTCTGCTTGATGGAGTAACAGTCTGAGTAGGA | 225 |
| | Gc | |
| z241 | ctcatacCTTCTGCTTGATGCCAGAGTAACAGTCTGAGTA | 226 |
| | GGAGc | |
| z242 | ctcatacCTTCTGCTTGATGCAGTCTGAGTAGGAGctaaa | 227 |

In some embodiments, the target sequence that binds human DMD exon 51 comprises the sequence set forth in SEQ ID NO: 206. In some embodiments, the target sequence that binds human DMD exon 51 comprises the sequence set forth in SEQ ID NO: 207. In some embodiments, the target sequence that binds human DMD exon 51 comprises the sequence set forth in SEQ ID NO: 208. In some embodiments, the target sequence that binds human DMD exon 51 comprises the sequence set forth in SEQ ID NO: 209. In some embodiments, the target sequence that binds human DMD exon 51 comprises the sequence set forth in SEQ ID NO: 210. In some embodiments, the target sequence that binds human DMD exon 51 comprises the sequence set forth in SEQ ID NO: 211. In some embodiments, the target sequence that binds human DMD exon 51 comprises the sequence set forth in SEQ ID NO: 212. In some embodiments, the target sequence that binds human DMD exon 51 comprises the sequence set forth in SEQ ID NO: 213. In some embodiments, the target sequence that binds human DMD exon 51 comprises the sequence set forth in SEQ ID NO: 214. In some embodiments, the target sequence that binds human DMD exon 51 comprises the sequence set forth in SEQ ID NO: 215. In some embodiments, the target sequence that binds human DMD exon 51 comprises the sequence set forth in SEQ ID NO: 216. In some embodiments, the target sequence that binds human DMD exon 51 comprises the sequence set forth in SEQ ID NO: 217. In some embodiments, the target sequence that binds human DMD exon 51 comprises the sequence set forth in SEQ ID NO: 218. In some embodiments, the target sequence that binds human DMD exon 51 comprises the sequence set forth in SEQ ID NO: 219. In some embodiments, the target sequence that binds human DMD exon 51 comprises the sequence set forth in SEQ ID NO: 220. In some embodiments, the target sequence that binds human DMD exon 51 comprises the sequence set forth in SEQ ID NO: 221. In some embodiments, the target sequence that binds human DMD exon 51 comprises the sequence set forth in SEQ ID NO: 222. In some embodiments, the target sequence that binds human DMD exon 51 comprises the sequence set forth in SEQ ID NO: 223. In some embodiments, the target sequence that binds human DMD exon 51 comprises the sequence set forth in SEQ ID NO: 224. In some embodiments, the target sequence that binds human DMD exon 51 comprises the sequence set forth in SEQ ID NO: 225. In some embodiments, the target sequence that binds human DMD exon 51 comprises the sequence set forth in SEQ ID NO: 226. In some embodiments, the target sequence that binds human DMD exon 51 comprises the sequence set forth in SEQ ID NO: 227.

Target sequences that bind DMD exon 53 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 5, which follows:

TABLE 5
DMD Exon 53 RNA Targeting Sequences

| | target | | SEQ ID |
|---|---|---|---|
| UID | site | Sequence | NO: |
| SR220202; | | GUUGCCUCCGGUUCUGAAGGUGUUCUUG | 237 |
| z1 | | | |
| SR220203; | | CUCCGGUUCUGAAGGUGUUCUUGUA | 238 |
| z2 | | | |
| SR220204; | | AUUUCAUUCAACUGUUGCCUCCGGUUCU | 239 |
| z3 | | | |
| SR220205; | | UGAAGGUGUUCUUGUACUUCAUCCC | 240 |
| z4 | | | |
| SR220206; | | GUUGCCUCCGGUUCUGAAGGUGUUC | 241 |
| z5 | | | |
| SR220207; | | CUGAAGGUGUUCUUGUACUUCAUCC | 242 |
| z6 | | | |
| SR220208; | | CUGUUGCCUCCGGUUCUGAAGGUGUUCUUG | 243 |
| z7 | | | |
| SR220209; | | CAACUGUUGCCUCCGGUUCUGAAGGUGUUC | 244 |
| z8 | | UUG | |
| SR220210; | | CUGAUUCUGAAUUCUUUCAACUAGAAU | 245 |
| z9 | | | |
| SR220211; | | CUGAAUUCUUUCAACUAGAAUAAAAGG | 246 |
| z10 | | | |
| SR220212; | | GGUAUCUUUGAUACUAACCUUGGUUUCUG | 247 |
| z11 | | | |
| SR220213; | | CUUUGAUACUAACCUUGGUUUCUGUGAU | 248 |
| z12 | | | |
| SR220243; | | CAUUCAACUGUUGCCUCCGGUUCUGAAGGU | 249 |
| z13z16 | | GUCAUCCCACUGAUUCUGAAUUCUUUCAAC | |
| fusion | | UA | |
| SR220244; | | UCAUCCCACUGAUUCUGAAUUCUUUCAACU | 250 |
| z16z13 | | ACAUUCAACUGUUGCCUCCGGUUCUGAAGG | |
| fusion | | UG | |
| SR220214; | | CAUUCAACUGUUGCCUCCGGUUCUGAAGGU | 251 |
| z13 | | G | |
| SR220285; | | UCAUCCCACUGAUUCUGAAUUCUUUCCAUU | 252 |
| z18z13 | | CAACUGUUGCCUCCGGUUCUGAAGGUG | |
| fusion | | | |
| SR220286; | | CAUUCAACUGUUGCCUCCGGUUCUGAAGGU | 253 |
| z13z19 | | GGGUUUCUGUGAUUUUCUUUUGGAUUGC | |
| fusion | | | |
| SR220287; | | CAUUCAACUGUUGCCUCCGGUUCUGAAGGU | 254 |
| z13z20 | | GGCUCAGCUUCUUCCUUAGCUUCCAGCCAU | |
| fusion | | U | |
| SR220288; | | CAUUCAACUGUUGCCUCCGGUUCUGAAGGU | 255 |
| z13z21 | | GCUUCCUUAGCUUCCAGCCAUUGUGUUGAA | |
| fusion | | U | |
| SR220289; | | CAUUCAACUGUUGCCUCCGGUUCUGAAGGU | 256 |
| z13z22 | | GCUCUGGCCUGUCCUAAGACCUGCUCAGCU | |
| fusion | | U | |
| SR220290; | | CAUUCAACUGUUGCCUCCGGUUCUGAAGGU | 257 |
| z13z23 | | GACCUGCUCAGCUUCUUCCUUAGCUUC | |
| fusion | | | |
| SR220291; | | CAUUCAACUGUUGCCUCCGGUUCUGAAGGU | 258 |
| z13z24 | | GAGCUUCUUCCUUAGCUUCCAGCCAUU | |
| fusion | | | |
| SR220292; | | UUUCAUUCAACUGUUGCCUCCGGUUCUGAA | 259 |
| z25 | | | |
| SR220293; | | GAAGGUGUUCUUGUACUUCAUCCCACUGAU | 260 |
| z26 | | | |
| SR220294; | | UUCAUUCAACUGUUGCCUCCGGUUCUGAAG | 261 |
| z27 | | | |
| SR220295; | | CAUUCAACUGUUGCCUCCGGUUCUGAAGGU | 262 |
| z13z28 | | GAUUGCAUCUACUGUAUAGGGACCCUC | |
| fusion | | | |
| SR220296; | | CAUUCAACUGUUGCCUCCGGUUCUGAAGGU | 263 |
| z13 | | G | |
| SR220322; | | AUUGCAUCUACUGUAUAGGGACCCUCCAUU | 264 |
| z28z13 | | CAACUGUUGCCUCCGGUUCUGAAGGUG | |
| fusion | | | |
| SR220323; | | GCUCAGCUUCUUCCUUAGCUUCCAGCCAUU | 265 |
| z20z13 | | CAUUCAACUGUUGCCUCCGGUUCUGAAGGU | |
| fusion | | G | |
| SR220324; | | CAUUCAACUGUUGCCUCCGGUUCUGAAGGU | 266 |
| z13z16 | | GUCAUCCCACUGAUUCUGAAUUCUUUCAAC | |
| fusion | | UA | |
| SR220325; | | AUUGCAUCUACUGUAUAGGGACCCUCUCAU | 267 |
| z28z16 | | CCCACUGAUUCUGAAUUCUUUCAACUA | |
| fusion | | | |
| SR220326; | | UUUCAUUCAACUGUUGCCUCCGGUUCUGAA | 268 |
| z25z16 | | UCAUCCCACUGAUUCUGAAUUCUUUCAACU | |
| fusion | | A | |
| SR220327; | | CAUUCAACUGUUGCCUCCGGUUCUGAAGGU | 269 |
| z13z29 | | GAUUGCAUCUACUGUAUAGGGACCCUCCUU | |
| fusion | | CC | |
| SR220328; | | CAUUCAACUGUUGCCUCCGGUUCUGAAGGU | 270 |
| z13z30 | | GGGAUUGCAUCUACUGUAUAGGGACCCUCC | |
| | | U | |
| SR220329; | | CAUUCAACUGUUGCCUCCGGUUCUGAAGGU | 271 |
| z13z31 | | GUUGGAUUGCAUCUACUGUAUAGGGACCCU | |
| fusion | | C | |
| SR220330; | | CAUUCAACUGUUGCCUCCGGUUCUGAAGGU | 272 |
| z13z32 | | GUCUACUGUAUAGGGACCCUCCUUCCAUGA | |
| fusion | | C | |
| SR220343; | | AUUGCAUCUACUGUAUAGGGACCCUCCUUC | 273 |
| z29z13 | | CCAUUCAACUGUUGCCUCCGGUUCUGAAGG | |
| fusion | | UG | |
| sR220344; | | GGAUUGCAUCUACUGUAUAGGGACCCUCCU | 274 |
| z30z13 | | CAUUCAACUGUUGCCUCCGGUUCUGAAGGU | |
| fusion | | G | |
| SR220345; | | UUGGAUUGCAUCUACUGUAUAGGGACCCUC | 275 |
| z31z13 | | CAUUCAACUGUUGCCUCCGGUUCUGAAGGU | |
| fusion | | G | |
| SR220227; | | TCATCCCACTGATTCTGAATTCTTTCAACTA | 276 |
| z16 | | | |
| SR220228; | | ATACTAACCTTGGTTTCTGTGAT | 277 |
| z17 | | | |
| SR220380; | | ACCUUGGUUUCUGUGAUUUUCUUUUGGAUU | 278 |
| z39 | | GCAUC | |
| SR220381; | | CAUUCAACUGUUGCCUCCGGUUCUGAAGGU | 279 |
| z40 | | GUUCUUG | |
| SR220382; | | CCCACTGATTCTGAATTCTTTCAACTAGAATA | 280 |
| z41 | | AAAGG | |
| SR220383; | | CATCCCACTGATTCTGAATTCTTTCAACTAG | 281 |
| z42 | | | |
| SR220384; | | UCAUCCCACUGAUUCUGAAUUCUUUCAACU | 282 |
| z43 | | AGAA | |
| SR220385; | | GUACUUCAUCCCACUGAUUCUGAAUUCUUU | 283 |
| z44 | | CAACUAG | |
| SR220386; | | UACUUCAUCCCACUGAUUCUGAAUUCUUUC | 284 |
| z45 | | AACUA | |
| SR220387; | | UUUCAUUCAACUGUUGCCUCCGGUUCUGAA | 285 |
| z46 | | GGUGUUC | |
| SR220388; | | CAUUUCAUUCAACUGUUGCCUCCGGUUCUG | 286 |
| z47 | | AAGGUGUUC | |
| SR220389; | | AUUUCAUUCAACUGUUGCCUCCGGUUCUGA | 287 |
| z48 | | AGGUG | |
| SR220390; | | CUUGGCUCUGGCCUGUCCUAAGACCUGCUC | 288 |
| z49 | | AGCUU | |
| SR220391; | | CUCUGGCCUGUCCUAAGACCUGCUCAGCUU | 289 |
| z50 | | CUUCC | |
| SR220392; | | CUUGGCUCUGGCCUGUCCUAAGACCUGCUC | 290 |
| z51 | | AGCUUCUUCC | |
| SR220393; | | UGACUCAAGCUUGGCUCUGGCCUGUCCUAA | 291 |
| z52 | | | |
| SR220394; | | GACUCAAGCUUGGCUCUGGCCUGUCCUAAG | 292 |
| z53 | | ACCUGC | |
| SR220395; | | CAAGCUUGGCUCUGGCCUGUCCUAAGACCU | 293 |
| z54 | | GCUCA | |
| SR220396; | | CUUCCUUAGCUUCCAGCCAUUGUGUUGAAU | 294 |
| z55 | | CC | |
| SR220397; | | GCUUCUUCCUUAGCUUCCAGCCAUUGUGUU | 295 |
| z56 | | GAAUCC | |
| SR220398; | | CUUCCUUAGCUUCCAGCCAUUGUGUUGAAU | 296 |
| z57 | | CCUUUAAC | |
| SR220399; | | AGCUUGGCUCUGGCCUGUCCUAAGACCUGC | 297 |
| z58 | | UCAGC | |
| SR220400; | | GGAUUGCAUCUACUGUAUAGGGACCCUCCU | 298 |
| z59 | | UCC | |
| SR220401; | | AUUGCAUCUACUGUAUAGGGACCCUCCUUC | 299 |
| z60 | | CAUGACUCA | |
| SR220402; | | CAUCUACUGUAUAGGGACCCUCCUUCCAUG | 300 |
| z61 | | ACUCAAGCU | |
| SR220403; | | ACUGUAUAGGGACCCUCCUUCCAUGACUCA | 301 |
| z62 | | AGCUUGGCUC | |
| SR220404; | | CUUUUGGAUUGCAUCUACUGUAUAGGGACC | 302 |
| z63 | | CUCCUUC | |
| SR220405; | | CAGCUUCUUCCUUAGCUUCCAGCCAUUGUG | 303 |
| z64 | | UUGAAUCCUU | |
| SR220406; | | CCCUCCUUCCAUGACUCAAGCUUGGCUCUG | 304 |
| z65 | | GCCUGUCCUA | |
| SR220407; | | GGAUUGCAUCUACUGUAUAGGGACCCUCCU | 305 |
| z37 | | UCCAU | |
| SR220408; | | AUUGCAUCUACUGUAUAGGGACCCUC | 306 |
| z28 | | | |
| SR220428; | | UUUCAUUCAACUGUUGCCUCCGGUUCUGAA | 307 |
| z46z16 | | GGUGUUCUCAUCCCACUGAUUCUGAAUUCU | |
| fusion | | UUCAACUA | |
| SR220429; | | UUUCAUUCAACUGUUGCCUCCGGUUCUGAA | 308 |
| z46z45 | | GGUGUUCUACUUCAUCCCACUGAUUCUGAA | |
| fusion | | UUCUUUCAACUA | |
| SR220430; | | UUUCAUUCAACUGUUGCCUCCGGUUCUGAA | 309 |
| z46z45 (-1) | | GGUGUUCUACUUCAUCCCACUGAUUCUGAA | |
| fusion | | UUCUUUCAACU | |
| SR220431; | | UUUCAUUCAACUGUUGCCUCCGGUUCUGAA | 310 |
| z46z16 | | GGUGUUCUUGUACUUCAUCCCACUGAUUCU | |
| (continuou | | GAAUUCUUUCAACUA | |
| s) fusion | | | |
| SR220432; | | GGAUUGCAUCUACUGUAUAGGGACCCUCCU | 311 |
| z59z46 | | UCCUUUCAUUCAACUGUUGCCUCCGGUUCU | |
| fusion | | GAAGGUGUUC | |
| SR220433; | | CAGCUUCUUCCUUAGCUUCCAGCCAUUGUG | 312 |
| z64z46 | | UUGAAUCCUUUUUCAUUCAACUGUUGCCUC | |
| fusion | | CGGUUCUGAAGGUGUUC | |
| SR220434; | | UUUCAUUCAACUGUUGCCUCCGGUUCUGAA | 313 |
| z46z64 | | GGUGUUCCAGCUUCUUCCUUAGCUUCCAGC | |
| fusion | | CAUUGUGUUGAAUCCUU | |
| SR220435; | | CUUCCUUAGCUUCCAGCCAUUGUGUUGAAU | 314 |
| z57z46 | | CCUUUAACUUUCAUUCAACUGUUGCCUCCG | |
| fusion | | GUUCUGAAGGUGUUC | |
| SR220436; | | CUUCCUUAGCUUCCAGCCAUUGUGUUGAAU | 315 |
| z57z40 | | CCUUUAACCAUUCAACUGUUGCCUCCGGUU | |
| fusion | | CUGAAGGUGUUCUUG | |
| SR220437; | | GCUUCUUCCUUAGCUUCCAGCCAUUGUGUU | 316 |
| z56z46 | | GAAUCCUUUCAUUCAACUGUUGCCUCCGGU | |
| fusion | | UCUGAAGGUGUUC | |
| SR220438; | | CUUUUGGAUUGCAUCUACUGUAUAGGGACC | 317 |
| z63z46 | | CUCCUUCUUUCAUUCAACUGUUGCCUCCGG | |
| fusion | | UUCUGAAGGUGUUC | |
| SR220439; | | AUUGCAUCUACUGUAUAGGGACCCUCCUUC | 318 |
| z60z46 | | CAUGACUCAUUUCAUUCAACUGUUGCCUCC | |
| fusion | | GGUUCUGAAGGUGUUC | |
| SR220440; | | CUUUUGGAUUGCAUCUACUGUAUAGGGACC | 319 |
| z63z64 | | CUCCUUCCAGCUUCUUCCUUAGCUUCCAGC | |
| fusion | | CAUUGUGUUGAAUCCUU | |
| SR220441; | | AUUGCAUCUACUGUAUAGGGACCCUCCUUC | 320 |
| z60z64 | | CAUGACUCACAGCUUCUUCCUUAGCUUCCA | |
| fusion | | GCCAUUGUGUUGAAUCCUU | |
| SR220452; | | UUUCAUUCAACUGUUGCCUCCGGUUCUGAA | 321 |
| z46z16 | | GGUGUUCUCAUCCCACUGAUUCUGAAUUCU | |
| fusion | | UUCAACUA | |
| SR220453; | | UUUCAUUCAACUGUUGCCUCCGGUUCUGAA | 322 |
| z46z45 | | GGUGUUCUACUUCAUCCCACUGAUUCUGAA | |
| fusion | | UUCUUUCAACUA | |
| SR220454; | | UUUCAUUCAACUGUUGCCUCCGGUUCUGAA | 323 |
| z46z45 (-1) | | GGUGUUCUACUUCAUCCCACUGAUUCUGAA | |
| fusion | | UUCUUUCAACU | |
| SR220455; | | GGAUUGCAUCUACUGUAUAGGGACCCUCCU | 324 |
| z59z46 | | UCCUUUCAUUCAACUGUUGCCUCCGGUUCU | |
| fusion | | GAAGGUGUUC | |
| SR220456; | | CAGCUUCUUCCUUAGCUUCCAGCCAUUGUG | 325 |
| z64z46 | | UUGAAUCCUUUUUCAUUCAACUGUUGCCUC | |
| fusion | | CGGUUCUGAAGGUGUUC | |
| SR220457; | | UUUCAUUCAACUGUUGCCUCCGGUUCUGAA | 326 |
| z46z64 | | GGUGUUCCAGCUUCUUCCUUAGCUUCCAGC | |
| fusion | | CAUUGUGUUGAAUCCUU | |
| SR220458; | | CUUCCUUAGCUUCCAGCCAUUGUGUUGAAU | 327 |
| z57z46 | | CCUUUAACUUUCAUUCAACUGUUGCCUCCG | |
| fusion | | GUUCUGAAGGUGUUC | |
| SR220459; | | CUUCCUUAGCUUCCAGCCAUUGUGUUGAAU | 328 |
| z57z40 | | CCUUUAACCAUUCAACUGUUGCCUCCGGUU | |
| fusion | | CUGAAGGUGUUCUUG | |
| SR220460; | | GCUUCUUCCUUAGCUUCCAGCCAUUGUGUU | 329 |
| z56z46 | | GAAUCCUUUCAUUCAACUGUUGCCUCCGGU | |
| fusion | | UCUGAAGGUGUUC | |
| SR220461; | | CUUUUGGAUUGCAUCUACUGUAUAGGGACC | 330 |
| z63z46 | | CUCCUUCUUUCAUUCAACUGUUGCCUCCGG | |
| fusion | | UUCUGAAGGUGUUC | |
| SR220462; | | AUUGCAUCUACUGUAUAGGGACCCUCCUUC | 331 |
| z60z46 | | CAUGACUCAUUUCAUUCAACUGUUGCCUCC | |
| fusion | | GGUUCUGAAGGUGUUC | |
| SR220463; | | CUUUUGGAUUGCAUCUACUGUAUAGGGACC | 332 |
| z63z64 | | CUCCUUCCAGCUUCUUCCUUAGCUUCCAGC | |
| fusion | | CAUUGUGUUGAAUCCUU | |
| SR220464; | | AUUGCAUCUACUGUAUAGGGACCCUCCUUC | 333 |
| z60z64 | | CAUGACUCACAGCUUCUUCCUUAGCUUCCA | |
| fusion | | GCCAUUGUGUUGAAUCCUU | |

In some embodiments, the target sequence that binds human DMD exon 53 comprises the sequence set forth in SEQ ID NO: 237. In some embodiments, the target sequence that binds human DMD exon 53 comprises the sequence set forth in SEQ ID NO: 238. In some embodiments, the target sequence that binds human DMD exon 53 comprises the sequence set forth in SEQ ID NO: 239. In some embodiments, the target sequence that binds human DMD exon 53 comprises the sequence set forth in SEQ ID NO: 240. In some embodiments, the target sequence that binds human DMD exon 53 comprises the sequence set forth in SEQ ID NO: 241. In some embodiments, the target sequence that binds human DMD exon 53 comprises the sequence set forth in SEQ ID NO: 242. In some embodiments, the target sequence that binds human DMD exon 53 comprises the sequence set forth in SEQ ID NO: 243. In some embodiments, the target sequence that binds human DMD exon 53 comprises the sequence set forth in SEQ ID NO: 244. In some embodiments, the target sequence that binds human DMD exon 53 comprises the sequence set forth in SEQ ID NO: 245. In some embodiments, the target sequence that binds human DMD exon 53 comprises the sequence set forth in SEQ ID NO: 246. In some embodiments, the target sequence that binds human DMD exon 53 comprises the sequence set forth in SEQ ID NO: 247. In some embodiments, the target sequence that binds human DMD exon 53 comprises the sequence set forth in SEQ ID NO: 248. In some embodiments, the target sequence that binds human DMD exon 53 comprises the sequence set forth in SEQ ID NO: 249.

In some embodiments, the target sequence that binds human DMD exon 53 comprises the sequence set forth in SEQ ID NO: 250. In some embodiments, the target sequence that binds human DMD exon 53 comprises the sequence set forth in SEQ ID NO: 251. In some embodiments, the target sequence that binds human DMD exon 53 comprises the sequence set forth in SEQ ID NO: 252. In some embodiments, the target sequence that binds human DMD exon 53 comprises the sequence set forth in SEQ ID NO: 253. In some embodiments, the target sequence that binds human DMD exon 53 comprises the sequence set forth in SEQ ID NO: 254. In some embodiments, the target sequence that binds human DMD exon 53 comprises the sequence set forth in SEQ ID NO: 255. In some embodiments, the target sequence that binds human DMD exon 53 comprises the sequence set forth in SEQ ID NO: 256. In some embodiments, the target sequence that binds human DMD exon 53 comprises the sequence set forth in SEQ ID NO: 257. In some embodiments, the target sequence that binds human DMD exon 53 comprises the sequence set forth in SEQ ID NO: 258. In some embodiments, the target sequence that binds human DMD exon 53 comprises the sequence set forth in SEQ ID NO: 259.

In some embodiments, the target sequence that binds human DMD exon 53 comprises the sequence set forth in SEQ ID NO: 260. In some embodiments, the target sequence that binds human DMD exon 53 comprises the sequence set forth in SEQ ID NO: 261. In some embodiments, the target sequence that binds human DMD exon 53 comprises the sequence set forth in SEQ ID NO: 262. In some embodiments, the target sequence that binds human DMD exon 53 comprises the sequence set forth in SEQ ID NO: 263. In some embodiments, the target sequence that binds human DMD exon 53 comprises the sequence set forth in SEQ ID NO: 264. In some embodiments, the target sequence that binds human DMD exon 53 comprises the sequence set forth in SEQ ID NO: 265. In some embodiments, the target sequence that binds human DMD exon 53 comprises the sequence set forth in SEQ ID NO: 266. In some embodiments, the target sequence that binds human DMD exon 53 comprises the sequence set forth in SEQ ID NO: 267. In some embodiments, the target sequence that binds human DMD exon 53 comprises the sequence set forth in SEQ ID NO: 268. In some embodiments, the target sequence that binds human DMD exon 53 comprises the sequence set forth in SEQ ID NO: 269.

In some embodiments, the target sequence that binds human DMD exon 53 comprises the sequence set forth in SEQ ID NO: 270. In some embodiments, the target sequence that binds human DMD exon 53 comprises the sequence set forth in SEQ ID NO: 271. In some embodiments, the target sequence that binds human DMD exon 53 comprises the sequence set forth in SEQ ID NO: 272. In some embodiments, the target sequence that binds human DMD exon 53 comprises the sequence set forth in SEQ ID NO: 273. In some embodiments, the target sequence that binds human DMD exon 53 comprises the sequence set forth in SEQ ID NO: 274. In some embodiments, the target sequence that binds human DMD exon 53 comprises the sequence set forth in SEQ ID NO: 275. In some embodiments, the target sequence that binds human DMD exon 53 comprises the sequence set forth in SEQ ID NO: 276. In some embodiments, the target sequence that binds human DMD exon 53 comprises the sequence set forth in SEQ ID NO: 277. In some embodiments, the target sequence that binds human DMD exon 53 comprises the sequence set forth in SEQ ID NO: 278. In some embodiments, the target sequence that binds human DMD exon 53 comprises the sequence set forth in SEQ ID NO: 279.

In some embodiments, the target sequence that binds human DMD exon 53 comprises the sequence set forth in SEQ ID NO: 280. In some embodiments, the target sequence that binds human DMD exon 53 comprises the sequence set forth in SEQ ID NO: 281. In some embodiments, the target sequence that binds human DMD exon 53 comprises the sequence set forth in SEQ ID NO: 282. In some embodiments, the target sequence that binds human DMD exon 53 comprises the sequence set forth in SEQ ID NO: 283. In some embodiments, the target sequence that binds human DMD exon 53 comprises the sequence set forth in SEQ ID NO: 284. In some embodiments, the target sequence that binds human DMD exon 53 comprises the sequence set forth in SEQ ID NO: 285. In some embodiments, the target sequence that binds human DMD exon 53 comprises the sequence set forth in SEQ ID NO: 286. In some embodiments, the target sequence that binds human DMD exon 53 comprises the sequence set forth in SEQ ID NO: 287. In some embodiments, the target sequence that binds human DMD exon 53 comprises the sequence set forth in SEQ ID NO: 288. In some embodiments, the target sequence that binds human DMD exon 53 comprises the sequence set forth in SEQ ID NO: 289.

In some embodiments, the target sequence that binds human DMD exon 53 comprises the sequence set forth in SEQ ID NO: 290. In some embodiments, the target sequence that binds human DMD exon 53 comprises the sequence set forth in SEQ ID NO: 291. In some embodiments, the target sequence that binds human DMD exon 53 comprises the sequence set forth in SEQ ID NO: 292. In some embodiments, the target sequence that binds human DMD exon 53 comprises the sequence set forth in SEQ ID NO: 293. In some embodiments, the target sequence that binds human DMD exon 53 comprises the sequence set forth in SEQ ID NO: 294. In some embodiments, the target sequence that binds human DMD exon 53 comprises the sequence set forth in SEQ ID NO: 295. In some embodiments, the target sequence that binds human DMD exon 53 comprises the sequence set forth in SEQ ID NO: 296. In some embodiments, the target sequence that binds human DMD exon 53 comprises the sequence set forth in SEQ ID NO: 297. In some embodiments, the target sequence that binds human DMD exon 53 comprises the sequence set forth in SEQ ID NO: 298. In some embodiments, the target sequence that binds human DMD exon 53 comprises the sequence set forth in SEQ ID NO: 299.

In some embodiments, the target sequence that binds human DMD exon 53 comprises the sequence set forth in SEQ ID NO: 300. In some embodiments, the target sequence that binds human DMD exon 53 comprises the sequence set forth in SEQ ID NO: 301. In some embodiments, the target sequence that binds human DMD exon 53 comprises the sequence set forth in SEQ ID NO: 302. In some embodiments, the target sequence that binds human DMD exon 53 comprises the sequence set forth in SEQ ID NO: 303. In some embodiments, the target sequence that binds human DMD exon 53 comprises the sequence set forth in SEQ ID NO: 304. In some embodiments, the target sequence that binds human DMD exon 53 comprises the sequence set forth in SEQ ID NO: 305. In some embodiments, the target sequence that binds human DMD exon 53 comprises the sequence set forth in SEQ ID NO: 306. In some embodiments, the target sequence that binds human DMD exon 53 comprises the sequence set forth in SEQ ID NO: 307. In some embodiments, the target sequence that binds human DMD exon 53 comprises the sequence set forth in SEQ ID NO: 308. In some embodiments, the target sequence that binds human DMD exon 53 comprises the sequence set forth in SEQ ID NO: 309.

In some embodiments, the target sequence that binds human DMD exon 53 comprises the sequence set forth in SEQ ID NO: 310. In some embodiments, the target sequence that binds human DMD exon 53 comprises the sequence set forth in SEQ ID NO: 311. In some embodiments, the target sequence that binds human DMD exon 53 comprises the sequence set forth in SEQ ID NO: 312. In some embodiments, the target sequence that binds human DMD exon 53 comprises the sequence set forth in SEQ ID NO: 313. In some embodiments, the target sequence that binds human DMD exon 53 comprises the sequence set forth in SEQ ID NO: 314. In some embodiments, the target sequence that binds human DMD exon 53 comprises the sequence set forth in SEQ ID NO: 315. In some embodiments, the target sequence that binds human DMD exon 53 comprises the sequence set forth in SEQ ID NO: 316. In some embodiments, the target sequence that binds human DMD exon 53 comprises the sequence set forth in SEQ ID NO: 317. In some embodiments, the target sequence that binds human DMD exon 53 comprises the sequence set forth in SEQ ID NO: 318. In some embodiments, the target sequence that binds human DMD exon 53 comprises the sequence set forth in SEQ ID NO: 319.

In some embodiments, the target sequence that binds human DMD exon 53 comprises the sequence set forth in SEQ ID NO: 320. In some embodiments, the target sequence that binds human DMD exon 53 comprises the sequence set forth in SEQ ID NO: 321. In some embodiments, the target sequence that binds human DMD exon 53 comprises the sequence set forth in SEQ ID NO: 322. In some embodiments, the target sequence that binds human DMD exon 53 comprises the sequence set forth in SEQ ID NO: 323. In some embodiments, the target sequence that binds human DMD exon 53 comprises the sequence set forth in SEQ ID NO: 324. In some embodiments, the target sequence that binds human DMD exon 53 comprises the sequence set forth in SEQ ID NO: 325. In some embodiments, the target sequence that binds human DMD exon 53 comprises the sequence set forth in SEQ ID NO: 326. In some embodiments, the target sequence that binds human DMD exon 53 comprises the sequence set forth in SEQ ID NO: 327. In some embodiments, the target sequence that binds human DMD exon 53 comprises the sequence set forth in SEQ ID NO: 328. In some embodiments, the target sequence that binds human DMD exon 53 comprises the sequence set forth in SEQ ID NO: 329.

In some embodiments, the target sequence that binds human DMD exon 53 comprises the sequence set forth in SEQ ID NO: 330. In some embodiments, the target sequence that binds human DMD exon 53 comprises the sequence set forth in SEQ ID NO: 331. In some embodiments, the target sequence that binds human DMD exon 53 comprises the sequence set forth in SEQ ID NO: 332. In some embodiments, the target sequence that binds human DMD exon 53 comprises the sequence set forth in SEQ ID NO: 333.

### Engineered Stem Loops

The engineered snRNA (esnRNA) system disclosed herein can comprise an engineered stem loop (eSL) which includes compensatory modifications to a native snRNA stem loop. These modifications result in increased stability of the engineered small nuclear ribonuclear protein complex (esnRNP) compared to snRNP comprising an unmodified stem loop. An eSL disclosed herein can be derived from any snRNP such as U1-U12. In one embodiment, the eSL is a human or mouse U7 eSL. In one embodiment, the eSL is a human or mouse eSL. In some embodiments, the eSL is a human and mouse eSL. In some embodiments, the eSL is a non-human eSL selected from the group consisting of mouse, pig, sheep, goat, cow, dog, cat, horse, or a combination thereof. In some embodiments, the eSL is an eSL selected from the group consisting of human, mouse, pig, sheep, goat, cow, dog, cat, horse, or a combination thereof. In some embodiments, the eSL sequence is not a native stem loop sequence. In some embodiments, the nucleic acid sequence of the eSL is at least 65%, 70%, 75%, 80%, 85%, 90%, 95%, 96%, 97%, 98%, 99% or 100% (or any percentage in between) is not a native stem loop sequence. Engineered stem loops are described in WO2023168458, the contents of which are incorporated herein by reference in its entirety.

In some embodiments, a human eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 1))
ggctttctggctccttaccggaaagcc,
(SEQ ID NO: 2)
ggctttctgggaggttaccggaaagcc,
(SEQ ID NO: 3)
ggctttctggcctccttaccggaaagcc,
(SEQ ID NO: 4)
ggctttctggggaggttaccggaaagcc,
(SEQ ID NO: 5)
ggctttctggctggctaccggaaagcc,
(SEQ ID NO: 6)
ggctttctggcttccccggaaagcc,
(SEQ ID NO: 7)
ggctttctggcttcttcccggaaagcc,
(SEQ ID NO: 8)
ggctttctggcaacttaccggaaagcc,
(SEQ ID NO: 9)
ggctttctggttcggtaccggaaagcc,
(SEQ ID NO: 10)
ggctttctggaagccttaccggaaagcc,
(SEQ ID NO: 11)
ggctttctggcttcttaccggaaagcc,
or
(SEQ ID NO: 186)
GGCTTTCTGGCCTCCGCCGGAAAGCCCCT.

In some embodiments, a human eSL comprises the sequence set forth in SEQ ID NO: 1. In some embodiments, a human eSL comprises the sequence set forth in SEQ ID NO: 2. In some embodiments, a human eSL comprises the sequence set forth in SEQ ID NO: 3. In some embodiments, a human eSL comprises the sequence set forth in SEQ ID NO: 4. In some embodiments, a human eSL comprises the sequence set forth in SEQ ID NO: 5. In some embodiments, a human eSL comprises the sequence set forth in SEQ ID NO: 6. In some embodiments, a human eSL comprises the sequence set forth in SEQ ID NO: 7. In some embodiments, a human eSL comprises the sequence set forth in SEQ ID NO: 8. In some embodiments, a human eSL comprises the sequence set forth in SEQ ID NO: 9. In some embodiments, a human eSL comprises the sequence set forth in SEQ ID NO: 10. In some embodiments, a human eSL comprises the sequence set forth in SEQ ID NO: 111. In some embodiments, a human eSL comprises the sequence set forth in SEQ ID NO: 186.

In some embodiments, a murine eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 164)
ggctttctggctccttaccggaaagcccct
(SEQ ID NO: 146)
Ggttttctgacctccgtcggaaaacccct,
(SEQ ID NO: 147)
ggttttctgacctccttcggtoggaaaacccct,
(SEQ ID NO: 148)
Ggttttctgacctccgtcggaaaacc,
(SEQ ID NO: 228)
GGTTTTCTGACACTCCGTCGGAAAACCCCT,
(SEQ ID NO: 229)
GGTTTTCTGATCTCCATCGGAAAACCCCT,
or
(SEQ ID NO: 230)
GGTTTTCCGACCTCCGTCGGAAAACCCCT.

In some embodiments, a murine eSL comprises the sequence set forth in SEQ ID NO: 164. In some embodiments, a murine eSL comprises the sequence set forth in SEQ ID NO: 146. In some embodiments, a murine eSL comprises the sequence set forth in SEQ ID NO: 147. In some embodiments, a murine eSL comprises the sequence set forth in SEQ ID NO: 148. In some embodiments, a murine eSL comprises the sequence set forth in SEQ ID NO: 228. In some embodiments, a murine eSL comprises the sequence set forth in SEQ ID NO: 229. In some embodiments, a murine eSL comprises the sequence set forth in SEQ ID NO: 230.

In some embodiments, a human or murine eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 190)
GGCTTTCTGGCACTCCACCGGAAAGCCCCT,
(SEQ ID NO: 191)
GGCTTTCTGGCACTCCGCCGGAAAGCCCCT,
or
(SEQ ID NO: 192)
GGCTTTCTGGCCTCCACCGGAAAGCCCCT.

In some embodiments, a human or murine eSL comprises the sequence set forth in SEQ ID NO: 190. In some embodiments, a human or murine eSL comprises the sequence set forth in SEQ ID NO: 191. In some embodiments, a human or murine eSL comprises the sequence set forth in SEQ ID NO: 192.

In some embodiments, a dog or cat eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

GGTTTTCCGGTCTCCACCGGAAAGCCCCC (SEQ ID NO: 193). In some embodiments, a dog or cat eSL comprises the sequence set forth in SEQ ID NO: 193.

In some embodiments, a cow, sheep, or goat eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 194)
GGCTTTCCGGTCTCCACCGGAAAGCCCCT,
or
(SEQ ID NO: 195)
GGCTTTCCGGCCTCCGCCGGAAAGCCCCT.

In some embodiments, a cow, sheep, or goat eSL comprises the sequence set forth in SEQ ID NO: 194. In some embodiments, a cow, sheep, or goat eSL comprises the sequence set forth in SEQ ID NO: 195.

In some embodiments, a pig eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 196)
GGTTTTCCGGTCTCCACCGGAAAACCCTT,
(SEQ ID NO: 197)
GGTTTTCCGTGCTCCCACGGAAAACCCTT,
(SEQ ID NO: 198)
GGTTTTCCGGCCTCCGCCGGAAAACCCTT,
(SEQ ID NO: 199)
GGTTTTCCGTGACTCCCACGGAAAACCCTT,
or
(SEQ ID NO: 200)
GGTTTTCCGGCACTCCGCCGGAAAACCCTT.

In some embodiments, a pig eSL comprises the sequence set forth in SEQ ID NO: 198. In some embodiments, a pig eSL comprises the sequence set forth in SEQ ID NO: 199. In some embodiments, a pig eSL comprises the sequence set forth in SEQ ID NO: 200.

In some embodiments, a horse eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 201)
GGTCTTCCGGTCTCCTCCGGAAGGCCCCC,
or
(SEQ ID NO: 202)
GGTCTTCCGGCTCCCCGGAAGGCCCCC.

In some embodiments, a horse eSL comprises the sequence set forth in SEQ ID NO: 201. In some embodiments, a horse eSL comprises the sequence set forth in SEQ ID NO: 202.

In some embodiments, a sheep eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 203)
GGCTTTCCGTGCTCCCACGGAAAGCCCCT,
(SEQ ID NO: 204)
GGCTTTCCGTGACTCCCACGGAAAGCCCCT,
or
(SEQ ID NO: 205)
GGCTTTCCGGCACTCCGCCGGAAAGCCCCT.

In some embodiments, a sheep eSL comprises the sequence set forth in SEQ ID NO: 203. In some embodiments, a sheep eSL comprises the sequence set forth in SEQ ID NO: 204. In some embodiments, a sheep eSL comprises the sequence set forth in SEQ ID NO: 205.

In some embodiments, engineered stem loops provide for enhanced stability of an snRNA relative to an snRNA comprising a native stem loop. In some embodiments is a native snRNA stem loop comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 145)
Ggttttctgacttcggtcggaaaacccct,
(SEQ ID NO: 144)
ggttttctgacttcggtcggaaaacc,
(SEQ ID NO: 235)
Ggctttctggctttttaccggaaagcc,
(SEQ ID NO: 236)
ggctttctggctttttaccggaaagccCCT,
(SEQ ID NO: 142)
GGCTTTCCGGCCTCCGCCGGAAAGCCCCT,
or
(SEQ ID NO: 143)
GGCTTTCCGGCCTCCGCCGGAAAGCC.

In some embodiments, a native snRNA stem loop comprises the sequence set forth in SEQ ID NO: 145. In some embodiments, a native snRNA stem loop comprises the sequence set forth in SEQ ID NO: 144. In some embodiments, a native snRNA stem loop comprises the sequence set forth in SEQ ID NO: 235. In some embodiments, a native snRNA stem loop comprises the sequence set forth in SEQ ID NO: 236. In some embodiments, a native snRNA stem loop comprises the sequence set forth in SEQ ID NO: 142. In some embodiments, a native snRNA stem loop comprises the sequence set forth in SEQ ID NO: 143.

### 5′ Interaction Stability Domain

The eSL disclosed herein possesses more effective folding and annealing properties with a 5′ interaction stability domain (5′ISD) and this in turn results in increased stability of the esnRNA compared to a non-engineered snRNA. The 5′ ISD has nucleotides that are complementary to the nucleotides within the engineered SL, and without wishing to be bound by theory, an interaction between the 5′ISD and eSL is predicted to form secondary structure that protects the 5′ end of an snRNA. In some aspects the 5′ ISD anneals and/or hybridizes to an eSL of the disclosure. In some aspects the 5′ISD is a sequence having complementarity and/or reverse complementarity to a sequence present in an eSL of the disclosure. In some aspects a 5′ISD disclosed herein can be one of the 5′ISDs selected from the following nucleotide sequences:

(SEQ ID NO: 12)
ggagt,
(SEQ ID NO: 13)
cctct,
(SEQ ID NO: 14)
ggaggt,
(SEQ ID NO: 15)
cctcct,
(SEQ ID NO: 16)
agccag,
(SEQ ID NO: 17)
ggaag,
(SEQ ID NO: 18)
gaagaag,
(SEQ ID NO: 19)
gttg,
(SEQ ID NO: 20)
ccgaa,
(SEQ ID NO: 21)
taaggag,
(SEQ ID NO: 22)
gaag,
or
(SEQ ID NO: 23)
ggctt.

### Sm Binding Domains

The snRNA systems disclosed herein utilize an Sm binding domain (SmBD). The Sm protein ring that assembles around the Sm binding domain (SmBD) to form an snRNP includes SmB/B′, SmD1, SmD2, SmD3, SmE, SmF, and SmG. The U7 Sm binding site recruits endogenous RNA binding factors and can be replaced with a non-U7 snRNA to make the esnRNA more stable. In one embodiment, the SmBD is selected from the group consisting of U1, U2, U4, and U5 snRNAs. In another embodiment, the SmBD is derived from a pseudo snRNA. In another embodiment, the SmBD is a nucleotide sequence comprising SEQ ID NO: 31 (ATTTTT). In another embodiment, the SmBD comprises a nucleotide sequence selected from the group consisting of SEQ ID NO: 32 (AATTTTTGG), SEQ ID NO: 33 (AATTTGTGG), SEQ ID NO: 34 (AATTTGTGG), SEQ ID NO: 35 (AATTTCTGG), SEQ ID NO: 36 (GATTTTTGG), SEQ ID NO: 37 (AATTTTTGA), SEQ ID NO: 38 (AATTTTTTG), SEQ ID NO: 161 (AATTTTTGGAGCA), and SEQ ID NO: 163 (AATTTTTGGAGTA).

### Promoter Sequences

The snRNA systems disclosed herein comprise an snRNA promoter from any of U1-U12. In one embodiment, the snRNA promoter is a U7 promoter. In another embodiment, the U7 promoter is a human U7 promoter (hU7) or a mouse U7 promoter (mU7). In another embodiment, the U7 promoter is an endogenous human U7 promoter at least 65%, 70%, 75%, 80%, 85%, 90%, 95%, 96%, 97%, 98%, 99% or 100% (or any percentage in between) identical to SEQ ID NO: 39:

TACTGCCGAATCCAGGTCTCCGGGCTTAACAACAACGAAGGGGCTG
TGACTGGCTGCTTTCTCAACCAATCAGCACCGAACTCATTTGCAT
GGGCTGAGAACAAATGTTCGCGAACTCTAGAAATGAATGACTTAA
GTAAGTTCCTTAGAATATTATTTTTCCTACTGAAAGTTACCACAT
GCGTCGTTGTTTATACAGTAATAGGAACAAGAAAAAAGTCACCTA
AGCTCACCCTCATCAATTGTGGAGTTCCTTTATATCCCATCTTCT
CTCCAAACACATACGCA.

In one embodiment, the snRNA promoter is a U1 promoter. In another embodiment, the U1 promoter is a human U1 promoter or a mouse U1 promoter.

In another embodiment, the same snRNA promoter drives expression of each copy of an snRNA insert. In another embodiment, each copy of an snRNA insert is the same. In another embodiment, different snRNA promoters drive each copy of an snRNA insert. In one embodiment, a 2× snRNA comprises a mouse U7 promoter driving one copy of an snRNA insert and a mouse U1 promoter drives the other copy of an snRNA insert.

In other aspects, the snRNA promoter is a PolII promoter or a PolIII promoter. In other aspects, the snRNA promoter comprises, consists essentially of, or consists of a nucleic acid sequence at least 65%, 70%, 75%, 80%, 85%, 90%, 95%, 96%, 97%, 98%, 99% or 100% (or any percentage in between) identical to a promoter and/or promoter sequence listed in Table 6, which follows:

TABLE 6
Exemplary Promoter Table

| Exemplary | |
|---|---|
| Promoter | Promoter Sequence |
| hU1 | AAGGACCAGCTTCTTTGGGAGAGAACAGACGCAGGGGCGGGAGGG |
| | AAAAAGGGAGAGGCAGACGTCACTTCCCCTTGGCGGCTCTGGCAG |
| | CAGATTGGTCGGTTGAGTGGCAGAAAGGCAGACGGGGACTGGGCA |
| | AGGCACTGTCGGTGACATCACGGACAGGGCGACTTCTATGTAGAT |
| | GAGGCAGCGCAGAGGCTGACGTCTTCGCCACTTGCTGCTTCACCA |
| | CGAAGGAGTTCCCGTGCCCTGGGAGCGGGTTCAGGACCGCTGATC |
| | GGAAGTGAGAATCCCAGCTGTGTGTCAGGGCTGGAAAGGGCTCGG |
| | GAGTGCGCGGGGCAAGTGACCGTGTGTGTAAAGAGTGAGGCGTAT |
| | GAGGCTGTGTCGGGGCAGAGGCacaacgtttc |
| | (SEQ ID NO: 40) |
| hU2 | CCACGCCCTCTGTGAAAGGGCGGGGCATGCAAATTCGAAATGAAA |
| | GCCCGGGAACGCCGGAAGAAGCACGGGTGTAAGATTTCCCTTTTC |
| | AAAGGCAGAGAATAAGAAATCAGCCCGAGAGTGTAAGGGCGTCAA |
| | TAGCGCTGTGGACGAGACAGAGGGAATGGGGCAAGGAGCGAGGCT |
| | GGGGCTCTCACCGCGACTTGAATGTGGATGAGAGTGGGACGGTGA |
| | CGGCGGGCGCGAAGGCGAGCGC |
| | (SEQ ID NO: 41) |
| hU4 | TTCGCAGTCTCTGAATTAAGTCTATTAGCATGTTCCTCCCATAGT |
| | GCTTTGCTTCATATCAACAAAAACCTAGCTAAGTGAAATCAGCAA |
| | CGATATGCAGAAACCACCTACGCAGGTCACAAACATCTTTCTATG |
| | ATTGTATAATTTTCAAGCAAGCAATAAGTGAAGATTTTTCCATAG |
| | GCCCTAAACTCACCTTTGCGAAATAGGAAGCTGGTTTATTGGGAG |
| | TGATGAGCAGGGGGCGTAACAAATT |
| | (SEQ ID NO: 42) |
| hU5 | GCAGCAAGGCCTCCACTTCACCCCCTAAAGGTTGCCCCAAGAGCA |
| | CCGTGTGACTGCTAAGGTATTTCCGGAGTCTAAAGACGATTATTC |
| | AGGTCTCATTTGCATACCCATAATACACTGCAAACAGTATTTTTT |
| | TCGGAAAAACATTTATATATTGCTTGACATTTTTAAGTATGAGAA |
| | TTTTGCATGCAGAATTTTTTTGTATAAACTTTCTCAGGTAGTAAC |
| | CCTTGGGATTAGTAGACACCATCAGTGTACTAGGAATTGCAGTTA |
| | CCCGAAAATTGAGTTACAGAAGTAACTGGT |
| | (SEQ ID NO: 43) |
| hU6 | Gtttattacagggacagcagagatccagtttggttaattaaggta |
| | ccgagggcctatttcccatgattccttcatatttgcatatacgat |
| | acaaggctgttagagagataattagaattaatttgactgtaaaca |
| | caaagatattagtacaaaatacgtgacgtagaaagtaataatttc |
| | ttgggtagtttgcagttttaaaattatgttttaaaatggactatc |
| | atatgcttaccgtaacttgaaagtatttcgatttcttggctttat |
| | atatcttgtggaaaggacgaaacacc |
| | (SEQ ID NO: 44) |
| h7sk | CTGCAGTATTTAGCATGCCCCACCCATCTGCAAGGCATTCTGGAT |
| | AGTGTCAAAACAGCCGGAAATCAAGTCCGTTTATCTCAAACTTTA |
| | GCATTTTGGGAATAAATGATATTTGCTATGCTGGTTAAATTAGAT |
| | TTTAGTTAAATTTCCTGCTGAAGCTCTAGTACGATAAGTAACTTG |
| | ACCTAAGTGTAAAGTTGAGATTTCCTTCAGGTTTATATAGCTTGT |
| | GCGCCGCCTGGGTACCTC |
| | (SEQ ID NO: 45) |
| RNA(Val) | CAGGACTAGTCTTTTAGGTCAAAAAGAAGAAGCTTTGTAACCGTT |
| | GGTTTCCGTAGTGTAGTGGTTATCACGTTCGCCTAACACGCGAAA |
| | GGTCCCCGGTTCGAAG |
| | (SEQ ID NO: 46) |
| mU1 | TTGTTCCTCTTAGTGTTAATTCACACTAAAGACTGTGCATCCGAC |
| | TCCTACATTTATGAAAGTAAATGCCTGTTGTTAGAACAAAAAAGG |
| | CTACAGAACAAAAAACAAAGCGAAATACCATCTGCTTTAGGTTCA |
| | GTGTGGTATTTTCCCGCTGACAGGGAGGCGGGTTTTTGGGTACAG |
| | GAAACGAGTCACTATGGAGGCGGTACTATGTAGATGAGAATTCAG |
| | GTGCAAACTGGGAAAAGCAACTGCTTCCAAATATTTGTGATTTTT |
| | ACAGTGTAGTTTTGGAAAAACTCTTAGCCTACCAATTCTTCTAAG |
| | TGTTTTAAAATGTGGGAGCCAGTACACATGAAGTTATAGAGTGTT |
| | TTAATGAGGCTTAAATATTTACCGTAACTATGAAATGCTACGCAT |
| | ATCATGCTGTTCAGGCTCCGTGGCCACGCAACTC |
| | (SEQ ID NO: 47) |
| mU7+ | Taacaacataggagctgtgattggctgttttcagccaatcagcac |
| extra 3′ | tgActcatttgcatagcctttacaagcggtcacaaactcaagaaa |
| | cgagcggttttaatagtcttttagaatattgtttatcgaaccgaa |
| | taaggaactgtgctttgtgattcacatatcagtggaggggtgtgg |
| | aaatggcaccttgatctcaccctcatcgaaagtggagttgatgtc |
| | ctTccctggctcgctacagacgcacttccgcaa |
| | (SEQ ID NO: 48) |
| mU7 | taacaacataggagctgtgattggctgttttcagccaatcagcac |
| | tgActcatttgcatagcctttacaagcggtcacaaactcaagaaa |
| | cgagcggttttaatagtcttttagaatattgtttatcgaaccgaa |
| | taaggaactgtgctttgtgattcacatatcagtggaggggtgtgg |
| | aaatggcaccttgatctcaccctcatcgaaagtggagttgatgtc |
| | ctTccctggctcgctacagacgcacttccgc |
| | (SEQ ID NO: 151) |
| hU7 | TACTGCCGAATCCAGGTCTCCGGGCTTAACAACAACGAAGGGGCT |
| | GTGACTGGCTGCTTTCTCAACCAATCAGCACCGAACTCATTTGCA |
| | TGGGCTGAGAACAAATGTTCGCGAACTCTAGAAATGAATGACTTA |
| | AGTAAGTTCCTTAGAATATTATTTTTCCTACTGAAAGTTACCACA |
| | TGCGTCGTTGTTTATACAGTAATAGGAACAAGAAAAAAGTCACCT |
| | AAGCTCACCCTCATCAATTGTGGAGTTCCTTTATATCCCATCTTC |
| | TCTCCAAACACATACGCA |
| | (SEQ ID NO: 39) |
| mU5 | TGCCAACAGCTTTGAAATCCTCTGTGCTCTTGTGCCAATCCCACC |
| | CTCAGAACAGGGAGTCTGTCGGTTATGTTTTCGTCCTTTACAGTT |
| | CTTCGCCGTCTAATCCATTGGAAACCTCCTTTATTTAAAGGAAAC |
| | CGTGTCCAAATTCAGTGTACAGAGGTGCAAACCTCCGCCCCGCTC |
| | CTTGTAGGAAGCCCACGGGAACTGGGCCAATCGGACGGCGCTTAC |
| | TCGGCTCATTTACATACCCATAACACACCGCGGCTAATGCAAATA |
| | TTTTCGTGAAAAGATCTTCCATTACTCGAAGTTTTAAAACTAAAA |
| | ACAAAATATTATTTCAAAAGATCATGAAGAAATTTCTGTCGGTAA |
| | CAGCAGTTTCAATTGATAAATCACCATCACTTATCTAGGAGGTTC |
| | TGTTACTCTAGAAGTGAATTAAGCAGGACAGCTGT |
| | (SEQ ID NO: 152) |
| mU2 | Gggcggggcatgcaaataactgctctgtggaactctgggagcaaa |
| | aacaaaaaactgcaaccaaaacttctcggcctccttgaatcttac |
| | aggcttttcgtggcgtaaaggtggtgtactcaatgaagaggagag |
| | tctgtgttggctgcatgtttgagtcggttggttggtgactgtgaa |
| | tTAAAGGTGTGGtcggtgttgagtgtatggggcgtgtgggcgtag |
| | ttcggt (SEQ ID NO: 153) |
| mU6 | cgacgccgccatctctaggcccgcgccggccccctcgcacagact |
| | tgtgggagaagctcggctactcccctgccccggttaatttgcata |
| | taatatttcctagtaactatagaggcttaatgtgcgataaaagac |
| | agataatctgttctttttaatactagctacattttacatgatagg |
| | cttggatttctataagagatacaaatactaaattattattttaaa |
| | aaacagcacaaaaggaaactcaccctaactgtaaagtaattgtgt |
| | gttttgagactataaatatcccttggagaaaagccttgttt |
| | (SEQ ID NO: 154) |
| mH1 | CATgCAAATTACGCGCTgTGCTTTGTGGGAAATCACCCTAAACGT |
| | AAAATTTATTCCTCTTTcGAGCCTTATAGTGGGGCCGGTCTACAC |
| | CCTAAA |
| | (SEQ ID NO: 155) |
| U1 | TTACTTCATACTAAAGGCTGTGCATCCGACTCCTAAGTTGATGAA |
| | GGAAAATGCCTAGTGTTCTTGGAGGCTACAAAACAAAAGACAAAG |
| | CTAACTACCATCTGCTTATGGGTTCATTGGTATTTTCCAGCTGGC |
| | AGGGAGGCGGGTTTCCGAGTACAGGAAATGAGTCTCTATGGAGGC |
| | GGTGCTATGTAGATGAGAATTCAGGAGCAAACTGGGAAAAGCAGC |
| | TGCTTCCAAATATTTGTGATTTTTGGAGTGTAGTTTTGGGGAAAC |
| | TCGCAGCCTACCAATTCTCCTAAGTGCTTTAGAATATGGAGAGAC |
| | ACTGTACATAAAGATATAGAGCTTTTTAATGGAGGCTTAAATTTA |
| | TACCGTATCTACAAAATGCTACATTCACAATGCAGTTCAGGCTCT |
| | GTGGCATTGCAACTC |
| | (SEQ ID NO: 165) |
| bU7 | GCGCAGGAGCCGCCGAGCTCTTGCTGCGAAGCCTTGTCTGCGTTC |
| | TTAAAAACTAAAGGGGGCGTGACTGGCTTCCTTATCAGCCAATCG |
| | GCATCGGGTCATTTGCATAGGGCCCCATCCACCGTTCGTAAACTC |
| | TAGCCGCGAAGGATTTAAGAGAGTTCCTTAGAACACGGTCTTCCC |
| | CTCCGAAAGTCGCAGTCTGCCTCGTTATCTGTAGAGCAATAGGAG |
| | CTAGGAACCGGCCGTCGGGGCTCACCCTCACTGACAGCGGGGTTG |
| | GTGGCTATGCCGTGCGATCTCCGGGTGCTACGTCTG |
| | (SEQ ID NO: 166) |
| SU1 | CGCCGTGCCCTGCTCTGCCCTCCGCACGCTGCTCAGACTCCACAC |
| | CCGTAACGAAGCTCCACCGAATTTTTCGTCTGTCTGTGAAGACGA |
| | AGAATTGAAAAATCTCAGCTACACTTGGTCTTTGGGGTCGGCGCC |
| | AGGCAGAGCTAGAACGAACTAGACGCTTGTCCGAAAGCAGTGACG |
| | TCAAATGACAAGGGACAAGAGGTGGGGATATGTAGATGAGGGGAG |
| | CGGTGGCTGCTGGCTGGATTTCGTCTTTCGCCGGGTAGAATTTCC |
| | CCTGAACGTCCAAAGGATGTATCTTGGTCACTGTGAAATAAATAA |
| | TCCTTCAGGTTCCCATCGAAGGCCGAAATAGCTTGGAATATAATT |
| | GACCATAACTCTGTTGTGTGCTGAGTTCGAGGGTGACGTCCCAGA |
| | CTAAGCTTACAACTC |
| | (SEQ ID NO: 167) |
| eqU1 | TGCCGCGCAGAATGCAGACTCATCCTGACAAGTCCACGCCAGAAC |
| | CTTTGCGTCTGCCCGGAAAGGCAAGGAAGTTTAAAGCCTTTCCAG |
| | ATACCCCTATGGATGATTGTCTTTTAGATCGTCCTGAGGGCTAGC |
| | TAGAGCGAATTCACCGCTTCTCCGAGAAGCAGAATGACGTCGAGT |
| | GACGACAGTGATGACAAGAGGCGGGGATATGTAGATGAAAGACAC |
| | GGTGACTGCCGGTTGGAGTGAGTTCTTCATGGGGAACACTTTCCA |
| | CTGAATATCCAAAAGGATGTACCCTGATCTCTGTCATATAAATAA |
| | CCCTTCAATTTCGAACTTAAGGCCTAAAGAACTCGGAATATAATT |
| | GACCATGACTTTATAGTGTGTTGAAGACGATGGTGAGGTGCCAGG |
| | CGAGGGGAGCAACTC |
| | (SEQ ID NO: 168) |

In some embodiments, the snRNA promoter comprises the sequence set forth in SEQ ID NO: 40. In some embodiments, the snRNA promoter comprises the sequence set forth in SEQ ID NO: 41. In some embodiments, the snRNA promoter comprises the sequence set forth in SEQ ID NO: 42. In some embodiments, the snRNA promoter comprises the sequence set forth in SEQ ID NO: 43. In some embodiments, the snRNA promoter comprises the sequence set forth in SEQ ID NO: 44. In some embodiments, the snRNA promoter comprises the sequence set forth in SEQ ID NO: 45. In some embodiments, the snRNA promoter comprises the sequence set forth in SEQ ID NO: 46. In some embodiments, the snRNA promoter comprises the sequence set forth in SEQ ID NO: 47. In some embodiments, the snRNA promoter comprises the sequence set forth in SEQ ID NO: 48. In some embodiments, the snRNA promoter comprises the sequence set forth in SEQ ID NO: 151. In some embodiments, the snRNA promoter comprises the sequence set forth in SEQ ID NO: 39. In some embodiments, the snRNA promoter comprises the sequence set forth in SEQ ID NO: 152. In some embodiments, the snRNA promoter comprises the sequence set forth in SEQ ID NO: 153. In some embodiments, the snRNA promoter comprises the sequence set forth in SEQ ID NO: 154. In some embodiments, the snRNA promoter comprises the sequence set forth in SEQ ID NO: 155. In some embodiments, the snRNA promoter comprises the sequence set forth in SEQ ID NO: 165. In some embodiments, the snRNA promoter comprises the sequence set forth in SEQ ID NO: 166. In some embodiments, the snRNA promoter comprises the sequence set forth in SEQ ID NO: 167. In some embodiments, the snRNA promoter comprises the sequence set forth in SEQ ID NO: 168.

### Terminator Sequences

The snRNA systems disclosed herein comprise an snRNA downstream terminator (DT). Downstream terminators define the end of a transcriptional unit, such as an esnRNA or snRNA. In another embodiment the snRNA DT is a U7 DT comprising: CCTCTTATGATGTTTGTTGCCAATGATAGATTGTTTTCACTGTGCAAAAATTATGG GTAGTTTTGGTGGTCTTGATGCAGTTGTAAGCTTGGAG (SEQ ID NO: 49).

In one embodiment, the esnRNA comprises the eSL, one or more promoters, the TS targeting a DMD, the SmBD, the 5′ISD, and the DT. In one aspect, promoter and DT combinations are mixed and matched. In another embodiment, the snRNA comprises a native stem loop, one or more promoters, the TS targeting DMD, the SmBD, and the DT.

In some embodiments, the DT comprises, consists essentially of, or consists of a nucleic acid sequence at least 65%, 70%, 75%, 80%, 85%, 90%, 95%, 96%, 97%, 98%, 99% or 100% (or any percentage in between) identical to a DT sequence listed in Table 7, which follows:

TABLE 7
Exemplary DT Table

| Exemplary | |
|---|---|
| DT | DT Sequence |
| hU1 | ACTTTCTGGAGTTTCAAAAACAGACTGTACGCCAAGGGTC |
| | ATATCTTTTT(SEQ ID NO: 50) |
| hU2 | CCGGGGATACAACGTGTTTCCTAAAAGTAGAGGGAGGTAA |
| | GAGACGGTAG(SEQ ID NO: 51) |
| hU4 | CTGAATTTTCTTGCAGTTGAACAACAGAGGCTTTTTTTGT |
| | GTGTGTGGGG(SEQ ID NO: 52) |
| hU5 | ATATGTGGTAATCCAACAATAGAAATTATTTTTAAGTTTG |
| | TGTGTTCCTT (SEQ ID NO: 53) |
| hU6 | TTTTTT(SEQ ID NO: 54) |
| h7sk | TTTTTTSEQ ID NO: 55) |
| RNA | TTTTTT |
| (Val) | (SEQ ID NO: 56) |
| mU1 | GTTTACTTGGTTTTAAAAATAGCTTGCACTAGCGATACGG |
| | AATATGGTTATTAGGTTTGTTAGGCATCATGTCGTGTCTT |
| | ACTATAGAAAAATAACGTAGTGTTCATTTTAGCCTGCCTG |
| | TATGTGTTAATTTGTCCTTATTGCGCATTGTTCTTGTTAA |
| | GTCTTCTGTAAGGAGTTGCGGGTTTCAAACTGTCAGTCTG |
| | AGAGCA(SEQ ID NO: 57) |
| mU7 | Cccaatttcactggtctacaatgaaagcaaaacagttctc |
| | ttccccgctccccggtgtgtgagaggggctttgatccttc |
| | tctggtttcctaggaaacgcgtatgtgtac |
| | (SEQ ID NO: 58) |
| mU7 | Cccaatttcactggtctacaatgaaagcaaaacagttctc |
| | ttccccgctccccggtgtgtgagaggggctttgatccttc |
| | tctggtttcctaggaaacgcgtatgtg |
| | (SEQ ID NO: 156) |
| hU7 | CCTCTTATGATGTTTGTTGCCAATGATAGATTGTTTTCAC |
| | TGTGCAAAAATTATGGGTAGTTTTGGTGGTCTTGATGCAG |
| | TTGTAAGCTTGGAG |
| | (SEQ ID NO: 49) |
| hU7 | CTTATGATGTTTGTTGCCAATGATAGATTGTTTTCACTGT |
| | GCAAAAATTATGGGTAGTTTTGGTGGTCTTGATGCAGTTG |
| | TAAGCTTGGAG |
| | (SEQ ID NO: 234) |
| mU5 | CCACATTTTGTGTTTAAAAATAGAAATATTTAAGTGAGAT |
| | CAGTTTAAATCTGCTTTATCTAGGGTGTCTAACTGCTTGC |
| | ATCTTTTTAACTTTTCCTTATTTGTGAGAAGGTCTGTAAA |
| | ACTTATTATATGTTAGTACACTGTAGCTGTCTTCAGACAC |
| | TCCAGAAGAGGGATTCAGATCACCTTACAGATGGTTTTGA |
| | (SEQ ID NO: 157) |
| mU2 | CCCTCTGGGGAgtaaagttggttttaaagtcagagcatgg |
| | tgattgtagggcagtccaacttttttaaatatgctgtg |
| | (SEQ ID NO: 158) |
| mU6 | TTTTTT |
| mH1 | TTTTTT |
| rU1 | ATTTGTTTGGTACTAAAGATAGTTATCAGCCGAACCAGAA |
| | GGCTAAAATGGCTTCTGATACTTACTTGGCCAATGCCTTT |
| | TCCCTTTATACTGCTATTGCTTTGTATTCTGAAAAGCATC |
| | TTAGTGGTTTTTAACTTTCTCTACGTTCCCTCTGCACGTT |
| | GAGTCTTGAGTTATGTTAAATGGTACGGTACCTAGGATTG |
| | (SEQ ID NO: 169) |
| bU7 | CCCACACAAGTGTTTATGAACAATAGAATAATATGTGGGT |
| | GGGAAGCTTGGTTTTTTTTGATACGGGTTTTTTTTTTTTT |
| | TTTGGTCTACGTGTTTAGTCCGTTCTTGACGGAGAAGACC |
| | CGGGAGGTTGGGATTCTTGTCTGGAAATTTCCGGCTCTGT |
| | CTGATGTAGTTTCCCGTTCGCCTCCCTTCTTTGCTGCGAG |
| | (SEQ ID NO: 170) |
| SU1 | GCGTGGTTTGTGCAAGGAAAGACATAGATGCTAAGTTTGG |
| | AGCTCTGCTAATTTCCTGTTTTTGTAGTCGTTTTCGGGTT |
| | TATGAGGCAGAGGCAGTAAGCAGTTTTCCTGCAACATGGA |
| | TAAGCTAGTTTGGACTTTTGGTAAAGGATTTTTTTGTAAT |
| | GTGGGAGACTTGCTTTTGATCCCTGGTTTGGGAAATTCTC |
| | (SEQ ID NO: 171) |
| eqU1 | ACAAACTTGGTCTTAAAAGAAGACTACTTGTTTATGTTCT |
| | TCTTTCTCTAACTTTTATTTGGGGTTTAGTGGCTTTCTAA |
| | CATGGTCATTATGAGGTAAGACCTTGGGGTTGGCCTATTA |
| | ATTAACACCTTGGGGCTGGCCGTATGGCTGAGTGGTTAAG |
| | TTTGTGTGCTCTGCTTCGGGTTTGGCCTGTTCTAATTCTG |
| | (SEQ ID NO: 172) |

In some embodiments, the DT comprises the sequence set forth in SEQ ID NO: 50. In some embodiments, the DT comprises the sequence set forth in SEQ ID NO: 51. In some embodiments, the DT comprises the sequence set forth in SEQ ID NO: 52. In some embodiments, the DT comprises the sequence set forth in SEQ ID NO: 53. In some embodiments, the DT comprises the sequence set forth in SEQ ID NO: 54. In some embodiments, the DT comprises the sequence set forth in SEQ ID NO: 55. In some embodiments, the DT comprises the sequence set forth in SEQ ID NO: 56. In some embodiments, the DT comprises the sequence set forth in SEQ ID NO: 57. In some embodiments, the DT comprises the sequence set forth in SEQ ID NO: 58. In some embodiments, the DT comprises the sequence set forth in SEQ ID NO: 156. In some embodiments, the DT comprises the sequence set forth in SEQ ID NO: 49. In some embodiments, the DT comprises the sequence set forth in SEQ ID NO: 234. In some embodiments, the DT comprises the sequence set forth in SEQ ID NO: 157. In some embodiments, the DT comprises the sequence set forth in SEQ ID NO: 158. In some embodiments, the DT comprises the sequence set forth in SEQ ID NO: 169. In some embodiments, the DT comprises the sequence set forth in SEQ ID NO: 170. In some embodiments, the DT comprises the sequence set forth in SEQ ID NO: 171. In some embodiments, the DT comprises the sequence set forth in SEQ ID NO: 172. In some embodiments, the DT comprises the sequence set forth in SEQ ID NO: TTTTTT.

In one embodiment, the snRNA is delivered in a vector.

In one embodiment, the snRNA is delivered in an AAV vector.

In some embodiments, the AAV vector comprises multiple copies of the snRNA. In some embodiments, the multiple copies of the snRNA are 2, 3, 4, 5, 6, 7, 8, 9, or 10 copies (2×, 3×, 4×, 5×, 6×, 7×, 8×, 9× or 10×) of the snRNA. In some embodiments, the multiple copies of the snRNA are 4 or more copies of the snRNA.

In some embodiments, each snRNA of the multiple copies of snRNA is separated by a nucleic acid buffer sequence derived from human non-coding genomic sequences downstream of an snRNA. In one embodiment, the buffer sequence is derived from human genomic sequences downstream of U7.

In one embodiment, the buffer sequence is selected from the group consisting of the following nucleic acid sequences:
buffer 1 (30 bp) CAAACTACAGAGCCAAGTGCTATCCACAGA (SEQ ID NO: 24),
buffer 2 (30 bp) GAGCTTTCTGGGTTGCCATCTCAAGCAGAC (SEQ ID NO: 25),
buffer 3 (30 bp) TACAAGGCCATCAGCTCATACTCACAATTG (SEQ ID NO: 26), and a combination thereof.

In another embodiment, the buffer sequence is selected from the group consisting of the following nucleic acid sequence:

buffer 1 (100 bp)
(SEQ ID NO: 27)
CAAACTACAGAGCCAAGTGCTATCCACAGAGAGCTTTCTGGGT
TGCCATCTCAAGCAGACTACAAGGCCATCAGCTCATACTCACA
ATTGACTTTGAGAG,
buffer 2 (100 bp)
(SEQ ID NO: 28)
TTGACCACATACGTGCTCTTTCAAAGTTCTGTGTTTGAAGTTA
TGTTAGTAACAACTGATGCCCATCCTGCAATGACAAATCCAAT
TCTCAGTGCAGCTC,

and a combination thereof.

In another embodiment, the buffer sequence is selected from the group consisting of the following nucleic acid sequence:
buffer 1 (500 bp) CAAACTACAGAGCCAAGTGCTATCCACAGAGAGCTTTCTGGGTTGCCATCTCAAG CAGACTACAAGGCCATCAGCTCATACTCACAATTGACTTTGAGAGTCATTTTCCA ATGCTCCTACACACCCCTTCTTCACAATCCCCAACAAATCTGAGGCTGGAACTTG GTACCATAACAATCATTACATTATTTCACCAGAAGTACACCTTGCCTGGAAGATT GGCATTATAGCATCTTCTAACATTGTGAAAGTTAGTGACCAATGAGGAGATCCAA GTCAGTTCCAGTTGGATTTCTCTATACTCTATAATAAATATATATGGTGTCTTCAA CAATAGGACTTTGCCATCCAGTGATGCTAAAAATCAATAACAATGGCAATAACC TGCCCTGTTTGGAAAGCCTCTGGCTTCCATGACTAACAATTCAAGGCAGGTCTCC TATACCTAGTACTGAGATTTTTATTTGATAAACTATATCTTCTGGGAGGAGAAGC ATTGT (SEQ ID NO: 29),
buffer 2 (500 bp) TTGACCACATACGTGCTCTTTCAAAGTTCTGTGTTTGAAGTTATGTTAGTAACAAC TGATGCCCATCCTGCAATGACAAATCCAATTCTCAGTGCAGCTCTCTGAAATAGT TTTGCTTTCTCTCTCTAGGTCTGTTCTATACTCCTAACTCTCCAGGAGTTTACAAG GAATAAAATCTCTTCCAAATGCTTTCTGTTGCAACAACTGGACCATACTGAAAGC TGAGGCCCACAATTGCAATCTAGGTTAGCAGGTAATCATTGTTGGTGAGGTCCTC CCTTTCCCCAGGCTCGTGTTTGTATTGGGGAGCAGGAAATTTTTGCTAGAGCAGC ACTGCCATCTCTCTACACTCCACCTGATTGGTGGGATGGACCAGAGAAATGGACA TTCCCAACACAGTCCCTCCTTTCACATCTGCTCACCTGCCCACAGGATACTTTCCA CCATGCATACTGGGCTCTGCACCAACCATTCAGCAGTGATGAAGAGGAAACTTG AAC (SEQ ID NO: 30), and/or a combination thereof.

The 100 bp and 500 bp buffer 1 sequences are derived from a sequence starting 100 bp downstream of the Mus musculus U7 pseudogene 8 (Location Chromosome 14: 4,409,359-4,409,421 reverse strand. GRCm39:CM001007.3). The 100 bp and 500 bp buffer 2 sequences are derived from the sequence starting 130 bp downstream of human U7 pseudogene 5 (Chromosome X: 140,451,148-140,451,208 forward strand.GRCh38:CM000685.2). Both 100 bp buffers are the first 100 bp of the corresponding 500 bp buffer. The 30 bp buffers 1, 2, and 3, are sequential 30 bp sequences within “100 bp buffer 1”, downstream of the Mus musculus U7 pseudogene 8. These downstream sequences were selected due to the lack of any known regulatory sites or genes within or nearby to the sequence (using Gencode/Ensembl), in addition to lack of repetitive sequence, 40-60% GC content for total buffer, 40-60% GC content in the 20 bp region at both ends of the buffer, and minimal sequence complexity.

snRNA Sequences

Exemplary snRNA sequences of the disclosure can comprise any combination of esnRNA or snRNA features described herein.

### Vectors

Also provided herein are vectors (e.g., recombinant expression vectors) comprising snRNA(s) targeting DMD.

In some embodiments of the compositions and methods of the disclosure, a vector comprises an snRNA system targeting DMD provided herein. In some embodiments, the vector is a single or unitary vector.

In some embodiments, snRNA system is capable of targeting one or more DMD RNA sequences. In some aspects, the DMD RNA sequence is a DMD pre-mRNA sequence. In some aspects, the snRNA systems are capable of targeting multiple (i.e., two or more) RNAs of interest. In some embodiments, the two or more RNAs of interest can be the same pre-mRNA molecule but different sequences within the pre-mRNA molecule.

Within the context of a recombinant expression vector, the terminology “operably linked” is intended to mean that the hybrid promoter is linked to a nucleotide sequence of interest (NOI) in a manner permitting expression of the nucleotide sequence in, for example, a host cell when the vector is introduced into (or in contact with) the host cell.

In some embodiments of the compositions and methods of the disclosure, a vector comprises the snRNA targeting DMD. In some embodiments, the therapeutic snRNA is in a single or unitary vector.

In some embodiments of the compositions and methods of the disclosure, the RNA-binding snRNA systems are capable of targeting DMD RNA sequences. In some embodiments of the compositions and methods of the disclosure, the RNA-targeting systems are capable of targeting one or more DMD RNA sequences. In some aspects, the DMD RNA sequence is a DMD pre-mRNA sequence. In some aspects, the snRNA systems are capable of targeting multiple (i.e., two or more) RNAs of interest. In some embodiments, the two or more RNAs of interest can be the same pre-mRNA molecule but different sequences within the pre-mRNA molecule.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector. Vectors are capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes to which they are operatively-linked. Common expression vectors are often in the form of plasmids. In some embodiments, recombinant expression vectors comprise a nucleic acid provided herein such as e.g., an esnRNA in a form suitable for expression of an RNA molecule in a host cell. Recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell. Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., snRNAs, CRISPR transcripts, proteins, enzymes, mutant forms thereof, fusion proteins thereof, etc.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, the snRNA constructs disclosed herein comprise bidirectional snRNA promoters to express snRNAs.

In another embodiment, the vector configurations can comprise linker(s), signal sequence(s), and/or tag(s).

### Viral Vectors

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments, the vector further comprises one or more expression control elements operably linked to the polynucleotide. In some embodiments, the vector further comprises one or more selectable markers. In some embodiments, the vector has low toxicity. In some embodiments, the vector does not incorporate into the host genome, thereby having a low probability of causing insertional mutagenesis.

### Adeno-Associated Virus Vectors

An “AAV vector” as used herein refers to a vector comprising, consisting essentially of, or consisting of one or more nucleic acid molecules and one or more AAV inverted terminal repeat sequences (ITRs). In some aspects the nucleic acid molecule encodes for an esnRNA of the disclosure. Such AAV vectors can be replicated and packaged into infectious viral particles when present in a host cell that provides the functionality of rep and cap gene products, for example, by transfection of the host cell. In some aspects AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vectors may also contain elements for manufacturing purposes, e.g., antibiotic resistance genes, origin of replication sequences etc., but these are not encapsidated and thus do not form part of the AAV particle.

In some aspects, an AAV vector can comprise at least one nucleic acid encoding an snRNA or esnRNA composition of the disclosure. In some aspects, an AAV vector can comprise at least one regulatory sequence. In some aspects, an AAV vector can comprise at least one AAV inverted terminal (ITR) sequence. In some aspects, an AAV vector can comprise a first ITR sequence and a second ITR sequence. In some aspects, an AAV vector can comprise at least one promoter sequence. In some aspects, an AAV vector can comprise at least one enhancer sequence. In some aspects, an AAV vector can comprise at least one terminator sequence. In some aspects, an AAV vector can comprise at least one poly A sequence. In some aspects, an AAV vector can comprise at least one linker sequence. In some aspects, an AAV vector can comprise at least one buffer sequence. In some aspects, an AAV vector of the disclosure can comprise at least one nuclear localization signal, or nuclear export signal and/or both.

In some aspects, an AAV vector can comprise a first AAV ITR sequence, a promoter sequence, an snRNA sequence and/or esnRNA sequence, a terminator sequence and a second AAV ITR sequence. In some aspects, an AAV vector can comprise, in the 5′ to 3′ direction, a first AAV ITR sequence, a promoter sequence, an esnRNA sequence, a terminator sequence, and a second AAV ITR sequence. In some aspects, an AAV vector can comprise, in the 5′ to 3′ direction, a first AAV ITR sequence, a promoter sequence, an snRNA sequence, a terminator sequence, and a second AAV ITR sequence.

In some aspects, an AAV vector can comprise a first AAV ITR sequence, a first promoter sequence, a first snRNA sequence, a termination sequence, a second promoter sequence, second snRNA sequence, a second termination sequence and a second AAV ITR sequence. In some aspects, an AAV vector can comprise a first AAV ITR sequence, a first promoter sequence, a first snRNA sequence, a termination sequence, a second promoter sequence, a second snRNA sequence, a second termination sequence, a third promoter sequence, a third snRNA sequence, a third termination sequence, and a second AAV ITR sequence. In some aspects, an AAV vector can comprise a first AAV ITR sequence, a first promoter sequence, a first snRNA sequence, a termination, a second promoter sequence, second snRNA sequence, a second termination sequence and a second AAV ITR sequence. In some aspects, an AAV vector can comprise a first AAV ITR sequence, a first promoter sequence, a first snRNA sequence, a termination, a second promoter sequence, a second snRNA sequence, a second termination sequence, a third promoter sequence, a third snRNA sequence, a third termination sequence, a fourth promoter sequence, a fourth snRNA sequence, a fourth termination sequence, and a second AAV ITR sequence.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV (rAAV) genomes of the invention comprise, consist essentially of, or consist of a nucleic acid molecule encoding at least one esnRNA and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAVrh10, AAV11 or AAV12. In some embodiments, the AAV serotype is AAVrh.74. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the capsid is a ubiquitination resistant capsid. In another embodiment, the ubiquitination capsid is an AAV2 capsid comprising tyrosine (Y) and serine(S) mutations. In another embodiment, the AAV2 capsid comprises Y, S and threonine (T) mutations. In another embodiment, the AAV2 capsid includes, without limitation, AAV2 capsid mutants such as T455V, T491V, T550V, T659V, Y444+500+730F, and Y444+500+730F+T491V. In some embodiments, the viral vector is replication incompetent. In some embodiments, the viral vector is isolated or recombinant (rAAV). In some embodiments, the viral vector is self-complementary (scAAV). In some embodiments, the viral vector is single-stranded (ssAAV).

In some embodiments, the snRNAs provided herein are comprised within a single-stranded AAV (ssAAV). In some embodiments, the snRNAs provided herein are comprised within a self-complementary AAV (scAAV). The single-stranded nature of the parvoviral genome requires the use of cellular mechanisms to provide a complementary-strand for gene expression. This cellular recruitment activity is considered a rate-limiting factor in the efficiency of transduction and gene expression in parvoviruses and parvoviral particles. The use of an scAAV versus an ssAAV remedies this well known issue by packaging both strands as a single duplex DNA molecule (or inverted repeat genome) that can fold into dsDNA as a result of a self-complementary viral genome sequence. In this regard, the requirement for DNA synthesis or base-pairing between multiple viral genomes is eliminated.

### AAV ITR Sequences

In some embodiments of the compositions and methods of the disclosure, an AAV inverted terminal repeat sequence can comprise any AAV ITR sequence known in the art. In some aspects, an AAV ITR sequence can comprise or consist of an AAV1 ITR sequence, an AAV2 ITR sequence, an AAV3 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAVrh10 ITR sequence, an AAV11 ITR sequence, an AAV12 ITR sequence, an AAV13 ITR sequence, or an AAVrh74 ITR sequence.

In some aspects the ITR sequence can comprise a modified AAV ITR sequence.

In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 119 or SEQ ID NO: 166.

In some aspects, a first AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 119 or SEQ ID NO: 166 and a second AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 119 or SEQ ID NO: 166. In some aspects the first AAV ITR sequence is positioned at the 5′ of an AAV vector. In some aspects the second AAV ITR sequence is positioned at the 3′ of an AAV vector.

In some embodiments of the compositions and methods of the disclosure, the viral vector comprises a sequence isolated or derived from an adeno-associated virus (AAV). In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

### Lentiviral Vectors

In some embodiments, the vector is a lentiviral vector. In some embodiments, the lentiviral vector can encode a range of total polynucleotides from 8 kb to 10 kb. In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVSM) vector, a modified sooty mangabey simian immunodeficiency virus (SIVSM) vector, a African green monkey simian immunodeficiency virus (SIVAGM) vector, a modified African green monkey simian immunodeficiency virus (SIVAGM) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV) and any combination or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

Lentiviral vectors are well-known in the art (see, e.g., Trono D. (2002) Lentiviral vectors, New York: Spring-Verlag Berlin Heidelberg and Durand et al. (2011) Viruses 3(2): 132-159 doi: 10.3390/v3020132).

A lentiviral vector described herein may comprise, consist essentially of, or consist of one or more nucleic acid molecules and one or more lentiviral LTRs. In some aspects, the nucleic acid molecule encodes an snRNA and/or an esnRNA of the disclosure. Such lentiviral vectors can be replicated and packaged into infectious viral particles when present in a host cell that provides the functionality of rep and cap gene products, for example, by transfection of the host cell. In some aspects, lentiviral vectors contain a promoter, at least one nucleic acid that may encode at least one protein or RNA, and/or an enhancer and/or a terminator within the flanking LTRs that is packaged into the infectious lentiviral particle. The encapsidated nucleic acid portion may be referred to as the lentiviral vector genome. Plasmids containing lentiviral vectors may also contain elements for manufacturing purposes, e.g., antibiotic resistance genes, origin of replication sequences etc., but these are not encapsidated and thus do not form part of the lentiviral particle.

In some aspects, a lentiviral vector can comprise at least one nucleic acid encoding an snRNA and/or an esnRNA of the disclosure. In some aspects, a lentiviral vector can comprise at least one regulatory sequence. In some aspects, a lentiviral vector can comprise at least one lentiviral long terminal repeat (LTR) sequence. In some aspects, a lentiviral vector can comprise a first LTR sequence and a second LTR sequence. In some aspects, a lentiviral vector can comprise at least one promoter sequence. In some aspects, a lentiviral vector can comprise at least one enhancer sequence. In some aspects, a lentiviral vector can comprise at least one terminator sequence. In some aspects, a lentiviral vector can comprise at least one poly A sequence. In some aspects, a lentiviral vector can comprise at least one linker sequence. In some aspects, a lentiviral vector can comprise at least one buffer sequence. In some aspects, a lentiviral vector of the disclosure can comprise at least one nuclear localization signal, or nuclear export signal and/or both.

In some aspects, a lentiviral vector can comprise a first lentiviral LTR sequence, a promoter sequence, an snRNA and/or an esnRNA sequence, a terminator sequence and a second lentiviral LTR sequence. In some aspects, a lentiviral vector can comprise, in the 5′ to 3′ direction, a first lentiviral LTR sequence, a promoter sequence, an snRNA sequence, a terminator sequence, and a second lentiviral LTR sequence. In some aspects, a lentiviral vector can comprise, in the 5′ to 3′ direction, a first lentiviral LTR sequence, a promoter sequence, an esnRNA sequence, a terminator sequence, and a second lentiviral LTR sequence.

In some aspects, a lentiviral vector can comprise a first lentiviral LTR sequence, a first promoter sequence, a first snRNA sequence, a termination sequence, a second promoter sequence, second snRNA sequence, a second termination sequence and a second lentiviral LTR sequence. In some aspects, a lentiviral vector can comprise a first lentiviral LTR sequence, a first promoter sequence, a first snRNA sequence, a termination sequence, a second promoter sequence, a second snRNA sequence, a second termination sequence, a third promoter sequence, a third snRNA sequence, a third termination sequence, and a second lentiviral LTR sequence. In some aspects, a lentiviral vector can comprise a first lentiviral LTR sequence, a first promoter sequence, a first snRNA sequence, a termination, a second promoter sequence, a second snRNA sequence, a second termination sequence, a third promoter sequence, a third snRNA sequence, a third termination sequence, a fourth promoter sequence, a fourth snRNA sequence, a fourth termination sequence, and a second lentiviral LTR sequence.

### Lentiviral LTR Sequences

In some embodiments of the compositions and methods of the disclosure, a lentiviral long terminal repeat sequence can comprise any lentiviral LTR sequence known in the art. In some aspects, a lentiviral LTR sequence can comprise or consist of a human immunodeficiency virus (HIV) 1 LTR sequence, a modified human immunodeficiency virus (HIV) 1 LTR sequence, a human immunodeficiency virus (HIV) 2 LTR sequence, a modified human immunodeficiency virus (HIV) 2 LTR sequence, a sooty mangabey simian immunodeficiency virus (SIVSM) LTR sequence, a modified sooty mangabey simian immunodeficiency virus (SIVSM) LTR sequence, a African green monkey simian immunodeficiency virus (SIVAGM) LTR sequence, a modified African green monkey simian immunodeficiency virus (SIVAGM) LTR sequence, an equine infectious anemia virus (EIAV) LTR sequence, a modified equine infectious anemia virus (EIAV) LTR sequence, a feline immunodeficiency virus (FIV) LTR sequence, a modified feline immunodeficiency virus (FIV) LTR sequence, a Visna/maedi virus (VNV/VMV) LTR sequence, a modified Visna/maedi virus (VNV/VMV) LTR sequence, a caprine arthritis-encephalitis virus (CAEV) LTR sequence, a modified caprine arthritis-encephalitis virus (CAEV) LTR sequence, a bovine immunodeficiency virus (BIV) LTR sequence, or a modified bovine immunodeficiency virus (BIV) LTR sequence.

In some aspects the LTR sequence can comprise a modified lentiviral LTR sequence.

In some aspects, a lentiviral LTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 170 or SEQ ID NO: 171.

In some embodiments, a lentiviral vector provided herein comprises a first and a second lentiviral LTR sequence. In some aspects, a first lentiviral LTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 170 or SEQ ID NO: 171 and a second lentiviral LTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 170 or SEQ ID NO: 171. In some aspects the first lentiviral LTR sequence is positioned at the 5′ of an lentiviral vector. In some aspects the second lentiviral LTR sequence is positioned at the 3′ of an lentiviral vector.

In some aspects, a first lentiviral LTR sequence comprises the sequence set forth in SEQ ID NO: 173 or SEQ ID NO: 174. In some aspects, a second lentiviral LTR sequence comprises the sequence set forth in SEQ ID NO: 173 or SEQ ID NO: 174. In some embodiments, a lentiviral vector provided herein comprises a first lentiviral LTR sequence comprising the sequence set forth in SEQ ID NO: 173 and a second lentiviral LTR sequence comprising the sequence set forth in SEQ ID NO: 174. In some aspects, the first lentiviral LTR sequence is positioned at the 5′ of an lentiviral vector. In some aspects, the second lentiviral LTR sequence is positioned at the 3′ of an lentiviral vector.

In some embodiments of the compositions and methods of the disclosure, the viral vector comprises a sequence isolated or derived from lentivirus.

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

snRNA Vector Constructs

Also provided herein are vector constructs targeting DMD comprising the snRNA constructs described herein.

An exemplary AAV vector of the disclosure is A05014 targeting DMD exon 53. The elements of A05014 are set forth in Table 8. In some aspects a nucleic acid sequence encoding AAV vector A05014 encoding DMD exon 53 snRNA sequences comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 133.

TABLE 8
A05014 Nucleotide sequences of plasmid
elements in order 5′ to 3′

| Plasmid | |
|---|---|
| Element | Nucleotide Sequences |
| 5′ITR | ctgcgcgctcgctcgctcactgaggccgcccgggcaaagc |
| | ccgggcgtcgggcgacctttggtcgcccggcctcagtgag |
| | cgagcgagcgcgcagagagggagtggggtt |
| | (SEQ ID NO: 119) |
| Mouse U7 | taacaacataggagctgtgattggctgttttcagccaatc |
| Promoter | agcactgActcatttgcatagcctttacaagcggtcacaa |
| | actcaagaaacgagcggttttaatagtcttttagaatatt |
| | gtttatcgaaccgaataaggaactgtgctttgtgattcac |
| | atatcagtggaggggtgtggaaatggcaccttgatctcac |
| | cctcatcgaaagtggagttgatgtcctTccctggctcgct |
| | acagacgcacttccgc (SEQ ID NO: 151) |
| extra nt | aa |
| ISD | ggagt |
| Fusion | CATTCAACTGTTGCCTCCGGTTCTGAAGGTGTCATCCCAC |
| spacer | TGATTCTGAATTCTTTCAACTA |
| z13/z16 | (SEQ ID NO: 121) |
| SMOPT | AATTTTTGGAGca (SEQ ID NO: 161) |
| Stem loop | ggttttctgacctccgtcggaaaacccct |
| | (SEQ ID NO: 146) |
| Mouse U7 | cccaatttcactggtctacaatgaaagcaaaacagttctc |
| terminator | ttccccgctccccggtgtgtgagaggggctttgatcct |
| | tctctggtttcctaggaaacgcgtatgtg |
| | (SEQ ID NO: 156) |
| Mouse U1 | TTGTTCCTCTTAGTGTTAATTCACACTAAAGACTGTGCA |
| Promoter | TCCGACTCCTACATTTATGAAAGTAAATGCCTGTTGTTA |
| | GAACAAAAAAGGCTACAGAACAAAAAACAAAGCGAAATA |
| | CCATCTGCTTTAGGTTCAGTGTGGTATTTTCCCGCTGAC |
| | AGGGAGGCGGGTTTTTGGGTACAGGAAACGAGTCACTAT |
| | GGAGGCGGTACTATGTAGATGAGAATTCAGGTGCAAACT |
| | GGGAAAAGCAACTGCTTCCAAATATTTGTGATTTTTACA |
| | GTGTAGTTTTGGAAAAACTCTTAGCCTACCAATTCTTCT |
| | AAGTGTTTTAAAATGTGGGAGCCAGTACACATGAAGTTA |
| | TAGAGTGTTTTAATGAGGCTTAAATATTTACCGTAACTA |
| | TGAAATGCTACGCATATCATGCTGTTCAGGCTCCGTGGC |
| | CACGCAACTC |
| | (SEQ ID NO: 47) |
| ISD | ggagt |
| Fusion | CATTCAACTGTTGCCTCCGGTTCTGAAGGTGTCATCCCA |
| spacer | CTGATTCTGAATTCTTTCAACTA |
| z13/z16 | (SEQ ID NO: 121) |
| SMOPT | AATTTTTGGAGca |
| | (SEQ ID NO: 161) |
| Stem loop | ggttttctgacctccgtcggaaaacccct |
| | (SEQ ID NO: 146) |
| Mouse U1 | GTTTACTTGGTTTTAAAAATAGCTTGCACTAGCGATACG |
| terminator | GAATATGGTTATTAGGTTTGTTAGGCATCATGTCGTGTC |
| | TTACTATAGAAAAATAACGTAGTGTTCATTTTAGCCTGC |
| | CTGTATGTGTTAATTTGTCCTTATTGCGCATTGTTCTTG |
| | TTAAGTCTTCTGTAAGGAGTTGCGGGTTTCAAACTGTCA |
| | GTCTGAGAGCA |
| | (SEQ ID NO: 57) |
| 3′ITR | aggaacccctagtgatggagttggccactccctctctgc |
| | gcgctcgctcgctcactgaggccgggcgaccaaaggtcg |
| | cccgacgcccgggctttgcccgggcggcctcagtgagcg |
| | agcgagcgcgcagctgcctgcagg |
| | (SEQ ID NO: 127) |

A05014 Nucleotide Sequence (whole transgene from ITR to ITR):
ctgcgcgctcgctcgctcactgaggccgcccgggcaaagcccgggcgtcgggcgacctttggtcgcccggcctcagt gagcgagcgagcgcgcagagagggagtggggttCTTCGAAACACCGGTtaacaacataggagctgtgattggctgttt tcagccaatcagcactgActcatttgcatagcctttacaagcggtcacaaactcaagaaacgagcggttttaatagtcttttagaatattgt ttatcgaaccgaataaggaactgtgctttgtgattcacatatcagtggaggggtgtggaaatggcaccttgatctcaccctcatcgaaagt ggagttgatgtcctTccctggctcgctacagacgcacttccgcaaggagtCATTCAACTGTTGCCTCCGGTTCT GAAGGTGTCATCCCACTGATTCTGAATTCTTTCAACTAaATTTTTGGAGcaggttttctga cctccgtcggaaaacccctcccaatttcactggtctacaatgaaagcaaaacagttctcttccccgctccccggtgtgtgagaggggctt tgatccttctctggtttcctaggaaacgcgtatgtgTTGTTCCTCTTAGTGTTAATTCACACTAAAGACT GTGCATCCGACTCCTACATTTATGAAAGTAAATGCCTGTTGTTAGAACAAAAAAG GCTACAGAACAAAAAACAAAGCGAAATACCATCTGCTTTAGGTTCAGTGTGGTA TTTTCCCGCTGACAGGGAGGCGGGTTTTTGGGTACAGGAAACGAGTCACTATGG AGGCGGTACTATGTAGATGAGAATTCAGGTGCAAACTGGGAAAAGCAACTGCTT CCAAATATTTGTGATTTTTACAGTGTAGTTTTGGAAAAACTCTTAGCCTACCAATT CTTCTAAGTGTTTTAAAATGTGGGAGCCAGTACACATGAAGTTATAGAGTGTTTT AATGAGGCTTAAATATTTACCGTAACTATGAAATGCTACGCATATCATGCTGTTC AGGCTCCGTGGCCACGCAACTCggagtCATTCAACTGTTGCCTCCGGTTCTGAAGGT GTCATCCCACTGATTCTGAATTCTTTCAACTAaATTTTTGGAGcaggttttctgacctccgtcgg aaaacccctGTTTACTTGGTTTTAAAAATAGCTTGCACTAGCGATACGGAATATGGTT ATTAGGTTTGTTAGGCATCATGTCGTGTCTTACTATAGAAAAATAACGTAGTGTT CATTTTAGCCTGCCTGTATGTGTTAATTTGTCCTTATTGCGCATTGTTCTTGTTAA GTCTTCTGTAAGGAGTTGCGGGTTTCAAACTGTCAGTCTGAGAGCAGAATTCGAT ATCTAGATCTCGAGGTAACCACGTGCGGACCCAACGGCCGCaggaacccctagtgatggagtt ggccactccctctctgegcgctcgctcgctcactgaggccgggcgaccaaaggtcgcccgacgcccgggctttgcccgggcggcc tcagtgagcgagcgagcgcgcagctgcctgcagg (SEQ ID NO: 133). An exemplary AAV vector of the disclosure is A05211 targeting DMD exon 53. The elements of A05211 are set forth in Table 9. In some aspects a nucleic acid sequence encoding AAV vector A05211 encoding DMD exon 53 snRNA sequences comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 134.

TABLE 9
A05211: Nucleotide sequences of plasmid
elements in order 5′ to 3′

| Plasmid | |
|---|---|
| Element | Nucleotide Sequences |
| 5′ITR | ctgcgcgctcgctcgctcactgaggccgcccgggcaaagc |
| | ccgggcgtcgggcgacctttggtcgcccggcctcagtgag |
| | cgagcgagcgcgcagagagggagtggggtt |
| | (SEQ ID NO: 119) |
| Mouse U7 | taacaacataggagctgtgattggctgttttcagccaatc |
| Promoter | agcactgActcatttgcatagcctttacaagcggtcacaa |
| | actcaagaaacgagcggttttaatagtcttttagaatatt |
| | gtttatcgaaccgaataaggaactgtgctttgtgattcac |
| | atatcagtggaggggtgtggaaatggcaccttgatctcac |
| | cctcatcgaaagtggagttgatgtcctTccctggctcgct |
| | acagacgcacttccgc (SEQ ID NO: 151) |
| extra nt | aa |
| Fusion | CATTCAACTGTTGCCTCCGGTTCTGAAGGTGTCATCCCAC |
| spacer | TGATTCTGAATTCTTTCAACTA (SEQ ID NO: 121) |
| z13/z16 | |
| SMOPT | AATTTTTGGAGca (SEQ ID NO: 161) |
| Stem loop | ggttttctgacttcggtcggaaaacccct |
| | (SEQ ID NO: 145) |
| Mouse U7 | cccaatttcactggtctacaatgaaagcaaaacagttctc |
| terminator | ttccccgctccccggtgtgtgagaggggctttgatccttc |
| | tctggtttcctaggaaacgcgtatgtg (SEQ ID NO: |
| | 156) |
| Mouse U1 | TTGTTCCTCTTAGTGTTAATTCACACTAAAGACTGTGCAT |
| Promoter | CCGACTCCTACATTTATGAAAGTAAATGCCTGTTGTTAGA |
| | ACAAAAAAGGCTACAGAACAAAAAACAAAGCGAAATACCA |
| | TCTGCTTTAGGTTCAGTGTGGTATTTTCCCGCTGACAGGG |
| | AGGCGGGTTTTTGGGTACAGGAAACGAGTCACTATGGAGG |
| | CGGTACTATGTAGATGAGAATTCAGGTGCAAACTGGGAAA |
| | AGCAACTGCTTCCAAATATTTGTGATTTTTACAGTGTAGT |
| | TTTGGAAAAACTCTTAGCCTACCAATTCTTCTAAGTGTTT |
| | TAAAATGTGGGAGCCAGTACACATGAAGTTATAGAGTGTT |
| | TTAATGAGGCTTAAATATTTACCGTAACTATGAAATGCTA |
| | CGCATATCATGCTGTTCAGGCTCCGTGGCCACGCAACTC |
| | (SEQ ID NO: 47) |
| Fusion | CATTCAACTGTTGCCTCCGGTTCTGAAGGTGTCATCCCAC |
| spacer | TGATTCTGAATTCTTTCAACTA (SEQ ID NO: 121) |
| z13/z16 | |
| SMOPT | AATTTTTGGAGca (SEQ ID NO: 161) |
| Stem loop | ggttttctgacttcggtcggaaaacccct |
| | (SEQ ID NO: 145) |
| Mouse U1 | GTTTACTTGGTTTTAAAAATAGCTTGCACTAGCGATACGG |
| terminator | AATATGGTTATTAGGTTTGTTAGGCATCATGTCGTGTCTT |
| | ACTATAGAAAAATAACGTAGTGTTCATTTTAGCCTGCCTG |
| | TATGTGTTAATTTGTCCTTATTGCGCATTGTTCTTGTTAA |
| | GTCTTCTGTAAGGAGTTGCGGGTTTCAAACTGTCAGTCTG |
| | AGAGCA (SEQ ID NO: 57) |
| 3′ITR | aggaacccctagtgatggagttggccactccctctctgcg |
| | cgctcgctcgctcactgaggccgggcgaccaaaggtcgcc |
| | cgacgcccgggctttgcccgggcggcctcagtgagcgagc |
| | gagcgcgcagctgcctgcagg |
| | (SEQ ID NO: 127) |

A05211 Nucleotide Sequence (whole transgene from ITR to ITR):
(SEQ ID NO: 134)
ctgcgcgctcgctcgctcactgaggccgcccgggcaaagcccgggcgtcgggcgacctttggtcgcccggcctcagt
gagcgagcgagcgcgcagagagggagtggggttCTTCGAAACACCGGTtaacaacataggagctgtgattggctgttt
tcagccaatcagcactgActcatttgcatagcctttacaagcggtcacaaactcaagaaacgagcggttttaatagtcttttagaatatt
gtttatcgaaccgaataaggaactgtgctttgtgattcacatatcagtggaggggtgtggaaatggcaccttgatctcaccctcatcgaa
agtggagttgatgtcctTccctggctcgctacagacgcacttccgcaaCATTCAACTGTTGCCTCCGGTTCTGAA
GGTGTCATCCCACTGATTCTGAATTCTTTCAACTAaATTTTTGGAGcaggttttctgacttcgg
tcggaaaacccctcccaatttcactggtctacaatgaaagcaaaacagttctcttccccgctccccggtgtgtgagaggggctttgatcc
ttctctggtttcctaggaaacgcgtatgtgTTGTTCCTCTTAGTGTTAATTCACACTAAAGACTGTGC
ATCCGACTCCTACATTTATGAAAGTAAATGCCTGTTGTTAGAACAAAAAAGGCTA
CAGAACAAAAAACAAAGCGAAATACCATCTGCTTTAGGTTCAGTGTGGTATTTTC
CCGCTGACAGGGAGGCGGGTTTTTGGGTACAGGAAACGAGTCACTATGGAGGCG
GTACTATGTAGATGAGAATTCAGGTGCAAACTGGGAAAAGCAACTGCTTCCAAA
TATTTGTGATTTTTACAGTGTAGTTTTGGAAAAACTCTTAGCCTACCAATTCTTCT
AAGTGTTTTAAAATGTGGGAGCCAGTACACATGAAGTTATAGAGTGTTTTAATGA
GGCTTAAATATTTACCGTAACTATGAAATGCTACGCATATCATGCTGTTCAGGCT
CCGTGGCCACGCAACTCCATTCAACTGTTGCCTCCGGTTCTGAAGGTGTCATCCC
ACTGATTCTGAATTCTTTCAACTAaATTTTTGGAGcaggttttctgacttcggtcggaaaacccctGT
TTACTTGGTTTTAAAAATAGCTTGCACTAGCGATACGGAATATGGTTATTAGGTT
TGTTAGGCATCATGTCGTGTCTTACTATAGAAAAATAACGTAGTGTTCATTTTAG
CCTGCCTGTATGTGTTAATTTGTCCTTATTGCGCATTGTTCTTGTTAAGTCTTCTGT
AAGGAGTTGCGGGTTTCAAACTGTCAGTCTGAGAGCAGAATTCGATATCTAGATC
TCGAGGTAACCACGTGCGGACCCAACGGCCGCaggaacccctagtgatggagttggccactccctctct
gcgcgctcgctcgctcactgaggccgggcgaccaaaggtcgcccgacgcccgggctttgcccgggcggcctcagtgagcgagcg
agcgcgcagctgcctgcagg

An exemplary AAV vector of the disclosure is A05374 targeting DMD exon 44. The elements of A05374 are set forth in Table 10. In some aspects a nucleic acid sequence encoding AAV vector A05374 encoding DMD exon 44 targeting snRNA sequences comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 135.

TABLE 10
A05374: Nucleotide sequences of plasmid elements in order 5′ to 3′

| Plasmid | |
|---|---|
| Element | Nucleotide Sequences |
| 5′ITR | Ctgcgcgctcgctcgctcactgaggccgcccgggcaaagcccgggcgtcgggcgacctttggtcgcccggc |
| | ctcagtgagcgagcgagcgcgcagagagggagtggggtt (SEQ ID NO: 119) |
| Mouse | taacaacataggagctgtgattggctgttttcagccaatcagcactgActcatttgcatagcctttacaagcggtca |
| U7 | caaactcaagaaacgagcggttttaatagtcttttagaatattgtttatcgaaccgaataaggaactgtgctttgtgat |
| Promoter | tcacatatcagtggaggggtgtggaaatggcaccttgatctcaccctcatcgaaagtggagttgatgtcctTccct |
| | ggctcgctacagacgcacttccgc (SEQ ID NO: 151) |
| extra nt | aa |
| ISD | ggagt |
| Fusion | TTGTGTCTTTCTGAGAAACTGTTCAGCTTCTGTTAGCCACTGACATT |
| spacer | TCTCAACAGATCTGTCAAATCGCctgc (SEQ ID NO: 88) |
| z9/z4 | |
| SMOPT | AATTTTTGGAGca (SEQ ID NO: 161) |
| Stem | Ggttttctgacctccgtcggaaaacccct (SEQ ID NO: 146) |
| loop | |
| Mouse | Cccaatttcactggtctacaatgaaagcaaaacagttctcttccccgctccccggtgtgtgagaggggctttgatc |
| U7 | cttctctggtttcctaggaaacgcgtatgtg (SEQ ID NO: 156) |
| terminator | |
| Mouse | TTGTTCCTCTTAGTGTTAATTCACACTAAAGACTGTGCATCCGACTC |
| U1 | CTACATTTATGAAAGTAAATGCCTGTTGTTAGAACAAAAAAGGCTA |
| Promoter | CAGAACAAAAAACAAAGCGAAATACCATCTGCTTTAGGTTCAGTGT |
| | GGTATTTTCCCGCTGACAGGGAGGCGGGTTTTTGGGTACAGGAAAC |
| | GAGTCACTATGGAGGCGGTACTATGTAGATGAGAATTCAGGTGCAA |
| | ACTGGGAAAAGCAACTGCTTCCAAATATTTGTGATTTTTACAGTGT |
| | AGTTTTGGAAAAACTCTTAGCCTACCAATTCTTCTAAGTGTTTTAAA |
| | ATGTGGGAGCCAGTACACATGAAGTTATAGAGTGTTTTAATGAGGC |
| | TTAAATATTTACCGTAACTATGAAATGCTACGCATATCATGCTGTTC |
| | AGGCTCCGTGGCCACGCAACTC (SEQ ID NO: 47) |
| ISD | ggagt |
| Fusion | TTGTGTCTTTCTGAGAAACTGTTCAGCTTCTGTTAGCCACTGACATT |
| spacer | TCTCAACAGATCTGTCAAATCGCctgc (SEQ ID NO: 88) |
| z9/z4 | |
| SMOPT | AATTTTTGGAGca (SEQ ID NO: 161) |
| Stem | Ggttttctgacctccgtcggaaaacccct (SEQ ID NO: 146) |
| loop | |
| Mouse | GTTTACTTGGTTTTAAAAATAGCTTGCACTAGCGATACGGAATATG |
| U1 | GTTATTAGGTTTGTTAGGCATCATGTCGTGTCTTACTATAGAAAAAT |
| terminator | AACGTAGTGTTCATTTTAGCCTGCCTGTATGTGTTAATTTGTCCTTA |
| | TTGCGCATTGTTCTTGTTAAGTCTTCTGTAAGGAGTTGCGGGTTTCA |
| | AACTGTCAGTCTGAGAGCA (SEQ ID NO: 57) |
| 3′ITR | Aggaacccctagtgatggagttggccactccctctctgcgcgctcgctcgctcactgaggccgggcgaccaaa |
| | ggtcgcccgacgcccgggctttgcccgggcggcctcagtgagcgagcgagcgcgcagctgcctgcagg |
| | (SEQ ID NO: 127) |

A05374 ITR to ITR Sequence
(SEQ ID NO: 135)
ctgcgcgctcgctcgctcactgaggccgcccgggcaaagcccgggcgtcgggcgacctttggtcgcccggcctcagtgagcgagc
gagcgcgcagagagggagtggggttCTTCGAAACACCGGTtaacaacataggagctgtgattggctgttttcagccaat
cagcactgActcatttgcatagcctttacaagcggtcacaaactcaagaaacgagcggttttaatagtcttttagaatattgtttatcga
accgaataaggaactgtgctttgtgattcacatatcagtggaggggtgtggaaatggcaccttgatctcaccctcatcgaaagtggagtt
gatgtcctTccctggctcgctacagacgcacttccgcaaggagtTTGTGTCTTTCTGAGAAACTGTTCAGCTT
CTGTTAGCCACTGACATTTCTCAACAGATCTGTCAAATCGCctgcaATTTTTGGAGca
ggttttctgacctccgtcggaaaacccctcccaatttcactggtctacaatgaaagcaaaacagttctcttccccgctccccggtgtgtga
gaggggctttgatccttctctggtttcctaggaaacgcgtatgtgTTGTTCCTCTTAGTGTTAATTCACACTAA
AGACTGTGCATCCGACTCCTACATTTATGAAAGTAAATGCCTGTTGTTAGAACAA
AAAAGGCTACAGAACAAAAAACAAAGCGAAATACCATCTGCTTTAGGTTCAGTG
TGGTATTTTCCCGCTGACAGGGAGGCGGGTTTTTGGGTACAGGAAACGAGTCACT
ATGGAGGCGGTACTATGTAGATGAGAATTCAGGTGCAAACTGGGAAAAGCAACT
GCTTCCAAATATTTGTGATTTTTACAGTGTAGTTTTGGAAAAACTCTTAGCCTACC
AATTCTTCTAAGTGTTTTAAAATGTGGGAGCCAGTACACATGAAGTTATAGAGTG
TTTTAATGAGGCTTAAATATTTACCGTAACTATGAAATGCTACGCATATCATGCT
GTTCAGGCTCCGTGGCCACGCAACTCggagtTTGTGTCTTTCTGAGAAACTGTTCAG
CTTCTGTTAGCCACTGACATTTCTCAACAGATCTGTCAAATCGCctgcaATTTTTGGA
GcaggttttctgacctccgtcggaaaacccctGTTTACTTGGTTTTAAAAATAGCTTGCACTAGCGA
TACGGAATATGGTTATTAGGTTTGTTAGGCATCATGTCGTGTCTTACTATAGAAA
AATAACGTAGTGTTCATTTTAGCCTGCCTGTATGTGTTAATTTGTCCTTATTGCGC
ATTGTTCTTGTTAAGTCTTCTGTAAGGAGTTGCGGGTTTCAAACTGTCAGTCTGAG
AGCAGAATTCGATATCTAGATCTCGAGGTAACCACGTGCGGACCCAACGGCCGCa
ggaacccctagtgatggagttggccactccctctctgcgcgctcgctcgctcactgaggccgggcgaccaaaggtcgcccgacgcc
cgggctttgcccgggcggcctcagtgagcgagcgagcgcgcagctgcctgcagg.

An exemplary AAV vector of the disclosure is A05375 targeting DMD exon 44. The elements of A05375 are set forth in Table 11. In some aspects a nucleic acid sequence encoding AAV vector A05375 encoding DMD exon 44 targeting snRNA sequences comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 136.

TABLE 11
Nucleotide sequences of plasmid elements in order 5′ to 3′

| Plasmid | |
|---|---|
| Element | Nucleotide Sequences |
| 5′ITR | Ctgcgcgctcgctcgctcactgaggccgcccgggcaaagcccgggcgtcgggcgacctttggtcgcccggc |
| | ctcagtgagcgagcgagcgcgcagagagggagtggggtt (SEQ ID NO: 119) |
| Mouse | taacaacataggagctgtgattggctgttttcagccaatcagcactgActcatttgcatagcctttacaagcggtca |
| U7 | caaactcaagaaacgagcggttttaatagtcttttagaatattgtttatcgaaccgaataaggaactgtgctttgtgat |
| Promoter | tcacatatcagtggaggggtgtggaaatggcaccttgatctcaccctcatcgaaagtggagttgatgtcctTccct |
| | ggctcgctacagacgcacttccgc (SEQ ID NO: 151) |
| extra nt | aa |
| ISD | ggagt |
| Fusion | GTGTCTTTCTGAGAAACTGTTCAGCTTCTGTTAGCCCATTTCTCAAC |
| spacer | AGATCTGTCAAATCGCctgc (SEQ ID NO: 90) |
| z11/z4 | |
| SMOPT | AATTTTTGGAGca (SEQ ID NO: 161) |
| Stem | Ggttttctgacctccgtcggaaaacccct (SEQ ID NO: 146) |
| loop | |
| Mouse | Cccaatttcactggtctacaatgaaagcaaaacagttctcttccccgctccccggtgtgtgagaggggctttgatc |
| U7 | cttctctggtttcctaggaaacgcgtatgtg (SEQ ID NO: 156) |
| terminator | |
| Mouse | TTGTTCCTCTTAGTGTTAATTCACACTAAAGACTGTGCATCCGACTC |
| U1 | CTACATTTATGAAAGTAAATGCCTGTTGTTAGAACAAAAAAGGCTA |
| Promoter | CAGAACAAAAAACAAAGCGAAATACCATCTGCTTTAGGTTCAGTGT |
| | GGTATTTTCCCGCTGACAGGGAGGCGGGTTTTTGGGTACAGGAAAC |
| | GAGTCACTATGGAGGCGGTACTATGTAGATGAGAATTCAGGTGCAA |
| | ACTGGGAAAAGCAACTGCTTCCAAATATTTGTGATTTTTACAGTGT |
| | AGTTTTGGAAAAACTCTTAGCCTACCAATTCTTCTAAGTGTTTTAAA |
| | ATGTGGGAGCCAGTACACATGAAGTTATAGAGTGTTTTAATGAGGC |
| | TTAAATATTTACCGTAACTATGAAATGCTACGCATATCATGCTGTTC |
| | AGGCTCCGTGGCCACGCAACTC (SEQ ID NO: 47) |
| ISD | ggagt |
| Fusion | GTGTCTTTCTGAGAAACTGTTCAGCTTCTGTTAGCCCATTTCTCAAC |
| spacer | AGATCTGTCAAATCGCctgc (SEQ ID NO: 90) |
| z11/z4 | |
| SMOPT | AATTTTTGGAGca (SEQ ID NO: 161) |
| Stem | Ggttttctgacctccgtcggaaaacccct (SEQ ID NO: 146) |
| loop | |
| Mouse | GTTTACTTGGTTTTAAAAATAGCTTGCACTAGCGATACGGAATATG |
| U1 | GTTATTAGGTTTGTTAGGCATCATGTCGTGTCTTACTATAGAAAAAT |
| terminator | AACGTAGTGTTCATTTTAGCCTGCCTGTATGTGTTAATTTGTCCTTA |
| | TTGCGCATTGTTCTTGTTAAGTCTTCTGTAAGGAGTTGCGGGTTTCA |
| | AACTGTCAGTCTGAGAGCA (SEQ ID NO: 57) |
| 3′ITR | Aggaacccctagtgatggagttggccactccctctctgcgcgctcgctcgctcactgaggccgggcgaccaaa |
| | ggtcgcccgacgcccgggctttgcccgggcggcctcagtgagcgagcgagcgcgcagctgcctgcagg |
| | (SEQ ID NO: 127) |

A05375 ITR to ITR Sequence
(SEQ ID NO: 136)
ctgcgcgctcgctcgctcactgaggccgcccgggcaaagcccgggcgtcgggcgacctttggtcgcccggcctcagtgagcgagc
gagcgcgcagagagggagtggggttCTTCGAAACACCGGTtaacaacataggagctgtgattggctgttttcagccaat
cagcactgActcatttgcatagcctttacaagcggtcacaaactcaagaaacgagcggttttaatagtcttttagaatattgtttatcgaa
ccgaataaggaactgtgctttgtgattcacatatcagtggaggggtgtggaaatggcaccttgatctcaccctcatcgaaagtggagttga
tgtcctTccctggctcgctacagacgcacttccgcaaggagtGTGTCTTTCTGAGAAACTGTTCAGCTTCT
GTTAGCCCATTTCTCAACAGATCTGTCAAATCGCctgcaATTTTTGGAGcaggttttctgacct
ccgtcggaaaacccctcccaatttcactggtctacaatgaaagcaaaacagttctcttccccgctccccggtgtgtgagaggggctttg
atccttctctggtttcctaggaaacgcgtatgtgTTGTTCCTCTTAGTGTTAATTCACACTAAAGACTGT
GCATCCGACTCCTACATTTATGAAAGTAAATGCCTGTTGTTAGAACAAAAAAGGC
TACAGAACAAAAAACAAAGCGAAATACCATCTGCTTTAGGTTCAGTGTGGTATTT
TCCCGCTGACAGGGAGGCGGGTTTTTGGGTACAGGAAACGAGTCACTATGGAGG
CGGTACTATGTAGATGAGAATTCAGGTGCAAACTGGGAAAAGCAACTGCTTCCA
AATATTTGTGATTTTTACAGTGTAGTTTTGGAAAAACTCTTAGCCTACCAATTCTT
CTAAGTGTTTTAAAATGTGGGAGCCAGTACACATGAAGTTATAGAGTGTTTTAAT
GAGGCTTAAATATTTACCGTAACTATGAAATGCTACGCATATCATGCTGTTCAGG
CTCCGTGGCCACGCAACTCggagtGTGTCTTTCTGAGAAACTGTTCAGCTTCTGTTA
GCCCATTTCTCAACAGATCTGTCAAATCGCctgcaATTTTTGGAGcaggttttctgacctccgtcg
gaaaacccctGTTTACTTGGTTTTAAAAATAGCTTGCACTAGCGATACGGAATATGGTT
ATTAGGTTTGTTAGGCATCATGTCGTGTCTTACTATAGAAAAATAACGTAGTGTT
CATTTTAGCCTGCCTGTATGTGTTAATTTGTCCTTATTGCGCATTGTTCTTGTTAA
GTCTTCTGTAAGGAGTTGCGGGTTTCAAACTGTCAGTCTGAGAGCAGAATTCGAT
ATCTAGATCTCGAGGTAACCACGTGCGGACCCAACGGCCGCaggaacccctagtgatggagtt
ggccactccctctctgcgcgctcgctcgctcactgaggccgggcgaccaaaggtcgcccgacgcccgggctttgcccgggggcc
tcagtgagcgagcgagcgcgcagctgcctgcagg

An exemplary AAV vector of the disclosure is A05190 targeting DMD exon 45. The elements of A05190 are set forth in Table 12. In some aspects a nucleic acid sequence encoding AAV vector A05190 encoding DMD exon 45 targeting snRNA sequences comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 137.

TABLE 12
A05190: Stuffer-scAAV-2x_mU7prom-Z45-8/10-
mU7term_mU1prom-Z45-8/10-mU1term; mouseloop with 5′ ISD and eSL; derivative of
A04376

| Plasmid | |
|---|---|
| Element | Nucleotide Sequences |
| 5′ITR | ctgcgcgctcgctcgctcactgaggccgcccgggcaaagcccgggcgtcgggcgacctttggtcgcccggcct |
| | cagtgagcgagcgagcgcgcagagagggagtggggtt (SEQ ID NO: 119) |
| Mouse U7 | taacaacataggagctgtgattggctgttttcagccaatcagcactgActcatttgcatagcctttacaagcggtcac |
| Promoter | aaactcaagaaacgagcggttttaatagtcttttagaatattgtttatcgaaccgaataaggaactgtgctttgtgat |
| | tcacatatcagtggaggggtgtggaaatggcaccttgatctcaccctcatcgaaagtggagttgatgtcctTccctgg |
| | ctcgctacagacgcacttccgc (Scq ID NO: 151) |
| extra nt | aa |
| ISD | ggagt |
| Fusion | TTTGCAGACCTCCTGCCACCGCAGATTCAGAGTTTGCCGCTGCCCAA |
| spacer | TGCCATCCTGGAG (SEQ ID NO: 99) |
| z8/z10 | |
| SMOPT | aATTTTTGGAGca (SEQ ID NO: 161) |
| Enhanced | ggttttctgacctccgtcggaaaacccct (SEQ ID NO: 146) |
| Stem loop | |
| Mouse U7 | cccaatttcactggtctacaatgaaagcaaaacagttctcttccccgctccccggtgtgtgagaggggctttgatcct |
| terminator | tctctggtttcctaggaaacgcgtatgtg (SEQ ID NO: 156) |
| Mouse U1 | TTGTTCCTCTTAGTGTTAATTCACACTAAAGACTGTGCATCCGACTCC |
| Promoter | TACATTTATGAAAGTAAATGCCTGTTGTTAGAACAAAAAAGGCTAC |
| | AGAACAAAAAACAAAGCGAAATACCATCTGCTTTAGGTTCAGTGTG |
| | GTATTTTCCCGCTGACAGGGAGGCGGGTTTTTGGGTACAGGAAACG |
| | AGTCACTATGGAGGCGGTACTATGTAGATGAGAATTCAGGTGCAAA |
| | CTGGGAAAAGCAACTGCTTCCAAATATTTGTGATTTTTACAGTGTAG |
| | TTTTGGAAAAACTCTTAGCCTACCAATTCTTCTAAGTGTTTTAAAATG |
| | TGGGAGCCAGTACACATGAAGTTATAGAGTGTTTTAATGAGGCTTAA |
| | ATATTTACCGTAACTATGAAATGCTACGCATATCATGCTGTTCAGGC |
| | TCCGTGGCCACGCAACTC (SEQ ID NO: 47) |
| ISD | ggagt |
| Fusion | TTTGCAGACCTCCTGCCACCGCAGATTCAGAGTTTGCCGCTGCCCAA |
| spacer | TGCCATCCTGGAG (SEQ ID NO: 99) |
| z8/z10 | |
| SMOPT | aATTTTTGGAGca (SEQ ID NO: 161) |
| Enhanced | ggttttctgacctccgtcggaaaacccct (SEQ ID NO: 146) |
| Stem loop | |
| Mouse U1 | GTTTACTTGGTTTTAAAAATAGCTTGCACTAGCGATACGGAATATGG |
| terminator | TTATTAGGTTTGTTAGGCATCATGTCGTGTCTTACTATAGAAAAATA |
| | ACGTAGTGTTCATTTTAGCCTGCCTGTATGTGTTAATTTGTCCTTATT |
| | GCGCATTGTTCTTGTTAAGTCTTCTGTAAGGAGTTGCGGGTTTCAAA |
| | CTGTCAGTCTGAGAGCA (SEQ ID NO: 57) |
| 3′ITR | aggaacccctagtgatggagttggccactccctctctgcgcgctcgctcgctcactgaggccgggcgaccaaag |
| | gtcgcccgacgcccgggctttgcccgggcggcctcagtgagcgagcgagcgcgcagctgcctgcagg |
| | (SEQ ID NO: 127) |

A05190 Nucleotide Sequence (whole transgene from ITR to ITR):
(SEQ ID NO: 137)
ctgcgcgctcgctcgctcactgaggccgcccgggcaaagcccgggcgtcgggcgacctttggtcgcccggcctcagt
gagcgagcgagcgcgcagagagggagtggggttCTTCGAAACACCGGTtaacaacataggagctgtgattggctgttt
tcagccaatcagcactgActcatttgcatagcctttacaagcggtcacaaactcaagaaacgagcggttttaatagtcttttagaatattg
tttatcgaaccgaataaggaactgtgctttgtgattcacatatcagtggaggggtgtggaaatggcaccttgatctcaccctcatcgaaag
tggagttgatgtcctTccctggctcgctacagacgcacttccgcaaggagtTTTGCAGACCTCCTGCCACCGCAG
ATTCAGAGTTTGCCGCTGCCCAATGCCATCCTGGAGaATTTTTGGAGcaggttttctgacct
ccgtcggaaaacccctcccaatttcactggtctacaatgaaagcaaaacagttctcttccccgctccccggtgtgtgagaggggctttg
atccttctctggtttcctaggaaacgcgtatgtgTTGTTCCTCTTAGTGTTAATTCACACTAAAGACTGT
GCATCCGACTCCTACATTTATGAAAGTAAATGCCTGTTGTTAGAACAAAAAAGGC
TACAGAACAAAAAACAAAGCGAAATACCATCTGCTTTAGGTTCAGTGTGGTATTT
TCCCGCTGACAGGGAGGCGGGTTTTTGGGTACAGGAAACGAGTCACTATGGAGG
CGGTACTATGTAGATGAGAATTCAGGTGCAAACTGGGAAAAGCAACTGCTTCCA
AATATTTGTGATTTTTACAGTGTAGTTTTGGAAAAACTCTTAGCCTACCAATTCTT
CTAAGTGTTTTAAAATGTGGGAGCCAGTACACATGAAGTTATAGAGTGTTTTAAT
GAGGCTTAAATATTTACCGTAACTATGAAATGCTACGCATATCATGCTGTTCAGG
CTCCGTGGCCACGCAACTCggagtTTTGCAGACCTCCTGCCACCGCAGATTCAGAGT
TTGCCGCTGCCCAATGCCATCCTGGAGaATTTTTGGAGcaggttttctgacctccgtcggaaaaccc
ctGTTTACTTGGTTTTAAAAATAGCTTGCACTAGCGATACGGAATATGGTTATTAG
GTTTGTTAGGCATCATGTCGTGTCTTACTATAGAAAAATAACGTAGTGTTCATTTT
AGCCTGCCTGTATGTGTTAATTTGTCCTTATTGCGCATTGTTCTTGTTAAGTCTTCT
GTAAGGAGTTGCGGGTTTCAAACTGTCAGTCTGAGAGCAGAATTCGATATCTAG
ATCTCGAGGTAACCACGTGCGGACCCAACGGCCGCaggaacccctagtgatggagttggccactcc
ctctctgcgcgctcgctcgctcactgaggccgggcgaccaaaggtcgcccgacgcccgggctttgcccgggggcctcagtgagc
gagcgagcgcgcagctgcctgcagg

An exemplary AAV vector of the disclosure is A05189 targeting DMD exon 45. The elements of A05189 are set forth in Table 13. In some aspects a nucleic acid sequence encoding AAV vector A05189 encoding DMD exon 45 targeting snRNA sequences comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 138.

TABLE 13
A05189: Stuffer-scAAV-2x mU7prom-Z45-8/9-mU7term_mU1prom-Z45-8/9-
mU1term; mouseloop with 5′ ISD and eSL; derivative of A04376

| Plasmid | |
|---|---|
| Element | Nucleotide Sequences |
| 5′ITR | ctgcgcgctcgctcgctcactgaggccgcccgggcaaagcccgggcgtcgggcgacctttggtcgcccgg |
| | cctcagtgagcgagcgagcgcgcagagagggagtggggtt (SEQ ID NO: 119) |
| Mouse U7 | taacaacataggagctgtgattggctgttttcagccaatcagcactgActcatttgcatagcctttacaagcggt |
| Promoter | cacaaactcaagaaacgagcggttttaatagtcttttagaatattgtttatcgaaccgaataaggaactgtgctt |
| | tgtgattcacatatcagtggaggggtgtggaaatggcaccttgatctcaccctcatcgaaagtggagttgatgtc |
| | ctTccctggctcgctacagacgcacttccgc (SEQ ID NO: 151) |
| extra nt | aa |
| ISD | ggagt |
| Fusion spacer | CTGGCATCTGTTTTTGAGGATTGCTGAGTTTGCCGCTGCCCAATGC |
| z8/z9 | CATCCTGGAG (SEQ ID NO: 98) |
| SMOPT | AATTTTTGGAGca (SEQ ID NO: 161) |
| Stem loop | ggttttctgacctccgtcggaaaacccct (SEQ ID NO: 146) |
| Mouse U7 | cccaatttcactggtctacaatgaaagcaaaacagttctcttccccgctccccggtgtgtgagaggggctttgat |
| terminator | ccttctctggtttcctaggaaacgcgtatgtg (SEQ ID NO: 124) |
| Mouse U1 | TTGTTCCTCTTAGTGTTAATTCACACTAAAGACTGTGCATCCGACT |
| Promoter | CCTACATTTATGAAAGTAAATGCCTGTTGTTAGAACAAAAAAGGC |
| | TACAGAACAAAAAACAAAGCGAAATACCATCTGCTTTAGGTTCAG |
| | TGTGGTATTTTCCCGCTGACAGGGAGGCGGGTTTTTGGGTACAGG |
| | AAACGAGTCACTATGGAGGCGGTACTATGTAGATGAGAATTCAG |
| | GTGCAAACTGGGAAAAGCAACTGCTTCCAAATATTTGTGATTTTT |
| | ACAGTGTAGTTTTGGAAAAACTCTTAGCCTACCAATTCTTCTAAGT |
| | GTTTTAAAATGTGGGAGCCAGTACACATGAAGTTATAGAGTGTTT |
| | TAATGAGGCTTAAATATTTACCGTAACTATGAAATGCTACGCATA |
| | TCATGCTGTTCAGGCTCCGTGGCCACGCAACTC (SEQ ID NO: 47) |
| ISD | ggagt |
| Fusion spacer | CTGGCATCTGTTTTTGAGGATTGCTGAGTTTGCCGCTGCCCAATGC |
| z8/z9 | CATCCTGGAG (SEQ ID NO: 98) |
| SMOPT | AATTTTTGGAGca (SEQ ID NO: 161) |
| Stem loop | ggttttctgacctccgtcggaaaacccct (SEQ ID NO: 146) |
| Mouse U1 | GTTTACTTGGTTTTAAAAATAGCTTGCACTAGCGATACGGAATAT |
| terminator | GGTTATTAGGTTTGTTAGGCATCATGTCGTGTCTTACTATAGAAAA |
| | ATAACGTAGTGTTCATTTTAGCCTGCCTGTATGTGTTAATTTGTCC |
| | TTATTGCGCATTGTTCTTGTTAAGTCTTCTGTAAGGAGTTGCGGGT |
| | TTCAAACTGTCAGTCTGAGAGCA (SEQ ID NO: 57) |
| 3′ITR | aggaacccctagtgatggagttggccactccctctctgcgcgctcgctcgctcactgaggccgggcgaccaa |
| | aggtcgcccgacgcccgggctttgcccgggcggcctcagtgagcgagcgagcgcgcagctgcctgcagg |
| | (SEQ ID NO: 127) |

A05189 Nucleotide Sequence (whole transgene from ITR to ITR):
(SEQ ID NO: 138)
ctgcgcgctcgctcgctcactgaggccgcccgggcaaagcccgggcgtcgggcgacctttggtcgcccggcctcagt
gagcgagcgagcgcgcagagagggagtggggttCTTCGAAACACCGGTtaacaacataggagctgtgattggctgttt
tcagccaatcagcactgActcatttgcatagcctttacaagcggtcacaaactcaagaaacgagcggttttaatagtcttttagaatattg
tttatcgaaccgaataaggaactgtgctttgtgattcacatatcagtggaggggtgtggaaatggcaccttgatctcaccctcatcgaaag
tggagttgatgtcctTccctggctcgctacagacgcacttccgcaaggagtCTGGCATCTGTTTTTGAGGATTGC
TGAGTTTGCCGCTGCCCAATGCCATCCTGGAGaATTTTTGGAGcaggttttctgacctccgtcg
gaaaacccctcccaatttcactggtctacaatgaaagcaaaacagttctcttccccgctccccggtgtgtgagaggggctttgatccttct
ctggtttcctaggaaacgcgtatgtgTTGTTCCTCTTAGTGTTAATTCACACTAAAGACTGTGCAT
CCGACTCCTACATTTATGAAAGTAAATGCCTGTTGTTAGAACAAAAAAGGCTACA
GAACAAAAAACAAAGCGAAATACCATCTGCTTTAGGTTCAGTGTGGTATTTTCCC
GCTGACAGGGAGGCGGGTTTTTGGGTACAGGAAACGAGTCACTATGGAGGCGGT
ACTATGTAGATGAGAATTCAGGTGCAAACTGGGAAAAGCAACTGCTTCCAAATA
TTTGTGATTTTTACAGTGTAGTTTTGGAAAAACTCTTAGCCTACCAATTCTTCTAA
GTGTTTTAAAATGTGGGAGCCAGTACACATGAAGTTATAGAGTGTTTTAATGAGG
CTTAAATATTTACCGTAACTATGAAATGCTACGCATATCATGCTGTTCAGGCTCC
GTGGCCACGCAACTCggagtCTGGCATCTGTTTTTGAGGATTGCTGAGTTTGCCGCT
GCCCAATGCCATCCTGGAGaATTTTTGGAGcaggttttctgacctccgtcggaaaacccctGTTTACT
TGGTTTTAAAAATAGCTTGCACTAGCGATACGGAATATGGTTATTAGGTTTGTTA
GGCATCATGTCGTGTCTTACTATAGAAAAATAACGTAGTGTTCATTTTAGCCTGC
CTGTATGTGTTAATTTGTCCTTATTGCGCATTGTTCTTGTTAAGTCTTCTGTAAGG
AGTTGCGGGTTTCAAACTGTCAGTCTGAGAGCAGAATTCGATATCTAGATCTCGA
GGTAACCACGTGCGGACCCAACGGCCGCaggaacccctagtgatggagttggccactccctctctgcgcg
ctcgctcgctcactgaggccgggcgaccaaaggtcgcccgacgcccgggctttgcccgggggcctcagtgagcgagcgagcgc
gcagctgcctgcagg.

An exemplary AAV vector of the disclosure is A05178 designed for multi-exon skipping of exons 50 to 53 Comprising snRNA sequences targeting DMD exons 50, 51, 52, and 53. The elements of A05178 are set forth in Table 14. In some aspects a nucleic acid sequence encoding AAV vector A05178 comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 130.

TABLE 14
A05178 multi-exon targeting vector comprising snRNA sequences
targeting exons 50, 51, 52, and 53

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 119) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 151) |
| extra nt | aa |
| ISD | ggagt |
| Exon 53 spacer | CATTCAACTGTTGCCTCCGGTTCTGAAGGTGTCATCC |
| | CACTGATTCTGAATTCTTTCAACTA (SEQ ID NO: 121) |
| SMOPT (mouse) | AATTTTTGGAGca (SEQ ID NO: 161) |
| Stem loop (mouse) | Ggttttctgacctccgtcggaaaacccct (SEQ ID NO: 146) |
| Mouse U7 terminator | Cccaatttcactggtctacaatgaaagcaaaacagttctcttccccgctccccggtgtgt |
| | gagaggggctttgatccttctctggtttcctaggaaacgcgtatgtg (SEQ ID NO: |
| | 156) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 47) |
| ISD | ggagt |
| Exon 51 spacer | GTAACCACAGGTTGTGTCACCAGAGTAACATACCTC |
| (Z42/z38) | CAACATCAAGGAAGATGGCA (SEQ ID NO: 215) |
| SMOPT (human) | AATTTTTGGAGTA (SEQ ID NO: 163) |
| Stem loop (human) | ggctttctggctccttaccggaaagcc (SEQ ID NO: 1) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 57) |
| Human U1 promoter | TAAGGACCAGCTTCTTTGGGAGAGAACAGACGCAGG |
| | GGCGGGAGGGAAAAAGGGAGAGGCAGACGTCACTT |
| | CCCCTTGGCGGCTCTGGCAGCAGATTGGTCGGTTGAG |
| | TGGCAGAAAGGCAGACGGGGACTGGGCAAGGCACT |
| | GTCGGTGACATCACGGACAGGGCGACTTCTATGTAG |
| | ATGAGGCAGCGCAGAGGCTGACGTCTTCGCCACTTG |
| | CTGCTTCACCACGAAGGAGTTCCCGTGCCCTGGGAG |
| | CGGGTTCAGGACCGCTGATCGGAAGTGAGAATCCCA |
| | GCTGTGTGTCAGGGCTGGAAAGGGCTCGGGAGTGCG |
| | CGGGGCAAGTGACCGTGTGTGTAAAGAGTGAGGCGT |
| | ATGAGGCTGTGTCGGGGCAGAGGCacaacgtttc (SEQ ID |
| | NO: 40) |
| ISD | ggagt |
| Exon 52 spacer | TTGCTGGTCTTGTTTTTCCAACTTGGGACGCCTCTGTT |
| | CCAAA (SEQ ID NO: |
| SMOPT (human) | AATTTTTGGAGTA (SEQ ID NO: 163) |
| Stem loop (human) | Ggctttctggctccttaccggaaagcc (SEQ ID NO: 122) |
| Human Ul terminator | ACTTTCTGGAGTTTCAAAAACAGACTGTACGCCAAG |
| | GGTCATATCTTTTTTTGTATTGGTTTGTGTCTTGGTTG |
| | GTGTCT (SEQ ID NO: 123) |
| Human U5 promoter | GCAGCAAGGCCTCCACTTCACCCCCTAAAGGTTGCCC |
| | CAAGAGCACCGTGTGACTGCTAAGGTATTTCCGGAG |
| | TCTAAAGACGATTATTCAGGTCTCATTTGCATACCCA |
| | TAATACACTGCAAACAGTATTTTTTTCGGAAAAACAT |
| | TTATATATTGCTTGACATTTTTAAGTATGAGAATTTT |
| | GCATGCAGAATTTTTTTGTATAAACTTTCTCAGGTAG |
| | TAACCCTTGGGATTAGTAGACACCATCAGTGTACTAG |
| | GAATTGCAGTTACCCGAAAATTGAGTTACAGAAGTA |
| | ACTGGT (SEQ ID NO: 43) |
| ISD | ggagt |
| Exon 50 spacer | GGCTGCTTTGCCCTCCTCAGAGCTCAGATCTT (SEQ ID |
| | NO: 124) |
| SMOPT (mouse) | aATTTTTGGAGca (SEQ ID NO: 161) |
| Stem loop (mouse) | Ggttttctgacctccgtcggaaaacccct (SEQ ID NO: 146) |
| U5 terminator | ATATGTGGTAATCCAACAATAGAAATTATTTTTAAGT |
| | TTGTGTGTTCCTTTTTCTGTTCAATGGTGCTTTTGATA |
| | TTGTT (SEQ ID NO: 125) |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEO ID NO: 127) |

A05178 Nucleotide Sequence (whole transgene from ITR to ITR):
(SEQ ID NO: 130)
ctgcgcgctcgctcgctcactgaggccgcccgggcaaagcccgggcgtcgggcgacctttggtcgcccggcctcagtgagcgagc
gagcgcgcagagagggagtggggttCTTCGAAACACCGGTtaacaacataggagctgtgattggctgttttcagccaat
cagcactgActcatttgcatagcctttacaagcggtcacaaactcaagaaacgagcggttttaatagtcttttagaatattgtttatcgaa
ccgaataaggaactgtgctttgtgattcacatatcagtggaggggtgtggaaatggcaccttgatctcaccctcatcgaaagtggagttga
tgtcctTccctggctcgctacagacgcacttccgcaaggagtCATTCAACTGTTGCCTCCGGTTCTGAAGG
TGTCATCCCACTGATTCTGAATTCTTTCAACTAaATTTTTGGAGcaggttttctgacctccgtcg
gaaaacccctcccaatttcactggtctacaatgaaagcaaaacagttctcttccccgctccccggtgtgtgagaggggctttgatccttct
ctggtttcctaggaaacgcgtatgtgTTGTTCCTCTTAGTGTTAATTCACACTAAAGACTGTGCAT
CCGACTCCTACATTTATGAAAGTAAATGCCTGTTGTTAGAACAAAAAAGGCTACA
GAACAAAAAACAAAGCGAAATACCATCTGCTTTAGGTTCAGTGTGGTATTTTCCC
GCTGACAGGGAGGCGGGTTTTTGGGTACAGGAAACGAGTCACTATGGAGGCGGT
ACTATGTAGATGAGAATTCAGGTGCAAACTGGGAAAAGCAACTGCTTCCAAATA
TTTGTGATTTTTACAGTGTAGTTTTGGAAAAACTCTTAGCCTACCAATTCTTCTAA
GTGTTTTAAAATGTGGGAGCCAGTACACATGAAGTTATAGAGTGTTTTAATGAGG
CTTAAATATTTACCGTAACTATGAAATGCTACGCATATCATGCTGTTCAGGCTCC
GTGGCCACGCAACTCggagtGTAACCACAGGTTGTGTCACCAGAGTAACATACCTC
CAACATCAAGGAAGATGGCAAATTTTTGGAGTAggctttctggctccttaccggaaagccGTTTA
CTTGGTTTTAAAAATAGCTTGCACTAGCGATACGGAATATGGTTATTAGGTTTGT
TAGGCATCATGTCGTGTCTTACTATAGAAAAATAACGTAGTGTTCATTTTAGCCT
GCCTGTATGTGTTAATTTGTCCTTATTGCGCATTGTTCTTGTTAAGTCTTCTGTAA
GGAGTTGCGGGTTTCAAACTGTCAGTCTGAGAGCAGAATTCTAAGGACCAGCTTC
TTTGGGAGAGAACAGACGCAGGGGGGGGAGGGAAAAAGGGAGAGGCAGACGTC
ACTTCCCCTTGGCGGCTCTGGCAGCAGATTGGTCGGTTGAGTGGCAGAAAGGCA
GACGGGGACTGGGCAAGGCACTGTCGGTGACATCACGGACAGGGCGACTTCTAT
GTAGATGAGGCAGCGCAGAGGCTGACGTCTTCGCCACTTGCTGCTTCACCACGA
AGGAGTTCCCGTGCCCTGGGAGCGGGTTCAGGACCGCTGATCGGAAGTGAGAAT
CCCAGCTGTGTGTCAGGGCTGGAAAGGGCTCGGGAGTGCGCGGGGCAAGTGACC
GTGTGTGTAAAGAGTGAGGCGTATGAGGCTGTGTCGGGGCAGAGGCacaacgtttcgga
gtTTGCTGGTCTTGTTTTTCCAACTTGGGACGCCTCTGTTCCAAAAATTTTTGGAGT
AggctttctggctccttaccggaaagccACTTTCTGGAGTTTCAAAAACAGACTGTACGCCAAGG
GTCATATCTTTTTTTGTATTGGTTTGTGTCTTGGTTGGTGTCTGCAGCAAGGCCTC
CACTTCACCCCCTAAAGGTTGCCCCAAGAGCACCGTGTGACTGCTAAGGTATTTC
CGGAGTCTAAAGACGATTATTCAGGTCTCATTTGCATACCCATAATACACTGCAA
ACAGTATTTTTTTCGGAAAAACATTTATATATTGCTTGACATTTTTAAGTATGAGA
ATTTTGCATGCAGAATTTTTTTGTATAAACTTTCTCAGGTAGTAACCCTTGGGATT
AGTAGACACCATCAGTGTACTAGGAATTGCAGTTACCCGAAAATTGAGTTACAG
AAGTAACTGGTggagtGGCTGCTTTGCCCTCCTCAGAGCTCAGATCTTaATTTTTGGA
GcaggttttctgacctccgtcggaaaacccctATATGTGGTAATCCAACAATAGAAATTATTTTTAA
GTTTGTGTGTTCCTTTTTCTGTTCAATGGTGCTTTTGATATTGTTGATATCTAGATC
TCGAGGTAACCACGTGCGGACCCAACGGCCGCaggaacccctagtgatggagttggccactccctctct
gcgcgctcgctcgctcactgaggccgggcgaccaaaggtcgcccgacgcccgggctttgcccgggcggcctcagtgagcgagcg
agcgcgcagctgcctgcagg.

An exemplary AAV vector of the disclosure is A06107 designed for multi-exon skipping of exons 44 and 45 comprising snRNA sequences targeting DMD exons 44 and 45. The elements of A06107 are set forth in Table 15. In some aspects a nucleic acid sequence encoding AAV vector A06107 comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 131.

TABLE 15
A06107 Nucleotide sequences of plasmid elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 119) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 151) |
| extra nt | aa |
| ISD | ggagt |
| Fusion spacer exon 44 | CATTTCTCAACAGATCTGTCAAATCGCctgcgacttacCTT |
| (z4/z6) | AAGATACCATTTGTATTTAGCATGTT (SEQ ID NO: 85) |
| SMOPT | AATTTTTGGAGca (SEQ ID NO: 161) |
| Stem loop | Ggttttctgacctccgtcggaaaacccct (SEQ ID NO: 146) |
| Mouse U7 terminator | Cccaatttcactggtctacaatgaaagcaaaacagttctcttccccgctccccggtgtgt |
| | gagaggggctttgatccttctctggtttcctaggaaacgcgtatgtg (SEQ ID NO: |
| | 156) |
| Mouse Ul Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 47) |
| ISD | ggagt |
| Fusion spacer exon 45 | ccctacCTCTTTTTTCTGTCTGACAGCAGTTTGCCGCTGC |
| (z2/z8) | CCAATGCCATCCTGGAG (SEQ ID NO: 96) |
| SMOPT | AATTTTTGGAGca (SEQ ID NO: 161) |
| Stem loop | ggttttctgacctccgtcggaaaacccct(SEQ ID NO: 146) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 57) |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 127) |

A06107 Nucleotide Sequence (whole transgene from ITR to ITR):
(SEQ ID NO: 131)
ctgcgcgctcgctcgctcactgaggccgcccgggcaaagcccgggcgtcgggcgacctttggtcgcccggcctcagtgagcgagc
gagcgcgcagagagggagtggggttCTTCGAAACACCGGTtaacaacataggagctgtgattggctgttttcagccaat
cagcactgActcatttgcatagcctttacaagcggtcacaaactcaagaaacgagcggttttaatagtcttttagaatattgtttatcga
accgaataaggaactgtgctttgtgattcacatatcagtggaggggtgtggaaatggcaccttgatctcaccctcatcgaaagtggagtt
gatgtcctTccctggctcgctacagacgcacttccgcaaggagtCATTTCTCAACAGATCTGTCAAATCGCctg
cgacttacCTTAAGATACCATTTGTATTTAGCATGTTaATTTTTGGAGcaggttttctgacctccgt
cggaaaacccctcccaatttcactggtctacaatgaaagcaaaacagttctcttccccgctccccggtgtgtgagaggggctttgatcct
tctctggtttcctaggaaacgcgtatgtgTTGTTCCTCTTAGTGTTAATTCACACTAAAGACTGTGC
ATCCGACTCCTACATTTATGAAAGTAAATGCCTGTTGTTAGAACAAAAAAGGCTA
CAGAACAAAAAACAAAGCGAAATACCATCTGCTTTAGGTTCAGTGTGGTATTTTC
CCGCTGACAGGGAGGCGGGTTTTTGGGTACAGGAAACGAGTCACTATGGAGGCG
GTACTATGTAGATGAGAATTCAGGTGCAAACTGGGAAAAGCAACTGCTTCCAAA
TATTTGTGATTTTTACAGTGTAGTTTTGGAAAAACTCTTAGCCTACCAATTCTTCT
AAGTGTTTTAAAATGTGGGAGCCAGTACACATGAAGTTATAGAGTGTTTTAATGA
GGCTTAAATATTTACCGTAACTATGAAATGCTACGCATATCATGCTGTTCAGGCT
CCGTGGCCACGCAACTCggagtccctacCTCTTTTTTCTGTCTGACAGCAGTTTGCCGCT
GCCCAATGCCATCCTGGAGaATTTTTGGAGcaggttttctgacctccgtcggaaaacccctGTTTACT
TGGTTTTAAAAATAGCTTGCACTAGCGATACGGAATATGGTTATTAGGTTTGTTA
GGCATCATGTCGTGTCTTACTATAGAAAAATAACGTAGTGTTCATTTTAGCCTGC
CTGTATGTGTTAATTTGTCCTTATTGCGCATTGTTCTTGTTAAGTCTTCTGTAAGG
AGTTGCGGGTTTCAAACTGTCAGTCTGAGAGCAGAATTCGATATCTAGATCTCGA
GGTAACCACGTGCGGACCCAACGGCCGCaggaacccctagtgatggagttggccactccctctctgcgcg
ctcgctcgctcactgaggccgggcgaccaaaggtcgcccgacgcccgggctttgcccgggcggcctcagtgagcgagcgagcgc
gcagctgcctgcagg.

An exemplary AAV vector of the disclosure is A06108 designed for multi-exon skipping of exons 44 and 45 comprising snRNA sequences targeting DMD exons 44 and 45. The elements of A06108 are set forth in Table 16. In some aspects a nucleic acid sequence encoding AAV vector A06108 comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 132.

TABLE 16
A06108 Nucleotide sequences of plasmid elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 119) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 151) |
| extra nt | aa |
| ISD | ggagt |
| Fusion spacer exon 44 | GTGTCTTTCTGAGAAACTGTTCAGCTTCTGCATTTCTC |
| (z5/z4) | AACAGATCTGTCAAATCGCctgc (SEQ ID NO: 82)5014 |
| SMOPT | AATTTTTGGAGca (SEQ ID NO: 161) |
| Stem loop | Ggttttctgacctccgtcggaaaacccct (SEQ ID NO: 146) |
| Mouse U7 terminator | Cccaatttcactggtctacaatgaaagcaaaacagttctcttccccgctccccggtgtgt |
| | gagaggggctttgatccttctctggtttcctaggaaacgcgtatgtg (SEQ ID NO: |
| | 156) |
| Mouse Ul Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 47) |
| ISD | ggagt |
| Fusion spacer exon 45 | TTTGCAGACCTCCTGCCACCGCAGATTCAGAGTTTGC |
| (z10/z8) | CGCTGCCCAATGCCATCCTGGAG (SEQ ID NO: 99) |
| SMOPT | AATTTTTGGAGca (SEQ ID NO: 161) |
| Stem loop | Ggttttctgacctccgtcggaaaacccct (SEQ ID NO: 146) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 57) |
| 3′ITR | Aggaacccctagtgatggagttggccactccctctctgcgcgctcgctcgctcactgag |
| | gccgggcgaccaaaggtcgcccgacgcccgggctttgcccgggggcctcagtgag |
| | cgagcgagcgcgcagctgcctgcagg (SEQ ID NO: 127) |

A06108 Nucleotide Sequence (whole transgene from ITR to ITR):
(SEQ ID NO: 132)
ctgcgcgctcgctcgctcactgaggccgcccgggcaaagcccgggcgtcgggcgacctttggtcgcccggcctcagtga
gcgagcgagcgcgcagagagggagtggggttCTTCGAAACACCGGTtaacaacataggagctgtgattggctgttttc
agccaatcagcactgActcatttgcatagcctttacaagcggtcacaaactcaagaaacgagcggttttaatagtcttttagaatattgt
ttatcgaaccgaataaggaactgtgctttgtgattcacatatcagtggaggggtgtggaaatggcaccttgatctcaccctcatcgaaag
tggagttgatgtcctTccctggctcgctacagacgcacttccgcaaggagtGTGTCTTTCTGAGAAACTGTTCAG
CTTCTGCATTTCTCAACAGATCTGTCAAATCGCctgcaATTTTTGGAGcaggttttctgacctcc
gtcggaaaacccctcccaatttcactggtctacaatgaaagcaaaacagttctcttccccgctccccggtgtgtgagaggggctttgatc
cttctctggtttcctaggaaacgcgtatgtgTTGTTCCTCTTAGTGTTAATTCACACTAAAGACTGTG
CATCCGACTCCTACATTTATGAAAGTAAATGCCTGTTGTTAGAACAAAAAAGGCT
ACAGAACAAAAAACAAAGCGAAATACCATCTGCTTTAGGTTCAGTGTGGTATTTT
CCCGCTGACAGGGAGGCGGGTTTTTGGGTACAGGAAACGAGTCACTATGGAGGC
GGTACTATGTAGATGAGAATTCAGGTGCAAACTGGGAAAAGCAACTGCTTCCAA
ATATTTGTGATTTTTACAGTGTAGTTTTGGAAAAACTCTTAGCCTACCAATTCTTC
TAAGTGTTTTAAAATGTGGGAGCCAGTACACATGAAGTTATAGAGTGTTTTAATG
AGGCTTAAATATTTACCGTAACTATGAAATGCTACGCATATCATGCTGTTCAGGC
TCCGTGGCCACGCAACTCggagtTTTGCAGACCTCCTGCCACCGCAGATTCAGAGTT
TGCCGCTGCCCAATGCCATCCTGGAGaATTTTTGGAGcaggttttctgacctccgtcggaaaacccct
GTTTACTTGGTTTTAAAAATAGCTTGCACTAGCGATACGGAATATGGTTATTAGG
TTTGTTAGGCATCATGTCGTGTCTTACTATAGAAAAATAACGTAGTGTTCATTTTA
GCCTGCCTGTATGTGTTAATTTGTCCTTATTGCGCATTGTTCTTGTTAAGTCTTCTG
TAAGGAGTTGCGGGTTTCAAACTGTCAGTCTGAGAGCAGAATTCGATATCTAGAT
CTCGAGGTAACCACGTGCGGACCCAACGGCCGCaggaacccctagtgatggagttggccactccctct
ctgcgcgctcgctcgctcactgaggccgggcgaccaaaggtcgcccgacgcccgggctttgcccgggcggcctcagtgagcgag
cgagcgcgcagctgcctgcagg

### Promoter Sequences

Gene therapy and RNA-targeting snRNA gene therapy compositions of the disclosure comprise promoter sequences derived from an snRNA.

A “promoter” is a regulatory sequence that is a region of a polynucleotide sequence at which initiation and rate of transcription are controlled. It may contain genetic elements at which regulatory proteins and molecules may bind such as RNA polymerase and other transcription factors.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, the snRNA constructs disclosed herein comprise bidirectional snRNA promoters to express snRNAs.

In another embodiment, the vector configurations can comprise linker(s), signal sequence(s), and/or tag(s).

In some embodiments, the vector is a viral vector. In some embodiments, the vector is an adenoviral vector, an adeno-associated viral (AAV) vector, or a lentiviral vector.

### Nucleic Acids

An NOI (nucleotide sequence of interest) includes, without limitation, any nucleotide sequence or transgene capable of being delivered by a vector. NOIs can be synthetic, derived from naturally occurring DNA or RNA, codon optimized, recombinant RNA/DNA, cDNA, partial genomic DNA, and/or combinations thereof. The NOI can be a coding region or partial coding region but need not be a coding region. An NOI can be RNA/DNA in a sense or anti-sense orientation. An NOI can be an snRNA. NOIs are also referred herein, without limitation, as transgenes, heterologous sequences, genes, therapeutic genes. An NOI may also encode an RNA (ribonucleoprotein complex) a POI (protein of interest), a partial POI, a mutated version or variant of a POI. A POI may be analogous to or correspond to a wild-type protein. A POI may also be a fusion protein or ribonucleoprotein complex such as an snRNP. In some aspects RNA sequences disclosed herein may be represented as DNA sequences and it is within the ability of the skilled artisan to derive the sequence of an RNA sequence from a DNA sequence. For example, spacer sequences of the disclosure can represent uracil bases as either a U or T. The skilled artisan would readily understand that an RNA sequence can interchangeably use a T or U to indicate a uracil.

### Codon Optimization

In some embodiments, NOIs or transgenes or GOIs such as nucleic acid sequences of the disclosure are codon optimized nucleic acid sequences. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased transcription or translation in a human subject relative to a wild-type or non-codon optimized nucleic acid sequence.

In some aspects a codon optimized nucleic acid sequence exhibits increased stability. In some aspects a codon optimized nucleic acid sequence exhibits increased stability through increased resistance to hydrolysis. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some aspects a codon optimized nucleic acid sequence can comprise no donor splice sites. In some aspects a codon optimized nucleic acid sequence can comprise no more than about one, or about two, or about three, or about four, or about five, or about six, or about seven, or about eight, or about nine, or about ten donor splice sites. In some aspects a codon optimized nucleic acid sequence comprises at least one, or at least two, or at least three, or at least four, or at least five, or at least six, or at least seven, or at least eight, or at least nine, or at least ten fewer donor splice sites as compared to a non-codon optimized nucleic acid sequence.

Without wishing to be bound by theory, the removal of donor splice sites in the codon optimized nucleic acid sequence can unexpectedly and unpredictably increase expression of protein of interest in vivo, as cryptic splicing is prevented. Moreover, cryptic splicing may vary between different subjects, meaning that the expression level of a protein comprising donor splice sites may unpredictably vary between different subjects. Such unpredictability is unacceptable in the context of human therapy. Accordingly, the codon optimized nucleic acid sequences which lacks donor splice sites, unexpectedly and surprisingly allows for increased expression of the protein in human subjects and regularizes expression of the protein across different human subjects.

In some aspects a codon optimized nucleic acid sequence can have a GC content that differs from the GC content of the non-codon optimized nucleic acid sequence. In some aspects the GC content of a codon optimized nucleic acid sequence is more evenly distributed across the entire nucleic acid sequence, as compared to the non-codon optimized nucleic acid sequence.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects a codon optimized nucleic acid sequence can have fewer repressive microRNA target binding sites as compared to the non-codon optimized nucleic acid sequence. In some aspects, a codon optimized nucleic acid sequence can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence.

Without wishing to be bound by theory, by having fewer repressive microRNA target binding sites, the codon optimized nucleic acid sequence unexpectedly exhibits increased expression in a human subject.

Provided herein are the nucleic acid sequences encoding the gene therapy compositions for use in gene transfer and expression techniques described herein. It should be understood, although not always explicitly stated that the sequences provided herein can be used to provide the expression product as well as substantially identical sequences that encode an RNA or express and produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The NOIs or nucleic acid sequences (e.g., polynucleotide sequences) disclosed herein may be codon-optimized which is a technique well known in the art. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences can be generated. In some embodiments, such a sequence is optimized for expression in a host or target cell, such as a host cell used to express the snRNA or a cell in which the disclosed methods are practiced (such as in a mammalian cell, e.g., a human cell). Codon preferences and codon usage tables for a particular species can be used to engineer isolated nucleic acid molecules encoding an snRNA that takes advantage of the codon usage preferences of that particular species. For example, the snRNA disclosed herein can be designed to have codons that are preferentially used by a particular organism of interest. In one example, a nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule (which can be part of a vector) includes at least one coding sequence that is codon optimized for expression in a eukaryotic cell, or at least one coding sequence codon optimized for expression in a human cell. In one embodiment, such a codon optimized coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes snRNA having at least 85%, at least 90%, at least 92%, at least 95%, at least 96%, at least 97%, at least 98%, at least 99%, or 100% sequence identity to its corresponding wild-type or originating sequence. In another embodiment, a variety of clones containing functionally equivalent nucleic acids may be routinely generated, such as nucleic acids which differ in sequence but which encode the same sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2× SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell. In one embodiment, a cell or cells of a patient treated with compositions disclosed herein include, without limitation, skeletal muscle (developing and mature muscle fibers and satellite cells), neuromuscular junction, cardiomyocytes, smooth muscle cells, peripheral nervous system (neurons), peripheral motor neurons, and/or sensory neurons.

In some embodiments of the compositions and methods of the disclosure, a somatic cell of the disclosure is a neuronal cell. In one embodiment, a cell or cells of a patient treated with compositions disclosed herein include, without limitation, central nervous system (neurons), peripheral nervous system (neurons), peripheral motor neurons, and/or sensory neurons. In one embodiment, a neuronal cell is a glial cell.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the disclosure, a somatic cell is an ocular cell. An ocular cell includes, without limitation, corneal epithelial cells, keratyocytes, retinal pigment epithelial (RPE) cells, lens epithelial cells, iris pigment epithelial cells, conjunctival fibroblasts, non-pigmented ciliary epithelial cells, trabecular meshwork cells, ocular choroid fibroblasts, conjunctival epithelial cells. In some embodiments, an ocular cell is a retinal cell or a corneal cell. In one embodiment, a retinal cell is a photoreceptor cell or a retinal pigment epithelial cell. In another embodiment, a retinal cell is a ganglion cell, an amacrine cell, a bipolar cell, a horizontal cell, a Müller glial cell, a rod cell, or a cone cell. In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods of Use

The disclosure provides a method of encoding an RNA or expressing an NOI in a cell using the snRNA systems disclosed herein. In one embodiment, the disclosure provides a method of modifying an RNA or the activity of a protein encoded by an RNA molecule comprising contacting the composition of the disclosure and the target RNA molecule under conditions suitable for binding to the RNA molecule.

The disclosure provides a method of modifying the level of expression of an RNA molecule of the disclosure of a protein encoded by the RNA molecule comprising contacting the composition of the disclosure and a cell comprising the RNA molecule under conditions suitable for binding to the RNA molecule. In some embodiments, the cell is in vivo, in vitro, ex vivo or in situ. In some embodiments, the composition of the disclosure comprises a vector comprising snRNA sequences. In some embodiments, the vector is an AAV.

The disclosure provides a method of modifying the level of expression of an RNA molecule of the disclosure or a protein encoded by the RNA molecule comprising contacting a composition of the disclosure and the RNA molecule under conditions suitable for knocking down, blocking, splicing, multi-targeting, restore frame, or editing the target RNA. In some embodiments, the composition of the disclosure comprises a vector comprising snRNA sequences. In some embodiments, the vector is an AAV.

The disclosure provides a method of modifying a target RNA or an activity of a protein encoded by a target RNA molecule comprising contacting a composition and a cell comprising the RNA molecule under conditions suitable knocking down, blocking, splicing, multi-targeting, restore frame, or editing the target RNA. In some embodiments, the cell is in vivo, in vitro, ex vivo or in situ. In some embodiments, the composition comprises a vector comprising the snRNA sequences disclosed herein. In some embodiments, the vector is an AAV.

The disclosure provides a method of treating a disease or disorder comprising administering to a subject a therapeutically effective amount of an snRNA composition of the disclosure.

The disclosure provides a method of treating a disease in a patient in need of such treatment comprising administering to the patient a therapeutically effective amount of an snRNA composition of the disclosure, wherein the composition comprises a vector comprising snRNA sequences disclosed herein, wherein the composition modifies, reduces, destroys, knocks down or ablates a level of expression of a targeted RNA (compared to the level of expression of a targeted RNA treated with a non-targeting (NT) control or compared to no treatment). In another embodiment, the level of reduction is 1-fold or greater. In another embodiment, the level of reduction is 2-fold, 3-fold, 4-fold, 5-fold, 6-fold, 7-fold, 8-fold, 9-fold or 10-fold. In another embodiment, the level of reduction is 10-fold or greater. In another embodiment, the level of reduction is between 10-fold and 20-fold. In another embodiment, the level of reduction is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 20%-100% destruction of the targeted RNA. In one embodiment, the % elimination of the targeted RNA is any of 20-99%, 25%-99%, 50%-99%, 80%-99%, 90%-99%, 95%-99%. In one embodiment, the % elimination is 90%, 91%, 92%, 93%, 94%, 95%, 96%, 97%, 98%, or 99%. In another embodiment, % elimination is complete elimination or 100% elimination of the targeted RNA.

The disclosure provides a method of treating a disease or disorder in a subject comprising administering an RNA-targeting nucleic acid molecule (i.e. an snRNA of the disclosure) or an AAV vector comprising an snRNA of the disclosure.

In some aspects the disease or disorder is Duchenne Muscular Dystrophy. In some aspects the RNA-targeting nucleic acid molecule or AAV vector targets an RNA sequence encoding dystrophin (DMD). In some aspects the RNA sequence encoding DMD comprises an intronic or exonic sequence. In some aspects the exonic sequence comprises exons 2, 44, 45, 51, 53, combinations thereof or a flanking region thereof, of DMD.

In some embodiments of the methods of the disclosure, a subject of the disclosure has been diagnosed with a disease to be treated. In some embodiments, the subject of the disclosure presents at least one sign or symptom of a disorder or disease to be treated. In some embodiments, the subject of the disclosure presents at least one sign or symptom of a disease.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure a subject of the disclosure is a mammal. In some embodiments, a subject of the disclosure is a non-human mammal.

In some embodiments of the methods of the disclosure a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject via intracerebral administration. In some embodiments, the composition of the disclosure is administered to the subject by an intrastriatal route. In some embodiments, the composition of the disclosure is administered to the subject by a stereotaxic injection or an infusion. In some embodiments, the composition is administered to skeletal muscle. In some embodiments, the composition is administered to the brain. In some embodiments of the methods of the disclosure a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intraparenchymal, intrathecal, intrastriatal, subcutaneous, intradermal, intraperitoneal, intratumoral, intravenous, intraocular, and/or parenteral administration. In certain embodiments, the compositions of the present disclosure are formulated for intracerebral or intrastriatal administration.

### EXAMPLES

### Example 1: AAV9 snRNA-Mediated Exon 53 Skipping In Vitro and In Vivo

### Materials and Methods

### Transfection of Myotubes:

Patient myoblasts were differentiated to myotubes for 4-5 days. Differentiated cells were transfected with synthetic U7 snRNAs (IDT) using Lipofectamine 2000 (Invitrogen) at a final dose of 25 nM, 50 nM or 100 nM. Cells were harvested 24 h post-transfection in 1 ml TRIzol reagent (Invitrogen). RNA was extracted with ZYMO Direct-zol RNA microprep kit (Zymo Research).

### Transduction of Myotubes:

Patient myoblasts were differentiated to myotubes for 3 days. On day 3 post-differentiation, cells were transduced with AAV9 virus at indicated MOIs. Media was supplemented 24 h post-transduction and replaced 48 h post-transduction. Cells were harvested 7 days post-transduction in 1 ml TRIzol reagent (Invitrogen). RNA was extracted with ZYMO Direct-zol RNA microprep kit (Zymo Research).

### In Vivo Evaluation of AAV9-A05014 and A05211:

6-week old del52hDMD/mdx mice were retro-orbitally administered 1E12 vg/animal of AAV9-A05014 or AAV9-A05211. Tissue was collected after 4-week survival, homogenized in TRIzol and extracted with ZYMO Direct-zol RNA microprep kit (Zymo Research).

### Splicing Assays:

50-500 ng of RNA was used for cDNA synthesis with qScript U1tra SuperMix (QuantaBio) following manufacturer's recommendations. PCR was performed on cDNA with GoTaq Green Master Mix (Promega). PCR products were run on 4200 TapeStation (Agilent) with D1000 ScreenTape (Agilent).

### Immunofluorescence:

Following 7-day treatment, cells were fixed with PFA and stained with primary antibody (mouse anti-dystrophin MANDYS106 MABT827 1:100) diluted in 1% BSA with 0.1% Triton-X 100 and incubated overnight at 4° C. Next day, cells were washed 3×5 min with PBS then incubated for 1 h at RT in the dark with secondary goat anti-mouse Alexa-Fluor 647 (1:1000) in same carrier solution as primary antibody. Cells were washed 3× with PBS then mounted with Prolong Gold Antifade solution with DAPI. After curing, slides were imaged with 20× object on an upright fluorescent microscope.

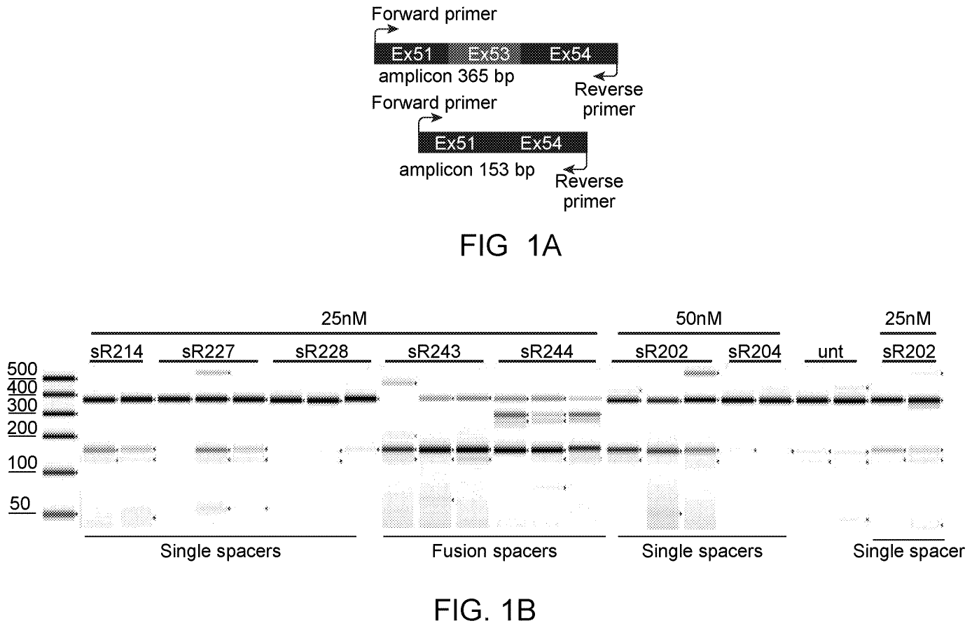

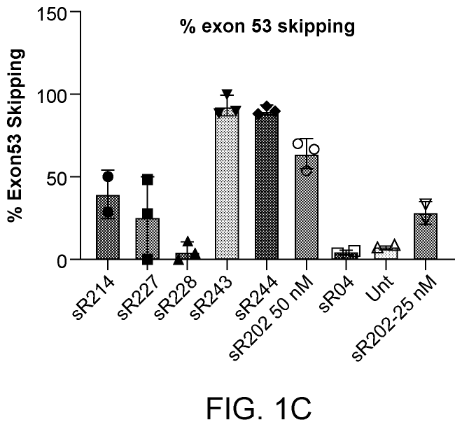

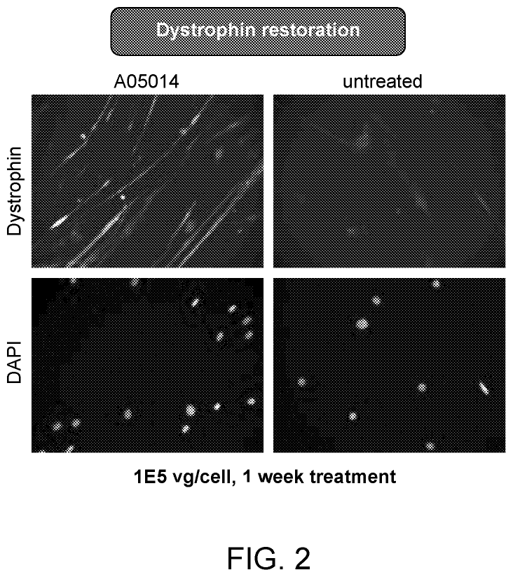

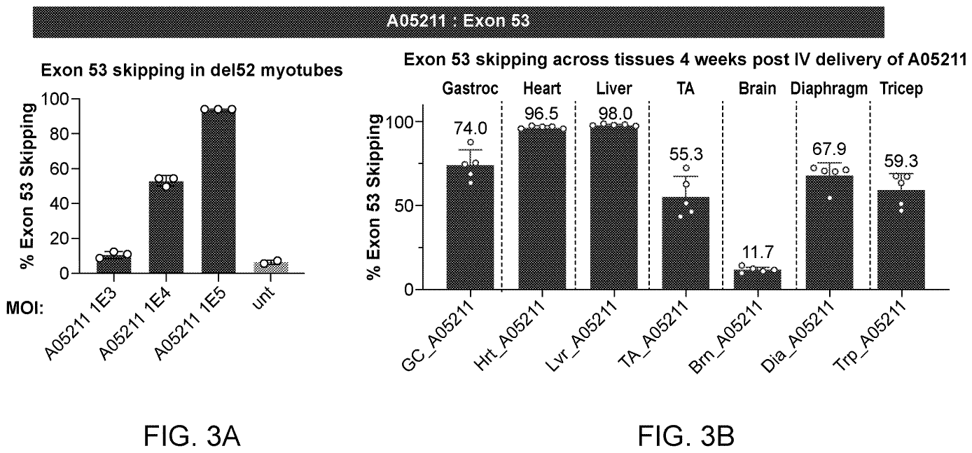

See FIGS. 1-3.

Summary: Synthetic snRNAs were screened in patient myotubes for exon 53 skipping. Top selected fusion spacers were cloned into AAV vectors, as 2× snRNA cassettes and packaged into scAAV9. Treatment of patient myotubes with scAAV9 A05014 results in dystrophin restoration by IF 7 days post transduction. Treatment of del52hDMD/mdx mice with scAAV-A05211 leads to robust exon 53 skipping throughout multiple skeletal muscle tissues 4-weeks post IV delivery (see FIGS. 1 to 3).

### Example 2: AAV9 snRNA-Mediated Exon 44 Skipping in Deletion 45 Patient Myotubes

### Materials and Methods

### Transfection of Myotubes:

Patient myoblasts were differentiated into myotubes for 4-5 days. Differentiated cells were transfected with synthetic U7 snRNAs (IDT) using Lipofectamine 2000 (Invitrogen) at a final dose of 25 nM. Cells were harvested 24 h post-transfection in 1 ml TRIzol reagent (Invitrogen). RNA was extracted with ZYMO Direct-zol RNA microprep kit (Zymo Research).

### Transduction of Myotubes:

Patient myoblasts were differentiated for 3 days. On day 3 post-differentiation, cells were transduced with virus at indicated MOIs. Media was supplemented 24 h post-transduction and replaced 48 h post-transduction. Cells were harvested 7 days post-transduction in 1 ml TRIzol reagent (Invitrogen). RNA was extracted with ZYMO Direct-zol RNA microprep kit (Zymo Research).

### Splicing Assays:

50-500 ng of RNA was used for cDNA synthesis with qScript U1tra SuperMix (QuantaBio) following manufacturer's recommendations. PCR was performed on cDNA with GoTaq Green Master Mix (Promega). PCR products were run on 4200 TapeStation (Agilent) with D1000 ScreenTape (Agilent).

### Protein Analysis:

Myotube protein lysate was run on Jess Simple Western (ProteinSimple) following manufacturer's instructions.

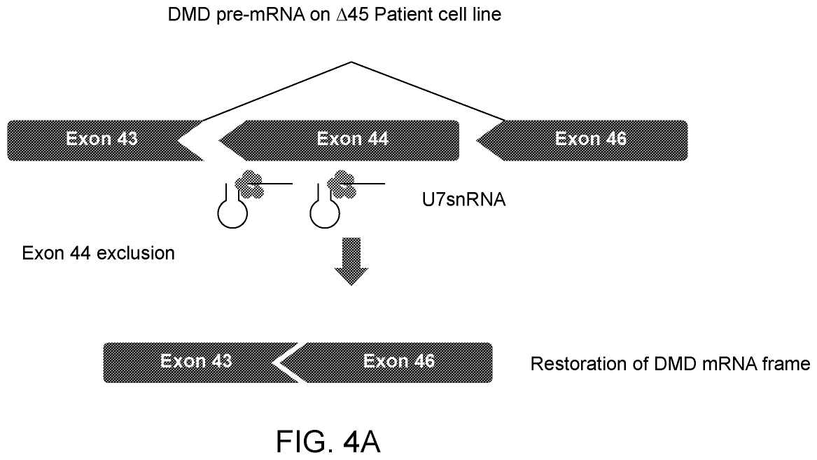

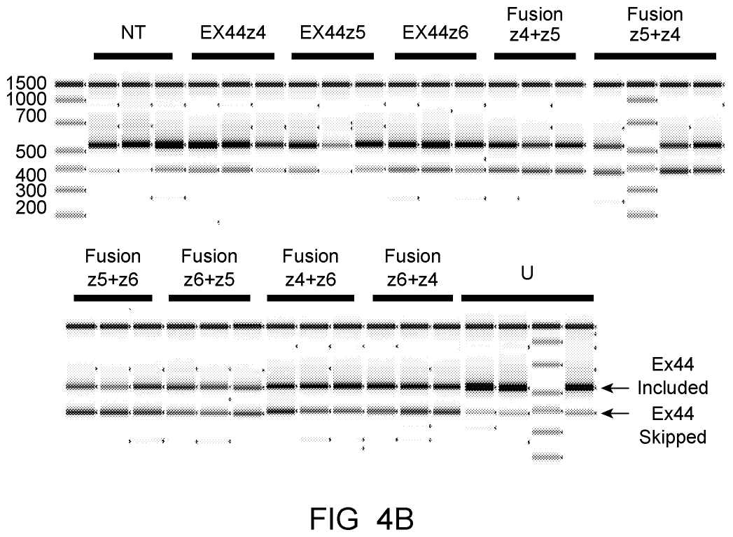

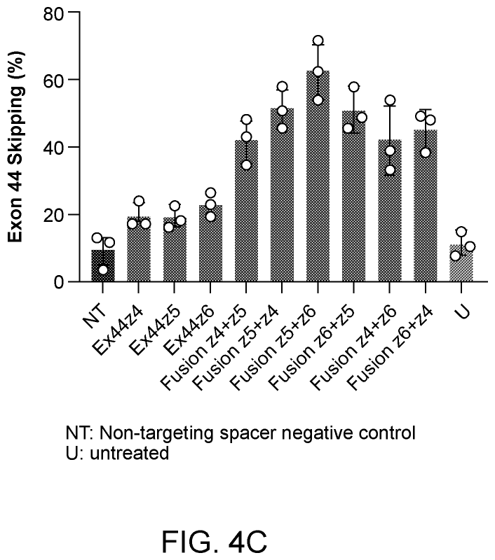

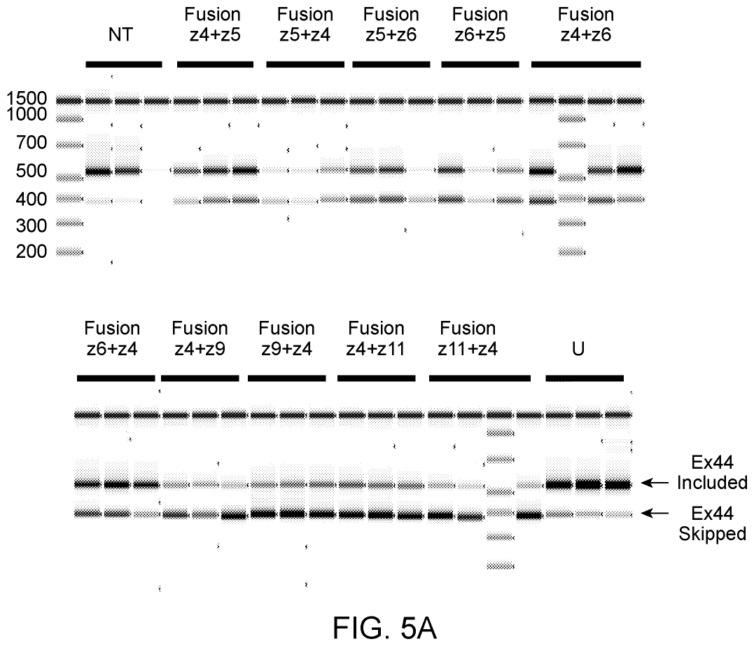

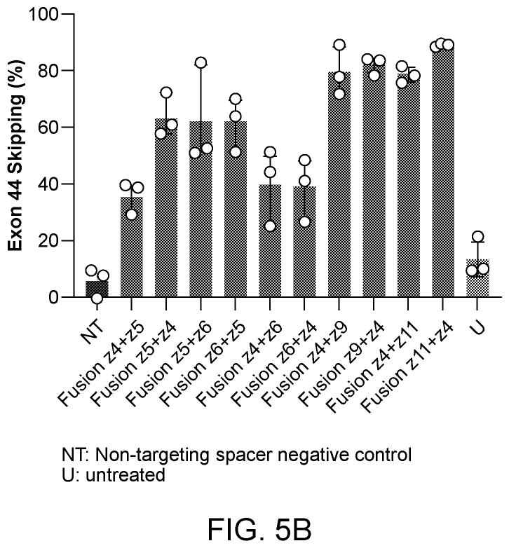

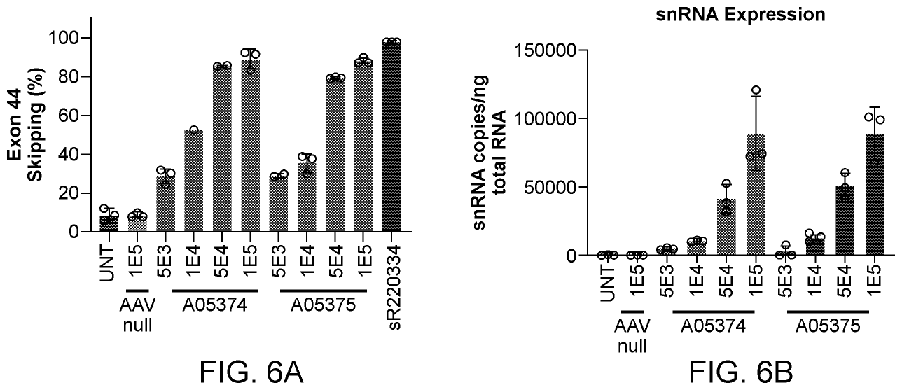

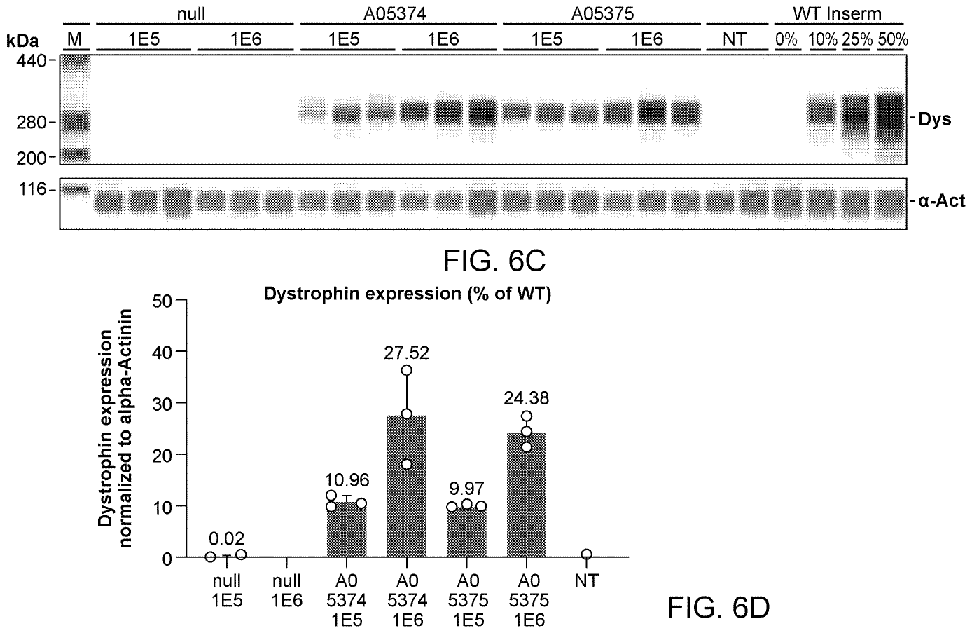

Summary: Synthetic snRNAs were screened in patient myotubes for exon 44 skipping. Top selected fusion spacers were cloned into AAV vectors, as 2× snRNA cassettes and packaged into scAAV9. scAAV9 A05374 and A05375 show dose-dependent exon 44 skipping and dystrophin restoration in del45 myotubes 7 days post transduction (see FIGS. 4 to 6).

### Example 3: AAV9 snRNA-Mediated Exon 45 Skipping in Deletion 44 Patient Myotubes

### Materials and Methods

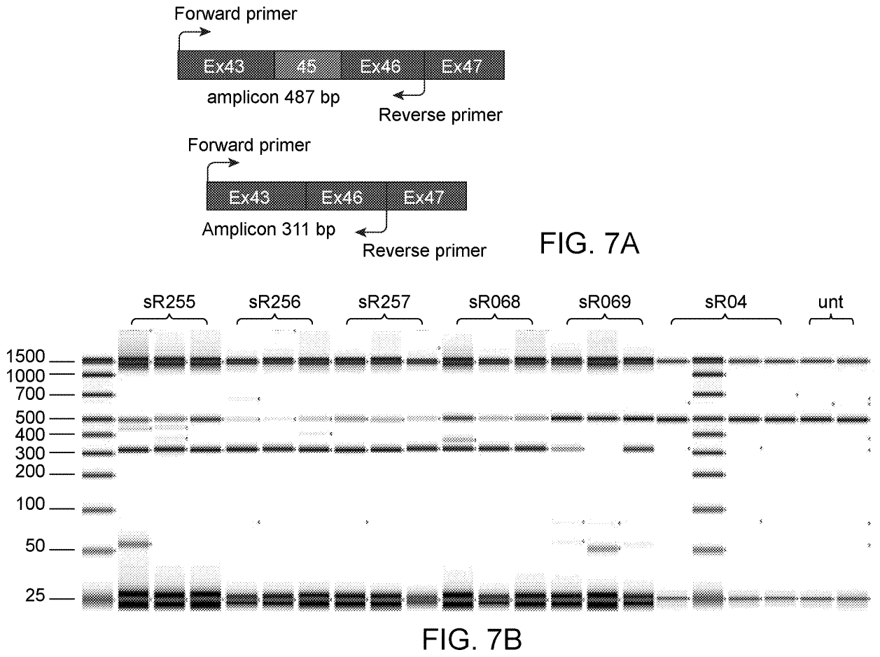

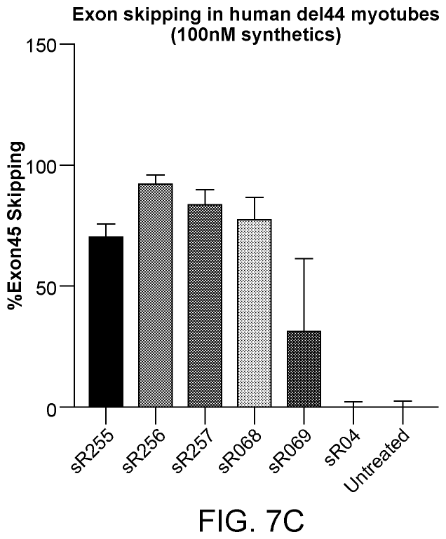

U7 snRNAs were engineered to bind splicing regulatory sequences (SA, splice acceptor; SD splice donor; and/or ESE, exon splice enhancer sequences) to promote skipping of exon 45 which will lead to restoration of DMD mRNA reading frame in the case of DMD patient A44 mutation, and restoration of dystrophin protein. RT-PCR was carried out to determine product sizes using primers in exon 43 and exons 46/47 following transfection of human myotubes with 100 nM of synthetic snRNA. The predicted size for exon 45 included is 487 bp while exon 45 excluded is 311 bp. See FIGS. 7A-C.

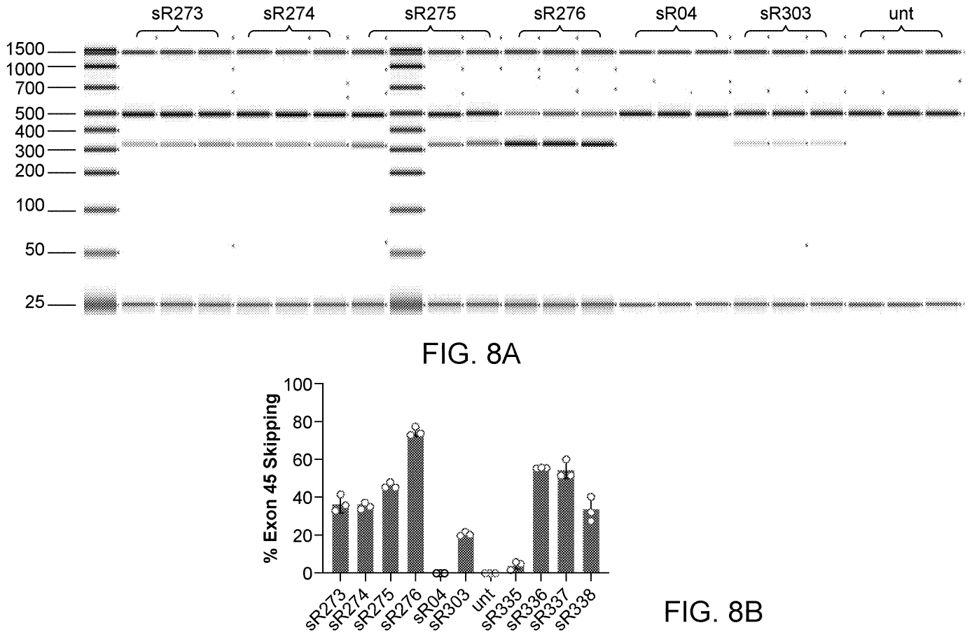

A screening of synthetic snRNAs to identify potent spacer sequences at a lower concentration of synthetic snRNA (25 nM) was performed. RT-PCR was carried out after transfecting U7 snRNA synthetic RNAs. Exon 45 included is predicted to be 487 bp while exon 45 excluded is predicted to be 311 bp. Exon 45 skipping was quantified and an alignment between human (top) and mouse (bottom) exon 45 sequences. Mapping of the targeting sequences contained in the synthetic snRNAs used in the screen was carried out. See FIGS. 8A-8C.

AAV9-mediated exon 45 skipping in human A44 (deletion of exon 44) myotubes was shown. Myotubes were transduced with AAV9 exon 45 targeting constructs and precent skipping was determined 7 days post transduction with varying MOIs of A05189 and A05190. ddPCR was carried out to determine the copies of snRNA per nanogram of total RNA expressed from A05189 and A05190.

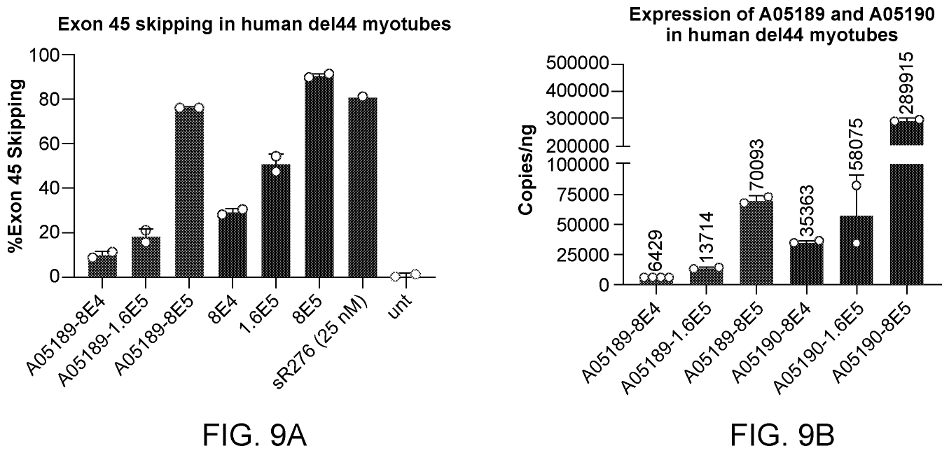

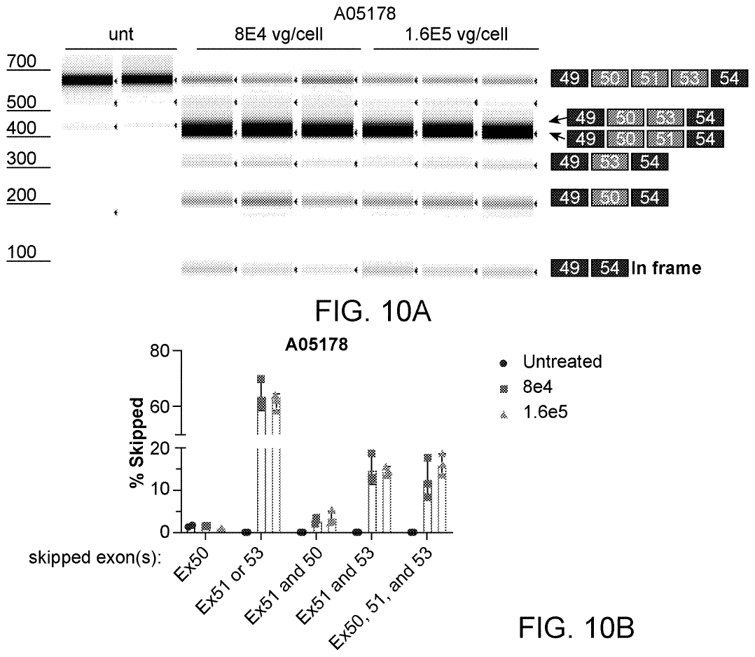

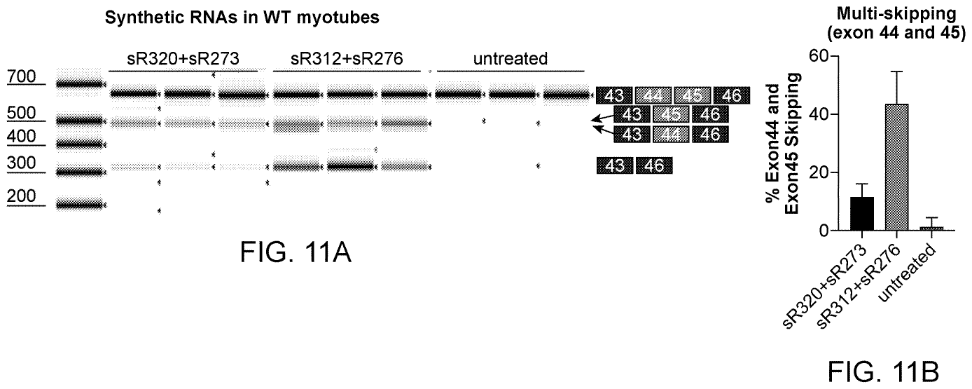

Summary: Synthetic snRNAs were screened in patient myotubes for exon 45 skipping. Top selected fusion spacers were cloned into AAV vectors, as 2× snRNA cassettes and packaged into scAAV9. scAAV9 A05189 and A05190 show dose dependent exon 45 skipping and robust snRNA expression in del44 myotubes 7 days post transduction (see FIGS. 7 to 9).

### Example 4: AAV-Mediated Multi-Exon Skipping of DMD Exons 50-53 in Human Deletion 52 Myotubes

Human del 52 myotubes were transduced with A05178, which carries engineered snRNAs that target exon 50, exon 51, exon 52, and exon 53. After 7 days, RNA was extracted and RT-PCR was performed to identify exon skipping. Exon skipped bands were quantified by dividing the peak molarity of the amplicon (representing single or multi-exon skipping) by the signal for all DMD amplicons.

Summary: Treatment of del52 myotubes with A05178, carrying 4× snRNAs targeting exons 50-53, leads to single and multi-exon skipped DMD 7 days post-transduction.

### Example 5: Multi-Exon Skipping of Exons 44 and 45 Strategy in Human Wild-Type Myotubes

Healthy human (wild-type, WT) myotubes were co-transfection with combinations of synthetic snRNAs targeting exon 44 and exon 45. After 24 hours, RNA as extracted and RT-PCR was performed to identify exon skipping. To determine % multi-skipping, the signal for each of exon 44 and exon 45 skipped band was divided by the signal for all the DMD amplicons.

Summary: Treatment of WT human myotubes with combinations of synthetic snRNAs targeting exon 44 and exon 45 leads to accumulation of the DMD amplicon lacking both exon 44 and exon 45 suggesting that this strategy can be used for multi-exon skipping of exons 44-45.

### CONCLUSIONS

These Examples describe a therapeutic strategy employing engineered U7 snRNAs with unique spacers for targeting exon splicing enhancers within various DMD single and multi-exon strategies. This work is underpinned by our earlier DMD exon 51 (LBIO-115) work. At the high dose, exon 51 skipping was >95% in heart and up to 73% in skeletal muscle following 3 weeks delivery to del52hDMD/mdx mice by intravenous administration. Skipping was sustained across skeletal and heart tissue over 12 weeks, while DMD protein doubles between weeks 4 and 12. Now, herein we have described AAV snRNA constructs comprising novel spacer sequences targeting splicing regulatory regions for exons 53, 44 and 45. These constructs showed comparably high levels of full-length genomes (90%+). Treated patient-derived cells showed robust dose-dependent skipping of the intended exon, and restoration of dystrophin protein expression 7 days post-transduction scAAV9 dual snRNA cassettes. The scAAV9-exon 53 construct was delivered intravenously to del52hDMD/mdx mice, which are also amendable to exon 53 skipping, and demonstrated >95% exon skipping in heart and up to 75% in skeletal muscle following 4 weeks. A total of 35% of DMD patients could be treated with exon skipping approaches for exon 51, exon 53, exon 45 and exon 44, while deployment of this strategy to skip each disease relevant exon can treat up to ~70% of DMD patients.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodimented herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. An RNA-targeting nucleic acid molecule comprising a small nuclear RNA (snRNA), wherein the snRNA comprises a targeting sequence that binds a dystrophin (DMD) RNA sequence, wherein the DMD RNA sequence comprises at least one splicing regulatory sequence selected from: a splice acceptor sequence, a splice donor sequence, and an exon splice enhancer sequence.

2. The RNA-targeting nucleic acid molecule of claim 1, wherein the DMD RNA sequence is at least one of exon 2, exon 44, exon 45, exon 51, and/or exon 53.

3. The RNA-targeting nucleic acid molecule of claim 1, wherein the targeting sequence comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one of SEQ ID NO: 59-118, 126, 206-227 and 237-1344.

4. The RNA-targeting nucleic acid molecule of claim 1, wherein the snRNA comprises a stem loop (SL) comprises a nucleic acid sequence at least 65%, 70%, 75%, 80%, 85%, 90%, 95%, 96%, 97%, 98%, 99% or 100% (or any percentage in between) identical to one or more nucleic acid sequences set forth in any one of SEQ ID NO: 1-SEQ ID NO: 11, SEQ ID NO: 144-SEQ ID NO: 148, SEQ ID NO: 164, SEQ ID NO: 186, SEQ ID NO: 190-SEQ ID NO: 205, SEQ ID NO: 228-SEQ ID NO: 230, SEQ ID NO: 235, or SEQ ID NO: 236.

5. The RNA-targeting nucleic acid molecule of claim 1, wherein the stem loop (eSL) is an engineered stem loop that comprises a nucleic acid sequence at least 65%, 70%, 75%, 80%, 85%, 90%, 95%, 96%, 97%, 98%, 99% or 100% (or any percentage in between) identical to one or more nucleic acid sequences set forth in any one of SEQ ID NO: 1-SEQ ID NO: 11

6. The RNA-targeting nucleic acid molecule of claim 1, wherein DMD RNA sequence is a pre-mRNA or mRNA sequence.

7. The RNA-targeting nucleic acid molecule of claim 1, wherein the snRNA comprises two targeting sequences that target two RNAs of interest.

8. The RNA-targeting nucleic acid molecule of claim 7, wherein the two targeting sequences are a fusion sequence.

9. The RNA-targeting nucleic acid molecule of claim 1, wherein the snRNA comprises an Sm binding domain (SmBD) selected from the group consisting of a U1, U2, U4, and U5 SmBD.

10. The RNA-targeting nucleic acid molecule of claim 9, wherein the SmBD comprises a nucleic acid sequence set forth in any one of SEQ ID NO: 31-SEQ ID NO: 38.

11. The RNA-targeting nucleic acid molecule of claim 1, wherein the snRNA comprises a 5′ interaction stabilizer domain (5′ISD) comprising a nucleotide sequence selected from any one of SEQ ID NO: 12-SEQ ID NO: 23.

12. A vector comprising one or more snRNA of claim 1.

13. The vector of claim 12, wherein the vector is an AAV vector.

14. The AAV vector of claim 13, wherein the snRNA is operably linked to a promoter.

15. The AAV vector of claim 13, wherein the snRNA is operably linked to a U7 promoter or a U1 promoter.

16. The AAV vector of claim 13, wherein the snRNA is operably linked to a downstream terminator (DT).

17. The AAV vector of claim 13, wherein the snRNA is operably linked to a U7 downstream terminator or a U1 downstream terminator.

18. The AAV vector of claim 13, wherein the vector comprises at least one, at least two, at least three, at least four, or at least five snRNA.

19. The AAV vector of claim 18, wherein the least one, at least two, at least three, at least four, or at least five snRNA each target the same target RNA sequences.

20. The AAV vector of claim 18, wherein the least one, at least two, at least three, at least four, or at least five snRNA target two or more target RNA sequences

21. The AAV vector of claim 18, wherein each snRNA is separated by a buffer sequence.

22. The AAV vector of claim 21, wherein the buffer sequence comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one SEQ ID NO: 24-SEQ ID NO: 30.

23. The AAV vector of claim 13, wherein the vector comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one of SEQ ID NO: 130-SEQ ID NO: 138.

24. A method of targeting one or more target RNAs of interest and blocking, knocking down, editing, exon-skipping or splicing the one or more target RNAs, comprising contacting the snRNA of claim 1 with a cell comprising the one or more target RNAs.

25. A DMD RNA-targeting nucleic acid molecule comprising a targeting sequence set forth in any one of SEQ ID NO: 59-118, 206-227 or 237-333.

26. A method of treating a disease or disorder in a subject comprising administering an RNA-targeting nucleic acid molecule of claim 1 or an AAV vector of claim 12.

27. The method of claim 26, wherein the disease or disorder is Duchenne muscular dystrophy.

28. The method of claim 26, wherein the administration is administration is intravenous, intramuscular, subpial, intrathecal, intraparenchymal, intrathecal, intrastriatal, subcutaneous, intradermal, intraperitoneal, intratumoral, intravenous, intraocular, and/or parenteral administration.
